SEQUENCE LISTING (143 sequences)

SEQ ID NO: 1 - protein, 189 aa, Homo sapiens [sequence omitted]

SEQ ID NO: 2 - protein, 188 aa, Homo sapiens
TEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 3 - protein, 188 aa, Homo sapiens [sequence omitted]

SEQ ID NO: 4 - protein, 187 aa, Homo sapiens
TEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 5 - protein, 189 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 6 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 7 - protein, 189 aa, Homo sapiens
MTEYKLVVVGARGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 8 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 9 - protein, 189 aa, Homo sapiens
MTEYKLVVVGAAGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 10 - protein, 189 aa, Homo sapiens
MTEYKLVVVGASGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 11 - protein, 189 aa, Homo sapiens
MTEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 12 - protein, 189 aa, Homo sapiens
MTEYKLVVVGAGGVGKSALTIQLIQNHFVDEYDPTIEDSYRKQVVIDGETCLLDILDTAGHEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 13 - protein, 188 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 14 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 15 - protein, 188 aa, Homo sapiens
MTEYKLVVVGARGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 16 - protein, 188 aa, Homo sapiens [sequence omitted]

SEQ ID NO: 17 - protein, 188 aa, Homo sapiens [sequence omitted]

SEQ ID NO: 18 - protein, 188 aa, Homo sapiens [sequence omitted]

SEQ ID NO: 19 - protein, 188 aa, Homo sapiens [sequence omitted]

SEQ ID NO: 20 - protein, 188 aa, Homo sapiens [sequence omitted]

SEQ ID NO: 21 - protein, 189 aa, Homo sapiens; identical to SEQ ID NO: 5

SEQ ID NO: 22 - protein, 189 aa, Homo sapiens; identical to SEQ ID NO: 6

SEQ ID NO: 23 - protein, 189 aa, Homo sapiens; identical to SEQ ID NO: 7

SEQ ID NO: 24 - protein, 189 aa, Homo sapiens; identical to SEQ ID NO: 8

SEQ ID NO: 25 - protein, 189 aa, Homo sapiens; identical to SEQ ID NO: 9

SEQ ID NO: 26 - protein, 189 aa, Homo sapiens; identical to SEQ ID NO: 10

SEQ ID NO: 27 - protein, 189 aa, Homo sapiens; identical to SEQ ID NO: 11

SEQ ID NO: 28 - protein, 189 aa, Homo sapiens; identical to SEQ ID NO: 12

SEQ ID NO: 29 - protein, 188 aa, Homo sapiens; identical to SEQ ID NO: 13

SEQ ID NO: 30 - protein, 188 aa, Homo sapiens; identical to SEQ ID NO: 14

SEQ ID NO: 31 - protein, 188 aa, Homo sapiens; identical to SEQ ID NO: 15

SEQ ID NO: 32 - protein, 188 aa, Homo sapiens [sequence omitted]

SEQ ID NO: 33 - protein, 188 aa, Homo sapiens [sequence omitted]

SEQ ID NO: 34 - protein, 188 aa, Homo sapiens [sequence omitted]

SEQ ID NO: 35 - protein, 188 aa, Homo sapiens [sequence omitted]

SEQ ID NO: 36 - protein, 188 aa, Homo sapiens [sequence omitted]

SEQ ID NO: 37 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 38 - protein, 1210 aa, Homo sapiens [sequence omitted]

SEQ ID NO: 39 - protein, 5 aa, synthetic construct
NYGVH

SEQ ID NO: 40 - protein, 16 aa, synthetic construct
VIWSGGNTDYNTPFTS

SEQ ID NO: 41 - protein, 11 aa, synthetic construct
ALTYYDYEFAY

SEQ ID NO: 42 - protein, 11 aa, synthetic construct
RASQSIGTNIH

SEQ ID NO: 43 - protein, 7 aa, synthetic construct
YASESIS

SEQ ID NO: 44 - protein, 9 aa, synthetic construct
QQNNNWPTT

SEQ ID NO: 45 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 46 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 47 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 7 aa, synthetic construct
SGDYYWT

SEQ ID NO: 51 - protein, 16 aa, synthetic construct
HIYYSGNTNYNPSLKS

SEQ ID NO: 52 - protein, 9 aa, synthetic construct
DRVTGAFDI

SEQ ID NO: 53 - protein, 11 aa, synthetic construct
QASQDISNYLN

SEQ ID NO: 54 - protein, 7 aa, synthetic construct
DASNLET

SEQ ID NO: 55 - protein, 9 aa, synthetic construct
QHFDHLPLA

SEQ ID NO: 56 - protein, 119 aa, synthetic construct [sequence omitted]

SEQ ID NO: 57 - protein, 107 aa, synthetic construct [sequence omitted]

SEQ ID NO: 58 - protein, 445 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 444 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 60 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 361 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 62 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 63 - protein, 394 aa, Homo sapiens
LSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 64 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 65 - protein, 389 aa, Homo sapiens
LSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 66 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 67 - protein, 470 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 68 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 69 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 70 - protein, 545 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 71 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 72 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 73 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 74 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 75 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKR

SEQ ID NO: 76 - protein, 12 aa, synthetic construct
EPKSCDKTHTCP

SEQ ID NO: 77 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 78 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 106 aa, synthetic construct [sequence omitted]

SEQ ID NO: 80 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 330 aa, synthetic construct [sequence omitted]

SEQ ID NO: 87 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 91 - protein, 12 aa, synthetic construct
ESKYGPPCPSCP

SEQ ID NO: 92 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 93 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 106 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 95 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 216 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 97 - protein, 223 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 222 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 99 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 101 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 103 - protein, 107 aa, synthetic construct [sequence omitted]

SEQ ID NO: 104 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 105 - protein, 4 aa, synthetic construct
GGGS

SEQ ID NO: 106 - protein, 8 aa, synthetic construct
GGGSGGGS

SEQ ID NO: 107 - protein, 12 aa, synthetic construct
GGGSGGGSGGGS

SEQ ID NO: 108 - protein, 16 aa, synthetic construct
GGGSGGGSGGGSGGGS

SEQ ID NO: 109 - protein, 5 aa, synthetic construct
GGGGS

SEQ ID NO: 110 - protein, 10 aa, synthetic construct
GGGGSGGGGS

SEQ ID NO: 111 - protein, 15 aa, synthetic construct
GGGGSGGGGSGGGGS

SEQ ID NO: 112 - protein, 20 aa, synthetic construct
GGGGSGGGGSGGGGSGGGGS

SEQ ID NO: 113 - protein, 8 aa, synthetic construct
GGGGGGGS

SEQ ID NO: 114 - protein, 16 aa, synthetic construct
GGGGGGGSGGGGGGGS

SEQ ID NO: 115 - protein, 24 aa, synthetic construct
GGGGGGGSGGGGGGGSGGGGGGGS

SEQ ID NO: 116 - protein, 32 aa, synthetic construct
GGGGGGGSGGGGGGGSGGGGGGGSGGGGGGGS

SEQ ID NO: 117 - protein, 5 aa, synthetic construct
SGGGG

SEQ ID NO: 118 - protein, 10 aa, synthetic construct
SGGGGSGGGG

SEQ ID NO: 119 - protein, 15 aa, synthetic construct
SGGGGSGGGGSGGGG

SEQ ID NO: 120 - protein, 20 aa, synthetic construct
SGGGGSGGGGSGGGGSGGGG

SEQ ID NO: 121 - protein, 7 aa, synthetic construct
GGGGGGS

SEQ ID NO: 122 - protein, 14 aa, synthetic construct
GGGGGGSGGGGGGS

SEQ ID NO: 123 - protein, 21 aa, synthetic construct
GGGGGGSGGGGGGSGGGGGGS

SEQ ID NO: 124 - protein, 28 aa, synthetic construct
GGGGGGSGGGGGGSGGGGGGSGGGGGGS

SEQ ID NO: 125 - protein, 448 aa, synthetic construct; identical to SEQ ID NO: 48

SEQ ID NO: 126 - protein, 366 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 127 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 366 aa, synthetic construct; identical to SEQ ID NO: 126

SEQ ID NO: 129 - protein, 449 aa, synthetic construct; identical to SEQ ID NO: 47

SEQ ID NO: 130 - protein, 366 aa, synthetic construct; identical to SEQ ID NO: 126

SEQ ID NO: 131 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 366 aa, synthetic construct; identical to SEQ ID NO: 126

SEQ ID NO: 133 - protein, 600 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 134 - protein, 214 aa, synthetic construct; identical to SEQ ID NO: 49

SEQ ID NO: 135 - protein, 600 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 136 - protein, 214 aa, synthetic construct; identical to SEQ ID NO: 49

SEQ ID NO: 137 - protein, 601 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 138 - protein, 214 aa, synthetic construct; identical to SEQ ID NO: 49

SEQ ID NO: 139 - protein, 601 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 140 - protein, 214 aa, synthetic construct; identical to SEQ ID NO: 49

SEQ ID NO: 141 - protein, 376 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 18 aa, synthetic construct
  SITE 7: note = (S)-2-(4-pentenyl)alanine
  SITE 11: note = (S)-2-(4-pentenyl)alanine
  SITE 7..11: note = Residues form a covalent bridge
RRFFGIXLTNXLKTEEGN

SEQ ID NO: 143 - protein, 19 aa, synthetic construct
  SITE 1: note = Acetylated residue
  SITE 5..15: note = Intrachain disulfide bridge
  SITE 19: note = Amidated residue
RRRRCPLYISYDPVCRRRR

(19) United States
(12) Patent Application Publication
(10) Pub. No.: US 2024/0343813 A1
(43) Pub. Date: Oct. 17, 2024

(54) COMBINATION THERAPY AND RELATED METHODS

(71) Applicant: Bicara Therapeutics Inc., Boston, MA (US)

(72) Inventors: Rachel SALAZAR, Wilmington, MA (US); Pradip NAIR, Bangalore (IN); Srinivas Reddy BOREDDY, Bengaluru (IN); Prashant Kumar PANDEY, Sant Akbir Nagar (IN); Prashantha KUMARA M. V, Davanagere District (IN)

(21) Appl. No.: 18/433,814

(22) Filed: Feb. 6, 2024

Related U.S. Application Data
(60) Provisional application No. 63/483,399, filed on Feb. 6, 2023.

(51) Int. Cl. C07K 16/28; A61K 31/519; A61K 39/00; A61K 45/06; A61P 35/00; C07K 16/22

(52) U.S. Cl. CPC C07K 16/2863; A61K 31/519; A61K 45/06; A61P 35/00; C07K 16/22; A61K 2039/505; C07K 2317/31; C07K 2317/565; C07K 2317/76

(57) ABSTRACT

Provided herein are methods comprising the use of KRAS (e.g., hKRAS) inhibitors in combination with an agent(s) that specifically binds to EGFR (e.g., hEGFR) and TGFβ (e.g., hTGFβ), as well as compositions and kits comprising the same. The methods provided herein are useful in methods of treating cancer, (e.g., KRAS (e.g., hKRAS) variant cancers). Further provided herein are combination regimens, combination compositions, pharmaceutical compositions, and kits comprising a KRAS (e.g., hKRAS) inhibitor and an agent(s) that specifically binds to EGFR (e.g., hEGFR) and TGFβ (e.g., hTGFβ).

### RELATED APPLICATIONS

This application claims priority to U.S. Ser. No. 63/483,399, filed Feb. 6, 2023, the entire contents of which is incorporated herein by reference.

### 1. FIELD

This disclosure relates to methods comprising the use of KRAS (e.g., hKRAS) inhibitors in combination with an agent(s) that specifically binds to EGFR (e.g., hEGFR) and TGFβ (e.g., hTGFβ). The disclosure further relates to compositions and kits comprising the same.

### 2. BACKGROUND

KRAS is a small GTPase that regulates diverse cellular processes including e.g., cell proliferation, survival, differentiation, and migration. KRAS cycles through a GTP-bound active state and a GDP-bound inactive state, with the cycling largely regulated by guanine nucleotide exchange factors. Active KRAS mediates its functional effects through downstream signaling pathways, e.g., through the MAPK and PI3K kinases. KRAS variants comprising one or more amino acid modification (e.g., amino acid substitutions (e.g., single amino acid substitutions)) that impair hydrolyzation of GTP to GDP, hence permanently locking KRAS in an active state, have been reported in various cancers, including, e.g., various solid tumors (e.g., pancreatic, lung, colon, rectal, and colorectal cancers). These KRAS variant cancers are typically associated with increased tumorigenesis, aggressive disease, and poor prognosis.

### 3. SUMMARY

Provided herein are, inter alia, methods of utilizing KRAS (e.g., hKRAS) inhibitors in combination with an agent(s) that specifically binds to EGFR (e.g., hEGFR) and TGFβ (e.g., hTGFβ), including e.g., methods of treating cancer, methods of inhibiting the KRAS (e.g., hKRAS) pathway and the TGFβ (e.g., hTGFβ) pathway, methods of restoring sensitivity of a cancer to a KRAS (e.g., hKRAS) inhibitor, and methods of suppressing or preventing resistance of a cancer to a KRAS (e.g., hKRAS) inhibitor, along with e.g., diagnostic methods, combination regimens, combination compositions, pharmaceutical compositions, and kits.

In one aspect, provided herein are methods of treating cancer in a subject in need thereof, the method comprising: administering to the subject (a) a kirsten rat sarcoma virus homolog (KRAS) (e.g., human KRAS (hKRAS)) inhibitor; in combination with (b) a fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)), to thereby treat cancer in the subject in need thereof.

In one aspect, provided herein are methods of treating a KRAS (e.g., hKRAS) variant cancer in a subject in need thereof, the method comprising: (a) receiving test results showing that the KRAS (e.g., hKRAS) variant cancer is resistant to a KRAS inhibitor; in combination with (b) administering to the subject the KRAS (e.g., hKRAS) inhibitor; and a fusion protein comprising: (i) a first moiety that specifically binds EGFR (e.g., hEGFR) operably connected to (ii) a second moiety that specifically binds TGFβ (e.g., hTGFβ), to thereby treat the KRAS (e.g., hKRAS) variant cancer in the subject.

In one aspect, provided herein are methods of treating a KRAS (e.g., hKRAS) variant cancer in a subject in need thereof, the method comprising: (a) administering to the subject a KRAS (e.g., hKRAS) inhibitor; (b) receiving testing results showing that the KRAS (e.g., hKRAS) variant cancer has developed resistance to the KRAS (e.g., hKRAS) inhibitor; and (c) administering to the subject the KRAS (e.g., hKRAS) inhibitor; in combination with a fusion protein comprising: (i) a first moiety that specifically binds EGFR (e.g., hEGFR) operably connected to (ii) a second moiety that specifically binds TGFβ (e.g., hTGFβ), to thereby treat the KRAS (e.g., hKRAS) variant cancer in the subject.

In one aspect, provided herein are methods of treating a KRAS (e.g., hKRAS) variant cancer in a subject in need thereof, the method comprising: (a) determining that the KRAS (e.g., hKRAS) variant cancer is resistant to a KRAS (e.g., hKRAS) inhibitor; and (b) administering to the subject a KRAS (e.g., hKRAS) inhibitor; in combination with a fusion protein comprising: (i) a first moiety that specifically binds EGFR (e.g., hEGFR) operably connected to (ii) a second moiety that specifically binds TGFβ (e.g., hTGFβ), to thereby treat the KRAS (e.g., hKRAS) variant cancer in the subject.

In one aspect, provided herein are methods of delivering a KRAS (e.g., hKRAS) inhibitor and a fusion protein to a subject in need thereof, the method comprising administering (a) a KRAS (e.g., hKRAS) inhibitor; and (b) a fusion protein comprising: (i) a first moiety that specifically binds EGFR) (e.g., hEGFR) operably connected to (ii) a second moiety that specifically binds TGFβ (e.g., hTGFβ), to thereby deliver the KRAS (e.g., hKRAS) inhibitor and the fusion protein to the subject.

In one aspect, provided herein are methods of inhibiting the KRAS (e.g., hKRAS) pathway and the TGFβ (e.g., hTGFβ) pathway in a subject in need thereof, the method comprising administering (a) a KRAS (e.g., hKRAS) inhibitor; in combination with (b) a fusion protein comprising: (i) a first moiety that specifically binds EGFR (e.g., hEGFR) operably connected to (ii) a second moiety that specifically binds TGFβ (e.g., hTGFβ), to thereby inhibit the KRAS (e.g., hKRAS) pathway and the TGFβ (e.g., hTGFβ) pathway in the subject.

In one aspect, provided herein are methods of restoring sensitivity to a KRAS (e.g., hKRAS) inhibitor in a subject in need thereof, the method comprising administering to the subject a fusion protein comprising: (i) a first moiety that specifically binds EGFR (e.g., hEGFR) operably connected to (ii) a second moiety that specifically binds TGFβ (e.g., hTGFβ), to thereby restore sensitivity to a KRAS (e.g., hKRAS) inhibitor in the subject.

In one aspect, provided herein are methods of suppressing or preventing resistance to a KRAS (e.g., hKRAS) inhibitor in a subject in need thereof, the method comprising administering to the subject (a) a KRAS (e.g., hKRAS) inhibitor; in combination with (b) a fusion protein comprising: (i) a first moiety that specifically binds EGFR (e.g., hEGFR) operably connected to (ii) a second moiety that specifically binds TGFβ (e.g., hTGFβ), to thereby suppress or prevent resistance to a KRAS (e.g., hKRAS) inhibitor in the subject.

In one aspect, provided herein are pharmaceutical compositions comprising (a) a KRAS (e.g., hKRAS) inhibitor (e.g., a KRAS (e.g., hKRAS) inhibitor described herein); and (b) a fusion protein comprising: (i) a first moiety that specifically binds EGFR (e.g., hEGFR) operably connected to (ii) a second moiety that specifically binds TGFβ (e.g., hTGFβ) (e.g., a fusion protein described herein); and (c) a pharmaceutically acceptable excipient.

In one aspect, provided herein are kits comprising a (ii) (a) a KRAS (e.g., hKRAS) inhibitor (e.g., a KRAS (e.g., hKRAS) inhibitor described herein), or a pharmaceutical composition thereof; and (b) a fusion protein comprising: (i) a first moiety that specifically binds EGFR (e.g., hEGFR) operably connected to (ii) a second moiety that specifically binds TGFβ (e.g., hTGFβ) (e.g., a fusion protein described herein), or a pharmaceutical composition thereof; (ii) a pharmaceutical composition described herein; (iii) a combination regimen described herein; or (iv) a combination composition described herein.

For the sake of clarity, the following exemplary embodiments are applicable to each of the foregoing aspects as if recited individually after each aspect.

In some embodiments, the KRAS (e.g., hKRAS) inhibitor and the fusion protein are administered in an amount and for a time sufficient to treat or prevent the cancer in the subject.

In some embodiments, the cancer is a KRAS (e.g., hKRAS) variant cancer. In some embodiments, the cancer has been determined to contain a KRAS (e.g., hKRAS) variant. In some embodiments, the KRAS (e.g., hKRAS) variant comprises a KRAS (e.g., hKRAS) activating amino acid modification (e.g., substitution). In some embodiments, the KRAS (e.g., hKRAS) variant comprises an amino acid modification (e.g., substitution) at amino acid position G12, G13, or Q61, numbering relative to the amino acid sequence of SEQ ID NO: 3. In some embodiments, the KRAS (e.g., hKRAS) variant comprises an amino acid modification (e.g., substitution) at amino acid position G12 or G13, numbering relative to the amino acid sequence of SEQ ID NO: 3. In some embodiments, the KRAS (e.g., hKRAS) variant comprises any one or more of the following amino acid substitutions G12C, G12V, G12R, G12D, G12A, G12S, G13D, or Q61H, numbering relative to the amino acid sequence of SEQ ID NO: 3. In some embodiments, the KRAS (e.g., hKRAS) variant comprises any one or more of the following amino acid substitutions G12C, G12V, G12R, G12D, G12A, G12S, or G13D, numbering relative to the amino acid sequence of SEQ ID NO: 3. In some embodiments, the KRAS (e.g., hKRAS) variant comprises any one of the following amino acid substitutions G12C, G12V, G12R, G12A, G12S, or G12D, numbering relative to the amino acid sequence of SEQ ID NO: 3. In some embodiments, the KRAS (e.g., hKRAS) variant comprises a G12C amino acid substitution, numbering relative to the amino acid sequence of SEQ ID NO: 3.

In some embodiments, the cancer is resistant to the KRAS (e.g., hKRAS) inhibitor. In some embodiments, the cancer has been determined to be resistant to the KRAS (e.g., hKRAS) inhibitor.

In some embodiments, the KRAS (e.g., hKRAS) inhibitor has previously been administered to the subject. In some embodiments, the KRAS (e.g., hKRAS) inhibitor has previously been administered to the subject in the absence of the fusion protein.

In some embodiments, the cancer cells overexpress EGFR (e.g., hEGFR) relative to non-cancer cells. In some embodiments, the cancer cells have been determined to overexpress EGFR (e.g., hEGFR) relative to non-cancer cells. In some embodiments, the cancer cells express an EGFR (e.g., hEGFR) variant. In some embodiments, the cancer cells have been determined to express an EGFR (e.g., hEGFR) variant. In some embodiments, the cancer cells overexpress hTGFβ (e.g., hTGFβ) relative to non-cancer cells. In some embodiments, the cancer cells have been determined to overexpress TGFβ (e.g., hTGFβ) relative to non-cancer cells.

In some embodiments, the cancer is local, locally advanced, or metastatic. In some embodiments, the cancer is a carcinoma (e.g., adenocarcinoma, squamous cell carcinoma). In some embodiments, the cancer is a lung cancer (e.g., non-small cell lung cancer (NSCLC), lung adenocarcinoma, lung squamous carcinoma), colorectal cancer (e.g., colorectal adenocarcinoma), pancreatic cancer (e.g., pancreatic ductal adenocarcinoma), breast cancer (e.g., invasive ductal carcinoma), stomach cancer (e.g., stomach adenocarcinoma), endometrial cancer (e.g., undifferentiated endometrial carcinoma), uterine cancer (e.g., uterine endometrial carcinoma), testicular cancer (e.g., testicular germ cell cancer), cervical cancer (e.g., cervical squamous carcinoma), bile duct cancer (e.g., cholangiocarcinoma), a myelodysplastic cancer, or esophageal cancer (e.g., esophageal adenocarcinoma, gastroesophageal junction cancer). In some embodiments, the cancer is a lung cancer (e.g., non-small cell lung cancer (NSCLC), lung adenocarcinoma, lung squamous carcinoma), colorectal cancer (e.g., colorectal adenocarcinoma), or a pancreatic cancer (e.g., pancreatic ductal adenocarcinoma). In some embodiments, the cancer is a non-small cell lung cancer (NSCLC), lung adenocarcinoma, lung squamous carcinoma, colorectal adenocarcinoma, or pancreatic ductal adenocarcinoma.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor.

In some embodiments, the cancer in the subject is resistant to a KRAS (e.g., hKRAS) inhibitor (e.g., the KRAS (e.g., hKRAS) inhibitor).

In some embodiments, the KRAS (e.g., hKRAS) inhibitor is capable of selectively inhibiting a KRAS (e.g., hKRAS) variant. In some embodiments, the KRAS (e.g., hKRAS) variant comprises a KRAS (e.g., hKRAS) activating amino acid modification. In some embodiments, the KRAS (e.g., hKRAS) variant comprises an amino acid modification at amino acid position G12, G13, or Q61, numbering relative to the amino acid sequence of SEQ ID NO: 3. In some embodiments, the KRAS (e.g., hKRAS) variant comprises an amino acid modification at amino acid position G12 or G13, numbering relative to the amino acid sequence of SEQ ID NO: 3. In some embodiments, the KRAS (e.g., hKRAS) variant comprises any one or more of the following amino acid substitutions G12C, G12V, G12R, G12D, G12A, G13D, or Q61H, numbering relative to the amino acid sequence of SEQ ID NO: 3. In some embodiments, the KRAS (e.g., hKRAS) variant comprises any one or more of the following amino acid substitutions G12C, G12V, G12R, G12D, G12A, or G13D, numbering relative to the amino acid sequence of SEQ ID NO: 3. In some embodiments, the KRAS (e.g., hKRAS) variant comprises any one of the following amino acid substitutions G12C, G12V, G12R, G12A, or G12D, numbering relative to the amino acid sequence of SEQ ID NO: 3. In some embodiments, the KRAS (e.g., hKRAS) variant comprises a G12C amino acid substitution, numbering relative to the amino acid sequence of SEQ ID NO: 3.

In some embodiments, the KRAS (e.g., hKRAS) inhibitor comprises or consists of a small molecule, protein (e.g., an antibody or functional fragment or variant thereof), nucleic acid, carbohydrate, a lipid, a metal, or a toxin. In some embodiments, the KRAS (e.g., hKRAS) inhibitor comprises or consists of a small molecule. In some embodiments, the KRAS (e.g., hKRAS) inhibitor comprises a KRAS (e.g., hKRAS) inhibitor in Table 2. In some embodiments, the KRAS (e.g., hKRAS) inhibitor comprises sotorasib (AMG-510).

In some embodiments, the first moiety comprises an antibody, or functional fragment or functional variant thereof. In some embodiments, the first moiety comprises a full-length antibody, a single chain variable fragment (scFv), a Fab, or a single domain antibody (sdAb). In some embodiments, the first moiety comprises a full-length antibody.

In some embodiments, the first moiety comprises a variable heavy chain (VH) region that comprises three complementarity determining regions: VH CDR1, VH CDR2, and VH CDR3; and a variable light chain (VL) region that comprises three complementarity determining regions: VL CDR1, VL CDR2, and VL CDR3.

In some embodiments, (a) the amino acid sequence of VH CDR1 comprises the amino acid sequence SEQ ID NO: 39, or the amino acid sequence of SEQ ID NO: 39 comprising 1, 2, or 3 amino acid modifications; (b) the amino acid sequence of VH CDR2 comprises the amino acid sequence SEQ ID NO: 40, or the amino acid sequence of SEQ ID NO: 40 comprising 1, 2, or 3 amino acid modifications; (c) the amino acid sequence of VH CDR3 comprises the amino acid sequence SEQ ID NO: 41, or the amino acid sequence of SEQ ID NO: 41 comprising 1, 2, or 3 amino acid modifications; and (d) the amino acid sequence of VL CDR1 comprises the amino acid sequence SEQ ID NO: 42, or the amino acid sequence of SEQ ID NO: 42 comprising 1, 2, or 3 amino acid modifications; (e) the amino acid sequence of VL CDR2 comprises the amino acid sequence SEQ ID NO: 43, or the amino acid sequence of SEQ ID NO: 43 comprising 1, 2, or 3 amino acid modifications; and (f) the amino acid sequence of VL CDR3 comprises the amino acid sequence SEQ ID NO: 44, or the amino acid sequence of SEQ ID NO: 44 comprising 1, 2, or 3 amino acid modifications.

In some embodiments, the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46.

In some embodiments, the first moiety comprises a heavy chain (HC) and a light chain (LC), wherein the amino acid sequence of the HC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and the amino acid sequence of the LC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49.

In some embodiments, the second moiety comprises an antibody, or functional fragment or functional variant thereof. In some embodiments, the second moiety comprises a full-length antibody, a single chain variable fragment (scFv), a scFv2, a scFv-Fc, a Fab, a Fab′, a F(ab′)2, or a F(v).

In some embodiments, the second moiety comprises or consists of at least a portion of the extracellular domain (ECD) of a transforming growth factor-beta receptor (TGFβR) (e.g., hTGFβR). In some embodiments, the second moiety comprises or consists of at least a portion of the ECD of transforming growth factor-beta receptor II (TGFβRII) (e.g., hTGFβRII). In some embodiments, the second moiety comprises or consists of the ECD of TGFβRII (e.g., hTGFβRII). In some embodiments, the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.

In some embodiments, the fusion protein comprises (iii) a third moiety that specifically binds TGFβ (e.g., hTGFβ) and is operably connected to the first moiety.

In some embodiments, the third moiety comprises an antibody, or functional fragment or functional variant thereof. In some embodiments, the third moiety comprises a full-length antibody, a single chain variable fragment (scFv), a scFv2, a scFv-Fc, a Fab, a Fab′, a F(ab′)2, or a F(v).

In some embodiments, the third moiety comprises or consists of at least a portion of the extracellular domain (ECD) of a TGFβR (e.g., hTGFβR). In some embodiments, the third moiety comprises or consists of at least a portion of the ECD of TGFβRII (e.g., hTGFβRII). In some embodiments, the third moiety comprises or consists of the ECD of TGFβRII (e.g., hTGFβRII). In some embodiments, the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73. In some embodiments, the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73; and the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.

In some embodiments, the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of the third moiety.

In some embodiments, the first moiety comprises a full-length antibody and wherein the N-terminus of a second moiety is operably connected (e.g., optionally via a linker) to the C-terminus of the first light chain.

In some embodiments, the first moiety comprises a full-length antibody and wherein the N-terminus of a second moiety is operably connected (e.g., optionally via a linker) to the C-terminus of the first heavy chain.

In some embodiments, the first moiety comprises a full-length antibody and wherein the N-terminus of a second moiety is operably connected (e.g., optionally via a linker) to the C-terminus of the first light chain and the N-terminus of the third moiety is operably connected (e.g., optionally via a linker) to the C-terminus of the second light chain.

In some embodiments, the first moiety comprises a full-length antibody and wherein the N-terminus of a second moiety is operably connected (e.g., optionally via a linker) to the C-terminus of the first heavy chain and the N-terminus of the third moiety is operably connected (e.g., optionally via a linker) to the C-terminus of the second heavy chain.

In some embodiments, the first moiety is directly operably connected to the second moiety. In some embodiments, the first moiety is indirectly operably connected to the second moiety. In some embodiments, the first moiety is indirectly operably connected to the second moiety through a first peptide linker. In some embodiments, the first peptide linker comprises or consists of glycine or glycine and serine amino acid residues. In some embodiments, the amino acid sequence of the first peptide linker comprises or consists of (a) the amino acid sequence of any one of SEQ ID NOS: 105-124; or (b) the amino acid sequence of any one of SEQ ID NOS: 105-124 comprising or consisting of 1, 2, or 3 amino acid modifications. In some embodiments, the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.

In some embodiments, the first moiety is directly operably connected to the third moiety. In some embodiments, the first moiety is indirectly operably connected to the third moiety. In some embodiments, the first moiety is indirectly operably connected to the third moiety through a second peptide linker. In some embodiments, the second peptide linker comprises or consists of glycine or glycine and serine amino acid residues. In some embodiments, the amino acid sequence of the second peptide linker comprises or consists of (a) the amino acid sequence of any one of SEQ ID NO: 111; or (b) the amino acid sequence of any one of SEQ ID NO: 111 comprising or consisting of 1, 2, or 3 amino acid modifications. In some embodiments, the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications. In some embodiments, the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of the second peptide linker.

In some embodiments, the amino acid sequence of the first peptide linker is 100% identical to the amino acid sequence of the second peptide linker.

In some embodiments, (a) the first moiety comprises or consists of a full-length antibody; (b) the second moiety comprises a comprises or consists of the ECD of TGFβRII (e.g., hTGFβRII); (c) the third moiety comprises a comprises or consists of the ECD of TGFβRII (e.g., hTGFβRII); (d) the second moiety is operably connected to the first moiety through a first peptide linker; and (c) the third moiety is operably connected to the first moiety through a second peptide linker.

In some embodiments, the N-terminus of the second moiety is operably connected to the C-terminus of the first light chain of the full-length antibody and the N-terminus of the third moiety is operably connected to the C-terminus of the second light chain of the full-length antibody.

In some embodiments, (a) (i) the amino acid sequence of VH CDR1 comprises the amino acid sequence SEQ ID NO: 39, or the amino acid sequence of SEQ ID NO: 39 comprising 1, 2, or 3 amino acid modifications, the amino acid sequence of VH CDR2 comprises the amino acid sequence SEQ ID NO: 40, or the amino acid sequence of SEQ ID NO: 40 comprising 1, 2, or 3 amino acid modifications; the amino acid sequence of VH CDR3 comprises the amino acid sequence SEQ ID NO: 41, or the amino acid sequence of SEQ ID NO: 41 comprising 1, 2, or 3 amino acid modifications; the amino acid sequence of VL CDR1 comprises the amino acid sequence SEQ ID NO: 42, or the amino acid sequence of SEQ ID NO: 42 comprising 1, 2, or 3 amino acid modifications; the amino acid sequence of VL CDR2 comprises the amino acid sequence SEQ ID NO: 43, or the amino acid sequence of SEQ ID NO: 43 comprising 1, 2, or 3 amino acid modifications; and the amino acid sequence of VL CDR3 comprises the amino acid sequence SEQ ID NO: 44, or the amino acid sequence of SEQ ID NO: 44 comprising 1, 2, or 3 amino acid modifications; (ii) the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and/or (iii) the amino acid sequence of the heavy chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and wherein the amino acid sequence of the light chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49; (b) the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73; (c) the amino acid sequence of the third moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73; (d) the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111; and (e) the amino acid sequence of the second peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111.

In some embodiments, the fusion protein comprises: (a) a first polypeptide, wherein the amino acid sequence of the first polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (b) a second polypeptide, wherein the amino acid sequence of the second polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (c) a third polypeptide, wherein the amino acid sequence of the third polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126; and (d) a fourth polypeptide, wherein the amino acid sequence of the fourth polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.

In one aspect, provided herein are methods of treating a KRAS (e.g., hKRAS) variant cancer in a subject in need thereof, the method comprising: (a) receiving test results showing that the KRAS (e.g., hKRAS) variant cancer is resistant to a KRAS inhibitor; in combination with (b) administering to the subject the KRAS (e.g., hKRAS) inhibitor; and a fusion protein comprising: (i) a first moiety that specifically binds EGFR (e.g., hEGFR) operably connected to (ii) a second moiety that specifically binds TGFβ (e.g., hTGFβ), to thereby treat the KRAS (e.g., hKRAS) variant cancer in the subject.

In some embodiments, the KRAS (e.g., hKRAS) inhibitor has been previously administered to the subject.

In some embodiments, the KRAS (e.g., hKRAS) inhibitor and the fusion protein are administered in an amount and for a time sufficient to treat the KRAS (e.g., hKRAS) variant cancer in the subject.

In some embodiments, the cancer is a KRAS (e.g., hKRAS) variant cancer.

In some embodiments, the cancer has been determined to contain a KRAS (e.g., hKRAS) variant.

In some embodiments, the KRAS (e.g., hKRAS) variant comprises a KRAS (e.g., hKRAS) activating amino acid modification (e.g., substitution).

In some embodiments, the KRAS (e.g., hKRAS) variant comprises an amino acid modification (e.g., substitution) at amino acid position G12, G13, or Q61, numbering relative to the amino acid sequence of SEQ ID NO: 3.

In some embodiments, the KRAS (e.g., hKRAS) variant comprises an amino acid modification (e.g., substitution) at amino acid position G12 or G13, numbering relative to the amino acid sequence of SEQ ID NO: 3.

In some embodiments, the KRAS (e.g., hKRAS) variant comprises any one or more of the following amino acid substitutions G12C, G12V, G12R, G12D, G12A, G12S, G13D, or Q61H, numbering relative to the amino acid sequence of SEQ ID NO: 3.

In some embodiments, the KRAS (e.g., hKRAS) variant comprises any one or more of the following amino acid substitutions G12C, G12V, G12R, G12D, G12A, G12S, or G13D, numbering relative to the amino acid sequence of SEQ ID NO: 3.

In some embodiments, the KRAS (e.g., hKRAS) variant comprises any one of the following amino acid substitutions G12C, G12V, G12R, G12A, G12S, or G12D, numbering relative to the amino acid sequence of SEQ ID NO: 3.

In some embodiments, the KRAS (e.g., hKRAS) variant comprises a G12C amino acid substitution, numbering relative to the amino acid sequence of SEQ ID NO: 3.

In some embodiments, the cancer is resistant to the KRAS (e.g., hKRAS) inhibitor. In some embodiments, the cancer has been determined to be resistant to the KRAS (e.g., hKRAS) inhibitor.

In some embodiments, the KRAS (e.g., hKRAS) inhibitor has previously been administered to the subject. In some embodiments, the KRAS (e.g., hKRAS) inhibitor has previously been administered to the subject in the absence of the fusion protein.

In some embodiments, the cancer cells overexpress EGFR (e.g., hEGFR) relative to non-cancer cells. In some embodiments, the cancer cells have been determined to overexpress EGFR (e.g., hEGFR) relative to non-cancer cells. In some embodiments, the cancer cells express an EGFR (e.g., hEGFR) variant. In some embodiments, the cancer cells have been determined to express an EGFR (e.g., hEGFR) variant. In some embodiments, the cancer cells overexpress hTGFβ (e.g., hTGFβ) relative to non-cancer cells. In some embodiments, the cancer cells have been determined to overexpress TGFβ (e.g., hTGFβ) relative to non-cancer cells.

In some embodiments, the cancer is local, locally advanced, or metastatic.

In some embodiments, the cancer is a carcinoma (e.g., adenocarcinoma, squamous cell carcinoma).

In some embodiments, the cancer is a lung cancer (e.g., non-small cell lung cancer (NSCLC), lung adenocarcinoma, lung squamous carcinoma), colorectal cancer (e.g., colorectal adenocarcinoma), pancreatic cancer (e.g., pancreatic ductal adenocarcinoma), breast cancer (e.g., invasive ductal carcinoma), stomach cancer (e.g., stomach adenocarcinoma), endometrial cancer (e.g., undifferentiated endometrial carcinoma), uterine cancer (e.g., uterine endometrial carcinoma), testicular cancer (e.g., testicular germ cell cancer), cervical cancer (e.g., cervical squamous carcinoma), bile duct cancer (e.g., cholangiocarcinoma), a myelodysplastic cancer, or esophageal cancer (e.g., esophageal adenocarcinoma, gastroesophageal junction cancer).

In some embodiments, the cancer is a lung cancer (e.g., non-small cell lung cancer (NSCLC), lung adenocarcinoma, lung squamous carcinoma), colorectal cancer (e.g., colorectal adenocarcinoma), or a pancreatic cancer (e.g., pancreatic ductal adenocarcinoma).

In some embodiments, the cancer is a non-small cell lung cancer (NSCLC), lung adenocarcinoma, lung squamous carcinoma, colorectal adenocarcinoma, or pancreatic ductal adenocarcinoma.

In some embodiments, the subject has been previously administered a KRAS (e.g., hKRAS) inhibitor (e.g., the KRAS (e.g., hKRAS) inhibitor).

In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor.

In some embodiments, the cancer in the subject is resistant to a KRAS (e.g., hKRAS) inhibitor (e.g., the KRAS (e.g., hKRAS) inhibitor).

In some embodiments, the KRAS (e.g., hKRAS) inhibitor is capable of selectively inhibiting a KRAS (e.g., hKRAS) variant.

In some embodiments, the KRAS (e.g., hKRAS) variant comprises a KRAS (e.g., hKRAS) activating amino acid modification. In some embodiments, the KRAS (e.g., hKRAS) variant comprises an amino acid modification at amino acid position G12, G13, or Q61, numbering relative to the amino acid sequence of SEQ ID NO: 3. In some embodiments, the KRAS (e.g., hKRAS) variant comprises an amino acid modification at amino acid position G12 or G13, numbering relative to the amino acid sequence of SEQ ID NO: 3. In some embodiments, the KRAS (e.g., hKRAS) variant comprises any one or more of the following amino acid substitutions G12C, G12V, G12R, G12D, G12A, G13D, or Q61H, numbering relative to the amino acid sequence of SEQ ID NO: 3. In some embodiments, the KRAS (e.g., hKRAS) variant comprises any one or more of the following amino acid substitutions G12C, G12V, G12R, G12D, G12A, or G13D, numbering relative to the amino acid sequence of SEQ ID NO: 3. In some embodiments, the KRAS (e.g., hKRAS) variant comprises any one of the following amino acid substitutions G12C, G12V, G12R, G12A, or G12D, numbering relative to the amino acid sequence of SEQ ID NO: 3. In some embodiments, the KRAS (e.g., hKRAS) variant comprises a G12C amino acid substitution, numbering relative to the amino acid sequence of SEQ ID NO: 3.

In some embodiments, the KRAS (e.g., hKRAS) inhibitor comprises or consists of a small molecule, protein (e.g., an antibody or functional fragment or variant thereof), nucleic acid, carbohydrate, a lipid, a metal, or a toxin. In some embodiments, the KRAS (e.g., hKRAS) inhibitor comprises or consists of a small molecule. In some embodiments, the KRAS (e.g., hKRAS) inhibitor comprises a KRAS (e.g., hKRAS) inhibitor in Table 2. In some embodiments, the KRAS (e.g., hKRAS) inhibitor comprises sotorasib (AMG-510).

In some embodiments, the first moiety comprises an antibody, or functional fragment or functional variant thereof. In some embodiments, the first moiety comprises a full-length antibody, a single chain variable fragment (scFv), a Fab, or a single domain antibody (sdAb). In some embodiments, the first moiety comprises a full-length antibody.

In some embodiments, the first moiety comprises a variable heavy chain (VH) region that comprises three complementarity determining regions: VH CDR1, VH CDR2, and VH CDR3; and a variable light chain (VL) region that comprises three complementarity determining regions: VL CDR1, VL CDR2, and VL CDR3.

In some embodiments, (a) the amino acid sequence of VH CDR1 comprises the amino acid sequence SEQ ID NO: 39, or the amino acid sequence of SEQ ID NO: 39 comprising 1, 2, or 3 amino acid modifications; (b) the amino acid sequence of VH CDR2 comprises the amino acid sequence SEQ ID NO: 40, or the amino acid sequence of SEQ ID NO: 40 comprising 1, 2, or 3 amino acid modifications; (c) the amino acid sequence of VH CDR3 comprises the amino acid sequence SEQ ID NO: 41, or the amino acid sequence of SEQ ID NO: 41 comprising 1, 2, or 3 amino acid modifications; and (d) the amino acid sequence of VL CDR1 comprises the amino acid sequence SEQ ID NO: 42, or the amino acid sequence of SEQ ID NO: 42 comprising 1, 2, or 3 amino acid modifications; (e) the amino acid sequence of VL CDR2 comprises the amino acid sequence SEQ ID NO: 43, or the amino acid sequence of SEQ ID NO: 43 comprising 1, 2, or 3 amino acid modifications; and (f) the amino acid sequence of VL CDR3 comprises the amino acid sequence SEQ ID NO: 44, or the amino acid sequence of SEQ ID NO: 44 comprising 1, 2, or 3 amino acid modifications.

In some embodiments, the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46.

In some embodiments, the first moiety comprises a heavy chain (HC) and a light chain (LC), wherein the amino acid sequence of the HC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and the amino acid sequence of the LC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49.

In some embodiments, the second moiety comprises an antibody, or functional fragment or functional variant thereof.

In some embodiments, the second moiety comprises a full-length antibody, a single chain variable fragment (scFv), a scFv2, a scFv-Fc, a Fab, a Fab′, a F(ab′)2, or a F(v).

In some embodiments, the second moiety comprises or consists of at least a portion of the extracellular domain (ECD) of a transforming growth factor-beta receptor (TGFβR) (e.g., hTGFβR). In some embodiments, the second moiety comprises or consists of at least a portion of the ECD of transforming growth factor-beta receptor II (TGFβRII) (e.g., hTGFβRII). In some embodiments, the second moiety comprises or consists of the ECD of TGFβRII (e.g., hTGFβRII). In some embodiments, the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.

In some embodiments, the fusion protein comprises (iii) a third moiety that specifically binds TGFβ (e.g., hTGFβ) and is operably connected to the first moiety.

In some embodiments, the third moiety comprises an antibody, or functional fragment or functional variant thereof.

In some embodiments, the third moiety comprises a full-length antibody, a single chain variable fragment (scFv), a scFv2, a scFv-Fc, a Fab, a Fab′, a F(ab′)2, or a F(v).

In some embodiments, the third moiety comprises or consists of at least a portion of the extracellular domain (ECD) of a TGFβR (e.g., hTGFβR). In some embodiments, the third moiety comprises or consists of at least a portion of the ECD of TGFβRII (e.g., hTGFβRII). In some embodiments, the third moiety comprises or consists of the ECD of TGFβRII (e.g., hTGFβRII). In some embodiments, the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73. In some embodiments, the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73; and the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.

In some embodiments, the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of the third moiety.

In some embodiments, the first moiety comprises a full-length antibody and wherein the N-terminus of a second moiety is operably connected (e.g., optionally via a linker) to the C-terminus of the first light chain.

In some embodiments, the first moiety comprises a full-length antibody and wherein the N-terminus of a second moiety is operably connected (e.g., optionally via a linker) to the C-terminus of the first heavy chain.

In some embodiments, the first moiety comprises a full-length antibody and wherein the N-terminus of a second moiety is operably connected (e.g., optionally via a linker) to the C-terminus of the first light chain and the N-terminus of the third moiety is operably connected (e.g., optionally via a linker) to the C-terminus of the second light chain.

In some embodiments, the first moiety comprises a full-length antibody and wherein the N-terminus of a second moiety is operably connected (e.g., optionally via a linker) to the C-terminus of the first heavy chain and the N-terminus of the third moiety is operably connected (e.g., optionally via a linker) to the C-terminus of the second heavy chain.

In some embodiments, the first moiety is directly operably connected to the second moiety. In some embodiments, the first moiety is indirectly operably connected to the second moiety. In some embodiments, the first moiety is indirectly operably connected to the second moiety through a first peptide linker. In some embodiments, the first peptide linker comprises or consists of glycine or glycine and serine amino acid residues. In some embodiments, the amino acid sequence of the first peptide linker comprises or consists of (a) the amino acid sequence of any one of SEQ ID NOS: 105-124; or (b) the amino acid sequence of any one of SEQ ID NOS: 105-124 comprising or consisting of 1, 2, or 3 amino acid modifications. In some embodiments, the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.

In some embodiments, the first moiety is directly operably connected to the third moiety.

In some embodiments, the first moiety is indirectly operably connected to the third moiety. In some embodiments, the first moiety is indirectly operably connected to the third moiety through a second peptide linker. In some embodiments, the second peptide linker comprises or consists of glycine or glycine and serine amino acid residues. In some embodiments, the amino acid sequence of the second peptide linker comprises or consists of (a) the amino acid sequence of any one of SEQ ID NO: 111; or (b) the amino acid sequence of any one of SEQ ID NO: 111 comprising or consisting of 1, 2, or 3 amino acid modifications. In some embodiments, the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications. In some embodiments, the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of the second peptide linker.

In some embodiments, the amino acid sequence of the first peptide linker is 100% identical to the amino acid sequence of the second peptide linker.

In some embodiments, (a) the first moiety comprises or consists of a full-length antibody; (b) the second moiety comprises a comprises or consists of the ECD of TGFβRII (e.g., hTGFβRII); (c) the third moiety comprises a comprises or consists of the ECD of TGFβRII (e.g., hTGFβRII); (d) the second moiety is operably connected to the first moiety through a first peptide linker; and (e) the third moiety is operably connected to the first moiety through a second peptide linker.

In some embodiments, the N-terminus of the second moiety is operably connected to the C-terminus of the first light chain of the full-length antibody and the N-terminus of the third moiety is operably connected to the C-terminus of the second light chain of the full-length antibody.

In some embodiments, (a) (i) the amino acid sequence of VH CDR1 comprises the amino acid sequence SEQ ID NO: 39, or the amino acid sequence of SEQ ID NO: 39 comprising 1, 2, or 3 amino acid modifications, the amino acid sequence of VH CDR2 comprises the amino acid sequence SEQ ID NO: 40, or the amino acid sequence of SEQ ID NO: 40 comprising 1, 2, or 3 amino acid modifications; the amino acid sequence of VH CDR3 comprises the amino acid sequence SEQ ID NO: 41, or the amino acid sequence of SEQ ID NO: 41 comprising 1, 2, or 3 amino acid modifications; the amino acid sequence of VL CDR1 comprises the amino acid sequence SEQ ID NO: 42, or the amino acid sequence of SEQ ID NO: 42 comprising 1, 2, or 3 amino acid modifications; the amino acid sequence of VL CDR2 comprises the amino acid sequence SEQ ID NO: 43, or the amino acid sequence of SEQ ID NO: 43 comprising 1, 2, or 3 amino acid modifications; and the amino acid sequence of VL CDR3 comprises the amino acid sequence SEQ ID NO: 44, or the amino acid sequence of SEQ ID NO: 44 comprising 1, 2, or 3 amino acid modifications; (ii) the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46; and/or (iii) the amino acid sequence of the heavy chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and wherein the amino acid sequence of the light chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49; (b) the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (c) the amino acid sequence of the third moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (d) the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111; and (e) the amino acid sequence of the second peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111.

In some embodiments, the fusion protein comprises: (a) a first polypeptide, wherein the amino acid sequence of the first polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (b) a second polypeptide, wherein the amino acid sequence of the second polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (c) a third polypeptide, wherein the amino acid sequence of the third polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126; and (d) a fourth polypeptide, wherein the amino acid sequence of the fourth polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.

### 4. BRIEF DESCRIPTION OF THE FIGURES

FIG. 1A is a line graph showing the relative cytotoxicity of each of the indicated treatments (AMG510+BCA101 (10 μg/mL)); AMG510; and BCA101 (10 μg/mL)) in the H358 in vitro lung cancer model. Synergistic activity was calculated using a Bliss model as indicated in the line graph.

FIG. 1B is a line graph showing the relative cytotoxicity of each of the indicated treatments (AMG510+BCA101 (50 μg/mL)); AMG510; and BCA101 (50 μg/mL)) in the H1792 in vitro lung cancer model. Synergistic activity was calculated using a Bliss model as indicated in the line graph.

FIG. 1C is a line graph showing the relative cytotoxicity of each of the indicated treatments (AMG510+BCA101 (50 μg/mL)); AMG510; and BCA101 (50 μg/mL)) in the SW837 in vitro colorectal cancer model. Synergistic activity was calculated using a Bliss model as indicated in the line graph.

FIG. 1D is a line graph showing the relative cytotoxicity of each of the indicated treatments (AMG510+BCA101 (50 μg/mL)); AMG510; and BCA101 (50 μg/mL)) in the SW1463 in vitro colorectal cancer model. Synergistic activity was calculated using a Bliss model as indicated in the line graph.

FIG. 2 is a bar graph showing the percent viability of H358 lung cancer cells in vitro in each of the indicated treatments (Control; AMG510, BCA101, AMG510+BCA101; Cetixumab; AMG510+Cetuximab; TGFβRIIFC; AMG510+TGFβRIIFC; Cetixumab+TGFβRIIFC; and AMG510+Cetuximab+TGFβRIIFC).

FIG. 3 is a line graph showing the percent viability of each of the indicated in vitro H1792 lung cancer cell cultures in the indicated treatments (H1792 wt′ H1792-TGFB1; H1792-TGFβ1+Cetuximab; and H1792-TGFβ1+BCA101).

FIG. 4 is a bar graph showing the percent viability of each of the indicated in vitro H358 lung cancer cell cultures (H358 wt; H358 AMG510 resistant) in the indicated treatment groups (AMG510 at 0 nM, 1.28 nM, 6.4 nM, 32 nM, or 160 nM).

FIG. 5A is a series of histograms showing the mean fluorescent intensity (MFI) of cell surface expression of EGFR assessed by flow cytometry in the KRASG12C mutated lung and colorectal cancer cell lines: NCI-H1792 (G12C homozygous), NCI-H358 (G12C heterozygous), and SW1463 (G12C homozygous).

FIG. 5B is a bar graph presenting the MFI of cell surface expression of EGFR data assessed by flow cytometry presented in FIG. 5A in the KRASG12C mutated lung and colorectal cancer cell lines: NCI-H1792 (G12C homozygous), NCI-H358 (G12C heterozygous), and SW1463 (G12C homozygous).

FIG. 6 is a bar graph showing the expression level of TGF-β from the KRASG12C mutated lung and colorectal cancer cell lines: NCI-H1792 (G12C homozygous) (lung cancer), NCI-H358 (G12C heterozygous) (lung cancer), and SW1463 (G12C homozygous) (colon cancer) as assessed by ELISA.

FIG. 7 is a bar graph showing the level of TGF-β1 expressed by KRASG12C inhibitor (KRASG12Ci) resistant NCI-H358 (denoted as H358) and NCI-H1792 (denoted as H1792) treated with a KRASi or untreated control (as measured by ELISA).

FIG. 8 is a bar graph showing the fold change in the level of TGF-β1 expressed by SW1463 KRASG12Ci resistant cells/SW1463 parental.

FIG. 9 is a bar graph showing the fold change in the level of cell surface EGFR expressed by H1792 KRASG12Ci resistant cells/parental and SW1463 KRASG12Ci resistant cells/parental. The graph is plotted as fold change of EGFR median fluorescence intensity between KRASG12Ci resistant and parental cells.

FIG. 10 is a line graph showing the percent viability of each of the indicated in vitro H1792 lung cancer cell cultures in the indicated treatment groups (H1792, H1792 KRASG12Ci resistant, H1792 KRASG12Ci resistant+Cetuximab; and H1792 KRASG12Ci resistant+BCA101).

FIG. 11 is a line graph showing the percent viability of each of the indicated in vitro SW1463 colon cancer cell cultures in the indicated treatment groups (SW1463, SW1463 KRASG12Ci resistant; and SW1463 KRASG12Ci resistant+BCA101).

FIG. 12 is a line graph showing mean tumor volume+SEM (mm³) of NCI-H358 xenograft tumors in nude mice treated with in indicated agent(s) (BCA101 alone (5 mg/kg), AMG510 alone (5 mg/kg), and AMG510 (5 mg/kg)+BCA101 (5 mg/kg)) or placebo group. n=10 mice per group. Statistical significance was calculated by two-way ANOVA with Tukey's multiple comparisons test comparing mean tumor volume for each treatment with every other treatment. *p≤0.05, **p≤0.01.

FIG. 13 is an enlarged image of the boxed region of FIG. 12 excluding the placebo group.

### 5. DETAILED DESCRIPTION

KRAS (e.g., hKRAS) inhibitors have been evaluated, and in some instances approved, for the treatment of specific KRAS (e.g., hKRAS) variant cancers. However, a significant number of subjects treated with KRAS (e.g., hKRAS) inhibitors acquire resistance to the KRAS (e.g., hKRAS) inhibitor, eventually limiting the clinical use of the therapy. The inventors have, inter alia, identified that administration of an agent(s) (e.g., fusion protein) that specifically binds EGFR (e.g., hEGFR) and TGFβ, inter alia, synergistically improves the cytotoxicity of KRAS (e.g., hKRAS) inhibitors and rescues TGFβ-induced KRAS (e.g., hKRAS) inhibitor resistance. Accordingly, the methods, pharmaceutical compositions, and kits described herein are good candidates for the treatment of cancer (e.g., KRAS (e.g., hKRAS) variant cancer, EGFR (e.g., hEGFR) expressing cancer). As such, the current disclosure provides, inter alia, novel methods, pharmaceutical compositions, and kits for use, e.g., in the treatment of cancer (e.g., KRAS (e.g., hKRAS) variant cancer, EGFR (e.g., hEGFR) expressing cancer).

Table of Contents

| | |
|---|---|
| 5.1 | Definitions |
| 5.2 | KRAS Inhibitors |
| 5.3 | EGFR Binding Moieties |
| 5.4 | TGFβ Binding Moieties |
| 5.5 | Fusion Proteins & Polypeptides |
| 5.5.1 | Ig Constant Regions |
| 5.5.2 | Linkers |
| 5.5.3 | Orientation |
| 5.5.3.1 | Homodimeric Full-Length Antibody - C-Terminal Light Chain |
| | Fusion Proteins |
| 5.5.3.2 | Homodimeric Full-Length Antibody - C-Terminal Heavy Chain |
| | Fusion Proteins |
| 5.5.4 | Exemplary Fusion Proteins |
| 5.5.5 | Affinity of Fusion Proteins and Antigen Binding Moieties |
| | Thereof |
| 5.6 | Methods of Making Fusion Proteins & Components Thereof |
| 5.7 | Combination Regimens |
| 5.8 | Combination Compositions |
| 5.9 | Polynucleotides, Vectors, Carriers, & Host Cells |
| 5.10 | Pharmaceutical Compositions |
| 5.11 | Methods of Use |
| 5.11.1 | Methods of Delivery |
| 5.11.2 | Methods of Inhibiting the KRAS and TGFβ Pathways |
| 5.11.3 | Methods of Restoring Sensitivity to a KRAS Inhibitor |
| 5.11.4 | Methods of Suppressing or Preventing Resistance to a KRAS |
| | Inhibitor |
| 5.11.5 | Methods of Treating Cancer |
| 5.11.5.1 | Cancers |
| 5.12 | Kits |
| 5.13 | Exemplary Embodiments |
| 5.1 | Definitions |

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided and vice versa.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” should be assumed to be within an acceptable error range for that particular value or composition.

Where proteins and/or polypeptides are described herein, it is understood that polynucleotides (e.g., RNA (e.g., mRNA) or DNA polynucleotides) encoding the protein or polypeptide are also provided herein.

Where proteins, polypeptides, polynucleotides, cells, expression vectors, etc. are described herein, it is understood that isolated forms of the proteins, polypeptides, polynucleotides, cells, expression vectors, etc. are also provided herein.

Where proteins, polypeptides, polynucleotides, etc. are described herein, it is understood that recombinant forms of the proteins, polypeptides, polynucleotides, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

As used herein, the term “administering” refers to the physical introduction of an agent, e.g., a therapeutic agent (or a precursor of the therapeutic agent that is metabolized or altered within the body of the subject to produce the therapeutic agent in vivo) to a subject, using any of the various methods and delivery systems known to those skilled in the art. Administering can also be performed, for example, once, a plurality of times, and/or over one or more extended periods.

As used herein, the term “affinity” refers to the strength of the binding of one protein (e.g., an Antibody) to another protein (e.g., an Antigen). The affinity of a protein is measured by the dissociation constant Kd, defined as [Antibody]×[Antigen]/[Antibody-Antigen] where [Antibody-Antigen] is the molar concentration of the Antibody-Antigen complex, [Antibody] is the molar concentration of the unbound Antibody and [Antigen] is the molar concentration of the unbound Antigen. The affinity constant Ka is defined by 1/Kd. Standard methods of measuring affinity are known to the person of ordinary skill in the art. Exemplary methods of measuring affinity are described herein, see for example, § 5.5.5.

As used herein, the term “antibody” or “antibodies” is used in the broadest sense and encompasses various immunoglobulin (Ig) (e.g., human Ig (hIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methos known in the art, e.g., recombinant production in host cells, see, e.g., § 5.6; or synthetic production.

As used herein, the term “derived from,” with reference to a polynucleotide refers to a polynucleotide that has at least 70% sequence identity to a reference polynucleotide (e.g., a naturally occurring polynucleotide) or a fragment thereof. The term “derived from,” with reference to a polypeptide or protein refers to a polypeptide or protein that comprises an amino acid sequence that has at least 70% sequence identity to the amino acid sequence of a reference polypeptide or protein (e.g., a naturally occurring polypeptide or protein). The term “derived from” as used herein does not denote any specific process or method for obtaining the polynucleotide, polypeptide, or protein. For example, the polynucleotide, polypeptide, or protein can be recombinantly produced or chemically synthesized.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

As used herein, the term “Fc region” refers to the C-terminal region of an Ig heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated by reference herein for all purposes).

As used herein, the term “EGFR” or “epidermal growth factor receptor” refers to the transmembrane receptor member of the ErbB family of receptors that serves as a receptor for the EGF family of extracellular proteins. The amino acid sequence of an exemplary reference mature human EGFR (hEGFR) protein is set forth in SEQ ID NO: 37.

As used herein, the term “EGFR variant” or “variant EGFR” refers to an EGFR protein or polypeptide that comprises at least one amino acid modification (e.g., amino acid substitution) relative to a reference (e.g., wildtype (e.g., SEQ ID NO: 37)) EGFR protein or polypeptide. In some embodiments, the at least one amino acid modification is an amino acid substitution. In some embodiments, the EGFR variant exhibits increased activity.

As used herein, the term “full-length antibody” refers to an antibody having a structure substantially similar to a native antibody structure (i) a first immunoglobulin (Ig) light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the two heavy chains comprise a substantially identical amino acid sequence; and the two light chains comprise a substantially identical amino acid sequence. In some embodiments, the two heavy chains comprise a substantially identical amino acid sequence except for one or more amino acid modifications that promote heterodimerization of the correct heavy chains (e.g., as described herein); and the two light chains comprise a substantially identical amino acid sequence. Antibody chains may be substantially identical but not entirely identical if they differ due to post-translational modifications, such as C-terminal cleavage of lysine residues, alternative glycosylation patterns, etc.

The term “functional variant” as used herein in reference to a polypeptide or protein refers to a polypeptide or protein that comprises at least one but no more than 15%, not more than 12%, no more than 10%, no more than 8% amino acid modification (e.g., substitution, deletion, addition) compared to the amino acid sequence of a reference polypeptide or protein, wherein the polypeptide or protein retains at least one particular function of the reference polypeptide or protein. Not all functions of the reference polypeptide or protein (e.g., wild type) need be retained by the functional variant of the protein. In some instances, one or more functions are selectively reduced or eliminated. In some embodiments, the reference polypeptide or protein is a wild type protein. For example, a functional variant of an antibody that specifically binds EGFR can refer to the antibody that specifically binds EGFR comprising one or more amino acid substitution as compared to a reference antibody that retains the ability to specifically bind EGFR.

The term “functional fragment” as used herein in reference to a polypeptide or protein refers to a fragment of a reference polypeptide or protein that retains at least one particular function. Not all functions of the reference polypeptide or protein need be retained by a functional fragment of the polypeptide or protein. In some instances, one or more functions are selectively reduced or eliminated. In some embodiments, the reference polypeptide or protein is a wild type protein. In some embodiments, the functional fragment polypeptide or protein comprises at least one but no more than 15%, not more than 12%, no more than 10%, no more than 8% amino acid deletion compared to the amino acid sequence of a reference polypeptide or protein. For example, a functional fragment of an antibody that specifically binds EGFR can refer to a fragment of the antibody that retains the ability to specifically bind EGFR.

As used herein, the term “fuse” and grammatical equivalents thereof refer to the operable connection of at least a first polypeptide (or protein) to a second polypeptide (or protein), wherein the first and second polypeptides (or proteins) are not naturally found operably connected together. For example, the first and second polypeptides (or proteins) can be derived from different proteins. The term fuse encompasses both a direct connection of the at least two polypeptides through a peptide bond, and the indirect connection through a linker (e.g., a peptide linker).

As used herein, the term “fusion polypeptide” or “fusion protein” and grammatical equivalents thereof refers to a polypeptide or protein that comprises at least one polypeptide (or protein) operably connected to another polypeptide (or protein), wherein the first and second polypeptides (or proteins) are not naturally found operably connected together. For example, in some embodiments, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “in combination with” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen (e.g., a prime-boost vaccine regimen). In some embodiments, administration of two or more agents or treatments in combination is such that the reduction in a symptom, or other parameter related to the condition is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes.

As used herein, the term “KRAS” or “KRAS” or “kirsten rat sarcoma viral oncogene homolog” refers collectively to the group of KRAS human GTPases, including KRAS isoform 2A and KRAS isoform 2B. The term also includes naturally occurring and engineered variants of KRAS, e.g., variants described herein, e.g., KRAS G12C, G12A, etc. The amino acid sequence of an exemplary reference mature human KRAS (hKRAS) Isoform 2A protein is set forth in SEQ ID NO: 1; and the amino acid sequence of an exemplary reference mature hKRAS Isoform 2B protein is set forth in SEQ ID NO: 3.

As used herein, the term “KRAS inhibitor” or “KRASi” refers to any moiety (e.g., a small molecule compound, protein, polypeptide, nucleic acid, etc.) that is capable of negatively modulating or completely inhibit all or a portion of the enzymatic activity of KRAS (e.g., hKRAS). Exemplary KRAS inhibitors include, but are not limited to, any one or more of the compounds disclosed herein (see, e.g., § 5.2), a pharmaceutically acceptable salt thereof, a hydrate thereof, a solvate thereof, a polymorph thereof, and a stereochemically isomeric form thereof. The term “KRAS inhibitor” includes both specific KRAS inhibitors and pan KRAS inhibitors.

As used herein, the term “specifically inhibits” when used in reference to a KRAS inhibitor refers to the ability of a KRAS inhibitor to preferentially negatively modulating or completely inhibit all or a portion of the enzymatic activity of one or more defined KRAS (e.g., hKRAS) proteins (e.g., one or more defined KRAS variants (e.g., KRAS variant G12C)) relative to other KRAS proteins (e.g., reference KRAS (e.g., wildtype KRAS), other KRAS variants). For example, a KRAS inhibitor that is capable of specifically inhibiting KRAS G12C, refers to a hKRAS inhibitor that is capable of preferentially negatively modulating or completely inhibit all or a portion of the enzymatic activity of hKRAS G12C relative to e.g., a reference KRAS (e.g., wildtype KRAS (e.g., hKRAS comprising the amino acid sequence set forth in SEQ ID NO: 1 or 3)). In some embodiments, a specific KRAS inhibitor may be denoted by KRAS followed by the variant that the KRAS inhibitor specifically inhibits followed “inhibitor” or “i”. For example, a KRAS inhibitor selective for a KRAS variant comprising a G12C substitution may be referred to herein as a “KRAS-G12Ci” or “KRAS-G12C inhibitor” and the like (see, e.g., Examples 9-11).

As used herein, the term “KRAS pathway” refers to the signaling pathway and downstream effects that are initiated by active KRAS (e.g., KRAS bound to GTP).

As used herein, the term “isolated” with reference to a polypeptide, protein, or polynucleotide refers to a polypeptide, protein, or polynucleotide that is substantially free of other cellular components with which it is associated in the natural state.

As used herein, the term “KRAS variant” or “variant KRAS” refers to a KRAS protein or polypeptide that comprises at least one amino acid modification (e.g., amino acid substitution) relative to a reference (e.g., wildtype (e.g., SEQ ID NO: 1 or 3)) KRAS protein or polypeptide. In some embodiments, the at least one amino acid modification is an amino acid substitution. In some embodiments, the KRAS variant exhibits increased activity. In some embodiments, the KRAS variant is constitutively in an active state. Exemplary KRAS variants include KRAS proteins or polypeptides comprising the amino acid sequence of SEQ ID NO: 1 or 3, and also containing a G12C, G12V, G12R, G12D, G12A, G13D, or Q61H amino acid substitution (amino acid numbering relative to SEQ ID NO: 1).

As used herein, the term “KRAS activating amino acid modification” refers to an amino acid modification in the amino acid sequence of a KRAS protein relative to the amino acid sequence of a reference (e.g., wildtype (e.g., SEQ ID NO: 1 or 3)) KRAS protein or polypeptide that locks the KRAS protein in a constitutively active state.

As used herein, the term “KRAS variant cancer” refers to a cancer that is associated with, mediated by, or determined to express a KRAS variant. A non-limiting example of a KRAS variant cancer is a cancer determined to express KRAS G12C (i.e., a cancer determined to express a KRAS protein or polypeptide comprising the amino acid sequence of SEQ ID NO: 1 or 3, and also containing a G12C amino acid substitution (amino acid numbering relative to SEQ ID NO: 1).

As used herein, the term “modification,” with reference to a polynucleotide, refers to a polynucleotide that comprises at least one substitution, alteration, inversion, addition, or deletion of nucleotide compared to a reference polynucleotide (e.g., one or more amino acid substitutions). Modifications can include the inclusion of non-naturally occurring nucleotide residues. As used herein, the term “modification,” with reference to an amino acid sequence refers to an amino acid sequence that comprises at least one substitution, alteration, inversion, addition, or deletion of an amino acid residue compared to a reference amino acid sequence. Modifications can include the inclusion of non-naturally occurring amino acid residues. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein.

As used herein, the term “moiety” is used generically to describe any macro or micro molecule that can be incorporated into a fusion protein or polypeptides described herein. The moieties of a fusion protein or polypeptide are operably connected. Exemplary moieties include, but are not limited protein, polypeptides, polynucleotides (e.g., DNA, RNA), small molecules, carbohydrates, lipids, synthetic polymers (e.g., polymers of PEG). In some embodiments, the moiety is a polypeptide. In some embodiments, the moiety is a protein.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein or polypeptide described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein or polypeptide).

The determination of “percent identity” between two sequences (e.g., peptide or protein (amino acid sequences) or polynucleotide (nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, the term “pharmaceutically acceptable salt” generally has its art-recognized meaning and refers to derivatives of the compounds provided herein wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds provided herein include the conventional non-toxic salts of the parent compound formed, for example, from non-toxic inorganic or organic acids. The pharmaceutically acceptable salts of the compounds provided herein can be synthesized from the parent compound which contains a basic or acidic moiety by conventional chemical methods. Generally, such salts can be prepared by combining the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile may be used. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), the full contents of each of which is incorporated herein by reference for all purposes.

The terms “polynucleotide” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As A used herein, the term “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” are used interchangeably herein.

As used herein, the term “protein” refers to a polypeptide or a set (i.e., at least two) polypeptides. In embodiments where the protein comprises a set of polypeptides, the set of polypeptides associate to form a functional unit (i.e., quaternary structure). In some embodiments, the polypeptide or set of polypeptides are folded into their three-dimensional structure (i.e., tertiary or quaternary structure). Where polypeptides or sets of polypeptides are contemplated herein, it should be understood that proteins comprising the polypeptides or sets of polypeptides folded into their three-dimensional structure (i.e., tertiary or quaternary structure) are also provided herein and vice versa.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “obtaining a sample” refers to the acquisition of a sample. The term includes the direct acquisition from a subject and the indirect acquisition through one or more third parties wherein one of the third parties directly acquired the sample from the subject.

As used herein, the term “sample” encompass a variety of biological specimens obtained from a subject. Exemplary sample types include, e.g., solid tissue samples such as biopsies (or cells derived therefrom and the progeny thereof), tissue cultures (or cells derived therefrom and the progeny thereof), and cell cultures (or cells derived therefrom and the progeny thereof), blood and other liquid samples of biological origin (including, but not limited to, whole-blood, peripheral blood mononuclear cells (PBMCs), serum, plasma, urine, saliva, amniotic fluid, stool, synovial fluid, etc.), nasopharyngeal swabs. The term also includes samples that have been manipulated in any way after their procurement from a subject, such as by centrifugation, filtration, washing, precipitation, dialysis, chromatography, lysis, treatment with reagents, enriched for certain cell populations, refrigeration, freezing, staining, etc. In some embodiments, the sample comprises cancer cells. In some embodiments, the sample is a tissue sample that contains cancer cells.

The term “scFv” or “single chain variable fragment” as used herein refers an antigen binding protein or polypeptide that comprises a VH operably connected (e.g., via a peptide linker) to a VL. In some embodiments, the VH is operably connected to the VL via a peptide linker. The components of the scFv can be in any orientation, for example, the scFv can comprise from N- to C-terminus a VH, a peptide linker, and a VL; or from N- to C-terminus a VL, a peptide linker, and a VH.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “specifically binds” refers to preferential interaction, i.e., significantly higher binding affinity, between a first protein (e.g., a ligand) and a second protein (e.g., the ligand's cognate receptor) relative to other amino acid sequences. Herein, when a first protein or polypeptide is said to “specifically bind” to a second protein or polypeptide, it is understood that the first protein or polypeptide specifically binds to an epitope of the second protein or polypeptide. The term “epitope” refers to the portion of the second protein or polypeptide that the first protein or polypeptide specifically recognizes. The term specifically binds includes molecules that are cross reactive with the same epitope of a different species. For example, an antibody or protein that specifically binds human EGFR may be cross reactive with EGFR of another species (e.g., cynomolgus, murine, etc.), and still be considered herein to specifically bind human EGFR.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Palcaognathac (e.g., ostriches, emus), Columbiformes (e.g., pigeons, doves), or Psittaciformes (e.g., parrots).

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the term “TGFβ” or “transforming growth factor β” refers to the multifunctional 25-kDa dimeric cytokine belonging to the transforming growth factor superfamily. The amino acid sequence of an exemplary reference mature human TGFβ1 (hTGFβ1) protein is set forth in SEQ ID NO: 61; the amino acid sequence of an exemplary reference mature human TGFβ-2 (hTGFβ2) protein is set forth in SEQ ID NO: 63; and the amino acid sequence of an exemplary reference mature human TGFβ-3 (hTGFβ3) protein is set forth in SEQ ID NO: 65.

As used herein, the term “TGFβRI” or “transforming growth factor β receptor I” refers to the transmembrane protein that has a protein kinase domain, forms a heterodimeric complex with TGFβRII, and binds TGFβ. The amino acid sequence of an exemplary reference mature human TGFβRI (hTGFβRI) protein is set forth in SEQ ID NO: 67.

As used herein, the term “TGFβRII” or “transforming growth factor β receptor II” refers to the transmembrane protein that has a protein kinase domain, forms a heterodimeric complex with TGFβRI, and binds TGFβ. The amino acid sequence of an exemplary reference mature human TGFβRII (hTGFβRII) protein is set forth in SEQ ID NO: 70.

As used herein, the term “TGFβ pathway” refers to the signaling pathway and downstream effects that are initiated by the binding of TGFβ to the TGFβR (e.g., heterotetramer composed of two TβRI:TβRII heterodimers (e.g., two hTβRI:hTβRII heterodimers)).

As used herein, the terms “treat,” treating,” “treatment,” and the like refer to reducing or ameliorating a disease or infection and/or symptom(s) associated therewith or obtaining a desired pharmacologic and/or physiologic effect. It will be appreciated that, although not precluded, treating a disease or infection does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

The terms “VL” and “VL domain” are used interchangeably to refer to the light chain variable region of an antibody.

The terms “VH” and “VH domain” are used interchangeably to refer to the heavy chain variable region of an antibody.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The term “(VHH)₂” as used herein refers to an antibody that comprises a first and a second VHH operably connected (e.g., via a peptide linker). The first and the second VHH can specifically bind the same or different antigens. In some embodiments, the first and second VHH are operably connected by a peptide linker.

The term “VHH-Fc” as used herein refers to an antibody that comprises a VHH operably linked (e.g., via a peptide linker) to an Fc domain or a subunit of an Fc domain. In some embodiments, a VHH is operably connected to only a first Fc domain of a first and a second Fc domain pair. In some embodiments, a first VHH is operably connected to a first Fc domain and a second VHH is operably connected to a second Fc domain of a first Fc and a second Fc pair.

The term “(VHH)₂-Fc” as used herein refers to (VHH)₂ operably linked (e.g., via a peptide linker) to an Fc domain or a subunit of an Fc domain. In some embodiments, a (VHH)₂ is operably connected to only a first Fc domain of a first and a second Fc domain pair. In some embodiments, a first (VHH)₂ is operably connected to a first Fc domain and a second (VHH)₂ is operably connected to a second Fc domain of a first Fc and a second Fc pair.

### 5.2 KRAS Inhibitors

Kirsten Rat Sarcoma Viral Oncogene Homolog (KRAS) is a small guanosine triphosphatase (GTPase) that functions largely by coupling membrane growth factor receptors with intracellular signaling pathways and transcription factors.

KRAS proteins primarily bind to guanosine diphosphate (GDP) and are in an inactive conformation maintained by the intrinsic guanosine triphosphate (GTP) hydrolytic activity. KRAS interacts with GTPase activating protein (GAP) accelerating GTP toward conversion of GDP, while guanine nucleotide exchange factor (GEF) binding with KRAS results in the KRAS passively loading with the GTP. GTP binding to KRAS shifts the active site from an open to a closed conformation, allowing multiple downstream effector pathways to interact and activate, including the mitogen-activated protein kinase (MAPK) and phosphatidylinositol 3-kinase (PI3K) pathways. The activated state of KRAS accumulating in vivo results in the activation of downstream signaling pathways and is associated with tumorigenesis, aggressive disease, and poor prognosis. Upstream signaling pathways of KRAS mainly include cell surface receptors, such as EGFR (ERBB1), human epithelial growth factor receptor 2 (HER2 (ERBB2)), HER3 (ERBB3), and ERBB4.

KRAS variants that lock the protein the active state are found in different types of cancer, including, but not limited to, e.g., colorectal, colon, rectal, lung, and pancreatic cancer. More common active state KRAS mutations are at positions G12 and G13 (numbering relative SEQ ID NO: 3). Additional KRAS variants associated with cancer are known in the art and also described herein. See, e.g., Adachi Y, Ito K, Hayashi Y, et al. Epithelial-to-Mesenchymal Transition is a Cause of Both Intrinsic and Acquired Resistance to KRAS G12C Inhibitor in KRAS G12C-Mutant Non-Small Cell Lung Cancer. Clin Cancer Res. 2020; 26 (22): 5962-5973. doi: 10.1158/1078-0432.CCR-20-2077; Huang, L., Guo, Z., Wang, F. et al. KRAS mutation: from undruggable to druggable in cancer. Sig Transduct T Target Ther 6, 386 (2021). https://doi.org/10.1038/s41392-021-00780-4; and Ryan M B, Coker O, Sorokin A, et al. KRASG12C-independent feedback activation of wild-type RAS constrains KRASG12C inhibitor efficacy. Cell Rep. 2022; 39 (12): 110993. doi: 10.1016/j.celrep.2022.110993; the entire contents of each of which are incorporated herein by reference for all purposes.

There are two splice isoforms of human KRAS mRNA that result in two variants of the KRAS protein. The variant commonly referred to as KRAS isoform B is the predominant isoform The amino acid sequence of a reference hKRAS Isoform 2A polypeptide and a reference hKRAS Isoform 2B is set forth in SEQ ID NOS: 1 and 3, respectively. The amino acid sequence of exemplary hKRAS 2A variants G12C, G12V, G12R, G12D, G12A, G12S, G13D, and Q61H are also provided in SEQ ID NOS: 5, 6, 7, 8, 9, 10, 11, and 12, respectively. The amino acid sequence of exemplary hKRAS 2B variants G12C, G12V, G12R, G12D, G12A, G12S, G13D, and Q61H are also provided in SEQ ID NOS: 13, 14, 15, 16, 17, 18, 19, and 20, respectively. See Table 1, herein.

Both isoforms of KRAS contain an N-terminal initiator methionine. It is known in the art that KRAS may be further processed by the cell resulting in the cleavage of the N-terminal initiator methionine. See Dharmaiah, S., Tran, T. H., Messing, S. et al. Structures of N-terminally processed KRAS provide insight into the role of N-acetylation. Sci Rep 9, 10512 (2019). https://doi.org/10.1038/s41598-019-46846-w, the full contents of which is incorporated by reference herein for all purposes. The amino acid sequence of a reference hKRAS Isoform 2A polypeptide without the initiator methionine and a reference hKRAS Isoform 2B without the initiator methionine is set forth in SEQ ID NOS: 2 and 4, respectively. The amino acid sequence of exemplary hKRAS 2A variants G12C, G12V, G12R, G12D, G12A, G12S, G13D, and Q61H without the initiator methionine are also provided in SEQ ID NOS: 21, 22, 23, 24, 25, 26, 27, and 28, respectively. The amino acid sequence of exemplary hKRAS 2B variants G12C, G12V, G12R, G12D, G12A, G12S, G13D, and Q61H without the initiator methionine are also provided in SEQ ID NOS: 29, 30, 31, 32, 33, 34, 35, and 36, respectively. See Table 1, herein.

Unless otherwise noted, the numbering of amino acids in KRAS is relative to the KRAS isoforms containing the initiator methionine (i.e., SEQ ID NO: 1-hKRAS Isoform 2A; SEQ ID NO: 3-hKRAS Isoform 2B).

TABLE 1
The Amino Acid Sequence of Reference KRAS Polypeptides
and KRAS Variants.

| | | SEQ |
|---|---|---|
| | | ID |
| Description | Amino Acid Sequence | NO |
| hKRAS Isoform 2A | MTEYKLVVVGAGGVGKSALTIQLIQNHFVDEYDPTIEDSYR | 1 |
| With initiator | KQVVIDGETCLLDILDTAGQEEYSAMRDQYMRTGEGFLCVF | |
| methionine | AINNTKSFEDIHHYREQIKRVKDSEDVPMVLVGNKCDLPSR | |
| | TVDTKQAQDLARSYGIPFIETSAKTRQRVEDAFYTLVREIR | |
| | QYRLKKISKEEKTPGCVKIKKCIIM | |
| hKRAS Isoform 2A | TEYKLVVVGAGGVGKSALTIQLIQNHFVDEYDPTIEDSYRK | 2 |
| Without initiator | QVVIDGETCLLDILDTAGQEEYSAMRDQYMRTGEGFLCVFA | |
| methionine | INNTKSFEDIHHYREQIKRVKDSEDVPMVLVGNKCDLPSRT | |
| | VDTKQAQDLARSYGIPFIETSAKTRQRVEDAFYTLVREIRQ | |
| | YRLKKISKEEKTPGCVKIKKCIIM | |
| hKRAS Isoform 2B | MTEYKLVVVGAGGVGKSALTIQLIQNHFVDEYDPTIEDSYR | 3 |
| With initiator | KQVVIDGETCLLDILDTAGQEEYSAMRDQYMRTGEGFLCVF | |
| methionine | AINNTKSFEDIHHYREQIKRVKDSEDVPMVLVGNKCDLPSR | |
| | TVDTKQAQDLARSYGIPFIETSAKTRQGVDDAFYTLVREIR | |
| | KHKEKMSKDGKKKKKKSKTKCVIM | |
| hKRAS Isoform 2B | TEYKLVVVGAGGVGKSALTIQLIQNHFVDEYDPTIEDSYRK | 4 |
| Without initiator | QVVIDGETCLLDILDTAGQEEYSAMRDQYMRTGEGFLCVFA | |
| methionine | INNTKSFEDIHHYREQIKRVKDSEDVPMVLVGNKCDLPSRT | |
| | VDTKQAQDLARSYGIPFIETSAKTRQGVDDAFYTLVREIRK | |
| | HKEKMSKDGKKKKKKSKTKCVIM | |
| hKRAS 2A G12C | MTEYKLVVVGACGVGKSALTIQLIQNHFVDEYDPTIEDSYR | 5 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12V | MTEYKLVVVGAVGVGKSALTIQLIQNHFVDEYDPTIEDSYR | 6 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12R | MTEYKLVVVGARGVGKSALTIQLIQNHFVDEYDPTIEDSYR | 7 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12D | MTEYKLVVVGADGVGKSALTIQLIQNHFVDEYDPTIEDSYR | 8 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12A | MTEYKLVVVGAAGVGKSALTIQLIQNHFVDEYDPTIEDSYR | 9 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12S | MTEYKLVVVGASGVGKSALTIQLIQNHFVDEYDPTIEDSYR | 10 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G13D | MTEYKLVVVGAGDVGKSALTIQLIQNHFVDEYDPTIEDSYR | 11 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A Q61H | MTEYKLVVVGAGGVGKSALTIQLIQNHFVDEYDPTIEDSYR | 12 |
| Variant | KQVVIDGETCLLDILDTAGHEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2B G12C | MTEYKLVVVGACGVGKSALTIQLIQNHFVDEYDPTIEDSYR | 13 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12V | MTEYKLVVVGAVGVGKSALTIQLIQNHFVDEYDPTIEDSYR | 14 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12R | MTEYKLVVVGARGVGKSALTIQLIQNHFVDEYDPTIEDSYR | 15 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12D | MTEYKLVVVGADGVGKSALTIQLIQNHFVDEYDPTIEDSYR | 16 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12A | MTEYKLVVVGAAGVGKSALTIQLIQNHFVDEYDPTIEDSYR | 17 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12S | MTEYKLVVVGASGVGKSALTIQLIQNHFVDEYDPTIEDSYR | 18 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G13D | MTEYKLVVVGAGDVGKSALTIQLIQNHFVDEYDPTIEDSYR | 19 |
| Variant | KQVVIDGETCLLDILDTAGQEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B Q61H | MTEYKLVVVGAGGVGKSALTIQLIQNHFVDEYDPTIEDSYR | 20 |
| Variant | KQVVIDGETCLLDILDTAGHEEYSAMRDQYMRTGEGFLCVF | |
| With initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2A G12C | MTEYKLVVVGACGVGKSALTIQLIQNHFVDEYDPTIEDSYR | 21 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGC | |
| | VKIKKCIIM | |
| hKRAS 2A G12V | MTEYKLVVVGAVGVGKSALTIQLIQNHFVDEYDPTIEDSYR | 22 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12R | MTEYKLVVVGARGVGKSALTIQLIQNHFVDEYDPTIEDSYR | 23 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12D | MTEYKLVVVGADGVGKSALTIQLIQNHFVDEYDPTIEDSYR | 24 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12A | MTEYKLVVVGAAGVGKSALTIQLIQNHFVDEYDPTIEDSYR | 25 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G12S | MTEYKLVVVGASGVGKSALTIQLIQNHFVDEYDPTIEDSYR | 26 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A G13D | MTEYKLVVVGAGDVGKSALTIQLIQNHFVDEYDPTIEDSYR | 27 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2A Q61H | MTEYKLVVVGAGGVGKSALTIQLIQNHFVDEYDPTIEDSYR | 28 |
| Variant | KQVVIDGETCLLDILDTAGHEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQRVEDAFYTLVREIR | |
| | QYRLKKISKEEKTPGCVKIKKCIIM | |
| hKRAS 2B G12C | MTEYKLVVVGACGVGKSALTIQLIQNHFVDEYDPTIEDSYR | 29 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12V | MTEYKLVVVGAVGVGKSALTIQLIQNHFVDEYDPTIEDSYR | 30 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12R | MTEYKLVVVGARGVGKSALTIQLIQNHFVDEYDPTIEDSYR | 31 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12D | MTEYKLVVVGADGVGKSALTIQLIQNHFVDEYDPTIEDSYR | 32 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12A | MTEYKLVVVGAAGVGKSALTIQLIQNHFVDEYDPTIEDSYR | 33 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G12S | MTEYKLVVVGASGVGKSALTIQLIQNHFVDEYDPTIEDSYR | 34 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B G13D | MTEYKLVVVGAGDVGKSALTIQLIQNHFVDEYDPTIEDSYR | 35 |
| Variant | KQVVIDGETCLLDILDTAGQEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |
| hKRAS 2B Q61H | MTEYKLVVVGAGGVGKSALTIQLIQNHFVDEYDPTIEDSYR | 36 |
| Variant | KQVVIDGETCLLDILDTAGHEEYSAMRDQYMRTGEGFLCVF | |
| Without initiator | AINNTKSFEDIHHYREQIKRVKDSEDVPMVLVGNKCDLPSR | |
| methionine | TVDTKQAQDLARSYGIPFIETSAKTRQGVDDAFYTLVREIR | |
| | KHKEKMSKDGKKKKKKSKTKCVIM | |

In some embodiments, the KRAS (e.g., hKRAS) inhibitor is capable of inhibiting a KRAS (e.g., hKRAS) variant. In some embodiments, the KRAS (e.g., hKRAS) inhibitor is capable of inhibiting more than one KRAS (e.g., hKRAS) variant. In some embodiments, the KRAS (e.g., hKRAS) inhibitor is capable of inhibiting at least one KRAS (e.g., hKRAS) variant. In some embodiments, the KRAS (e.g., hKRAS) inhibitor is capable of inhibiting multiple KRAS variants.

In some embodiments, the KRAS (e.g., hKRAS) inhibitor is capable of inhibiting a KRAS variant comprising a KRAS activating amino acid modification.

In some embodiments, the KRAS (e.g., hKRAS) inhibitor is capable of inhibiting a KRAS (e.g., hKRAS) variant comprising an amino acid substitution at amino acid position G12, G13, or Q61, numbering relative to the amino acid sequence of SEQ ID NO: 1. In some embodiments, the KRAS (e.g., hKRAS) inhibitor is capable of inhibiting a KRAS (e.g., hKRAS) variant comprising an amino acid substitution at amino acid position G12 or G13, numbering relative to the amino acid sequence of SEQ ID NO: 1. In some embodiments, the KRAS (e.g., hKRAS) inhibitor is capable of inhibiting a KRAS (e.g., hKRAS) variant comprising an amino acid substitution at amino acid position G12, numbering relative to the amino acid sequence of SEQ ID NO: 1.

In some embodiments, the KRAS (e.g., hKRAS) inhibitor is capable of inhibiting a KRAS (e.g., hKRAS) variant comprising one or more of the following amino acid substitutions G12C, G12V, G12R, G12D, G12A, G12S, G13D, or Q61H, numbering relative to the amino acid sequence of SEQ ID NO: 1. In some embodiments, the KRAS (e.g., hKRAS) inhibitor is capable of inhibiting a KRAS (e.g., hKRAS) variant comprising one of the following amino acid substitutions G12C, G12V, G12R, G12D, G12S, G12A, or G13D, numbering relative to the amino acid sequence of SEQ ID NO: 1. In some embodiments, the KRAS (e.g., hKRAS) inhibitor is capable of inhibiting a KRAS (e.g., hKRAS) variant comprising one of the following amino acid substitutions G12C, G12V, G12R, G12D, G12S, or G12A, numbering relative to the amino acid sequence of SEQ ID NO: 1. In some embodiments, the KRAS (e.g., hKRAS) inhibitor is capable of inhibiting a KRAS (e.g., hKRAS) variant comprising a G12C amino acid substitution, numbering relative to the amino acid sequence of SEQ ID NO: 1.

In some embodiments, the KRAS (e.g., hKRAS) inhibitor is capable of selectively inhibiting a KRAS (e.g., hKRAS) variant. In some embodiments, the KRAS (e.g., hKRAS) inhibitor is capable of selectively inhibiting more than one KRAS (e.g., hKRAS) variant. In some embodiments, the KRAS (e.g., hKRAS) inhibitor is capable of selectively inhibiting at least one KRAS (e.g., hKRAS) variant. In some embodiments, the KRAS (e.g., hKRAS) inhibitor is capable of selectively inhibiting multiple KRAS variants.

In some embodiments, the KRAS (e.g., hKRAS) inhibitor is capable of selectively inhibiting a KRAS variant comprising a KRAS activating amino acid modification.

In some embodiments, the KRAS (e.g., hKRAS) inhibitor is capable of selectively inhibiting a KRAS (e.g., hKRAS) variant comprising an amino acid substitution at amino acid position G12, G13, or Q61, numbering relative to the amino acid sequence of SEQ ID NO: 1. In some embodiments, the KRAS (e.g., hKRAS) inhibitor is capable of selectively inhibiting a KRAS (e.g., hKRAS) variant comprising an amino acid substitution at amino acid position G12 or G13, numbering relative to the amino acid sequence of SEQ ID NO: 1. In some embodiments, the KRAS (e.g., hKRAS) inhibitor is capable of selectively inhibiting a KRAS (e.g., hKRAS) variant comprising an amino acid substitution at amino acid position G12, numbering relative to the amino acid sequence of SEQ ID NO: 1.

In some embodiments, the KRAS (e.g., hKRAS) inhibitor is capable of selectively inhibiting a KRAS (e.g., hKRAS) variant comprising one or more of the following amino acid substitutions G12C, G12V, G12R, G12D, G12A, G12S, G13D, or Q61H, numbering relative to the amino acid sequence of SEQ ID NO: 1. In some embodiments, the KRAS (e.g., hKRAS) inhibitor is capable of selectively inhibiting a KRAS (e.g., hKRAS) variant comprising one of the following amino acid substitutions G12C, G12V, G12R, G12D, G12S, G12A, or G13D, numbering relative to the amino acid sequence of SEQ ID NO: 1. In some embodiments, the KRAS (e.g., hKRAS) inhibitor is capable of selectively inhibiting a KRAS (e.g., hKRAS) variant comprising one of the following amino acid substitutions G12C, G12V, G12R, G12D, G12S, or G12A, numbering relative to the amino acid sequence of SEQ ID NO: 1. In some embodiments, the KRAS (e.g., hKRAS) inhibitor is capable of selectively inhibiting a KRAS (e.g., hKRAS) variant comprising a G12C amino acid substitution, numbering relative to the amino acid sequence of SEQ ID NO: 1.

In some embodiments, the KRAS (e.g., hKRAS) variant comprises a KRAS (e.g., hKRAS) activating amino acid modification.

In some embodiments, the KRAS (e.g., hKRAS) variant comprises an amino acid modification at amino acid position G12, G13, or Q61, numbering relative to the amino acid sequence of SEQ ID NO: 1. In some embodiments, the KRAS (e.g., hKRAS) variant comprises an amino acid modification at amino acid position G12 or G13, numbering relative to the amino acid sequence of SEQ ID NO: 1.

In some embodiments, the KRAS (e.g., hKRAS) variant comprises any one or more of the following amino acid substitutions G12C, G12V, G12R, G12D, G12A, G12S, G13D, or Q61H, numbering relative to the amino acid sequence of SEQ ID NO: 1. In some embodiments, the KRAS (e.g., hKRAS) variant comprises any one or more of the following amino acid substitutions G12C, G12V, G12R, G12D, G12A, G12S, or G13D, numbering relative to the amino acid sequence of SEQ ID NO: 1. In some embodiments, the KRAS (e.g., hKRAS) variant comprises any one of the following amino acid substitutions G12C, G12V, G12R, G12A, G12S, or G12D, numbering relative to the amino acid sequence of SEQ ID NO: 1. In some embodiments, the KRAS (e.g., hKRAS) variant comprises a G12C amino acid substitution, numbering relative to the amino acid sequence of SEQ ID NO: 1.

In some embodiments, the methods, pharmaceutical compositions, kits, etc. described herein utilize or comprise a KRAS (e.g., hKRAS) inhibitor. KRAS (e.g., hKRAS) inhibitors include, but are not limited to, small molecules, proteins (e.g., antibodies and functional fragments or variants thereof, e.g., a full-length antibody, a single chain variable fragment (scFv), a scFv2, a scFv-Fc, a Fab, a Fab′, a F(ab′)2, or a F(v)), nucleic acid, carbohydrate, a lipid, a metal, or a toxin. KRAS inhibitors are known the art and any suitable KRAS (e.g., hKRAS) inhibitor may be employed in the aspects and embodiments described herein. In some embodiments, the KRAS (e.g., hKRAS) inhibitor is a small molecule.

In some embodiments, the KRAS (e.g., hKRAS) inhibitor is comprises sotorasib (AMG-510), MRTX849, ARS-1620, GDC-6036, LY3499446, JAB-21822, GFH925, JDQ443, HBI-2438, or YL-15293. In some embodiments, the KRAS inhibitor comprises sotorasib (AMG-510), GDC-6036, LY3499446, JAB-21822, GFH925, JDQ443, HBI-2438, YL-15293, sotorasib (AMG-510), adagrasib (MRTX849), garsorasib, MRTX-1257, D-1553, BI-1823911, ARS-853, ARS-1620, HS-10370, MK-1084, BPI-421286, or GH35. In some embodiments, the KRAS (e.g., hKRAS) inhibitor comprises sotorasib (AMG-510), adagrasib (MRTX849), or ARS-1620.

In some embodiments, the KRAS (e.g., hKRAS) inhibitor comprises sotorasib (AMG-510). The chemical name of sotorasib is 6-fluoro-7-(2-fluoro-6-hydroxyphenyl)-(1M)-1-[4-methyl-2-(propan-2-yl)pyridin-3-yl]-4-[(2S)-2-methyl-4-(prop-2enoyl) piperazin-1-yl]pyrido[2,3-d]pyrimidin-2 (1H)-one.

Additional exemplary KRAS (e.g., hKRAS) inhibitors, include, but are not limited to JNJ-74699157 (ARS-3248) (Janssen Research & Development, LLC), LY3537982 (Eli Lilly and Company), D-1553 (InventisBio Inc.), BI 1823911 (Boehringer Ingelheim), JAB21822 (Jacobio Pharmaceuticals Co., Ltd.), GFH925 (Genfleet Therapeutics Co., Ltd.), HS-10370 (Jiangsu Hansoh Pharmaceutical Co., Ltd.), MK-1084 (Merck Sharp & Dohme LLC), BPI-421286 (Betta Pharmaceuticals Co., Ltd.), GH35 (Suzhou Genhouse Bio Co., Ltd.), D3S-001 (D3 Bio (Wuxi) Co., Ltd.), IBI-351 (Innovent Biologics, Inc.), RMC-6291 (Revolution Medicines), HBI2438 (Huyabio International, LLC), YL15293 (YingLi Pharmaceutical Co. Ltd.), JAB-22000 (Jacobio Pharmaceuticals Co., Ltd.), HRS-4642 (Jiangsu Hengrui Medicine), ASP-3082 (Astellas Pharma), RMC-6236 (Revolution Medicines), or BI-1701963 (Boehringer Ingelheim).

Exemplary hKRAS inhibitors are provided in Table 2. The hKRAS inhibitors are intended to be exemplary only and are not intended to be limiting. See Table 2, herein.

TABLE 2
Exemplary hKRAS Inhibitors.

| | | Putative Target |
|---|---|---|
| Description | Structure | KRAS Variant |
| AMG510 (sotorasib) | 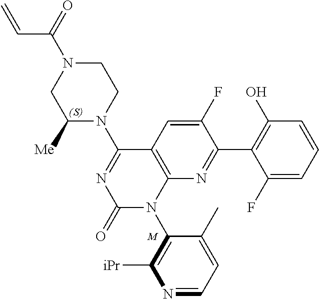 | G12C |
| Sotorasib racemate | 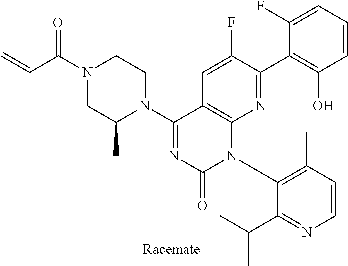 [SMILES: C=CC(=O)N1CCN(c2nc(=O)n(-c3c(C)ccnc3C(C)C)c3nc(-c4c(O)cccc4F)c(F)cc23)[C@@H](C)C1] | G12C |
| | CAS No.: 2252403-56-6 | |
| MRTX-849 (adagrasib) | 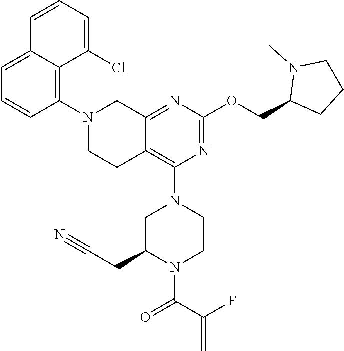 [SMILES: C=C(F)C(=O)N1CCN(c2nc(OC[C@@H]3CCCN3C)nc3c2CCN(c2cccc4cccc(Cl)c24)C3)C[C@@H]1CC#N] | G12C |
| | CAS No.: 2326521-71-3 | |
| MRTX-849 acid | 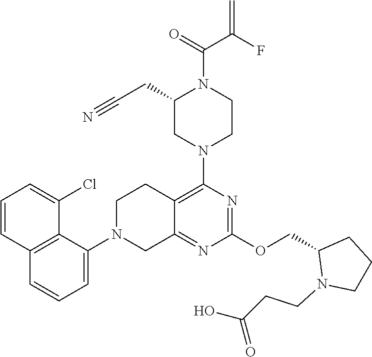 [SMILES: C=C(F)C(=O)N1CCN(c2nc(OC[C@@H]3CCCN3CCC(=O)O)nc3c2CCN(c2cccc4cccc(Cl)c24)C3)C[C@@H]1CC#N] | G12C |
| | CAS No.: 2561529-96-0 | |
| MRTX849 ethoxypropanoic acid | 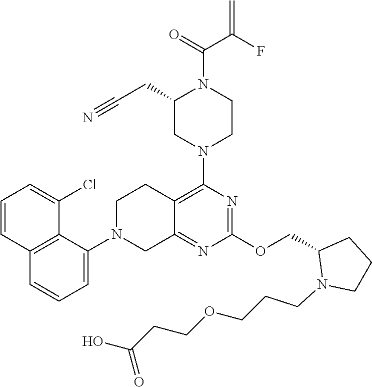 [SMILES: C=C(F)C(=O)N1CCN(c2nc(OC[C@@H]3CCCN3CCCOCCC(=O)O)nc3c2CCN(c2cccc4cccc(Cl)c24)C3)C[C@@H]1CC#N] | G12C |
| | CAS No.: 2561529-98-2 | |
| Garsorasib | 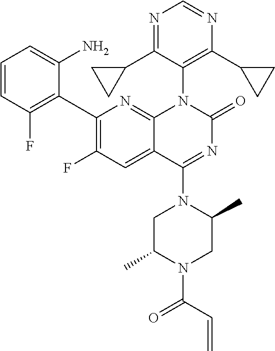 [SMILES: C=CC(=O)N1C[C@H](C)N(c2nc(=O)n(-c3c(C4CC4)ncnc3C3CC3)c3nc(-c4c(N)cccc4F)c(F)cc23)C[C@H]1C] | G12C |
| | CAS No.: 2559761-14-5 | |
| MRTX-1257 | 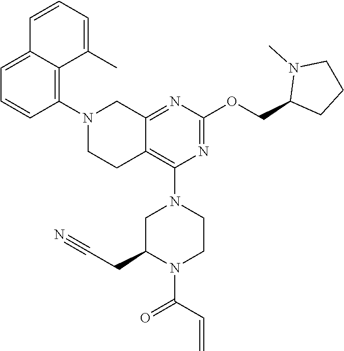 [SMILES: C=CC(=O)N1CCN(c2nc(OC[C@@H]3CCCN3C)nc3c2CCN(c2cccc4cccc(C)c24)C3)C[C@@H]1CC#N] | G12C |
| | CAS No.: 2206736-04-9 | |
| LY3499446 | 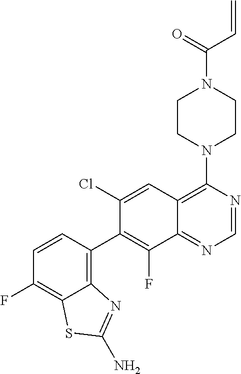 [SMILES: C=CC(=O)N1CCN(c2ncnc3c(F)c(-c4ccc(F)c5sc(N)nc45)c(Cl)cc23)CC1] | G12C |
| | CAS No.: 2409131-50-4 | |
| GDC-6036 | 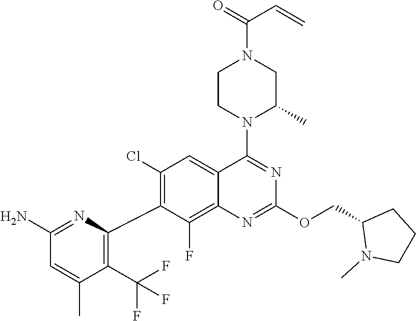 | G12C |
| | CAS No.: 2417987-45-0 | |
| GDC-6036-NH | 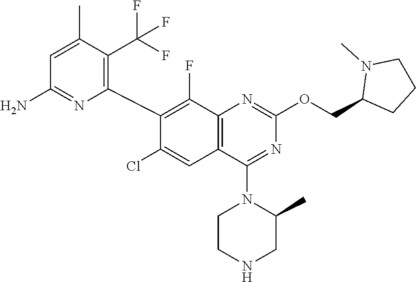 [SMILES: Cc1cc(N)nc(-c2c(Cl)cc3c(N4CCNC[C@@H]4C)nc(OC[C@@H]4CCCN4C)nc3c2F)c1C(F)(F)F] | G12C |
| | CAS No.: 2417918-80-8 | |
| ARS-853 | 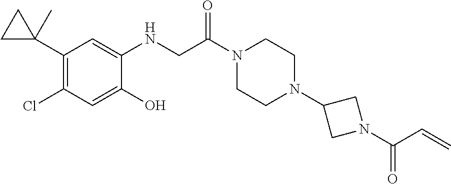 [SMILES: C=CC(=O)N1CC(N2CCN(C(=O)CNc3cc(C4(C)CC4)c(Cl)cc3O)CC2)C1] | G12C |
| ARS-1620 | 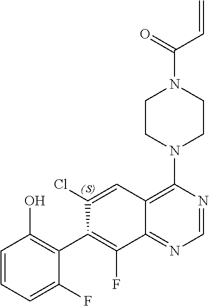 | G12C |
| | CAS No.: 1698055-85-4 | |
| JDQ-443 | 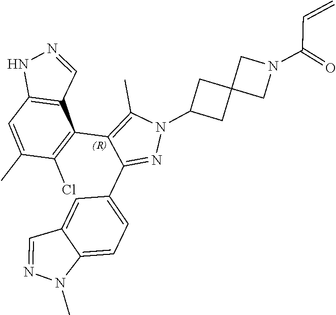 | G12C |
| | CAS No.: 2653994-08-0 | |
| Compound 12 | 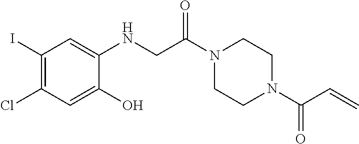 [SMILES: C=CC(=O)N1CCN(C(=O)CNc2cc(I)c(Cl)cc2O)CC1] | G12C |
| AZD4625 | 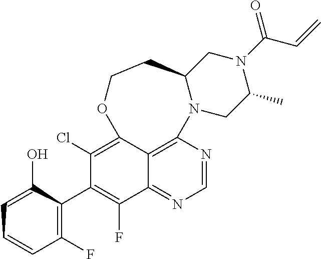 | G12C |
| BI-0474 | 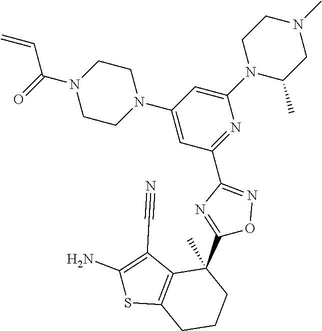 [SMILES: C=CC(=O)N1CCN(c2cc(-c3noc([C@@]4(C)CCCc5sc(N)c(C#N)c54)n3)nc(N3CCN(C)C[C@@H]3C)c2)CC1] | G12C |
| | CAS No.: 2750570-55-7 | |
| LC-2 | 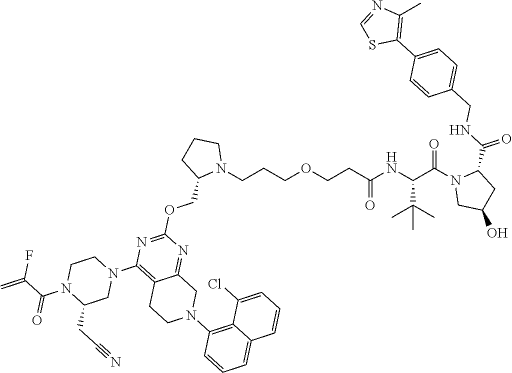 [SMILES: C=C(F)C(=O)N1CCN(c2nc(OC[C@@H]3CCCN3CCCOCCC(=O)N[C@H](C(=O)N3C[C@H](O)C[C@H]3C(=O)NCc3ccc(-c4scnc4C)cc3)C(C)(C)C)nc3c2CCN(c2cccc4cccc(Cl)c24)C3)C[C@@H]1CC#N] | G12C |
| | CAS No.: 2502156-03-6 | |
| RM-018 | 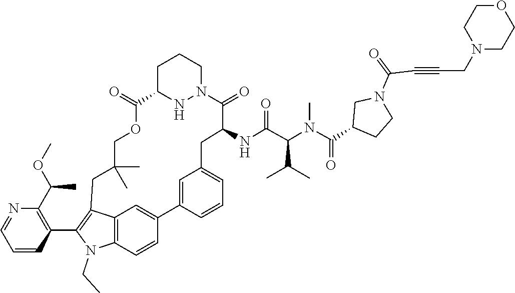 | G12C (Also has ability to bind and inhibit KRASG12C/Y96D) |
| | CAS No.: 2641993-55-5 | |
| ARS-1323 | 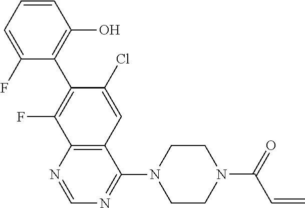 [SMILES: C=CC(=O)N1CCN(c2ncnc3c(F)c(-c4c(O)cccc4F)c(Cl)cc23)CC1] | G12C |
| | CAS No.: 1698024-73-5 | |
| ARS-1323- alkyne | 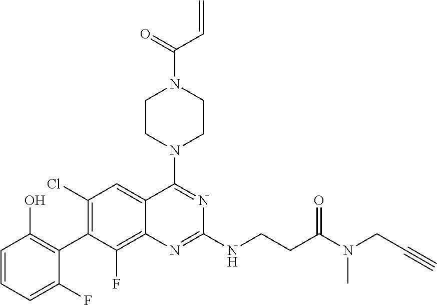 [SMILES: C#CCN(C)C(=O)CCNc1nc(N2CCN(C(=O)C=C)CC2)c2cc(Cl)c(-c3c(O)cccc3F)c(F)c2n1] | G12C |
| | CAS No.: 2436544-27-1 | |
| ARS-1630 | 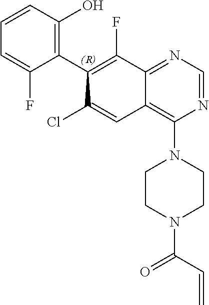 | G12C |
| | CAS No.: 1698055-86-5 | |
| ASP2453 | 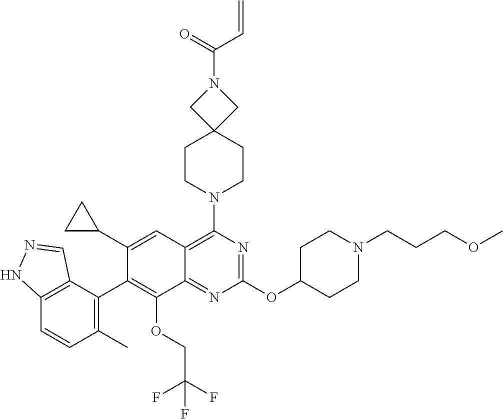 [SMILES: C=CC(=O)N1CC2(CCN(c3nc(OC4CCN(CCCOC)CC4)nc4c(OCC(F)(F)F)c(-c5c(C)ccc6[nH]ncc56)c(C5CC5)cc34)CC2)C1] | G12C |
| | CAS No.: 2241719-73-1 | |
| K-Ras G12C- IN-2 | 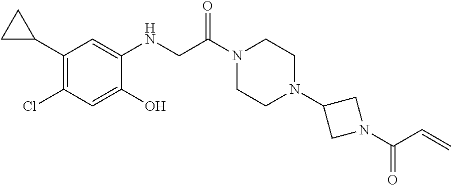 [SMILES: C=CC(=O)N1CC(N2CCN(C(=O)CNc3cc(C4CC4)c(Cl)cc3O)CC2)C1] | G12C |
| | CAS No.: 1629267-75-9 | |
| KRAS inhibitor- 6 | 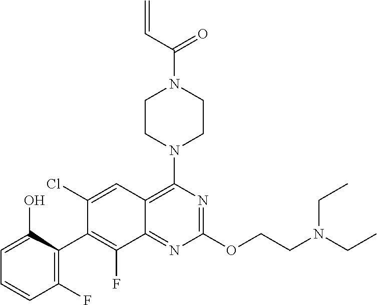 | G12C |
| | CAS No.: 2022986-61-2 | |
| KRAS inhibitor- 7 | 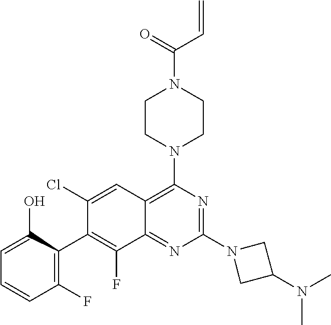 | G12C |
| | CAS No.: 2022986-68-9 | |
| KRAS inhibitor- 8 | 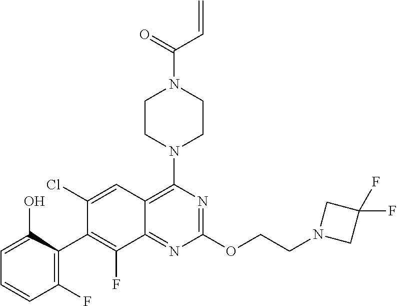 | G12C |
| | CAS No.: 2022986-70-3 | |
| KRAS inhibitor- 9 | 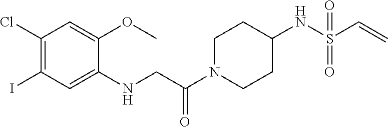 [SMILES: C=CS(=O)(=O)NC1CCN(C(=O)CNc2cc(I)c(Cl)cc2OC)CC1] | G12C |
| | CAS No. 1469337-91-4 | |
| KRAS G12C inhibitor 28 | 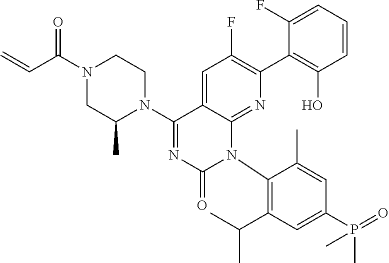 [SMILES: C=CC(=O)N1CCN(c2nc(=O)n(-c3c(C)cc(P(C)(C)=O)cc3C(C)C)c3nc(-c4c(O)cccc4F)c(F)cc23)[C@@H](C)C1] | G12C |
| | CAS No.: 2649004-88-4 | |
| KRAS G12C inhibitor 27 | 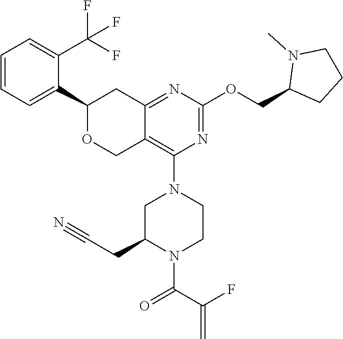 [SMILES: C=C(F)C(=O)N1CCN(c2nc(OC[C@@H]3CCCN3C)nc3c2CO[C@@H](c2ccccc2C(F)(F)F)C3)C[C@@H]1CC#N] | G12C |
| | CAS No.: 2648584-51-2 | |
| KRAS G12C inhibitor 15 | 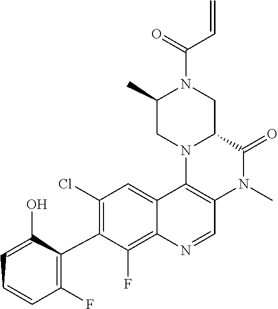 | G12C |
| | CAS No.: 2349393-21-9 | |
| KRAS G12C inhibitor 32 | 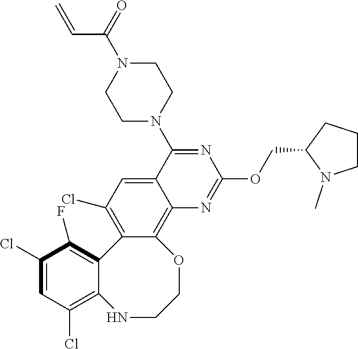 | G12C |
| KRAS G12C inhibitor 5 | 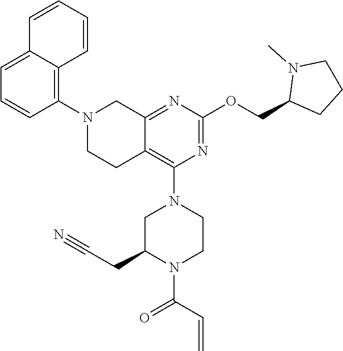 [SMILES: C=CC(=O)N1CCN(c2nc(OC[C@@H]3CCCN3C)nc3c2CCN(c2cccc4ccccc24)C3)C[C@@H]1CC#N] | G12C |
| | CAS No.: 2158297-63-1 | |
| KRAS G12C inhibitor 18 | 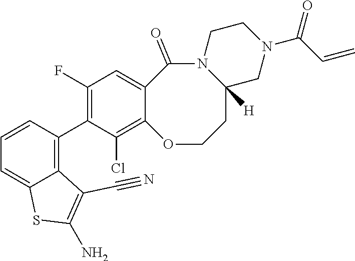 [SMILES: C=CC(=O)N1CCN2C(=O)c3cc(F)c(-c4cccc5sc(N)c(C#N)c45)c(Cl)c3OCC[C@H]2C1] | G12C |
| | CAS No.: 2649788-45-2 | |
| KRAS G12C inhibitor 13 | 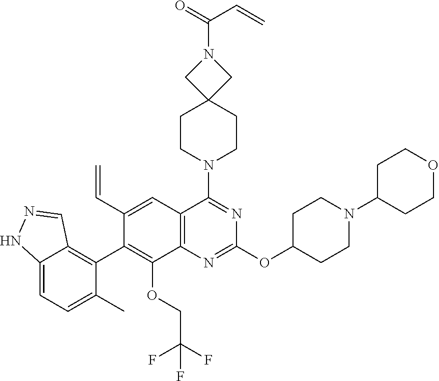 [SMILES: C=CC(=O)N1CC2(CCN(c3nc(OC4CCN(C5CCOCC5)CC4)nc4c(OCC(F)(F)F)c(-c5c(C)ccc6[nH]ncc56)c(C=C)cc34)CC2)C1] | G12C |
| | CAS No.: 2241719-75-3 | |
| KRAS inhibitor- 20 | 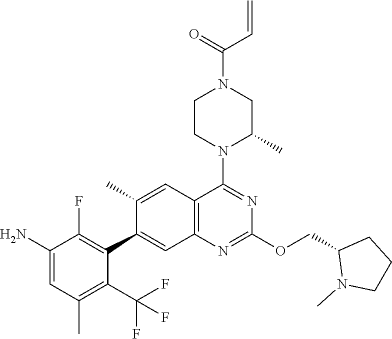 | G12C |
| | CAS No.: 2411786-32-6 | |
| KRAS inhibitor- 16 | 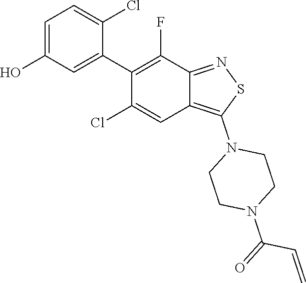 [SMILES: C=CC(=O)N1CCN(c2snc3c(F)c(-c4cc(O)ccc4Cl)c(Cl)cc23)CC1] | G12C |
| | CAS No.: 2230873-67-1 | |
| KRAS G12C inhibitor 17 | 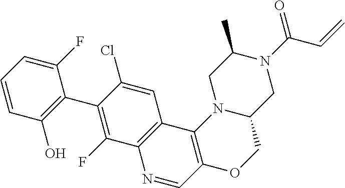 [SMILES: C=CC(=O)N1C[C@@H]2COc3cnc4c(F)c(-c5c(O)cccc5F)c(Cl)cc4c3N2C[C@H]1C] | G12C |
| | CAS No.: 2349393-04-8 | |
| KRAS inhibitor- 14 | 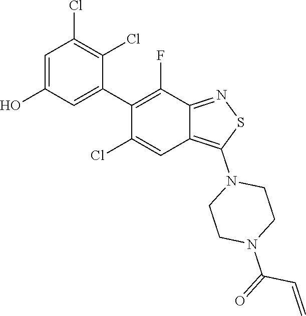 [SMILES: C=CC(=O)N1CCN(c2snc3c(F)c(-c4cc(O)cc(Cl)c4Cl)c(Cl)cc23)CC1] | G12C |
| | CAS No.: 2230873-78-4 | |
| KRAS G12C inhibitor 16 | 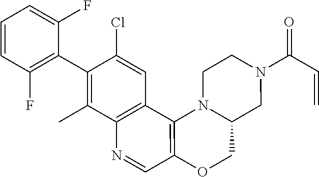 [SMILES: C=CC(=O)N1CCN2c3c(cnc4c(C)c(-c5c(F)cccc5F)c(Cl)cc34)OC[C@H]2C1] | G12C |
| | CAS No.: 2349392-79-4 | |
| KRAS G12C inhibitor 14 | 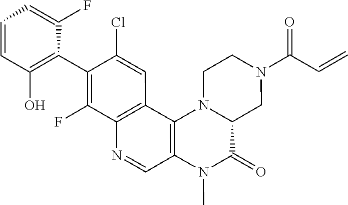 | G12C |
| | CAS No.: 2349393-95-7 | |
| KRAS G12C inhibitor 49 | 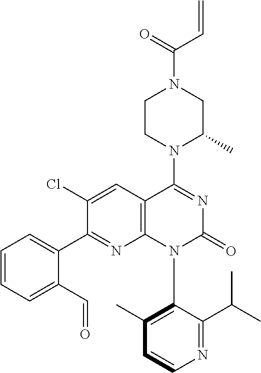 | G12C |
| | CAS No.: 2761380-32-7 | |
| KRAS inhibitor- 13 | 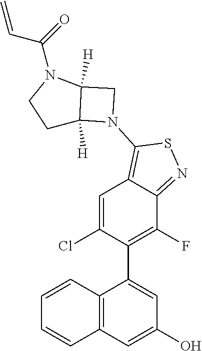 [SMILES: C=CC(=O)N1CC[C@H]2[C@@H]1CN2c1snc2c(F)c(-c3cc(O)cc4ccccc34)c(Cl)cc12] | G12C |
| | CAS No.: 2230873-96-6 | |
| KRAS G12C inhibitor 44 | 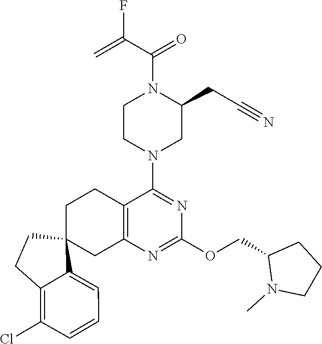 [SMILES: C=C(F)C(=O)N1CCN(c2nc(OC[C@@H]3CCCN3C)nc3c2CC[C@@]2(CCc4c(Cl)cccc42)C3)C[C@@H]1CC#N] | G12C |
| KRAS G12C inhibitor 52 | 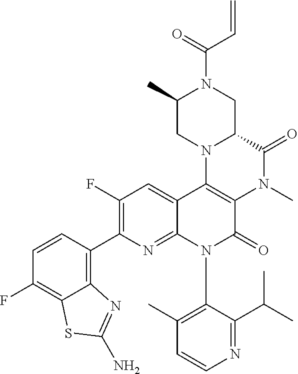 [SMILES: C=CC(=O)N1C[C@@H]2C(=O)N(C)c3c(c4cc(F)c(-c5ccc(F)c6sc(N)nc56)nc4n(-c4c(C)ccnc4C(C)C)c3=O)N2C[C@H]1C] | G12C |
| | CAS No.: 2761204-87-7 | |
| KRAS inhibitor- 12 | 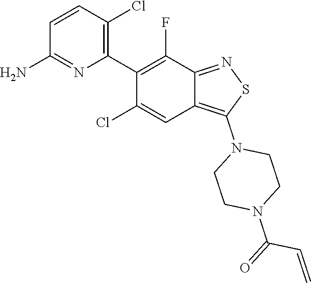 [SMILES: C=CC(=O)N1CCN(c2snc3c(F)c(-c4nc(N)ccc4Cl)c(Cl)cc23)CC1] | G12C |
| | CAS No.: 2230874-00-5 | |
| KRAS G12C inhibitor 46 | 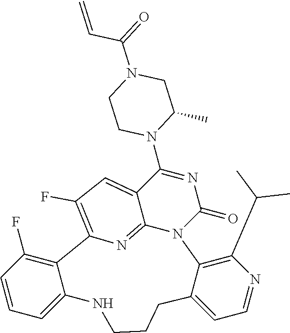 [SMILES: C=CC(=O)N1CCN(c2nc(=O)n3c4nc(c(F)cc24)-c2c(F)cccc2NCCCc2ccnc(C(C)C)c2-3)[C@@H](C)C1] | G12C |
| | CAS No.: 2573769-23-8 | |
| KRAS G12C inhibitor 47 | 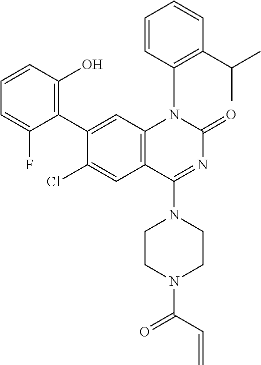 [SMILES: C=CC(=O)N1CCN(c2nc(=O)n(-c3ccccc3C(C)C)c3cc(-c4c(O)cccc4F)c(Cl)cc23)CC1] | G12C |
| | CAS No.: 2253119-24-1 | |
| KRAS inhibitor- 15 | 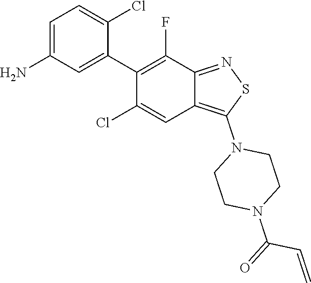 [SMILES: C=CC(=O)N1CCN(c2snc3c(F)c(-c4cc(N)ccc4Cl)c(Cl)cc23)CC1] | G12C |
| | CAS No.: 2230873-75-1 | |
| KRas G12C inhibitor 4 | 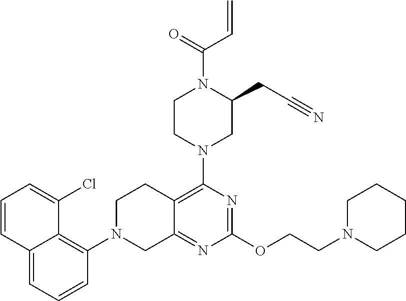 [SMILES: C=CC(=O)N1CCN(c2nc(OCCN3CCCCC3)nc3c2CCN(c2cccc4cccc(Cl)c24)C3)C[C@@H]1CC#N] | G12C |
| | CAS No.: 2206736-07-2 | |
| KRas G12C inhibitor 1 | 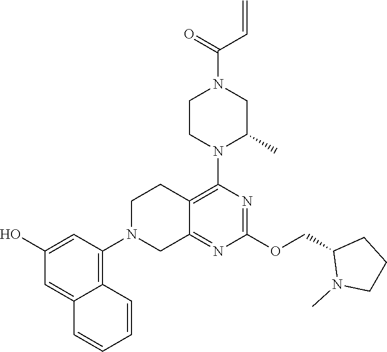 [SMILES: C=CC(=O)N1CCN(c2nc(OC[C@@H]3CCCN3C)nc3c2CCN(c2cc(O)cc4ccccc24)C3)[C@@H](C)C1] | G12C |
| | CAS No.: 2158297-28-8 | |
| KRAS G12C inhibitor 48 | 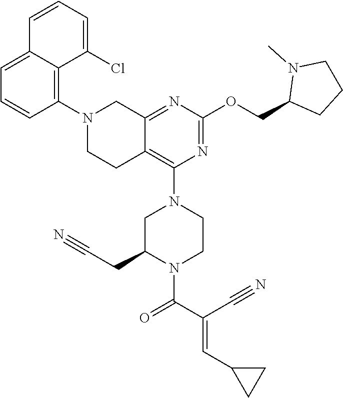 [SMILES: CN1CCC[C@H]1COc1nc2c(c(N3CCN(C(=O)/C(C#N)=C/C4CC4)[C@@H](CC#N)C3)n1)CCN(c1cccc3cccc(Cl)c13)C2] | G12C |
| KRAS G12C inhibitor 23 | 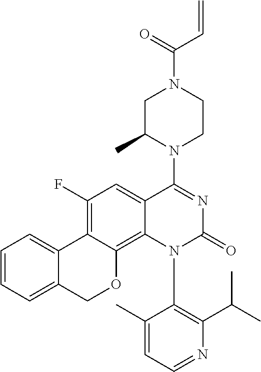 [SMILES: C=CC(=O)N1CCN(c2nc(=O)n(-c3c(C)ccnc3C(C)C)c3c4c(c(F)cc23)-c2ccccc2CO4)[C@@H](C)C1] | G12C |
| | CAS No.: 2735721-00-1 | |
| KRAS G12C inhibitor 25 | 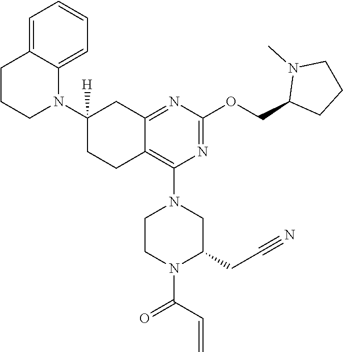 [SMILES: C=CC(=O)N1CCN(c2nc(OC[C@@H]3CCCN3C)nc3c2CC[C@@H](N2CCCc4ccccc42)C3)C[C@@H]1CC#N] | G12C |
| | CAS No.: 2734060-73-0 | |
| K-Ras(G12C) inhibitor 6 | 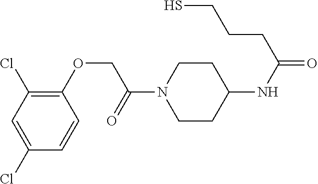 [SMILES: O=C(CCCS)NC1CCN(C(=O)COc2ccc(Cl)cc2Cl)CC1] | G12C |
| | CAS No.: 2060530-16-5 | |
| KRAS inhibitor- 18 | 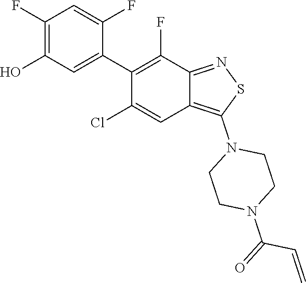 [SMILES: C=CC(=O)N1CCN(c2snc3c(F)c(-c4cc(O)c(F)cc4F)c(Cl)cc23)CC1] | G12C |
| | CAS No.: 2230873-66-0 | |
| KRAS G12C inhibitor 43 | 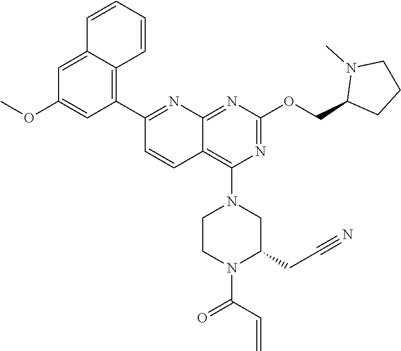 [SMILES: C=CC(=O)N1CCN(c2nc(OC[C@@H]3CCCN3C)nc3nc(-c4cc(OC)cc5ccccc45)ccc23)C[C@@H]1CC#N] | G12C |
| | CAS No.: 2648808-69-7 | |
| KRAS G12C inhibitor 50 | 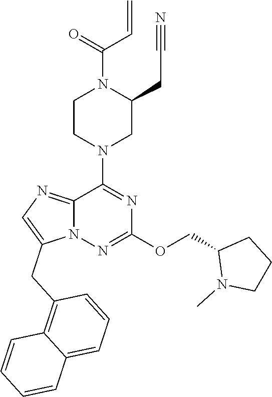 [SMILES: C=CC(=O)N1CCN(c2nc(OC[C@@H]3CCCN3C)nn3c(Cc4cccc5ccccc45)cnc23)C[C@@H]1CC#N] | G12C |
| | CAS No.: 2760354-12-7 | |
| KRAS G12C inhibitor 55 | 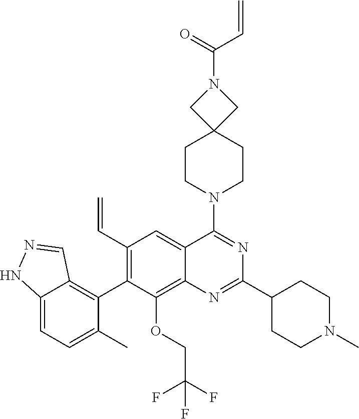 [SMILES: C=CC(=O)N1CC2(CCN(c3nc(C4CCN(C)CC4)nc4c(OCC(F)(F)F)c(-c5c(C)ccc6[nH]ncc56)c(C=C)cc34)CC2)C1] | G12C |
| | CAS No.: 2508134-76-5 | |
| KRAS G12C inhibitor 51 | 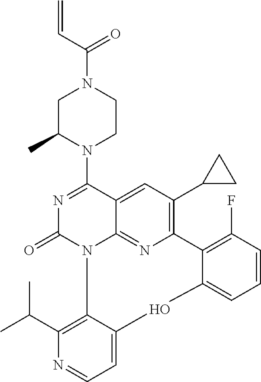 [SMILES: C=CC(=O)N1CCN(c2nc(=O)n3c4nc(c(C5CC5)cc24)-c2c(F)cccc2Oc2ccnc(C(C)C)c2-3)[C@@H](C)C1] | G12C |
| | CAS No.: 2762632-78-8 | |
| KRas G12C inhibitor 3 | 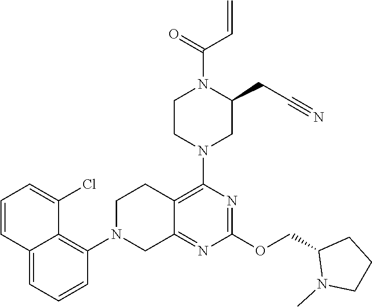 [SMILES: C=CC(=O)N1CCN(c2nc(OC[C@@H]3CCCN3C)nc3c2CCN(c2cccc4cccc(Cl)c24)C3)C[C@@H]1CC#N] | G12C |
| | CAS No.: 2206735-75-1 | |
| K20 | 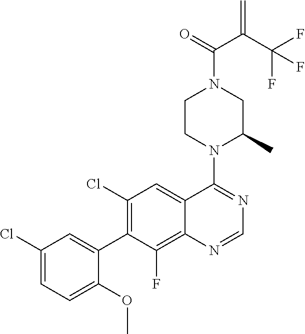 [SMILES: C=C(C(=O)N1CCN(c2ncnc3c(F)c(-c4cc(Cl)ccc4OC)c(Cl)cc23)[C@H](C)C1)C(F)(F)F] | G12C |
| KRAS G12C inhibitor 54 | 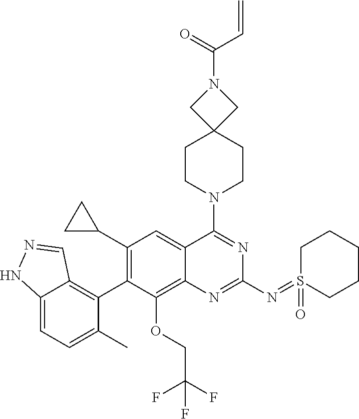 [SMILES: C=CC(=O)N1CC2(CCN(c3nc(N=S4(=O)CCCCC4)nc4c(OCC(F)(F)F)c(-c5c(C)ccc6[nH]ncc56)c(C5CC5)cc34)CC2)C1] | G12C |
| | CAS No.: 2765580-17-2 | |
| KRAS inhibitor- 17 | 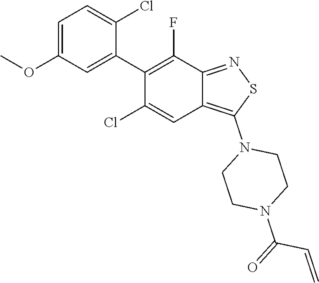 [SMILES: C=CC(=O)N1CCN(c2snc3c(F)c(-c4cc(OC)ccc4Cl)c(Cl)cc23)CC1] | G12C |
| | CAS No.: 2230873-65-9 | |
| KRAS G12C inhibitor 45 | 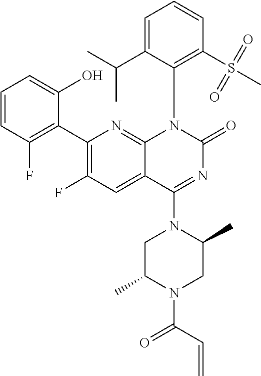 [SMILES: C=CC(=O)N1C[C@H](C)N(c2nc(=O)n(-c3c(C(C)C)cccc3S(C)(=O)=O)c3nc(-c4c(O)cccc4F)c(F)cc23)C[C@H]1C] | G12C |
| | CAS No.: 2670380-74-0 | |
| KRAS G12C inhibitor 2 | 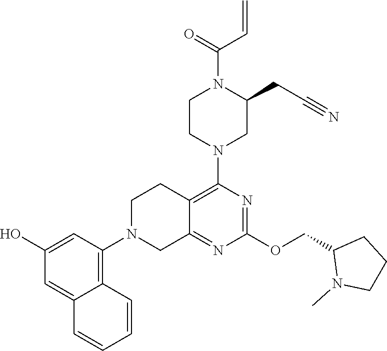 | G12C |
| | CAS No.: 2206735-61-5 | |
| ZG1077 | 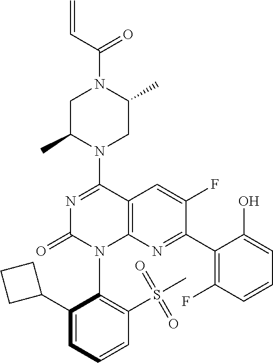 | G12C |
| | CAS No.: 2670380-82-0 | |
| KRAS G12C inhibitor 26 | 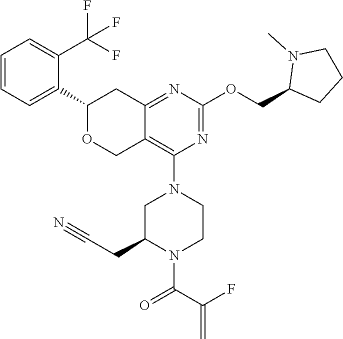 [SMILES: C=C(F)C(=O)N1CCN(c2nc(OC[C@@H]3CCCN3C)nc3c2CO[C@H](c2ccccc2C(F)(F)F)C3)C[C@@H]1CC#N] | G12C |
| | CAS No.: 2648584-52-3 | |
| KRAS G12C inhibitor 53 | 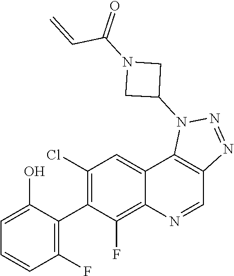 [SMILES: C=CC(=O)N1CC(n2nnc3cnc4c(F)c(-c5c(O)cccc5F)c(Cl)cc4c32)C1] | G12C |
| | CAS No.: 2761968-93-6 | |
| KRAS G12C inhibitor 24 | 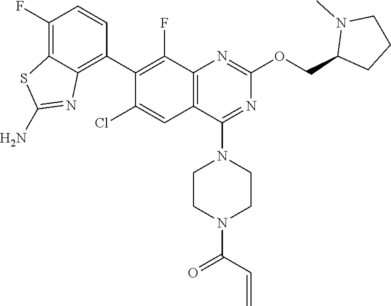 [SMILES: C=CC(=O)N1CCN(c2nc(OC[C@@H]3CCCN3C)nc3c(F)c(-c4ccc(F)c5sc(N)nc45)c(Cl)cc23)CC1] | G12C |
| | CAS No.: 2735742-75-1 | |
| 6H05 | 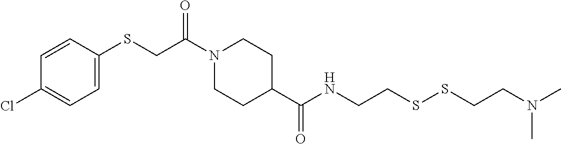 [SMILES: CN(C)CCSSCCNC(=O)C1CCN(C(=O)CSc2ccc(Cl)cc2)CC1] | G12C |
| | CAS No.: 1469338-01-9 | |
| MRTX1133 | 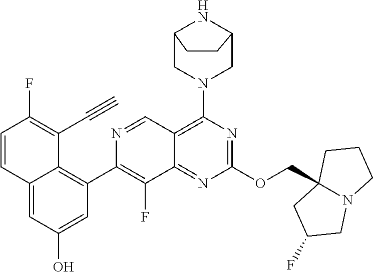 [SMILES: C#Cc1c(F)ccc2cc(O)cc(-c3ncc4c(N5CC6CCC(C5)N6)nc(OC[C@@]56CCCN5C[C@H](F)C6)nc4c3F)c12] | G12D |
| | CAS No.: 2621928-55-8 | |
| MRTX-EX185 formic | 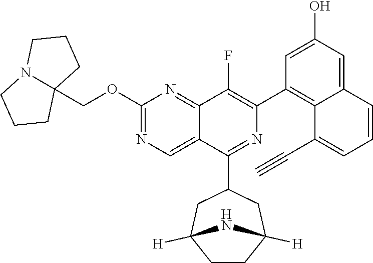 [SMILES: C#Cc1cccc2cc(O)cc(-c3nc(C4C[C@H]5CC[C@@H](C4)N5)c4cnc(OCC56CCCN5CCC6)nc4c3F)c12] | G12D |
| | 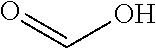 | |
| BI-2852 | 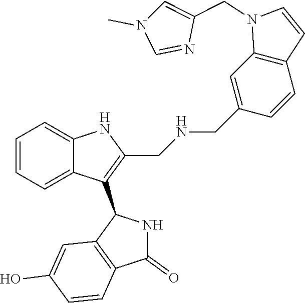 [SMILES: Cn1cnc(Cn2ccc3ccc(CNCc4[nH]c5ccccc5c4[C@H]4NC(=O)c5ccc(O)cc54)cc32)c1] | G12D |
| | CAS No.: 2375482-51-0 | |
| KRAS G12D inhibitor 3 | 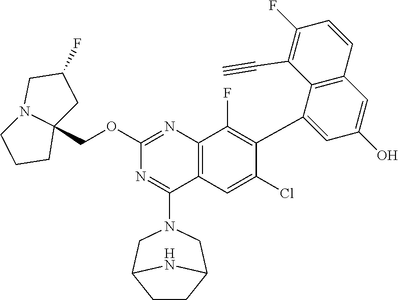 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3F)c12] | G12D |
| | CAS No.: 2757095-11-5 | |
| KRAS G12D inhibitor 14 | 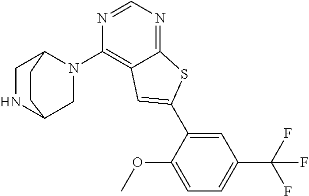 [SMILES: COc1ccc(C(F)(F)F)cc1-c1cc2c(N3CC4CCC3CN4)ncnc2s1] | G12D |
| | CAS No.: 2765254-39-3 | |
| KRAS G12D inhibitor 17 | 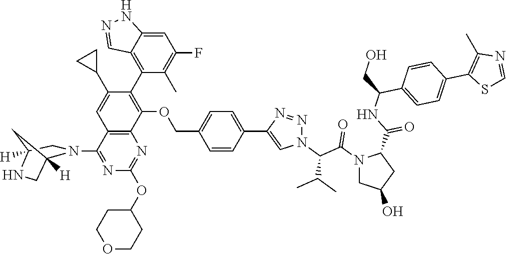 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] | G12D |
| | CAS No.: 2821793-99-9 | |
| KRAS G12D inhibitor 7 | 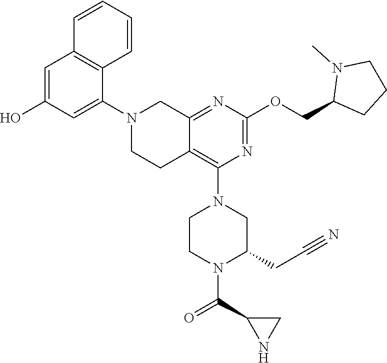 [SMILES: CN1CCC[C@H]1COc1nc2c(c(N3CCN(C(=O)[C@H]4CN4)[C@@H](CC#N)C3)n1)CCN(c1cc(O)cc3ccccc13)C2] | G12D |
| | CAS No.: 2648552-34-3 | |
| KRAS G12D inhibitor 16 | 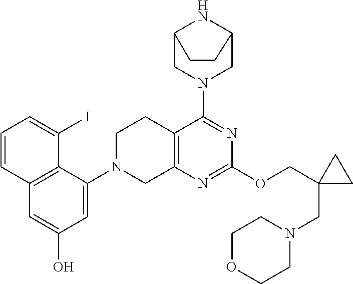 [SMILES: Oc1cc(N2CCc3c(nc(OCC4(CN5CCOCC5)CC4)nc3N3CC4CCC(C3)N4)C2)c2c(I)cccc2c1] | G12D |
| | CAS No.: 2648221-12-7 | |
| KRAS G12D inhibitor 3 TFA | 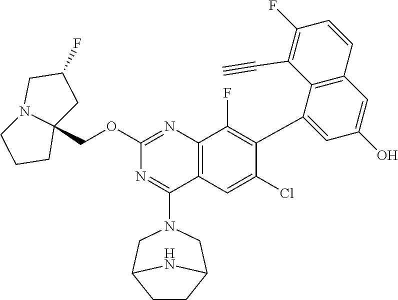 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3F)c12] | G12D |
| | 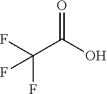 | |
| | CAS No.: 2757095-12-6 | |
| G12Si-5 formic | 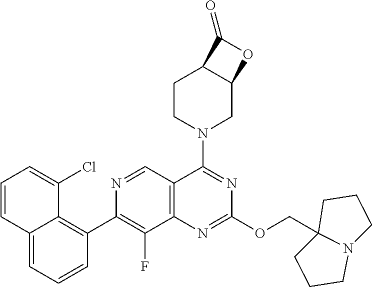 [SMILES: O=C1O[C@H]2CN(c3nc(OCC45CCCN4CCC5)nc4c(F)c(-c5cccc6cccc(Cl)c56)ncc34)CC[C@@H]12] | G12S |
| | 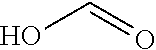 | |
| G12Si-1 | 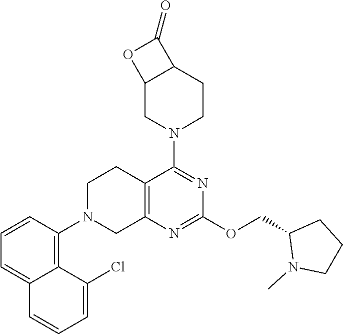 [SMILES: CN1CCC[C@H]1COc1nc2c(c(N3CCC4C(=O)OC4C3)n1)CCN(c1cccc3cccc(Cl)c13)C2] | G12S |
| G12Si-5 | 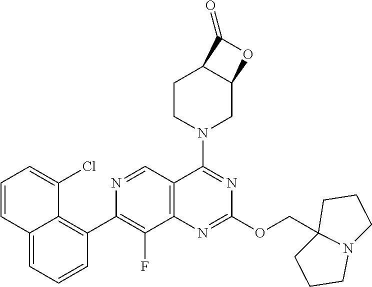 [SMILES: O=C1O[C@H]2CN(c3nc(OCC45CCCN4CCC5)nc4c(F)c(-c5cccc6cccc(Cl)c56)ncc34)CC[C@@H]12] | G12S |
| SOS1-IN-8 | 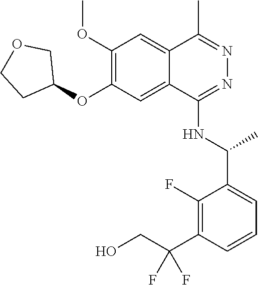 [SMILES: COc1cc2c(C)nnc(N[C@H](C)c3cccc(C(F)(F)CO)c3F)c2cc1O[C@H]1CCOC1] | SOS1 inhibitor for KRAS G12D-SOS1 and KRAS G12V- SOS1 |
| | CAS No.: 2759387-92-1 | |
| SOS1-IN-6 | 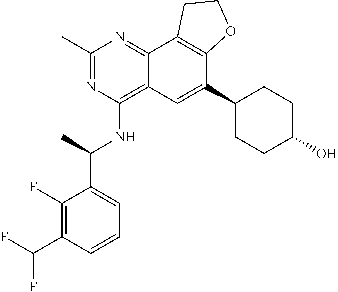 [SMILES: Cc1nc(N[C@H](C)c2cccc(C(F)F)c2F)c2cc([C@H]3CC[C@H](O)CC3)c3c(c2n1)CCO3] | SOS1 inhibitor for KRAS G12D-SOS1 and KRAS G12V- SOS1 |
| | CAS No.: 2751718-88-2 | |
| SOS1-IN-10 | 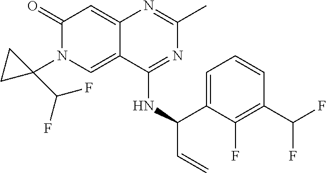 [SMILES: C=C[C@@H](Nc1nc(C)nc2cc(=O)n(C3(C(F)F)CC3)cc12)c1cccc(C(F)F)c1F] | SOS1 inhibitor for KRAS G12C-SOS1 |
| SOS1-IN-4 | 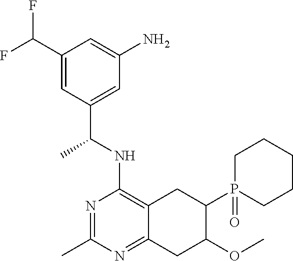 [SMILES: COC1Cc2nc(C)nc(N[C@H](C)c3cc(N)cc(C(F)F)c3)c2CC1P1(=O)CCCCC1] | SOS1 inhibitor for KRAS G12C-SOS1 |
| | CAS No.: 2738392-83-9 | |
| SOS1-IN-9 | 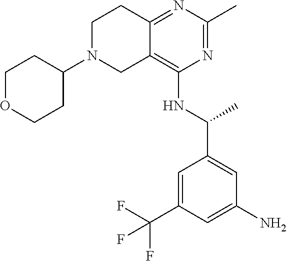 [SMILES: Cc1nc2c(c(N[C@H](C)c3cc(N)cc(C(F)(F)F)c3)n1)CN(C1CCOCC1)CC2] | SOS1 inhibitor for KRAS G12C-SOS1 |
| BI-3406 | 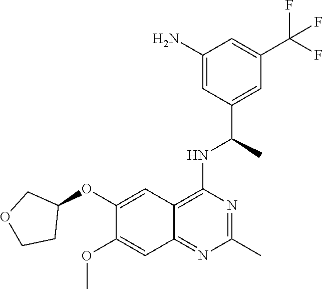 [SMILES: COc1cc2nc(C)nc(N[C@H](C)c3cc(N)cc(C(F)(F)F)c3)c2cc1O[C@H]1CCOC1] | Inhibits interaction of KRAS and SOS1 |
| | CAS No.: 2230836-55-0 | |
| BAY-293 | 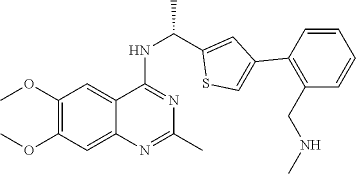 [SMILES: CNCc1ccccc1-c1csc([C@@H](C)Nc2nc(C)nc3cc(OC)c(OC)cc23)c1] | Blocks KRAS activation via disruption of the KRAS-SOS1 interaction |
| | CAS No.: 2244904-70-7 | |
| SOS1-IN-7 | 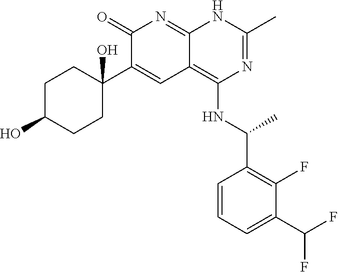 [SMILES: Cc1nc(N[C@H](C)c2cccc(C(F)F)c2F)c2cc([C@]3(O)CC[C@@H](O)CC3)c(=O)nc-2[nH]1] | SOS1 inhibitor |
| | CAS No.: 2755415-30-4 | |
| SAH-SOS1A | RRFFGI(Aaa)LTN(Aaa)LKTEEGN (Covalent bridge:Aaa₇-Aaa₁₁) | G12D, G12V, |
| | | G12C, G12S, and |
| | | Q61H |
| SAH-SOS1A | RRFFGI(Aaa)LTN(Aaa)LKTEEGN (Covalent bridge:Aaa₇-Aaa₁₁) (TFA salt) | G12D, G12V, |
| TFA | | G12C, G12S, and |
| | | Q61H |
| PROTAC K- Ras Degrader-1 | 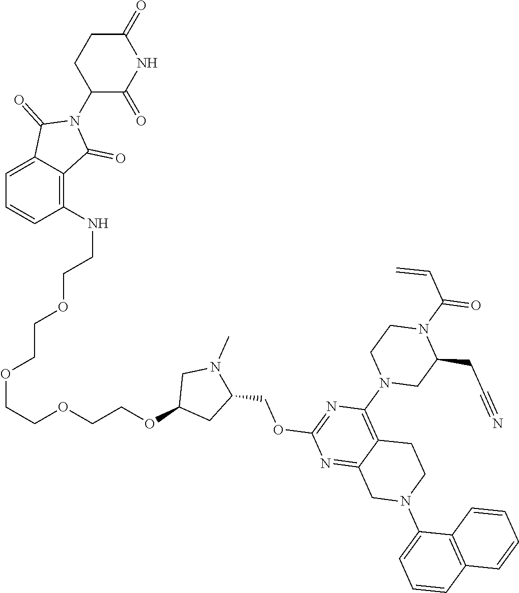 [SMILES: C=CC(=O)N1CCN(c2nc(OC[C@@H]3C[C@@H](OCCOCCOCCOCCNc4cccc5c4C(=O)N(C4CCC(=O)NC4=O)C5=O)CN3C)nc3c2CCN(c2cccc4ccccc24)C3)C[C@@H]1CC#N] | KRAS Degrader |
| | CAS No.: 2378258-52-5 | |
| PROTAC SOS1 degrader-2 | 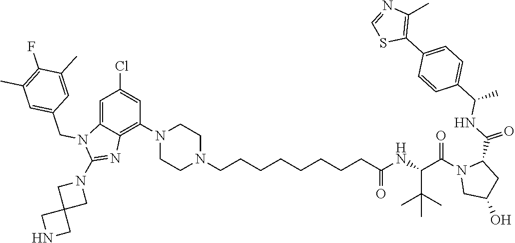 [SMILES: Cc1cc(Cn2c(N3CC4(CNC4)C3)nc3c(N4CCN(CCCCCCCCC(=O)N[C@H](C(=O)N5C[C@@H](O)C[C@H]5C(=O)N[C@@H](C)c5ccc(-c6scnc6C)cc5)C(C)(C)C)CC4)cc(Cl)cc32)cc(C)c1F] | SOS1 degrader |
| KRAS inhibitor- 10 | 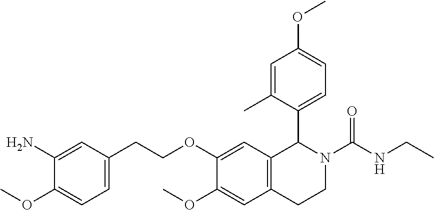 [SMILES: CCNC(=O)N1CCc2cc(OC)c(OCCc3ccc(OC)c(N)c3)cc2C1c1ccc(OC)cc1C] | |
| | CAS No.: 2578876-75-0 | |
| KRAS inhibitor- 11 | 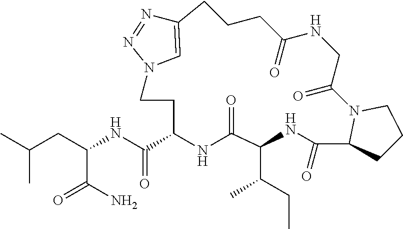 [SMILES: CC[C@H](C)[C@@H]1NC(=O)[C@@H]2CCCN2C(=O)CNC(=O)CCCc2cn(nn2)CC[C@@H](C(=O)N[C@@H](CC(C)C)C(N)=O)NC1=O] | |
| KRpep-2 | Ac-RRRRCPLYISYDPVCRRRR-NH₂ | |
| | (disulfide bridge: Cys₅-Cys₁₅) | |
| | CAS No.: 2098181-84-9 | |
| KRpep-2d TFA | Ac-RRRRCPLYISYDPVCRRRR-NH₂ | |
| | (disulfide bridge: Cys₅-Cys₁₅) (TFA salt) | |
| Pan KRas-IN-1 | 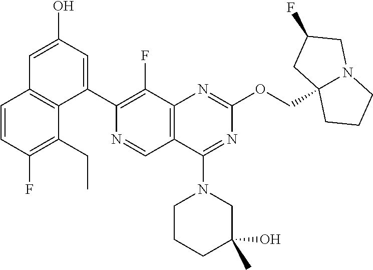 [SMILES: CCc1c(F)ccc2cc(O)cc(-c3ncc4c(N5CCC[C@@](C)(O)C5)nc(OC[C@@]56CCCN5C[C@H](F)C6)nc4c3F)c12] | Pan KRAS inhibitor |
| | CAS No.: 2791263-84- | |

In some embodiments, the KRAS (e.g., hKRAS) inhibitor comprises a KRAS (e.g., hKRAS) inhibitor set forth in Table 2. In some embodiments, the KRAS (e.g., hKRAS) inhibitor comprises a KRAS (e.g., hKRAS) inhibitor set forth in Table 2, that is capable of inhibiting KRAS (e.g., hKRAS) variant G12C. In some embodiments, the KRAS (e.g., hKRAS) inhibitor comprises a KRAS (e.g., hKRAS) inhibitor set forth in Table 2, that is capable of inhibiting KRAS (e.g., hKRAS) variant G12D. In some embodiments, the KRAS (e.g., hKRAS) inhibitor comprises a KRAS (e.g., hKRAS) inhibitor set forth in Table 2, that is capable of inhibiting KRAS (e.g., hKRAS) variant G12S. In some embodiments, the KRAS (e.g., hKRAS) inhibitor comprises a KRAS (e.g., hKRAS) inhibitor set forth in Table 2, that is capable of inhibiting KRAS (e.g., hKRAS) variant G12V. In some embodiments, the KRAS (e.g., hKRAS) inhibitor comprises a KRAS (e.g., hKRAS) inhibitor set forth in Table 2, that is capable of inhibiting KRAS (e.g., hKRAS) variant G12R. In some embodiments, the KRAS (e.g., hKRAS) inhibitor comprises a KRAS (e.g., hKRAS) inhibitor set forth in Table 2, that is capable of inhibiting KRAS (e.g., hKRAS) variant Q61H. In some embodiments, the KRAS inhibitor (e.g., hKRAS) comprises a KRAS (e.g., hKRAS) inhibitor set forth in Table 2, that is capable of inhibiting SOS1.

In some embodiments, the KRAS (e.g., hKRAS) inhibitor comprises a KRAS (e.g., hKRAS) inhibitor set forth in Table 2. In some embodiments, the KRAS (e.g., hKRAS) inhibitor comprises a KRAS (e.g., hKRAS) inhibitor set forth in Table 2, that is capable of selectively inhibiting KRAS (e.g., hKRAS) variant G12C. In some embodiments, the KRAS (e.g., hKRAS) inhibitor comprises a KRAS (e.g., hKRAS) inhibitor set forth in Table 2, that is capable of selectively inhibiting KRAS (e.g., hKRAS) variant G12D. In some embodiments, the KRAS (e.g., hKRAS) inhibitor comprises a KRAS (e.g., hKRAS) inhibitor set forth in Table 2, that is capable of selectively inhibiting KRAS (e.g., hKRAS) variant G12S. In some embodiments, the KRAS (e.g., hKRAS) inhibitor comprises a KRAS (e.g., hKRAS) inhibitor set forth in Table 2, that is capable of selectively inhibiting KRAS (e.g., hKRAS) variant G12V. In some embodiments, the KRAS (e.g., hKRAS) inhibitor comprises a KRAS (e.g., hKRAS) inhibitor set forth in Table 2, that is capable of selectively inhibiting KRAS (e.g., hKRAS) variant G12R. In some embodiments, the KRAS (e.g., hKRAS) inhibitor comprises a KRAS (e.g., hKRAS) inhibitor set forth in Table 2, that is capable of selectively inhibiting KRAS (e.g., hKRAS) variant Q61H. In some embodiments, the KRAS inhibitor (e.g., hKRAS) comprises a KRAS (e.g., hKRAS) inhibitor set forth in Table 2, that is capable of selectively inhibiting SOS1.

In some embodiments, the KRAS (e.g., hKRAS) inhibitor is any one or more of the compounds disclosed herein (see, e.g., § 5.2), a pharmaceutically acceptable salt thereof, a hydrate thereof, a solvate thereof, a polymorph thereof, and a stereochemically isomeric form thereof.

Exemplary salts include, but are not limited to, salts derived from such inorganic bases, e.g., aluminum, ammonium, calcium, copper (ic and ous), ferric, ferrous, lithium, magnesium, manganese (ic and ous), potassium, sodium, zinc and the like salts. Organic bases include salts of primary, secondary, and tertiary amines derived from both naturally occurring and synthetic source. Exemplary organic bases include ammonia, methylamine, ethylamine, propylamine, isopropylamine, a butylamine isomer, betaine, caffeine, choline, dimethylamine, diethylamine, diethanolamine, dipropylamine, diisopropylamine, di-n-butylamine, N, N′-dibenzylethylenediamine, pyrrolidine, piperidine, morpholine, trimethylamine, triethylamine, tripropylamine, tromethamine, 2-diethylaminocthanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, a polyamine resin, procaine, a purine, theobromine, a hydrabamine salt, and a salt with amino acids (e.g., histidine, arginine, and lysine).

In some embodiments, the KRAS (e.g., hKRAS) inhibitor is present as a solvate. In some embodiments, the solvent used to prepare the solvate is an aqueous solution, and the solvate is then often referred to as a hydrate. The KRAS (e.g., hKRAS) inhibitor can be present as a hydrate, which can be obtained, for example, by crystallization from a solvent or from aqueous solution. In this connection, one, two, three or any arbitrary number of solvent or water molecules can combine with the KRAS (e.g., hKRAS) inhibitor to form solvates and hydrates.

### 5.3 EGFR Binding Moieties

The epidermal growth factor receptor (EGFR) is a transmembrane protein that is a receptor for members of the epidermal growth factor family (EGF family) of extracellular protein ligands. Activation of EGFR (e.g., human EGFR (hEGFR)) leads to the activation of several downstream signaling proteins that initiate various signal transduction cascades, including the MAPK, Akt and JNK pathways, leading to DNA synthesis, cell proliferation, cell migration, and adhesion. A number of cancers (e.g., human cancers) are associated with overexpression of EGFR (e.g., hEGFR) relative to non-cancer cells, leading to e.g., overactivation of EGFR (e.g., hEGFR) and increased cell proliferation and growth.

The amino acid sequence of a reference mature hEGFR polypeptide and an immature hEGFR polypeptide is set forth in SEQ ID NOS: 37 and 38, respectively. See Table 3, herein.

TABLE 3
The Amino Acid Sequence of Reference EGFR
Polypeptides.

| | | SEQ |
|---|---|---|
| Descrip- | | ID |
| tion | Amino Acid Sequence | NO |
| hEGFR | LEEKKVCQGTSNKLTQLGTFEDHFLSLQRMF | 37 |
| Mature - | NNCEVVLGNLEITYVQRNYDLSFLKTIQEVA | |
| No | GYVLIALNTVERIPLENLQIIRGNMYYENSY | |
| Signal | ALAVLSNYDANKTGLKELPMRNLQEILHGAV | |
| Peptide | RFSNNPALCNVESIQWRDIVSSDFLSNMSMD | |
| | FQNHLGSCQKCDPSCPNGSCWGAGEENCQKL | |
| | TKIICAQQCSGRCRGKSPSDCCHNQCAAGCT | |
| | GPRESDCLVCRKFRDEATCKDTCPPLMLYNP | |
| | TTYQMDVNPEGKYSFGATCVKKCPRNYVVTD | |
| | HGSCVRACGADSYEMEEDGVRKCKKCEGPCR | |
| | KVCNGIGIGEFKDSLSINATNIKHFKNCTSI | |
| | SGDLHILPVAFRGDSFTHTPPLDPQELDILK | |
| | TVKEITGFLLIQAWPENRTDLHAFENLEIIR | |
| | GRTKQHGQFSLAVVSLNITSLGLRSLKEISD | |
| | GDVIISGNKNLCYANTINWKKLFGTSGQKTK | |
| | IISNRGENSCKATGQVCHALCSPEGCWGPEP | |
| | RDCVSCRNVSRGRECVDKCNLLEGEPREFVE | |
| | NSECIQCHPECLPQAMNITCTGRGPDNCIQC | |
| | AHYIDGPHCVKTCPAGVMGENNTLVWKYADA | |
| | GHVCHLCHPNCTYGCTGPGLEGCPTNGPKIP | |
| | SIATGMVGALLLLLVVALGIGLFMRRRHIVR | |
| | KRTLRRLLQERELVEPLTPSGEAPNQALLRI | |
| | LKETEFKKIKVLGSGAFGTVYKGLWIPEGEK | |
| | VKIPVAIKELREATSPKANKEILDEAYVMAS | |
| | VDNPHVCRLLGICLTSTVQLITQLMPFGCLL | |
| | DYVREHKDNIGSQYLLNWCVQIAKGMNYLED | |
| | RRLVHRDLAARNVLVKTPQHVKITDFGLAKL | |
| | LGAEEKEYHAEGGKVPIKWMALESILHRIYT | |
| | HQSDVWSYGVTVWELMTFGSKPYDGIPASEI | |
| | SSILEKGERLPQPPICTIDVYMIMVKCWMID | |
| | ADSRPKFRELIIEFSKMARDPQRYLVIQGDE | |
| | RMHLPSPTDSNFYRALMDEEDMDDVVDADEY | |
| | LIPQQGFFSSPSTSRTPLLSSLSATSNNSTV | |
| | ACIDRNGLQSCPIKEDSFLQRYSSDPTGALT | |
| | EDSIDDTFLPVPEYINQSVPKRPAGSVQNPV | |
| | YHNQPLNPAPSRDPHYQDPHSTAVGNPEYLN | |
| | TVQPTCVNSTFDSPAHWAQKGSHQISLDNPD | |
| | YQQDFFPKEAKPNGIFKGSTAENAEYLRVAP | |
| | QSSEFIGA | |
| hEGFR | MRPSGTAGAALLALLAALCPASRALEEKKVC | 38 |
| Immature - | QGTSNKLTQLGTFEDHFLSLQRMFNNCEVVL | |
| With | GNLEITYVQRNYDLSFLKTIQEVAGYVLIAL | |
| Signal | NTVERIPLENLQIIRGNMYYENSYALAVLSN | |
| Peptide | YDANKTGLKELPMRNLQEILHGAVRFSNNPA | |
| | LCNVESIQWRDIVSSDFLSNMSMDFQNHLGS | |
| | CQKCDPSCPNGSCWGAGEENCQKLTKIICAQ | |
| | QCSGRCRGKSPSDCCHNQCAAGCTGPRESDC | |
| | LVCRKFRDEATCKDTCPPLMLYNPTTYQMDV | |
| | NPEGKYSFGATCVKKCPRNYVVTDHGSCVRA | |
| | CGADSYEMEEDGVRKCKKCEGPCRKVCNGIG | |
| | IGEFKDSLSINATNIKHFKNCTSISGDLHIL | |
| | PVAFRGDSFTHTPPLDPQELDILKTVKEITG | |
| | FLLIQAWPENRTDLHAFENLEIIRGRTKQHG | |
| | QFSLAVVSLNITSLGLRSLKEISDGDVIISG | |
| | NKNLCYANTINWKKLFGTSGQKTKIISNRGE | |
| | NSCKATGQVCHALCSPEGCWGPEPRDCVSCR | |
| | NVSRGRECVDKCNLLEGEPREFVENSECIQC | |
| | HPECLPQAMNITCTGRGPDNCIQCAHYIDGP | |
| | HCVKTCPAGVMGENNTLVWKYADAGHVCHLC | |
| | HPNCTYGCTGPGLEGCPTNGPKIPSIATGMV | |
| | GALLLLLVVALGIGLFMRRRHIVRKRTLRRL | |
| | LQERELVEPLTPSGEAPNQALLRILKETEFK | |
| | KIKVLGSGAFGTVYKGLWIPEGEKVKIPVAI | |
| | KELREATSPKANKEILDEAYVMASVDNPHVC | |
| | RLLGICLTSTVQLITQLMPFGCLLDYVREHK | |
| | DNIGSQYLLNWCVQIAKGMNYLEDRRLVHRD | |
| | LAARNVLVKTPQHVKITDFGLAKLLGAEEKE | |
| | YHAEGGKVPIKWMALESILHRIYTHQSDVWS | |
| | YGVTVWELMTFGSKPYDGIPASEISSILEKG | |
| | ERLPQPPICTIDVYMIMVKCWMIDADSRPKF | |
| | RELIIEFSKMARDPQRYLVIQGDERMHLPSP | |
| | TDSNFYRALMDEEDMDDVVDADEYLIPQQGF | |
| | FSSPSTSRTPLLSSLSATSNNSTVACIDRNG | |
| | LQSCPIKEDSFLQRYSSDPTGALTEDSIDDT | |
| | FLPVPEYINQSVPKRPAGSVQNPVYHNQPLN | |
| | PAPSRDPHYQDPHSTAVGNPEYLNTVQPTCV | |
| | NSTFDSPAHWAQKGSHQISLDNPDYQQDFFP | |
| | KEAKPNGIFKGSTAENAEYLRVAPQSSEFIG | |
| | A | |

In some aspects and embodiments described herein a moiety that specifically binds EGFR (e.g., hEGFR) is utilized (also referred to herein as an EGFR binding moiety, an EGFR binding domain, or an anti-EGFR moiety or domain). For example, in some aspects and embodiments, a fusion protein (e.g., described herein) comprises a moiety that specifically binds EGFR (e.g., hEGFR).

In some embodiments, the EGFR (e.g., hEGFR) binding moiety comprises an antibody, or functional fragment or functional variant thereof. In some embodiments, the EGFR (e.g., hEGFR) binding moiety comprises a full-length antibody, a scFv, a (scFv)₂, a scFv-Fc, a (scFv)₂-Fc, a Fab, a Fab′, a F(ab′)2, a F(v), a single domain antibody, a single chain antibody, a VHH, a (VHH)₂, a VHH-Fc, or a (VHH)₂-Fc. In some embodiments, the EGFR (e.g., hEGFR) binding moiety comprises a full-length antibody, a scFv, a Fab, or a VHH. In some embodiments, the EGFR (e.g., hEGFR) binding moiety comprises full-length antibody, a single chain variable fragment (scFv), a Fab, or a single domain antibody (sdAb). In some embodiments, the EGFR (e.g., hEGFR) binding moiety comprises a full-length antibody.

In some embodiments, the EGFR (e.g., hEGFR) binding moiety comprises cetuximab (or a functional variant or fragment thereof). In some embodiments, the EGFR (e.g., hEGFR) binding moiety comprises panitumumab (or a functional variant or fragment thereof). In some embodiments, the EGFR (e.g., hEGFR) binding moiety cross-competes with cetuximab. In some embodiments, the EGFR (e.g., hEGFR) binding moiety specifically binds to the same epitope as cetuximab. In some embodiments, the amino acid sequence of the CDRs of the VH and VL of the EGFR (e.g., hEGFR) binding moiety are identical to the amino acid sequence of the CDRs of the VH and VL of cetuximab; or one or more of the CDRs of the VH and VL of the EGFR (e.g., hEGFR) binding moiety comprise or consist of 1, 2, or 3 amino acid modifications relative to the CDRs of the VH and VL of cetuximab.

In some embodiments, the EGFR (e.g., hEGFR) binding moiety comprises panitumumab (or a functional variant or fragment thereof). In some embodiments, the EGFR (e.g., hEGFR) binding moiety comprises panitumumab (or a functional variant or fragment thereof). In some embodiments, the EGFR (e.g., hEGFR) binding moiety cross-competes with panitumumab. In some embodiments, the EGFR (e.g., hEGFR) binding moiety specifically binds to the same epitope as panitumumab. In some embodiments, the amino acid sequence of the CDRs of the VH and VL of the EGFR (e.g., hEGFR) binding moiety are identical to the amino acid sequence of the CDRs of the VH and VL of panitumumab; or one or more of the CDRs of the VH and VL of the EGFR (e.g., hEGFR) binding moiety comprise or consist of 1, 2, or 3 amino acid modifications relative to the CDRs of the VH and VL of panitumumab.

The amino acid sequence of exemplary EGFR (e.g., hEGFR) binding moieties (and component thereof) is provided in Table 4. The CDRs are defined according to the Kabat.

TABLE 4
The Amino Acid Sequence of Exemplary EGFR
Binding Moieties.

| | | | SEQ |
|---|---|---|---|
| | | | ID |
| Description | | Amino Acid Sequence | NO |
| Cetuximab & | VH CDR1 | NYGVH | 39 |
| Variants | VH CDR2 | VIWSGGNTDYNTPFTS | 40 |
| | VH CDR3 | ALTYYDYEFAY | 41 |
| | VL CDR1 | RASQSIGTNIH | 42 |
| | VL CDR2 | YASESIS | 43 |
| | VL CDR3 | QQNNNWPTT | 44 |
| | VH | QVQLKQSGPGLVQPSQSLSIT | 45 |
| | | CTVSGFSLTNYGVHWVRQSPG | |
| | | KGLEWLGVIWSGGNTDYNTPF | |
| | | TSRLSINKDNSKSQVFFKMNS | |
| | | LQSNDTAIYYCARALTYYDYE | |
| | | FAYWGQGTLVTVSA | |
| | VL | DILLTQSPVILSVSPGERVSF | 46 |
| | | SCRASQSIGTNIHWYQQRTNG | |
| | | SPRLLIKYASESISGIPSRFS | |
| | | GSGSGTDFTLSINSVESEDIA | |
| | | DYYCQQNNNWPTTFGAGTKLE | |
| | | LK | |
| | HC | QVQLKQSGPGLVQPSQSLSIT | 47 |
| | | CTVSGFSLTNYGVHWVRQSPG | |
| | | KGLEWLGVIWSGGNTDYNTPF | |
| | | TSRLSINKDNSKSQVFFKMNS | |
| | | LQSNDTAIYYCARALTYYDYE | |
| | | FAYWGQGTLVTVSAASTKGPS | |
| | | VFPLAPSSKSTSGGTAALGCL | |
| | | VKDYFPEPVTVSWNSGALTSG | |
| | | VHTFPAVLQSSGLYSLSSVVT | |
| | | VPSSSLGTQTYICNVNHKPSN | |
| | | TKVDKRVEPKSCDKTHTCPPC | |
| | | PAPELLGGPSVFLFPPKPKDT | |
| | | LMISRTPEVTCVVVDVSHEDP | |
| | | EVKFNWYVDGVEVHNAKTKPR | |
| | | EEQYNSTYRVVSVLTVLHQDW | |
| | | LNGKEYKCKVSNKALPAPIEK | |
| | | TISKAKGQPREPQVYTLPPSR | |
| | | DELTKNQVSLTCLVKGFYPSD | |
| | | IAVEWESNGQPENNYKTTPPV | |
| | | LDSDGSFFLYSKLTVDKSRWQ | |
| | | QGNVFSCSVMHEALHNHYTQK | |
| | | SLSLSPGK | |
| | HC | QVQLKQSGPGLVQPSQSLSIT | 48 |
| | Variant | CTVSGFSLTNYGVHWVRQSPG | |
| | No C- | KGLEWLGVIWSGGNTDYNTPF | |
| | terminal | TSRLSINKDNSKSQVFFKMNS | |
| | Lysine | LQSNDTAIYYCARALTYYDYE | |
| | | FAYWGQGTLVTVSAASTKGPS | |
| | | VFPLAPSSKSTSGGTAALGCL | |
| | | VKDYFPEPVTVSWNSGALTSG | |
| | | VHTFPAVLQSSGLYSLSSVVT | |
| | | VPSSSLGTQTYICNVNHKPSN | |
| | | TKVDKRVEPKSCDKTHTCPPC | |
| | | PAPELLGGPSVFLFPPKPKDT | |
| | | LMISRTPEVTCVVVDVSHEDP | |
| | | EVKFNWYVDGVEVHNAKTKPR | |
| | | EEQYNSTYRVVSVLTVLHQDW | |
| | | LNGKEYKCKVSNKALPAPIEK | |
| | | TISKAKGQPREPQVYTLPPSR | |
| | | DELTKNQVSLTCLVKGFYPSD | |
| | | IAVEWESNGQPENNYKTTPPV | |
| | | LDSDGSFFLYSKLTVDKSRWQ | |
| | | QGNVFSCSVMHEALHNHYTQK | |
| | | SLSLSPG | |
| | LC | DILLTQSPVILSVSPGERVSF | 49 |
| | | SCRASQSIGTNIHWYQQRTNG | |
| | | SPRLLIKYASESISGIPSRFS | |
| | | GSGSGTDFTLSINSVESEDIA | |
| | | DYYCQQNNNWPTTFGAGTKLE | |
| | | LKRTVAAPSVFIFPPSDEQLK | |
| | | SGTASVVCLLNNFYPREAKVQ | |
| | | WKVDNALQSGNSQESVTEQDS | |
| | | KDSTYSLSSTLTLSKADYEKH | |
| | | KVYACEVTHQGLSSPVTKSFN | |
| | | RGEC | |
| Panitumumab | VH CDR1 | SGDYYWT | 50 |
| & Variants | VH CDR2 | HIYYSGNTNYNPSLKS | 51 |
| | VH CDR3 | DRVTGAFDI | 52 |
| | VL CDR1 | QASQDISNYLN | 53 |
| | VL CDR2 | DASNLET | 54 |
| | VL CDR3 | QHFDHLPLA | 55 |
| | VH | QVQLQESGPGLVKPSETLSLT | 56 |
| | | CTVSGGSVSSGDYYWTWIRQS | |
| | | PGKGLEWIGHIYYSGNTNYNP | |
| | | SLKSRLTISIDTSKTQFSLKL | |
| | | SSVTAADTAIYYCVRDRVTGA | |
| | | FDIWGQGTMVTVSS | |
| | VL | DIQMTQSPSSLSASVGDRVTI | 57 |
| | | TCQASQDISNYLNWYQQKPGK | |
| | | APKLLIYDASNLETGVPSRFS | |
| | | GSGSGTDFTFTISSLQPEDIA | |
| | | TYFCQHFDHLPLAFGGGTKVE | |
| | | IK | |
| | HC | QVQLQESGPGLVKPSETLSLT | 58 |
| | | CTVSGGSVSSGDYYWTWIRQS | |
| | | PGKGLEWIGHIYYSGNTNYNP | |
| | | SLKSRLTISIDTSKTQFSLKL | |
| | | SSVTAADTAIYYCVRDRVTGA | |
| | | FDIWGQGTMVTVSSASTKGPS | |
| | | VFPLAPCSRSTSESTAALGCL | |
| | | VKDYFPEPVTVSWNSGALTSG | |
| | | VHTFPAVLQSSGLYSLSSVVT | |
| | | VPSSNFGTQTYTCNVDHKPSN | |
| | | TKVDKTVERKCCVECPPCPAP | |
| | | PVAGPSVFLFPPKPKDTLMIS | |
| | | RTPEVTCVVVDVSHEDPEVQF | |
| | | NWYVDGVEVHNAKTKPREEQF | |
| | | NSTFRVVSVLTVVHQDWLNGK | |
| | | EYKCKVSNKGLPAPIEKTISK | |
| | | TKGQPREPQVYTLPPSREEMT | |
| | | KNQVSLTCLVKGFYPSDIAVE | |
| | | WESNGQPENNYKTTPPMLDSD | |
| | | GSFFLYSKLTVDKSRWQQGNV | |
| | | FSCSVMHEALHNHYTQKSLSL | |
| | | SPGK | |
| | HC | QVQLQESGPGLVKPSETLSLT | 59 |
| | Variant | CTVSGGSVSSGDYYWTWIRQS | |
| | No C- | PGKGLEWIGHIYYSGNTNYNP | |
| | terminal | SLKSRLTISIDTSKTQFSLKL | |
| | Lysine | SSVTAADTAIYYCVRDRVTGA | |
| | | FDIWGQGTMVTVSSASTKGPS | |
| | | VFPLAPCSRSTSESTAALGCL | |
| | | VKDYFPEPVTVSWNSGALTSG | |
| | | VHTFPAVLQSSGLYSLSSVVT | |
| | | VPSSNFGTQTYTCNVDHKPSN | |
| | | TKVDKTVERKCCVECPPCPAP | |
| | | PVAGPSVFLFPPKPKDTLMIS | |
| | | RTPEVTCVVVDVSHEDPEVQF | |
| | | NWYVDGVEVHNAKTKPREEQF | |
| | | NSTFRVVSVLTVVHQDWLNGK | |
| | | EYKCKVSNKGLPAPIEKTISK | |
| | | TKGQPREPQVYTLPPSREEMT | |
| | | KNQVSLTCLVKGFYPSDIAVE | |
| | | WESNGQPENNYKTTPPMLDSD | |
| | | GSFFLYSKLTVDKSRWQQGNV | |
| | | FSCSVMHEALHNHYTQKSLSL | |
| | | SPG | |
| | LC | DIQMTQSPSSLSASVGDRVTI | 60 |
| | | TCQASQDISNYLNWYQQKPGK | |
| | | APKLLIYDASNLETGVPSRFS | |
| | | GSGSGTDFTFTISSLQPEDIA | |
| | | TYFCQHFDHLPLAFGGGTKVE | |
| | | IKRTVAAPSVFIFPPSDEQLK | |
| | | SGTASVVCLLNNFYPREAKVQ | |
| | | WKVDNALQSGNSQESVTEQDS | |
| | | KDSTYSLSSTLTLSKADYEKH | |
| | | KVYACEVTHQGLSSPVTKSFN | |
| | | RGEC | |

In some embodiments, the EGFR (e.g., hEGFR) binding moiety comprises an EGFR (e.g., hEGFR) binding domain provided in Table 4. In some embodiments, the amino acid sequence of the EGFR (e.g., hEGFR) binding moiety comprises the amino acid sequence of the one or more EGFR (e.g., hEGFR) binding moiety components provided in Table 4. In some embodiments, the amino acid sequence of one or more components of the EGFR (e.g., hEGFR) binding moiety is at least 85%, 86%, 87%, 88%, 89%, 90%, 91%, 92%, 93%, 94%, 95%, 96%, 97%, 98%, 99%, or 100% identical to the amino acid sequence of one or more components of an EGFR (e.g., hEGFR) binding moiety in Table 4.

In some embodiments, the EGFR binding moiety comprises a VH that comprises: VH CDR1, VH CDR2, and VH CDR3.

In some embodiments, the amino acid sequence of VH CDR1 comprises or consists of the amino acid sequence of a VH CDR1 of a VH set forth in Table 4, or the amino acid sequence of a VH CDR1 of a VH set forth in Table 4 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 of a VH set forth in Table 4, or the amino acid sequence of a VH CDR2 of a VH set forth in Table 4 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 of a VH set forth in Table 4, or the amino acid sequence of a VH CDR3 of a VH set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 of a VH set forth in Table 4, or the amino acid sequence of a VH CDR1 of a VH set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 4, or the amino acid sequence of a VH CDR2 of a VH set forth in Table 4 comprising or consisting of 1, 2, or 3 amino acid modifications (e.g., substitution, deletion, addition, etc.); and the amino acid sequence of VH CDR3 comprises or consists of the amino acid sequence of a VH CDR3 of a VH set forth in Table 4, or the amino acid sequence of a VH CDR3 of a VH set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 4, or the amino acid sequence of a VH CDR1 set forth in Table 4 comprising or consisting of 1, 2, or 3 amino acid modifications (e.g., substitution, deletion, addition, etc.). In some embodiments, the amino acid sequence of VH CDR2 comprises or consists of the amino acid sequence of a VH CDR2 set forth in Table 4, or the amino acid sequence of a VH CDR2 set forth in Table 4 comprising or consisting of 1, 2, or 3 amino acid modifications (e.g., substitution, deletion, addition, etc.). In some embodiments, the amino acid sequence of VH CDR3 comprises or consists of the amino acid sequence of a VH CDR3 set forth in Table 4, or the amino acid sequence of a VH CDR3 set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 4, or the amino acid sequence of a VH CDR1 set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 4, or the amino acid sequence of a VH CDR2 set forth in Table 4 comprising or consisting of 1, 2, or 3 amino acid modifications (e.g., substitution, deletion, addition, etc.); and the amino acid sequence of VH CDR3 comprises or consists of the amino acid sequence of a VH CDR3 set forth in Table 4, or the amino acid sequence of a VH CDR3 set forth in Table 4 comprising or consisting of 1, 2, or 3 amino acid modifications (e.g., substitution, deletion, addition, etc.).

In some embodiments, the EGFR binding moiety comprises a VL that comprises: VL CDR1, VL CDR2, and VL CDR3.

In some embodiments, the amino acid sequence of VL CDR1 comprises or consists of the amino acid sequence of a VL CDR1 of a VL set forth in Table 4, or the amino acid sequence of a VL CDR1 of a VL set forth in Table 4 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 of a VL set forth in Table 4, or the amino acid sequence of a VL CDR2 of a VL set forth in Table 4 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 of a VL set forth in Table 4, or the amino acid sequence of a VL CDR3 of a VL set forth in Table 4 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 of a VL set forth in Table 4, or the amino acid sequence of a VL CDR1 of a VL set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 4, or the amino acid sequence of a VL CDR2 of a VL set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 4, or the amino acid sequence of a VL CDR3 of a VL set forth in Table 4 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 4, or the amino acid sequence of a VL CDR1 set forth in Table 4 comprising or consisting of 1, 2, or 3 amino acid modifications (e.g., substitution, deletion, addition, etc.). In some embodiments, the amino acid sequence of VL CDR2 comprises or consists of the amino acid sequence of a VL CDR2 set forth in Table 4, or the amino acid sequence of a VL CDR2 set forth in Table 4 comprising or consisting of 1, 2, or 3 amino acid modifications (e.g., substitution, deletion, addition, etc.). In some embodiments, the amino acid sequence of VL CDR3 comprises or consists of the amino acid sequence of a VL CDR3 set forth in Table 4, or the amino acid sequence of a VL CDR3 set forth in Table 4 comprising or consisting of 1, 2, or 3 amino acid modifications (e.g., substitution, deletion, addition, etc.).

In some embodiments, the amino acid sequence of VL CDR1 comprises or consists of the amino acid sequence of a VL CDR1 set forth in Table 4, or the amino acid sequence of a VL CDR1 set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 4, or the amino acid sequence of a VL CDR2 set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 4, or the amino acid sequence of a VL CDR3 set forth in Table 4 comprising or consisting of 1, 2, or 3 amino acid modifications (e.g., substitution, deletion, addition, etc.).

In some embodiments, the EGFR binding moiety comprises a VH that comprises: VH CDR1, VH CDR2, and VH CDR3; and a VL that comprises: VL CDR1, VL CDR2, and VL CDR3.

In some embodiments, the amino acid sequence of VH CDR1 comprises or consists of the amino acid sequence of a VH CDR1 of a VH set forth in Table 4, or the amino acid sequence of a VH CDR1 of a VH set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 of a VH set forth in Table 4, or the amino acid sequence of a VH CDR2 of a VH set forth in Table 4 comprising or consisting of 1, 2, or 3 amino acid modifications (e.g., substitution, deletion, addition, etc.); the amino acid sequence of VH CDR3 comprises or consists of the amino acid sequence of a VH CDR3 of a VH set forth in Table 4, or the amino acid sequence of a VH CDR3 of a VH set forth in Table 4 comprising or consisting of 1, 2, or 3 amino acid modifications (e.g., substitution, deletion, addition, etc.); the amino acid sequence of VL CDR1 comprises or consists of the amino acid sequence of a VL CDR1 of a VL set forth in Table 4, or the amino acid sequence of a VL CDR1 of a VL set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 of a VL set forth in Table 4, or the amino acid sequence of a VL CDR2 of a VL set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 of a VL set forth in Table 4, or the amino acid sequence of a VL CDR3 of a VL set forth in Table 4 comprising or consisting of 1, 2, or 3 amino acid modifications (e.g., substitution, deletion, addition, etc.).

In some embodiments, the amino acid sequence of VH CDR1 comprises or consists of the amino acid sequence of a VH CDR1 set forth in Table 4, or the amino acid sequence of a VH CDR1 set forth in Table 4 comprising or consisting of 1, 2, or 3 amino acid modifications (e.g., substitution, deletion, addition, etc.); the amino acid sequence of VH CDR2 comprises or consists of the amino acid sequence of a VH CDR2 set forth in Table 4, or the amino acid sequence of a VH CDR2 set forth in Table 4 comprising or consisting of 1, 2, or 3 amino acid modifications (e.g., substitution, deletion, addition, etc.); the amino acid sequence of VH CDR3 comprises or consists of the amino acid sequence of a VH CDR3 set forth in Table 4, or the amino acid sequence of a VH CDR3 set forth in Table 4 comprising or consisting of 1, 2, or 3 amino acid modifications (e.g., substitution, deletion, addition, etc.); the amino acid sequence of VL CDR1 comprises or consists of the amino acid sequence of a VL CDR1 set forth in Table 4, or the amino acid sequence CDR1 of a VL set forth in Table 4 comprising or consisting of 1, 2, or 3 amino acid modifications (e.g., substitution, deletion, addition, etc.); the amino acid sequence of VL CDR2 comprises or consists of the amino acid sequence of a VL CDR2 set forth in Table 4, or the amino acid sequence of a VL CDR2 set forth in Table 4 comprising or consisting of 1, 2, or 3 amino acid modifications (e.g., substitution, deletion, addition, etc.); and the amino acid sequence of VL CDR3 comprises or consists of the amino acid sequence of a VL CDR3 set forth in Table 4, or the amino acid sequence of a VL CDR3 set forth in Table 4 comprising or consisting of 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 4. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 4. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 4; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 4.

In some embodiments, the EGFR (e.g., hEGFR) binding moiety comprises a heavy chain (HC) that comprises or consists of an amino acid sequence at least 90%, 91%, 92%, 93%, 94%, 95%, 96%, 97%, 98%, 99%, or 100% identical to the amino acid sequence of a heavy chain in Table 4. In some embodiments, the EGFR (e.g., hEGFR) binding moiety comprises a light chain (LC) that comprises or consists of an amino acid sequence at least 90%, 91%, 92%, 93%, 94%, 95%, 96%, 97%, 98%, 99%, or 100% identical to the amino acid sequence of a heavy chain in Table 4. In some embodiments, the EGFR (e.g., hEGFR) binding moiety comprises a heavy chain (HC) that comprises or consists of an amino acid sequence at least 90%, 91%, 92%, 93%, 94%, 95%, 96%, 97%, 98%, 99%, or 100% identical to the amino acid sequence of a heavy chain in Table 4; and a light chain that comprises or consists of an amino acid sequence at least 90%, 91%, 92%, 93%, 94%, 95%, 96%, 97%, 98%, 99%, or 100% identical to the amino acid sequence of a heavy chain in Table 4.

In some embodiments, the amino acid sequence of VH CDR1 comprises the amino acid sequence set forth in SEQ ID NO: 39, or the amino acid sequence set forth in SEQ ID NO: 39 comprising 1, 2, or 3 amino acid modifications (e.g., substitution, deletion, addition, etc.). In some embodiments, the amino acid sequence of VH CDR2 comprises the amino acid sequence set forth in SEQ ID NO: 40, or the amino acid sequence set forth in SEQ ID NO: 40 comprising 1, 2, or 3 amino acid modifications (e.g., substitution, deletion, addition, etc.). In some embodiments, the amino acid sequence of VH CDR3 comprises the amino acid sequence set forth in SEQ ID NO: 41, or the amino acid sequence set forth in SEQ ID NO: 41 comprising 1, 2, or 3 amino acid modifications (e.g., substitution, deletion, addition, etc.).

In some embodiments, the amino acid sequence of VH CDR1 comprises the amino acid sequence set forth in SEQ ID NO: 39, or the amino acid sequence set forth in SEQ ID NO: 39 comprising 1, 2, or 3 amino acid modifications (e.g., substitution, deletion, addition, etc.); the amino acid sequence of VH CDR2 comprises the amino acid sequence set forth in SEQ ID NO: 40, or the amino acid sequence set forth in SEQ ID NO: 40 comprising 1, 2, or 3 amino acid modifications (e.g., substitution, deletion, addition, etc.); and the amino acid sequence of VH CDR3 comprises the amino acid sequence set forth in SEQ ID NO: 41, or the amino acid sequence set forth in SEQ ID NO: 41 comprising 1, 2, or 3 amino acid modifications (e.g., substitution, deletion, addition, etc.).

In some embodiments, the amino acid sequence of VL CDR1 comprises the amino acid sequence set forth in SEQ ID NO: 42, or the amino acid sequence set forth in SEQ ID NO: 42 comprising 1, 2, or 3 amino acid modifications (e.g., substitution, deletion, addition, etc.). In some embodiments, the amino acid sequence of VL CDR2 comprises the amino acid sequence set forth in SEQ ID NO: 43, or the amino acid sequence set forth in SEQ ID NO: 43 comprising 1, 2, or 3 amino acid modifications (e.g., substitution, deletion, addition, etc.). In some embodiments, the amino acid sequence of VL CDR3 comprises the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of VL CDR1 comprises the amino acid sequence set forth in SEQ ID NO: 42, or the amino acid sequence set forth in SEQ ID NO: 42 comprising 1, 2, or 3 amino acid modifications (e.g., substitution, deletion, addition, etc.); the amino acid sequence of VL CDR2 comprises the amino acid sequence set forth in SEQ ID NO: 43, or the amino acid sequence set forth in SEQ ID NO: 43 comprising 1, 2, or 3 amino acid modifications (e.g., substitution, deletion, addition, etc.); and the amino acid sequence of VL CDR3 comprises the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of VH CDR1 comprises the amino acid sequence set forth in SEQ ID NO: 39, or the amino acid sequence set forth in SEQ ID NO: 39 comprising 1, 2, or 3 amino acid modifications (e.g., substitution, deletion, addition, etc.); the amino acid sequence of VH CDR2 comprises the amino acid sequence set forth in SEQ ID NO: 40, or the amino acid sequence set forth in SEQ ID NO: 40 comprising 1, 2, or 3 amino acid modifications (e.g., substitution, deletion, addition, etc.); the amino acid sequence of VH CDR3 comprises the amino acid sequence set forth in SEQ ID NO: 41, or the amino acid sequence set forth in SEQ ID NO: 41 comprising 1, 2, or 3 amino acid modifications (e.g., substitution, deletion, addition, etc.); the amino acid sequence of VL CDR1 comprises the amino acid sequence set forth in SEQ ID NO: 42, or the amino acid sequence set forth in SEQ ID NO: 42 comprising 1, 2, or 3 amino acid modifications (e.g., substitution, deletion, addition, etc.); the amino acid sequence of VL CDR2 comprises the amino acid sequence set forth in SEQ ID NO: 43, or the amino acid sequence set forth in SEQ ID NO: 43 comprising 1, 2, or 3 amino acid modifications (e.g., substitution, deletion, addition, etc.); and the amino acid sequence of VL CDR3 comprises the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45. In some embodiments, the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46. In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46.

In some embodiments, the amino acid sequence of the heavy chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47. In some embodiments, the amino acid sequence of the heavy chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48. In some embodiments, the amino acid sequence of the light chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 49. In some embodiments, the amino acid sequence of the heavy chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47; and the amino acid sequence of the light chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 49. In some embodiments, the amino acid sequence of the heavy chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48; and the amino acid sequence of the light chain comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 49.

In some embodiments, the amino acid sequence of VH CDR1 consists of the amino acid sequence set forth in SEQ ID NO: 39, or the amino acid sequence set forth in SEQ ID NO: 39 comprising 1, 2, or 3 amino acid modifications (e.g., substitution, deletion, addition, etc.). In some embodiments, the amino acid sequence of VH CDR2 consists of the amino acid sequence set forth in SEQ ID NO: 40, or the amino acid sequence set forth in SEQ ID NO: 40 comprising 1, 2, or 3 amino acid modifications (e.g., substitution, deletion, addition, etc.). In some embodiments, the amino acid sequence of VH CDR3 consists of the amino acid sequence set forth in SEQ ID NO: 41, or the amino acid sequence set forth in SEQ ID NO: 41 comprising 1, 2, or 3 amino acid modifications (e.g., substitution, deletion, addition, etc.).

In some embodiments, the amino acid sequence of VH CDR1 consists of the amino acid sequence set forth in SEQ ID NO: 39, or the amino acid sequence set forth in SEQ ID NO: 39 comprising 1, 2, or 3 amino acid modifications (e.g., substitution, deletion, addition, etc.); the amino acid sequence of VH CDR2 consists of the amino acid sequence set forth in SEQ ID NO: 40, or the amino acid sequence set forth in SEQ ID NO: 40 comprising 1, 2, or 3 amino acid modifications (e.g., substitution, deletion, addition, etc.); and the amino acid sequence of VH CDR3 consists of the amino acid sequence set forth in SEQ ID NO: 41, or the amino acid sequence set forth in SEQ ID NO: 41 comprising 1, 2, or 3 amino acid modifications (e.g., substitution, deletion, addition, etc.).

In some embodiments, the amino acid sequence of VL CDR1 consists of the amino acid sequence set forth in SEQ ID NO: 42, or the amino acid sequence set forth in SEQ ID NO: 42 comprising 1, 2, or 3 amino acid modifications (e.g., substitution, deletion, addition, etc.). In some embodiments, the amino acid sequence of VL CDR2 consists of the amino acid sequence set forth in SEQ ID NO: 43, or the amino acid sequence set forth in SEQ ID NO: 43 comprising 1, 2, or 3 amino acid modifications (e.g., substitution, deletion, addition, etc.). In some embodiments, the amino acid sequence of VL CDR3 consists of the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of VL CDR1 consists of the amino acid sequence set forth in SEQ ID NO: 42, or the amino acid sequence set forth in SEQ ID NO: 42 comprising 1, 2, or 3 amino acid modifications (e.g., substitution, deletion, addition, etc.); the amino acid sequence of VL CDR2 consists of the amino acid sequence set forth in SEQ ID NO: 43, or the amino acid sequence set forth in SEQ ID NO: 43 comprising 1, 2, or 3 amino acid modifications (e.g., substitution, deletion, addition, etc.); and the amino acid sequence of VL CDR3 consists of the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of VH CDR1 consists of the amino acid sequence set forth in SEQ ID NO: 39, or the amino acid sequence set forth in SEQ ID NO: 39 comprising 1, 2, or 3 amino acid modifications (e.g., substitution, deletion, addition, etc.); the amino acid sequence of VH CDR2 consists of the amino acid sequence set forth in SEQ ID NO: 40, or the amino acid sequence set forth in SEQ ID NO: 40 comprising 1, 2, or 3 amino acid modifications (e.g., substitution, deletion, addition, etc.); the amino acid sequence of VH CDR3 consists of the amino acid sequence set forth in SEQ ID NO: 41, or the amino acid sequence set forth in SEQ ID NO: 41 comprising 1, 2, or 3 amino acid modifications (e.g., substitution, deletion, addition, etc.); the amino acid sequence of VL CDR1 consists of the amino acid sequence set forth in SEQ ID NO: 42, or the amino acid sequence set forth in SEQ ID NO: 42 comprising 1, 2, or 3 amino acid modifications (e.g., substitution, deletion, addition, etc.); the amino acid sequence of VL CDR2 consists of the amino acid sequence set forth in SEQ ID NO: 43, or the amino acid sequence set forth in SEQ ID NO: 43 comprising 1, 2, or 3 amino acid modifications (e.g., substitution, deletion, addition, etc.); and the amino acid sequence of VL CDR3 consists of the amino acid sequence set forth in SEQ ID NO: 44, or the amino acid sequence set forth in SEQ ID NO: 44 comprising 1, 2, or 3 amino acid modifications (e.g., substitution, deletion, addition, etc.).

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45. In some embodiments, the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46. In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 45; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 46.

In some embodiments, the amino acid sequence of the heavy chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47. In some embodiments, the amino acid sequence of the heavy chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48. In some embodiments, the amino acid sequence of the light chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 49. In some embodiments, the amino acid sequence of the heavy chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 47; and the amino acid sequence of the light chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 49. In some embodiments, the amino acid sequence of the heavy chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 48; and the amino acid sequence of the light chain consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 49.

In some embodiments, the amino acid sequence of VH CDR1 comprises or consists of the amino acid sequence set forth in SEQ ID NO: 50, or the amino acid sequence set forth in SEQ ID NO: 50 comprising 1, 2, or 3 amino acid modifications (e.g., substitution, deletion, addition, etc.). In some embodiments, the amino acid sequence of VH CDR2 comprises or consists of the amino acid sequence set forth in SEQ ID NO: 51, or the amino acid sequence set forth in SEQ ID NO: 51 comprising 1, 2, or 3 amino acid modifications (e.g., substitution, deletion, addition, etc.). In some embodiments, the amino acid sequence of VH CDR3 comprises or consists of the amino acid sequence set forth in SEQ ID NO: 52, or the amino acid sequence set forth in SEQ ID NO: 52 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 50, or the amino acid sequence set forth in SEQ ID NO: 50 comprising 1, 2, or 3 amino acid modifications (e.g., substitution, deletion, addition, etc.); the amino acid sequence of VH CDR2 comprises or consists of the amino acid sequence set forth in SEQ ID NO: 51, or the amino acid sequence set forth in SEQ ID NO: 51 comprising 1, 2, or 3 amino acid modifications (e.g., substitution, deletion, addition, etc.); and the amino acid sequence of VH CDR3 comprises or consists of the amino acid sequence set forth in SEQ ID NO: 52, or the amino acid sequence set forth in SEQ ID NO: 52 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 53, or the amino acid sequence set forth in SEQ ID NO: 53 comprising 1, 2, or 3 amino acid modifications (e.g., substitution, deletion, addition, etc.). In some embodiments, the amino acid sequence of VL CDR2 comprises or consists of the amino acid sequence set forth in SEQ ID NO: 54, or the amino acid sequence set forth in SEQ ID NO: 54 comprising 1, 2, or 3 amino acid modifications (e.g., substitution, deletion, addition, etc.). In some embodiments, the amino acid sequence of VL CDR3 comprises or consists of the amino acid sequence set forth in SEQ ID NO: 55, or the amino acid sequence set forth in SEQ ID NO: 55 comprising 1, 2, or 3 amino acid modifications (e.g., substitution, deletion, addition, etc.).

In some embodiments, the amino acid sequence of VL CDR1 comprises or consists of the amino acid sequence set forth in SEQ ID NO: 53, or the amino acid sequence set forth in SEQ ID NO: 53 comprising 1, 2, or 3 amino acid modifications (e.g., substitution, deletion, addition, etc.); the amino acid sequence of VL CDR2 comprises or consists of the amino acid sequence set forth in SEQ ID NO: 54, or the amino acid sequence set forth in SEQ ID NO: 54 comprising 1, 2, or 3 amino acid modifications (e.g., substitution, deletion, addition, etc.); and the amino acid sequence of VL CDR3 comprises or consists of the amino acid sequence set forth in SEQ ID NO: 55, or the amino acid sequence set forth in SEQ ID NO: 55 comprising 1, 2, or 3 amino acid modifications (e.g., substitution, deletion, addition, etc.).

In some embodiments, the amino acid sequence of VH CDR1 comprises or consists of the amino acid sequence set forth in SEQ ID NO: 50, or the amino acid sequence set forth in SEQ ID NO: 50 comprising 1, 2, or 3 amino acid modifications (e.g., substitution, deletion, addition, etc.); the amino acid sequence of VH CDR2 comprises or consists of the amino acid sequence set forth in SEQ ID NO: 51, or the amino acid sequence set forth in SEQ ID NO: 51 comprising 1, 2, or 3 amino acid modifications (e.g., substitution, deletion, addition, etc.); the amino acid sequence of VH CDR3 comprises or consists of the amino acid sequence set forth in SEQ ID NO: 52, or the amino acid sequence set forth in SEQ ID NO: 52 comprising 1, 2, or 3 amino acid modifications (e.g., substitution, deletion, addition, etc.); the amino acid sequence of VL CDR1 comprises or consists of the amino acid sequence set forth in SEQ ID NO: 53, or the amino acid sequence set forth in SEQ ID NO: 53 comprising 1, 2, or 3 amino acid modifications (e.g., substitution, deletion, addition, etc.); the amino acid sequence of VL CDR2 comprises or consists of the amino acid sequence set forth in SEQ ID NO: 54, or the amino acid sequence set forth in SEQ ID NO: 54 comprising 1, 2, or 3 amino acid modifications (e.g., substitution, deletion, addition, etc.); and the amino acid sequence of VL CDR3 comprises or consists of the amino acid sequence set forth in SEQ ID NO: 55, or the amino acid sequence set forth in SEQ ID NO: 55 comprising 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 56. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 57. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 56; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 57.

In some embodiments, the amino acid sequence of the heavy chain comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 58. In some embodiments, the amino acid sequence of the heavy chain comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 59. In some embodiments, the amino acid sequence of the light chain comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 60. In some embodiments, the amino acid sequence of the heavy chain comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 58; and the amino acid sequence of the light chain comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 60. In some embodiments, the amino acid sequence of the heavy chain comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 59; and the amino acid sequence of the light chain comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 60.

### 5.4 TGFβ Binding Moieties

Transforming growth factor β (TGFβ) is a multifunctional dimeric cytokine belonging to the transforming growth factor superfamily. There are three mammalian forms of human TGFβ (hTGFβ), namely hTGFβ-1, hTGFβ-2, and hTGFβ-3; and the bioactive form of each is a dimer. hTGFβ signaling is initiated by the binding of hTGFβ to its serine and threonine kinase receptors, the type I (TGFβRI) and type II (TGFβRII) receptors on the cell membrane. It is known in the art, that in certain contexts, TGFβ is pro-tumorigenic, functioning to, e.g., stimulating matrix deposition, perturbation of immune function, and the induction of epithelial-mesenchymal transition (EMT).

The amino acid sequence of a reference mature hTGFβ-1 polypeptide and an immature hTGFβ-1 polypeptide is set forth in SEQ ID NOS: 61 and 62, respectively. The amino acid sequence of a reference mature hTGFβ-2 polypeptide and an immature hTGFβ-2 polypeptide is set forth in SEQ ID NOS: 63 and 64, respectively. The amino acid sequence of a reference mature hTGFβ-3 polypeptide and an immature hTGFβ-3 polypeptide is set forth in SEQ ID NOS: 65 and 66, respectively. The amino acid sequence of a reference mature human TGFβRI (hTGFβRI) polypeptide, an immature hTGFβRI polypeptide, and the extracellular domain (ECD) of the reference hTGFβRI polypeptide the is set forth in SEQ ID NOS: 67, 68, and 69 respectively. The amino acid sequence of a reference mature human TGFβRII (hTGFβRII) polypeptide, an immature hTGFβRII polypeptide, and the ECD of the reference hTGFβRII polypeptide the is set forth in SEQ ID NOS: 70, 71, and 72 respectively. See Table 5, herein. In some embodiments, the extracellular domain of hTGFβRII excludes the N-terminal threonine residue (SEQ ID NO: 73).

TABLE 5
The Amino Acid Sequence of Reference
hTGFβ and hTGFβR Polypeptides.

| | | SEQ |
|---|---|---|
| Descrip- | | ID |
| tion | Amino Acid Sequence | NO |
| hTGFß-1 | LSTCKTIDMELVKRKRIEAIRGQILSKLRL | 61 |
| Mature - | ASPPSQGEVPPGPLPEAVLALYNSTRDRVA | |
| No | GESAEPEPEPEADYYAKEVTRVLMVETHNE | |
| Signal | IYDKFKQSTHSIYMFFNTSELREAVPEPVL | |
| Peptide | LSRAELRLLRLKLKVEQHVELYQKYSNNSW | |
| | RYLSNRLLAPSDSPEWLSFDVTGVVRQWLS | |
| | RGGEIEGFRLSAHCSCDSRDNTLQVDINGF | |
| | TTGRRGDLATIHGMNRPFLLLMATPLERAQ | |
| | HLQSSRHRRALDTNYCFSSTEKNCCVRQLY | |
| | IDFRKDLGWKWIHEPKGYHANFCLGPCPYI | |
| | WSLDTQYSKVLALYNQHNPGASAAPCCVPQ | |
| | ALEPLPIVYYVGRKPKVEQLSNMIVRSCKC | |
| | S | |
| hTGFß-1 | MPPSGLRLLLLLLPLLWLLVLTPGRPAAGL | 62 |
| Immature - | STCKTIDMELVKRKRIEAIRGQILSKLRLA | |
| With | SPPSQGEVPPGPLPEAVLALYNSTRDRVAG | |
| Signal | ESAEPEPEPEADYYAKEVTRVLMVETHNEI | |
| Peptide | YDKFKQSTHSIYMFFNTSELREAVPEPVLL | |
| | SRAELRLLRLKLKVEQHVELYQKYSNNSWR | |
| | YLSNRLLAPSDSPEWLSFDVTGVVRQWLSR | |
| | GGEIEGFRLSAHCSCDSRDNTLQVDINGFT | |
| | TGRRGDLATIHGMNRPFLLLMATPLERAQH | |
| | LQSSRHRRALDTNYCFSSTEKNCCVRQLYI | |
| | DFRKDLGWKWIHEPKGYHANFCLGPCPYIW | |
| | SLDTQYSKVLALYNQHNPGASAAPCCVPQA | |
| | LEPLPIVYYVGRKPKVEQLSNMIVRSCKCS | |
| hTGFß-2 | LSTCSTLDMDQFMRKRIEAIRGQILSKLKL | 63 |
| Mature - | TSPPEDYPEPEEVPPEVISIYNSTRDLLQE | |
| No | KASRRAAACERERSDEEYYAKEVYKIDMPP | |
| Signal | FFPSENAIPPTFYRPYFRIVREDVSAMEKN | |
| Peptide | ASNLVKAEFRVFRLQNPKARVPEQRIELYQ | |
| | ILKSKDLTSPTQRYIDSKVVKTRAEGEWLS | |
| | FDVTDAVHEWLHHKDRNLGFKISLHCPCCT | |
| | FVPSNNYIIPNKSEELEARFAGIDGTSTYT | |
| | SGDQKTIKSTRKKNSGKTPHLLLMLLPSYR | |
| | LESQQTNRRKKRALDAAYCFRNVQDNCCLR | |
| | PLYIDFKRDLGWKWIHEPKGYNANFCAGAC | |
| | PYLWSSDTQHSRVLSLYNTINPEASASPCC | |
| | VSQDLEPLTILYYIGKTPKIEQLSNMIVKS | |
| | CKCS | |
| hTGFß-2 | MHYCVLSAFLILHLVTVALSLSTCSTLDMD | 64 |
| Immature - | QFMRKRIEAIRGQILSKLKLTSPPEDYPEP | |
| With | EEVPPEVISIYNSTRDLLQEKASRRAAACE | |
| Signal | RERSDEEYYAKEVYKIDMPPFFPSENAIPP | |
| Peptide | TFYRPYFRIVRFDVSAMEKNASNLVKAEFR | |
| | VFRLQNPKARVPEQRIELYQILKSKDLTSP | |
| | TQRYIDSKVVKTRAEGEWLSFDVTDAVHEW | |
| | LHHKDRNLGFKISLHCPCCTFVPSNNYIIP | |
| | NKSEELEARFAGIDGTSTYTSGDQKTIKST | |
| | RKKNSGKTPHLLLMLLPSYRLESQQTNRRK | |
| | KRALDAAYCFRNVQDNCCLRPLYIDFKRDL | |
| | GWKWIHEPKGYNANFCAGACPYLWSSDTQH | |
| | SRVLSLYNTINPEASASPCCVSQDLEPLTI | |
| | LYYIGKTPKIEQLSNMIVKSCKCS | |
| hTGFß-3 | LSTCTTLDFGHIKKKRVEAIRGQILSKLRL | 65 |
| Mature - | TSPPEPTVMTHVPYQVLALYNSTRELLEEM | |
| No | HGEREEGCTQENTESEYYAKEIHKFDMIQG | |
| Signal | LAEHNELAVCPKGITSKVFRFNVSSVEKNR | |
| Peptide | TNLFRAEFRVLRVPNPSSKRNEQRIELFQI | |
| | LRPDEHIAKQRYIGGKNLPTRGTAEWLSFD | |
| | VTDTVREWLLRRESNLGLEISIHCPCHTFQ | |
| | PNGDILENIHEVMEIKFKGVDNEDDHGRGD | |
| | LGRLKKQKDHHNPHLILMMIPPHRLDNPGQ | |
| | GGQRKKRALDTNYCFRNLEENCCVRPLYID | |
| | FRQDLGWKWVHEPKGYYANFCSGPCPYLRS | |
| | ADTTHSTVLGLYNTLNPEASASPCCVPQDL | |
| | EPLTILYYVGRTPKVEQLSNMVVKSCKCS | |
| hTGFß-3 | MKMHLQRALVVLALLNFATVSLSLSTCTTL | 66 |
| Immature - | DFGHIKKKRVEAIRGQILSKLRLTSPPEPT | |
| With | VMTHVPYQVLALYNSTRELLEEMHGEREEG | |
| Signal | CTQENTESEYYAKEIHKFDMIQGLAEHNEL | |
| Peptide | AVCPKGITSKVFRFNVSSVEKNRTNLFRAE | |
| | FRVLRVPNPSSKRNEQRIELFQILRPDEHI | |
| | AKQRYIGGKNLPTRGTAEWLSFDVTDTVRE | |
| | WLLRRESNLGLEISIHCPCHTFQPNGDILE | |
| | NIHEVMEIKFKGVDNEDDHGRGDLGRLKKQ | |
| | KDHHNPHLILMMIPPHRLDNPGQGGQRKKR | |
| | ALDTNYCFRNLEENCCVRPLYIDFRQDLGW | |
| | KWVHEPKGYYANFCSGPCPYLRSADTTHST | |
| | VLGLYNTLNPEASASPCCVPQDLEPLTILY | |
| | YVGRTPKVEQLSNMVVKSCKCS | |
| hTGFßRI | LQCFCHLCTKDNFTCVTDGLCFVSVTETTD | 67 |
| Mature - | KVIHNSMCIAEIDLIPRDRPFVCAPSSKTG | |
| No | SVTTTYCCNQDHCNKIELPTTVKSSPGLGP | |
| Signal | VELAAVIAGPVCFVCISLMLMVYICHNRTV | |
| Peptide | IHHRVPNEEDPSLDRPFISEGTTLKDLIYD | |
| | MTTSGSGSGLPLLVQRTIARTIVLQESIGK | |
| | GRFGEVWRGKWRGEEVAVKIFSSREERSWF | |
| | REAEIYQTVMLRHENILGFIAADNKDNGTW | |
| | TQLWLVSDYHEHGSLFDYLNRYTVTVEGMI | |
| | KLALSTASGLAHLHMEIVGTQGKPAIAHRD | |
| | LKSKNILVKKNGTCCIADLGLAVRHDSATD | |
| | TIDIAPNHRVGTKRYMAPEVLDDSINMKHF | |
| | ESFKRADIYAMGLVEWEIARRCSIGGIHED | |
| | YQLPYYDLVPSDPSVEEMRKVVCEQKLRPN | |
| | IPNRWQSCEALRVMAKIMRECWYANGAARL | |
| | TALRIKKTLSQLSQQEGIKM | |
| hTGFßRI | MEAAVAAPRPRLLLLVLAAAAAAAAALLPG | 68 |
| Immature - | ATALQCFCHLCTKDNFTCVTDGLCFVSVTE | |
| With | TTDKVIHNSMCIAEIDLIPRDRPFVCAPSS | |
| Signal | KTGSVTTTYCCNQDHCNKIELPTTVKSSPG | |
| Peptide | LGPVELAAVIAGPVCFVCISLMLMVYICHN | |
| | RTVIHHRVPNEEDPSLDRPFISEGTTLKDL | |
| | IYDMTTSGSGSGLPLLVQRTIARTIVLQES | |
| | IGKGRFGEVWRGKWRGEEVAVKIFSSREER | |
| | SWFREAEIYQTVMLRHENILGFIAADNKDN | |
| | GTWTQLWLVSDYHEHGSLFDYLNRYTVTVE | |
| | GMIKLALSTASGLAHLHMEIVGTQGKPAIA | |
| | HRDLKSKNILVKKNGTCCIADLGLAVRHDS | |
| | ATDTIDIAPNHRVGTKRYMAPEVLDDSINM | |
| | KHFESFKRADIYAMGLVFWEIARRCSIGGI | |
| | HEDYQLPYYDLVPSDPSVEEMRKVVCEQKL | |
| | RPNIPNRWQSCEALRVMAKIMRECWYANGA | |
| | ARLTALRIKKTLSQLSQQEGIKM | |
| hTGFßRI | LQCFCHLCTKDNFTCVTDGLCFVSVTETTD | 69 |
| ECD | KVIHNSMCIAEIDLIPRDRPFVCAPSSKTG | |
| | SVTTTYCCNQDHCNKIELPTTVKSSPGLGP | |
| | VEL | |
| hTGFßRII | TIPPHVQKSVNNDMIVTDNNGAVKFPQLCK | 70 |
| Mature - | FCDVRFSTCDNQKSCMSNCSITSICEKPQE | |
| No | VCVAVWRKNDENITLETVCHDPKLPYHDFI | |
| Signal | LEDAASPKCIMKEKKKPGETFFMCSCSSDE | |
| Peptide | CNDNIIFSEEYNTSNPDLLLVIFQVTGISL | |
| | LPPLGVAISVIIIFYCYRVNRQQKLSSTWE | |
| | TGKTRKLMEFSEHCAIILEDDRSDISSTCA | |
| | NNINHNTELLPIELDTLVGKGRFAEVYKAK | |
| | LKQNTSEQFETVAVKIFPYEEYASWKTEKD | |
| | IFSDINLKHENILQFLTAEERKTELGKQYW | |
| | LITAFHAKGNLQEYLTRHVISWEDLRKLGS | |
| | SLARGIAHLHSDHTPCGRPKMPIVHRDLKS | |
| | SNILVKNDLTCCLCDFGLSLRLDPTLSVDD | |
| | LANSGQVGTARYMAPEVLESRMNLENVESF | |
| | KQTDVYSMALVLWEMTSRCNAVGEVKDYEP | |
| | PFGSKVREHPCVESMKDNVLRDRGRPEIPS | |
| | FWLNHQGIQMVCETLTECWDHDPEARLTAQ | |
| | CVAERFSELEHLDRLSGRSCSEEKIPEDGS | |
| | LNTTK | |
| hTGFßRII | MGRGLLRGLWPLHIVLWTRIASTIPPHVQK | 71 |
| Immature - | SVNNDMIVTDNNGAVKFPQLCKFCDVRFST | |
| With | CDNQKSCMSNCSITSICEKPQEVCVAVWRK | |
| Signal | NDENITLETVCHDPKLPYHDFILEDAASPK | |
| Peptide | CIMKEKKKPGETFFMCSCSSDECNDNIIFS | |
| | EEYNTSNPDLLLVIFQVTGISLLPPLGVAI | |
| | SVIIIFYCYRVNRQQKLSSTWETGKTRKLM | |
| | EFSEHCAIILEDDRSDISSTCANNINHNTE | |
| | LLPIELDTLVGKGRFAEVYKAKLKQNTSEQ | |
| | FETVAVKIFPYEEYASWKTEKDIFSDINLK | |
| | HENILQFLTAEERKTELGKQYWLITAFHAK | |
| | GNLQEYLTRHVISWEDLRKLGSSLARGIAH | |
| | LHSDHTPCGRPKMPIVHRDLKSSNILVKND | |
| | LTCCLCDFGLSLRLDPTLSVDDLANSGQVG | |
| | TARYMAPEVLESRMNLENVESFKQTDVYSM | |
| | ALVLWEMTSRCNAVGEVKDYEPPFGSKVRE | |
| | HPCVESMKDNVLRDRGRPEIPSFWLNHQGI | |
| | QMVCETLTECWDHDPEARLTAQCVAERFSE | |
| | LEHLDRLSGRSCSEEKIPEDGSLNTTK | |
| hTGFßRII | TIPPHVQKSVNNDMIVTDNNGAVKFPQLCK | 72 |
| ECD - A | FCDVRFSTCDNQKSCMSNCSITSICEKPQE | |
| | VCVAVWRKNDENITLETVCHDPKLPYHDFI | |
| | LEDAASPKCIMKEKKKPGETFFMCSCSSDE | |
| | CNDNIIFSEEYNTSNPD | |
| hTGFßRII | IPPHVQKSVNNDMIVTDNNGAVKFPQLCKF | 73 |
| ECD - B | CDVRESTCDNQKSCMSNCSITSICEKPQEV | |
| | CVAVWRKNDENITLETVCHDPKLPYHDFIL | |
| | EDAASPKCIMKEKKKPGETFFMCSCSSDEC | |
| | NDNIIFSEEYNTSNPD | |

In some aspects and embodiments described herein a moiety that specifically binds TGFβ (e.g., hTGFβ) is utilized (also referred to herein as a TGFβ binding moiety, TGFβ binding domain, or an anti-TGFβ binding domain or moiety). For example, in some aspects and embodiments, a fusion protein (e.g., described herein) comprises a moiety that specifically binds TGFβ (e.g., hTGFβ).

In some embodiments, the hTGFβ binding moiety specifically binds at least one of hTGFβ1, hTGFβ2, and hTGFβ3. In some embodiments, the hTGFβ binding moiety specifically binds hTGFβ1. In some embodiments, the hTGFβ binding moiety specifically binds hTGFβ2. In some embodiments, the hTGFβ binding moiety specifically binds hTGFβ3. In some embodiments, the hTGFβ binding moiety specifically binds hTGFβ1 and hTGFβ2. In some embodiments, the hTGFβ binding moiety specifically binds hTGFβ1 and hTGFβ3. In some embodiments, the hTGFβ binding moiety specifically binds hTGFβ2 and hTGFβ3. In some embodiments, the hTGFβ binding moiety specifically binds hTGFβ1, hTGFβ2, and hTGFβ2.

In some embodiments, the TGFβ (e.g., hTGFβ) binding moiety comprises an antibody, or functional fragment or functional variant thereof. In some embodiments, the TGFβ (e.g., hTGFβ) binding moiety comprises a full-length antibody, a scFv, a (scFv)₂, a scFv-Fc, a (scFv)₂-Fc, a Fab, a Fab′, a F(ab′)2, a F(v), a single domain antibody, a single chain antibody, a VHH, a (VHH)₂, a VHH-Fc, or a (VHH)₂-Fc. In some embodiments, the TGFβ (e.g., hTGFβ) binding moiety comprises a full-length antibody, a scFv, a Fab, or a VHH. In some embodiments, the EGFR binding moiety comprises full-length antibody, a single chain variable fragment (scFv), a Fab, or a single domain antibody (sdAb). In some embodiments, the TGFβ (e.g., hTGFβ) binding moiety comprises a full-length antibody.

In some embodiments, the TGFβ (e.g., hTGFβ) binding moiety comprises or consists of at least a portion of the extracellular domain (ECD) of a transforming growth factor-beta receptor (TGFβR) (e.g., hTGFβR). In some embodiments, the TGFβ (e.g., hTGFβ) binding moiety comprises or consists of at least a portion of the ECD of a TGFβR (e.g., hTGFβR), wherein the TGFβ (e.g., hTGFβ) binding moiety retains the ability to specifically bind TGFβ (e.g., hTGFβ). In some embodiments, the TGFβ (e.g., hTGFβ) binding moiety comprises or consists of at least a portion of the ECD of a TGFβRII (e.g., hTGFβRII). In some embodiments, the TGFβ (e.g., hTGFβ) binding moiety comprises or consists of at least a portion of the ECD of a TGFβRII (e.g., hTGFβRII), wherein the TGFβ (e.g., hTGFβ) binding moiety retains the ability to specifically bind TGFβ (e.g., hTGFβ).

In some embodiments, the TGFβ (e.g., hTGFβ) binding moiety comprises or consists of a sufficient portion of the ECD of a TGFβR (e.g., hTGFβR) to mediate binding to TGFβ (e.g., hTGFβ). In some embodiments, the TGFβ (e.g., hTGFβ) binding moiety comprises or consists of a sufficient portion of the ECD of a TGFβRII (e.g., hTGFβRII) to mediate binding to TGFβ (e.g., hTGFβ). In some embodiments, the TGFβ (e.g., hTGFβ) binding moiety comprises or consists of a sufficient portion of the ECD of a naturally occurring TGFβR (e.g., hTGFβR) to mediate binding to TGFβ (e.g., hTGFβ). In some embodiments, the TGFβ (e.g., hTGFβ) binding moiety comprises or consists of a sufficient portion of the ECD of a naturally occurring TGFβRII (e.g., hTGFβRII) to mediate binding to TGFβ (e.g., hTGFβ).

In some embodiments, the TGFβ (e.g., hTGFβ) binding moiety comprises or consists of at least a portion of the ECD of TGFβRII (e.g., hTGFβRII). In some embodiments, the TGFβ (e.g., hTGFβ) binding moiety comprises or consists of at least a portion of the ECD of TGFβRII (e.g., hTGFβRII), wherein the TGFβ (e.g., hTGFβ) binding moiety retains the ability to specifically bind TGFβ. In some embodiments, the TGFβ (e.g., hTGFβ) binding moiety comprises or consists of at least a portion of the ECD of TGFβRII (e.g., hTGFβRII).

In some embodiments, the amino acid sequence of the ECD of the TGFβR (e.g., hTGFβR) polypeptide is at least 85%, 86%, 87%, 88%, 89%, 90%, 91%, 92%, 93%, 94%, 95%, 96%, 97%, 98%, 99%, or 100% identical to the amino acid sequence of the ECD of a TGFβR (e.g., hTGFβR) set forth in Table 5. In some embodiments, the amino acid sequence of the ECD of the TGFβRII (e.g., hTGFβRII) polypeptide is at least 85%, 86%, 87%, 88%, 89%, 90%, 91%, 92%, 93%, 94%, 95%, 96%, 97%, 98%, 99%, or 100% identical to the amino acid sequence of the ECD of a TGFβRII (e.g., hTGFβRII) set forth in Table 5. In some embodiments, the ECD of the TGFβR (e.g., hTGFβR) polypeptide comprises or consist of an amino acid sequence that is at least 85%, 86%, 87%, 88%, 89%, 90%, 91%, 92%, 93%, 94%, 95%, 96%, 97%, 98%, 99%, or 100% identical to the amino acid sequence of the ECD of a TGFβR (e.g., hTGFβR) set forth in Table 5. In some embodiments, the ECD of the TGFβRII (e.g., hTGFβRII) polypeptide comprises or consist of an amino acid sequence that is at least 85%, 86%, 87%, 88%, 89%, 90%, 91%, 92%, 93%, 94%, 95%, 96%, 97%, 98%, 99%, or 100% identical to the amino acid sequence of the ECD of a TGFβRII (e.g., hTGFβRII) set forth in Table 5. In some embodiments, the amino acid sequence of the ECD of the TGFβR (e.g., hTGFβR) polypeptide comprises or consists of the amino acid sequence of the ECD of a TGFβR (e.g., hTGFβR) set forth in Table 5. In some embodiments, the amino acid sequence of the ECD of the TGFβRII (e.g., hTGFβRII) polypeptide comprises or consists of the amino acid sequence of the ECD of a TGFβRII (e.g., hTGFβRII) set forth in Table 5.

In some embodiments, the amino acid sequence of the ECD of the TGFβR (e.g., hTGFβR) polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72. In some embodiments, the amino acid sequence of the ECD of the TGFβRII (e.g., hTGFβRII) polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72. In some embodiments, the ECD of the TGFβR (e.g., hTGFβR) polypeptide comprises or consists of an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 73. In some embodiments, the ECD of the TGFβRII (e.g., hTGFβRII) polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 73.

In some embodiments, the ECD of the TGFβR (e.g., hTGFβR) polypeptide comprises an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 73. In some embodiments, the ECD of the TGFβRII (e.g., hTGFβRII) polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 73.

In some embodiments, the ECD of the TGFβR (e.g., hTGFβR) polypeptide consists of an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 73. In some embodiments, the ECD of the TGFβRII (e.g., hTGFβRII) polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 73.

In some embodiments, the amino acid sequence of the TGFβ (e.g., hTGFβ) binding moiety is at least 85%, 86%, 87%, 88%, 89%, 90%, 91%, 92%, 93%, 94%, 95%, 96%, 97%, 98%, 99%, or 100% identical to the amino acid sequence of the ECD of a TGFβR (e.g., hTGFβR) set forth in Table 5. In some embodiments, the amino acid sequence of the TGFβ (e.g., hTGFβ) binding moiety is at least 85%, 86%, 87%, 88%, 89%, 90%, 91%, 92%, 93%, 94%, 95%, 96%, 97%, 98%, 99%, or 100% identical to the amino acid sequence of the ECD of TGFβRII (e.g., hTGFβRII) set forth in Table 5.

In some embodiments, the amino acid sequence of the TGFβ (e.g., hTGFβ) binding moiety comprises or consists of the amino acid sequence of the ECD of a TGFβR (e.g., hTGFβR) set forth in Table 5, wherein the amino acid sequence of the TGFβ (e.g., hTGFβ) binding moiety comprises a deletion of one or more amino acids (e.g., 1-10, 1-9, 1-8, 1-7, 1-6, 1-5, 1-4, 1-3, 1-2 amino acids) at the N-terminus, C-terminus, or both the N- and C-terminus of the ECD and retains the ability to specifically bind TGFβ. In some embodiments, the amino acid sequence of the TGFβ (e.g., hTGFβ) binding moiety comprises or consists of the amino acid sequence of the ECD of a TGFβR (e.g., hTGFβR) set forth in Table 5, wherein the amino acid sequence of the TGFβ (e.g., hTGFβ) binding moiety comprises a deletion of 1-10, 1-9, 1-8, 1-7, 1-6, 1-5, 1-4, 1-3, or 1-2 amino acids at the N-terminus, C-terminus, or both the N- and C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of the ECD of a TGFβR set forth in Table 5, wherein the amino acid sequence of the TGFβ (e.g., hTGFβ) binding moiety comprises a deletion of 10, 9, 8, 7, 6, 5, 4, 3, 2, or 1 amino acid at the N-terminus, C-terminus, or both the N- and C-terminus of the ECD and retains the ability to specifically bind TGFβ (e.g., hTGFβ).

In some embodiments, the amino acid sequence of the TGFβ (e.g., hTGFβ) binding moiety comprises or consists of the amino acid sequence of the ECD of a TGFβR (e.g., hTGFβR) set forth in Table 5, wherein the amino acid sequence of the TGFβ (e.g., hTGFβ) binding moiety comprises an addition of one or more amino acids (e.g., 1-10, 1-9, 1-8, 1-7, 1-6, 1-5, 1-4, 1-3, 1-2 amino acids) at the C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of the ECD of a TGFβR (e.g., hTGFβR) set forth in Table 5, wherein the amino acid sequence of the TGFβ (e.g., hTGFβ) binding moiety comprises an addition of 1-10, 1-9, 1-8, 1-7, 1-6, 1-5, 1-4, 1-3, or 1-2 amino acids at the C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of the ECD of a TGFβR (e.g., hTGFβR) set forth in Table 5, wherein the amino acid sequence of the TGFβ (e.g., hTGFβ) binding moiety comprises an addition of 10, 9, 8, 7, 6, 5, 4, 3, 2, or 1 amino acid at the C-terminus of the ECD and retains the ability to specifically bind TGFβ (e.g., hTGFβ). In some embodiments, the additional amino acids correspond to the amino acid sequence of a naturally occurring TGFβR (e.g., hTGFβR).

In some embodiments, the amino acid sequence of the TGFβ (e.g., hTGFβ) binding moiety comprises or consists of the amino acid sequence of the ECD of a TGFβRII (e.g., hTGFβRII) set forth in Table 5, wherein the amino acid sequence of the TGFβ (e.g., hTGFβ) binding moiety comprises a deletion of one or more amino acids (e.g., 1-10, 1-9, 1-8, 1-7, 1-6, 1-5, 1-4, 1-3, 1-2) at the N-terminus, C-terminus, or both the N- and C-terminus of the ECD and retains the ability to specifically bind TGFβ. In some embodiments, the amino acid sequence of the TGFβ (e.g., hTGFβ) binding moiety comprises or consists of the amino acid sequence of the ECD of a TGFβRII set forth in Table 5, wherein the amino acid sequence of the TGFβ (e.g., hTGFβ) binding moiety comprises a deletion of 1-10, 1-9, 1-8, 1-7, 1-6, 1-5, 1-4, 1-3, or 1-2 amino acids at the N-terminus, C-terminus, or both the N- and C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of the ECD of a TGFβRII (e.g., hTGFβRII) set forth in Table 5, wherein the amino acid sequence of the TGFβ (e.g., hTGFβ) binding moiety comprises a deletion of 10, 9, 8, 7, 6, 5, 4, 3, 2, or 1 amino acid at the N-terminus, C-terminus, or both the N- and C-terminus of the ECD and retains the ability to specifically bind TGFβ (e.g., hTGFβ).

In some embodiments, the amino acid sequence of the TGFβ (e.g., hTGFβ) binding moiety comprises or consists of the amino acid sequence of the ECD of a TGFβRII (e.g., hTGFβRII) set forth in Table 5, wherein the amino acid sequence of the TGFβ (e.g., hTGFβ) binding moiety comprises an addition of one or more amino acids (e.g., 1-10, 1-9, 1-8, 1-7, 1-6, 1-5, 1-4, 1-3, 1-2 amino acids) at the C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of the ECD of a TGFβRII (e.g., hTGFβRII) set forth in Table 5, wherein the amino acid sequence of the TGFβ (e.g., hTGFβ) binding moiety comprises an addition of 1-10, 1-9, 1-8, 1-7, 1-6, 1-5, 1-4, 1-3, or 1-2 amino acids at the C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of the ECD of a TGFβRII (e.g., hTGFβRII) set forth in Table 5, wherein the amino acid sequence of the TGFβ (e.g., hTGFβ) binding moiety comprises an addition of 10, 9, 8, 7, 6, 5, 4, 3, 2, or 1 amino acid at the C-terminus of the ECD and retains the ability to specifically bind TGFβ (e.g., hTGFβ). In some embodiments, the additional amino acids correspond to the amino acid sequence of a naturally occurring TGFβRII (e.g., hTGFβRII).

In some embodiments, the amino acid sequence of the TGFβ (e.g., hTGFβ) binding moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72. In some embodiments, the amino acid sequence of the TGFβ (e.g., hTGFβ) binding moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 73.

In some embodiments, the amino acid sequence of the TGFβ (e.g., hTGFβ) binding moiety comprises or consists of an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72. In some embodiments, the amino acid sequence of the TGFβ (e.g., hTGFβ) binding moiety comprises or consists of an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 73.

In some embodiments, the amino acid sequence of the TGFβ (e.g., hTGFβ) binding moiety comprises an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72. In some embodiments, the amino acid sequence of the TGFβ (e.g., hTGFβ) binding moiety comprises an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 73.

In some embodiments, the amino acid sequence of the TGFβ (e.g., hTGFβ) binding moiety consists of an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72. In some embodiments, the amino acid sequence of the TGFβ (e.g., hTGFβ) binding moiety consists of an amino acid sequence that is at least 85%, 86%, 87%, 88%, 89%, 90%, 91%, 92%, 93%, 94%, 95%, 96%, 97%, 98%, 99%, or 100% identical to the amino acid sequence of SEQ ID NO: 73.

In some embodiments, the amino acid sequence of the TGFβ (e.g., hTGFβ) binding moiety comprises or consists of the amino acid sequence of SEQ ID NO: 72, wherein the amino acid sequence of the TGFβ (e.g., hTGFβ) binding moiety comprises a deletion of one or more amino acids (e.g., 1-10, 1-9, 1-8, 1-7, 1-6, 1-5, 1-4, 1-3, 1-2 amino acids) at the N-terminus, C-terminus, or both the N- and C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of SEQ ID NO: 73, wherein the amino acid sequence of the TGFβ (e.g., hTGFβ) binding moiety comprises a deletion of one or more amino acids (e.g., 1-10, 1-9, 1-8, 1-7, 1-6, 1-5, 1-4, 1-3, 1-2 amino acids) at the N-terminus, C-terminus, or both the N- and C-terminus of the ECD and retains the ability to specifically bind TGFβ (e.g., hTGFβ).

In some embodiments, the amino acid sequence of the TGFβ (e.g., hTGFβ) binding moiety comprises or consists of the amino acid sequence of SEQ ID NO: 72, wherein the amino acid sequence of the TGFβ (e.g., hTGFβ) binding moiety comprises a deletion of 1-10, 1-9, 1-8, 1-7, 1-6, 1-5, 1-4, 1-3, or 1-2 amino acids at the N-terminus, C-terminus, or both the N- and C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of SEQ ID NO: 73, wherein the amino acid sequence of the TGFβ (e.g., hTGFβ) binding moiety comprises a deletion of 1-10, 1-9, 1-8, 1-7, 1-6, 1-5, 1-4, 1-3, or 1-2 amino acids at the N-terminus, C-terminus, or both the N- and C-terminus of the ECD and retains the ability to specifically bind TGFβ (e.g., hTGFβ).

In some embodiments, the amino acid sequence of the TGFβ (e.g., hTGFβ) binding moiety comprises or consists of the amino acid sequence of SEQ ID NO: 72, wherein the amino acid sequence of the TGFβ (e.g., hTGFβ) binding moiety comprises a deletion of 10, 9, 8, 7, 6, 5, 4, 3, 2, or 1 amino acid at the N-terminus, C-terminus, or both the N- and C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of SEQ ID NO: 73, wherein the amino acid sequence of the TGFβ (e.g., hTGFβ) binding moiety comprises a deletion of 10, 9, 8, 7, 6, 5, 4, 3, 2, or 1 amino acid at the N-terminus, C-terminus, or both the N- and C-terminus of the ECD and retains the ability to specifically bind TGFβ (e.g., hTGFβ).

In some embodiments, the amino acid sequence of the TGFβ (e.g., hTGFβ) binding moiety comprises or consists of the amino acid sequence of SEQ ID NO: 72, wherein the amino acid sequence of the TGFβ (e.g., hTGFβ) binding moiety comprises an addition of one or more amino acids (e.g., −10, 1-9, 1-8, 1-7, 1-6, 1-5, 1-4, 1-3, 1-2 amino acids) at the C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of SEQ ID NO: 73, wherein the amino acid sequence of the TGFβ (e.g., hTGFβ) binding moiety comprises an addition of one or more amino acids (e.g., 1-10, 1-9, 1-8, 1-7, 1-6, 1-5, 1-4, 1-3, 1-2 amino acids) at the C-terminus of the ECD and retains the ability to specifically bind TGFβ (e.g., hTGFβ).

In some embodiments, the amino acid sequence of the TGFβ (e.g., hTGFβ) binding moiety comprises or consists of the amino acid sequence of SEQ ID NO: 72, wherein the amino acid sequence of the TGFβ (e.g., hTGFβ) binding moiety comprises an addition of 1-10, 1-9, 1-8, 1-7, 1-6, 1-5, 1-4, 1-3, or 1-2 amino acids at the C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of SEQ ID NO: 73, wherein the amino acid sequence of the TGFβ (e.g., hTGFβ) binding moiety comprises an addition of 1-10, 1-9, 1-8, 1-7, 1-6, 1-5, 1-4, 1-3, or 1-2 amino acids at the C-terminus of the ECD and retains the ability to specifically bind TGFβ (e.g., hTGFβ).

In some embodiments, the amino acid sequence of the TGFβ (e.g., hTGFβ) binding moiety comprises or consists of the amino acid sequence of SEQ ID NO: 72, wherein the amino acid sequence of the TGFβ (e.g., hTGFβ) binding moiety comprises an addition of 10, 9, 8, 7, 6, 5, 4, 3, 2, or 1 amino acid at the C-terminus of the ECD and retains the ability to specifically bind TGFβ (e.g., hTGFβ). In some embodiments, the amino acid sequence of the TGFβ (e.g., hTGFβ) binding moiety comprises or consists of the amino acid sequence of SEQ ID NO: 73, wherein the amino acid sequence of the TGFβ (e.g., hTGFβ) binding moiety comprises an addition of 10, 9, 8, 7, 6, 5, 4, 3, 2, or 1 amino acid at the C-terminus of the ECD and retains the ability to specifically bind TGFβ (e.g., hTGFβ).

### 5.5 Fusion Proteins & Polypeptides

In some aspects and embodiments described herein a fusion protein is utilized. Fusion proteins described herein comprise at least a first moiety (e.g., an EGFR (e.g., hEGFR) binding moiety (e.g., an EGFR (e.g., hEGFR) binding moiety described herein)) operably connected to a second moiety (e.g., a TGFβ (e.g., hTGFβ) binding moiety (e.g., a TGFβ (e.g., hTGFβ) binding moiety described herein)), wherein the second moiety (e.g., the TGFβ (e.g., hTGFβ) binding moiety) is heterologous to the first moiety (e.g., the EGFR (e.g., hEGFR) binding moiety). In some embodiments, the fusion protein comprises at least one EGFR (e.g., hEGFR) binding moiety (e.g., an EGFR (e.g., hEGFR) binding moiety described herein, see, e.g., § 5.3) operably connected to at least one TGFβ (e.g., hTGFβ) binding moiety (e.g., a TGFβ (e.g., hTGFβ)) binding moiety described herein, see, e.g., § 5.4).

In some embodiments, the fusion protein comprises or consists of two EGFR (e.g., hEGFR) binding moieties. In some embodiments, the fusion protein comprises or consists of two TGFβ (e.g., hTGFβ) binding moieties. In some embodiments, the fusion protein comprises or consists of two EGFR (e.g., hEGFR) binding moieties and two TGFβ (e.g., hTGFβ) binding moieties.

In some embodiments, the fusion protein comprises or consists of one EGFR (e.g., hEGFR) binding moiety. In some embodiments, the fusion protein comprises or consists of one TGFβ (e.g., hTGFβ) binding moiety. In some embodiments, the fusion protein comprises or consists of one EGFR (e.g., hEGFR) binding moiety and one TGFβ (e.g., hTGFβ) binding moiety.

In some embodiments, the fusion protein comprises or consists of two EGFR (e.g., hEGFR) binding moieties. In some embodiments, the fusion protein comprises or consists of two TGFβ (e.g., hTGFβ) binding moieties. In some embodiments, the fusion protein comprises or consists of two EGFR (e.g., hEGFR) binding moieties and two TGFβ (e.g., hTGFβ) binding moieties.

In some embodiments, the fusion protein comprises or consists of one EGFR (e.g., hEGFR) binding moiety and two TGFβ (e.g., hTGFβ) binding moieties. In some embodiments, the fusion protein comprises or consists of two EGFR (e.g., hEGFR) binding moieties and one TGFβ (e.g., hTGFβ) binding moiety. In some embodiments, the fusion protein comprises two EGFR (e.g., hEGFR) binding moieties and two TGFβ (e.g., hTGFβ) binding moiety.

In some embodiments, the fusion protein comprises an EGFR binding moiety that is a full-length antibody and two TGFβ (e.g., hTGFβ) binding moieties. In some embodiments, the fusion protein comprises an EGFR (e.g., hEGFR) binding moiety that is a full-length antibody and two TGFβ (e.g., hTGFβ) binding moieties, wherein one of the two TGFβ (e.g., hTGFβ) binding moieties is operably connected to one light chain of the full-length antibody and the second TGFβ (e.g., hTGFβ) binding moiety is operably connected to the second light chain of the full-length antibody.

In some embodiments, the fusion protein comprises (a) a first moiety that comprises or consists of a full-length antibody that specifically binds EGFR (e.g., hEGFR); (b) a second moiety that comprises a comprises or consists of the ECD of TGFβRII (e.g., hTGFβRII); (c) a third moiety that comprises a comprises or consists of the ECD of TGFβRII (e.g., hTGFβRII); wherein the second moiety is operably connected to the first moiety through a first peptide linker; and wherein (c) the third moiety is operably connected to the first moiety through a second peptide linker.

### 5.5.1 Ig Constant Regions

In some embodiments, the fusion proteins (or one or more moiety thereof) described herein comprise one or more Ig (e.g., hIg) heavy chain constant regions (e.g., a CH1 region, a hinge region, a CH2 region, a CH3 region, an Fc region). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the fusion proteins (or one or more moiety thereof) comprises a Fc region. In some embodiments, the Fc region is part of a full-length antibody. In some embodiments, the Fc region comprises or consists of at least a portion of a hinge region, a CH2 region, and a CH3 region. In some embodiments, the Fc region comprises or consists of a hinge region, a CH2 region, and a CH3 region. In some embodiments, the Fc region comprises or consists of at least a portion of a hIgG hinge region, a hIgG CH2 region, and a hIgG CH3 region. In some embodiments, the Fc region comprises or consists of a hIgG hinge region, a hIgG CH2 region, and a hIgG CH3 region. In some embodiments, the Fc region comprises or consists of at least a portion of a hIgG1 hinge region, a hIgG1 CH2 region, and a hIgG1 CH3 region. In some embodiments, the Fc region comprises or consists of a hIgG1 hinge region, a hIgG1 CH2 region, and a hIgG1 CH3 region. In some embodiments, the Fc region comprises or consists of at least a portion of a hIgG4 hinge region, a hIgG4 CH2 region, and a hIgG4 CH3 region. In some embodiments, the Fc region comprises or consists of a hIgG4 hinge region, a hIgG4 CH2 region, and a hIgG4 CH3 region.

The amino acid sequence of exemplary reference hIgG1 and hIgG4 heavy chain constant regions, which can be incorporated in one or more of the embodiments described herein (e.g., multispecific proteins (or one or more polypeptide thereof)), is provided in Table 6.

TABLE 6
The Amino Acid Sequence of Exemplary hIg constant region components.

| | | SEQ |
|---|---|---|
| | | ID |
| Description | Amino Acid Sequence | NO |
| hIgG1 CH1 Region | ASTKGPSVFPLAPSSKSTSGGTAALGCLVKDYFPEPVTVSWNSG | 74 |
| | ALTSGVHTFPAVLQSSGLYSLSSVVTVPSSSLGTQTYICNVNHK | |
| | PSNTKVDKKV | |
| hIgG1 CH1 Region | ASTKGPSVFPLAPSSKSTSGGTAALGCLVKDYFPEPVTVSWNSG | 75 |
| K222R | ALTSGVHTFPAVLQSSGLYSLSSVVTVPSSSLGTQTYICNVNHK | |
| | PSNTKVDKKR | |
| hIgG1 Hinge Region | EPKSCDKTHTCP | 76 |
| hIgG1 CH2 Region | PCPAPELLGGPSVFLFPPKPKDTLMISRTPEVTCVVVDVSHEDP | 77 |
| | EVKFNWYVDGVEVHNAKTKPREEQYNSTYRVVSVLTVLHQDWLN | |
| | GKEYKCKVSNKALPAPIEKTISKAK | |
| hIgG1 CH3 Region | GQPREPQVYTLPPSRDELTKNQVSLTCLVKGFYPSDIAVEWESN | 78 |
| With C-terminal | GQPENNYKTTPPVLDSDGSFFLYSKLTVDKSRWQQGNVFSCSVM | |
| Lysine | HEALHNHYTQKSLSLSPGK | |
| hIgG1 CH3 Region | GQPREPQVYTLPPSRDELTKNQVSLTCLVKGFYPSDIAVEWESN | 79 |
| Without C-terminal | GQPENNYKTTPPVLDSDGSFFLYSKLTVDKSRWQQGNVFSCSVM | |
| Lysine | HEALHNHYTQKSLSLSPG | |
| hIgG1 CH2 Region + | PCPAPELLGGPSVFLFPPKPKDTLMISRTPEVTCVVVDVSHEDP | 80 |
| CH3 Region | EVKFNWYVDGVEVHNAKTKPREEQYNSTYRVVSVLTVLHQDWLN | |
| With C-terminal | GKEYKCKVSNKALPAPIEKTISKAKGQPREPQVYTLPPSRDELT | |
| Lysine | KNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| hIgG1 CH2 Region + | PCPAPELLGGPSVFLFPPKPKDTLMISRTPEVTCVVVDVSHEDP | 81 |
| CH3 Region | EVKFNWYVDGVEVHNAKTKPREEQYNSTYRVVSVLTVLHQDWLN | |
| Without C-terminal | GKEYKCKVSNKALPAPIEKTISKAKGQPREPQVYTLPPSRDELT | |
| Lysine | KNQVSLTCLVKGFYPSDIAVEWESNGQPENNYKTTPPVLDSDGS | |
| | FFLYSKLTVDKSRWQQGNVFSCSVMHEALHNHYTQKSLSLSPG | |
| hIgG1 Partial Hinge | TCPPCPAPELLGGPSVFLFPPKPKDTLMISRTPEVTCVVVDVSH | 82 |
| Region + CH2 | EDPEVKFNWYVDGVEVHNAKTKPREEQYNSTYRVVSVLTVLHQD | |
| Region + | WLNGKEYKCKVSNKALPAPIEKTISKAKGQPREPQVYTLPPSRD | |
| CH3 Region | ELTKNQVSLTCLVKGFYPSDIAVEWESNGQPENNYKTTPPVLDS | |
| With C-terminal | DGSFFLYSKLTVDKSRWQQGNVFSCSVMHEALHNHYTQKSLSLS | |
| Lysine | PGK | |
| hIgG1 Partial Hinge | TCPPCPAPELLGGPSVFLFPPKPKDTLMISRTPEVTCVVVDVSH | 83 |
| Region + CH2 | EDPEVKFNWYVDGVEVHNAKTKPREEQYNSTYRVVSVLTVLHQD | |
| Region + | WLNGKEYKCKVSNKALPAPIEKTISKAKGQPREPQVYTLPPSRD | |
| CH3 Region | ELTKNQVSLTCLVKGFYPSDIAVEWESNGQPENNYKTTPPVLDS | |
| Without C-terminal | DGSFFLYSKLTVDKSRWQQGNVFSCSVMHEALHNHYTQKSLSLS | |
| Lysine | PG | |
| hIgG1 Hinge Region + | EPKSCDKTHTCPPCPAPELLGGPSVFLFPPKPKDTLMISRTPEV | 84 |
| CH2 Region + CH3 | TCVVVDVSHEDPEVKFNWYVDGVEVHNAKTKPREEQYNSTYRVV | |
| Region With | SVLTVLHQDWLNGKEYKCKVSNKALPAPIEKTISKAKGQPREPQ | |
| C-terminal Lysine | VYTLPPSRDELTKNQVSLTCLVKGFYPSDIAVEWESNGQPENNY | |
| | KTTPPVLDSDGSFFLYSKLTVDKSRWQQGNVFSCSVMHEALHNH | |
| | YTQKSLSLSPGK | |
| hIgG1 Hinge Region + | EPKSCDKTHTCPPCPAPELLGGPSVFLFPPKPKDTLMISRTPEV | 85 |
| CH2 Region + CH3 | TCVVVDVSHEDPEVKFNWYVDGVEVHNAKTKPREEQYNSTYRVV | |
| Region | SVLTVLHQDWLNGKEYKCKVSNKALPAPIEKTISKAKGQPREPQ | |
| Without C-terminal | VYTLPPSRDELTKNQVSLTCLVKGFYPSDIAVEWESNGQPENNY | |
| Lysine | KTTPPVLDSDGSFFLYSKLTVDKSRWQQGNVFSCSVMHEALHNH | |
| | YTQKSLSLSPG | |
| hIgG1 CH1+ Hinge | ASTKGPSVFPLAPSSKSTSGGTAALGCLVKDYFPEPVTVSWNSG | 86 |
| Region + CH2 | ALTSGVHTFPAVLQSSGLYSLSSVVTVPSSSLGTQTYICNVNHK | |
| Region + | PSNTKVDKKVEPKSCDKTHTCPPCPAPELLGGPSVFLFPPKPKD | |
| CH3 Region | TLMISRTPEVTCVVVDVSHEDPEVKFNWYVDGVEVHNAKTKPRE | |
| With C-terminal | EQYNSTYRVVSVLTVLHQDWLNGKEYKCKVSNKALPAPIEKTIS | |
| Lysine | KAKGQPREPQVYTLPPSRDELTKNQVSLTCLVKGFYPSDIAVEW | |
| | ESNGQPENNYKTTPPVLDSDGSFFLYSKLTVDKSRWQQGNVFIC | |
| | SVMHEALHNHYTQKSLSLSPGK | |
| hIgG1 CH1 + Hinge | ASTKGPSVFPLAPSSKSTSGGTAALGCLVKDYFPEPVTVSWNSG | 87 |
| Region + CH2 | ALTSGVHTFPAVLQSSGLYSLSSVVTVPSSSLGTQTYICNVNHK | |
| Region + | PSNTKVDKKVEPKSCDKTHTCPPCPAPELLGGPSVFLFPPKPKD | |
| CH3 Region | TLMISRTPEVTCVVVDVSHEDPEVKFNWYVDGVEVHNAKTKPRE | |
| Without C-terminal | EQYNSTYRVVSVLTVLHQDWLNGKEYKCKVSNKALPAPIEKTIS | |
| Lysine | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| hIgG1 CH1 (K222R) + | ASTKGPSVFPLAPSSKSTSGGTAALGCLVKDYFPEPVTVSWNSG | 88 |
| Hinge Region + CH2 | ALTSGVHTFPAVLQSSGLYSLSSVVTVPSSSLGTQTYICNVNHK | |
| Region + CH3 Region | PSNTKVDKRVEPKSCDKTHTCPPCPAPELLGGPSVFLFPPKPKD | |
| With C-terminal | TLMISRTPEVTCVVVDVSHEDPEVKFNWYVDGVEVHNAKTKPRE | |
| Lysine | EQYNSTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| hIgG1 CH1 (K222R) + | ASTKGPSVFPLAPSSKSTSGGTAALGCLVKDYFPEPVTVSWNSG | 89 |
| Hinge Region + CH2 | ALTSGVHTFPAVLQSSGLYSLSSVVTVPSSSLGTQTYICNVNHK | |
| Region + CH3 Region | PSNTKVDKRVEPKSCDKTHTCPPCPAPELLGGPSVFLFPPKPKD | |
| Without C-terminal | TLMISRTPEVTCVVVDVSHEDPEVKFNWYVDGVEVHNAKTKPRE | |
| Lysine | EQYNSTYRVVSVLTVLHQDWLNGKEYKCKVSNKALPAPIEKTIS | |
| | KAKGQPREPQVYTLPPSRDELTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPG | |
| hIgG4 CH1 Region | ASTKGPSVFPLAPCSRSTSESTAALGCLVKDYFPEPVTVSWNSG | 90 |
| | ALTSGVHTFPAVLQSSGLYSLSSVVTVPSSSLGTKTYTCNVDHK | |
| | PSNTKVDKRV | |
| hIgG4 Hinge Region | ESKYGPPCPSCP | 91 |
| hIgG4 CH2 Region | APEFLGGPSVFLFPPKPKDTLMISRTPEVTCVVVDVSQEDPEVQ | 92 |
| | FNWYVDGVEVHNAKTKPREEQFNSTYRVVSVLTVLHQDWLNGKE | |
| | YKCKVSNKGLPSSIEKTISKAK | |
| hIgG4 CH3 Region | GQPREPQVYTLPPSQEEMTKNQVSLTCLVKGFYPSDIAVEWESN | 93 |
| With C-terminal | GQPENNYKTTPPVLDSDGSFFLYSRLTVDKSRWQEGNVFSCSVM | |
| Lysine | HEALHNHYTQKSLSLSLGK | |
| hIgG4 CH3 Region | GQPREPQVYTLPPSQEEMTKNQVSLTCLVKGFYPSDIAVEWESN | 94 |
| Without C-terminal | GQPENNYKTTPPVLDSDGSFFLYSRLTVDKSRWQEGNVFSCSVM | |
| Lysine | HEALHNHYTQKSLSLSLG | |
| hIgG4 CH2 Region + | APEFLGGPSVFLFPPKPKDTLMISRTPEVTCVVVDVSQEDPEVQ | 95 |
| CH3 Region With | FNWYVDGVEVHNAKTKPREEQFNSTYRVVSVLTVLHQDWLNGKE | |
| C-terminal Lysine | YKCKVSNKGLPSSIEKTISKAKGQPREPQVYTLPPSQEEMTKNQ | |
| | VSLTCLVKGFYPSDIAVEWESNGQPENNYKTTPPVLDSDGSFFL | |
| | YSRLTVDKSRWQEGNVFSCSVMHEALHNHYTQKSLSLSLGK | |
| hIgG4 CH2 Region + | APEFLGGPSVFLFPPKPKDTLMISRTPEVTCVVVDVSQEDPEVQ | 96 |
| CH3 Region | FNWYVDGVEVHNAKTKPREEQFNSTYRVVSVLTVLHQDWLNGKE | |
| Without C-terminal | YKCKVSNKGLPSSIEKTISKAKGQPREPQVYTLPPSQEEMTKNQ | |
| Lysine | VSLTCLVKGFYPSDIAVEWESNGQPENNYKTTPPVLDSDGSFFL | |
| | YSRLTVDKSRWQEGNVFSCSVMHEALHNHYTQKSLSLSLG | |
| hIgG4 Partial Hinge | PCPSCPAPEFLGGPSVFLFPPKPKDTLMISRTPEVTCVVVDVSQ | 97 |
| Region + CH2 | EDPEVQFNWYVDGVEVHNAKTKPREEQFNSTYRVVSVLTVLHQD | |
| Region + | WLNGKEYKCKVSNKGLPSSIEKTISKAKGQPREPQVYTLPPSQE | |
| CH3 Region With | EMTKNQVSLTCLVKGFYPSDIAVEWESNGQPENNYKTTPPVLDS | |
| C-terminal Lysine | DGSFFLYSRLTVDKSRWQEGNVFSCSVMHEALHNHYTQKSLSLS | |
| | LGK | |
| hIgG4 Partial Hinge | PCPSCPAPEFLGGPSVFLFPPKPKDTLMISRTPEVTCVVVDVSQ | 98 |
| Region + CH2 | EDPEVQFNWYVDGVEVHNAKTKPREEQFNSTYRVVSVLTVLHQD | |
| Region + | WLNGKEYKCKVSNKGLPSSIEKTISKAKGQPREPQVYTLPPSQE | |
| CH3 Region | EMTKNQVSLTCLVKGFYPSDIAVEWESNGQPENNYKTTPPVLDS | |
| Without C-terminal | DGSFFLYSRLTVDKSRWQEGNVFSCSVMHEALHNHYTQKSLSLS | |
| Lysine | LG | |
| hIgG4 Hinge Region + | ESKYGPPCPSCPAPEFLGGPSVFLFPPKPKDTLMISRTPEVTCV | 99 |
| CH2 Region + CH3 | VVDVSQEDPEVQFNWYVDGVEVHNAKTKPREEQFNSTYRVVSVL | |
| Region With | TVLHQDWLNGKEYKCKVSNKGLPSSIEKTISKAKGQPREPQVYT | |
| C-terminal Lysine | LPPSQEEMTKNQVSLTCLVKGFYPSDIAVEWESNGQPENNYKTT | |
| | PPVLDSDGSFFLYSRLTVDKSRWQEGNVFSCSVMHEALHNHYTQ | |
| | KSLSLSLGK | |
| hIgG4 Hinge Region + | ESKYGPPCPSCPAPEFLGGPSVFLFPPKPKDTLMISRTPEVTCV | 100 |
| CH2 Region + CH3 | VVDVSQEDPEVQFNWYVDGVEVHNAKTKPREEQFNSTYRVVSVL | |
| Region | TVLHQDWLNGKEYKCKVSNKGLPSSIEKTISKAKGQPREPQVYT | |
| Without C-terminal | LPPSQEEMTKNQVSLTCLVKGFYPSDIAVEWESNGQPENNYKTT | |
| Lysine | PPVLDSDGSFFLYSRLTVDKSRWQEGNVESCSVMHEALHNHYTQ | |
| | KSLSLSLG | |
| hIgG4 CH1 + Hinge | ASTKGPSVFPLAPCSRSTSESTAALGCLVKDYFPEPVTVSWNSG | 101 |
| Region + CH2 | ALTSGVHTFPAVLQSSGLYSLSSVVTVPSSSLGTKTYTCNVDHK | |
| Region + | PSNTKVDKRVESKYGPPCPSCPAPEFLGGPSVFLFPPKPKDTLM | |
| CH3 Region With | ISRTPEVTCVVVDVSQEDPEVQFNWYVDGVEVHNAKTKPREEQF | |
| C-terminal Lysine | NSTYRVVSVLTVLHQDWLNGKEYKCKVSNKGLPSSIEKTISKAK | |
| | GQPREPQVYTLPPSQEEMTKNQVSLTCLVKGFYPSDIAVEWESN | |
| | GQPENNYKTTPPVLDSDGSFFLYSRLTVDKSRWQEGNVFSCSVM | |
| | HEALHNHYTQKSLSLSLGK | |
| hIgG4 CH1 + Hinge | ASTKGPSVFPLAPCSRSTSESTAALGCLVKDYFPEPVTVSWNSG | 102 |
| Region + CH2 | ALTSGVHTFPAVLQSSGLYSLSSVVTVPSSSLGTKTYTCNVDHK | |
| Region + | PSNTKVDKRVESKYGPPCPSCPAPEFLGGPSVFLFPPKPKDTLM | |
| CH3 Region | ISRTPEVTCVVVDVSQEDPEVQFNWYVDGVEVHNAKTKPREEQF | |
| Without C-terminal | NSTYRVVSVLTVLHQDWLNGKEYKCKVSNKGLPSSIEKTISKAK | |
| Lysine | GQPREPQVYTLPPSQEEMTKNQVSLTCLVKGFYPSDIAVEWESN | |
| | GQPENNYKTTPPVLDSDGSFFLYSRLTVDKSRWQEGNVFSCSVM | |
| | HEALHNHYTQKSLSLSLG | |
| Ig light chain kappa | RTVAAPSVFIFPPSDEQLKSGTASVVCLLNNFYPREAKVQWKVD | 103 |
| constant region | NALQSGNSQESVTEQDSKDSTYSLSSTLTLSKADYEKHKVYACE | |
| (κCL) | VTHQGLSSPVTKSFNRGEC | |
| Ig light chain kappa | GQPKANPTVTLFPPSSEELQANKATLVCLISDFYPGAVTVAWKA | 104 |
| constant region | DGSPVKAGVETTKPSKQSNNKYAASSYLSLTPEQWKSHRSYSCQ | |
| (λCL) | VTHEGSTVEKTVAPTECS | |

In some embodiments, the fusion protein (or one or more polypeptide thereof) comprises one or more hIg constant regions, wherein the amino acid sequence of the one or more hIg constant regions comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 6. In some embodiments, the amino acid sequence of the one or more hIg constant regions comprises or consists of an amino acid sequence set forth in Table 6.

In some embodiments, the amino acid sequence of the one or more hIg constant regions comprises or consists of an amino acid sequence set forth in Table 6, and further comprises 1 or more but less than 15% (less than 12%, less than 10%, less than 8%), amino acid modifications (e.g., amino acid substitutions, deletions, or additions). In some embodiments, the amino acid sequence of the one or more hIg constant regions comprises or consists of an amino acid sequence set forth in Table 6, comprising or consisting of at least about 1, 2, 3, 4, 5, 6, 7, 8, 9, 10, or more amino acid modifications (e.g., amino acid substitutions, deletions, or additions). In some embodiments, the amino acid sequence of the one or more hIg constant regions comprises or consists of an amino acid sequence set forth in Table 6, comprising or consisting of about 1, 2, 3, 4, 5, 6, 7, 8, 9, 10, or more amino acid modifications (e.g., amino acid substitutions, deletions, or additions). In some embodiments, the amino acid sequence of the one or more hIg constant regions comprises or consists of an amino acid sequence set forth in Table 6, comprising or consisting of about no more than 1, 2, 3, 4, 5, 6, 7, 8, 9, 10, or more amino acid modifications (e.g., amino acid substitutions, deletions, or additions).

In some embodiments, the amino acid sequence of the one or more hIg constant regions comprises or consists of an amino acid sequence set forth in Table 6, and further comprises 1 or more but less than 15% (less than 12%, less than 10%, less than 8%), amino acid substitutions. In some embodiments, the amino acid sequence of the one or more hIg constant regions comprises or consists of an amino acid sequence set forth in Table 6, comprising or consisting of at least about 1, 2, 3, 4, 5, 6, 7, 8, 9, 10, or more amino acid substitutions. In some embodiments, the amino acid sequence of the one or more hIg constant regions comprises or consists of an amino acid sequence set forth in Table 6, comprising or consisting of about 1, 2, 3, 4, 5, 6, 7, 8, 9, 10, or more amino acid substitutions. In some embodiments, the amino acid sequence of the one or more hIg constant regions comprises or consists of an amino acid sequence set forth in Table 6, comprising or consisting of about no more than 1, 2, 3, 4, 5, 6, 7, 8, 9, 10, or more amino acid substitutions.

In some embodiments, the amino acid sequence of the one or more hIg constant regions comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 86-89. In some embodiments, the amino acid sequence of the one or more hIg constant regions comprises or consists of the amino acid sequence of any one of SEQ ID NOS: 86-89.

In some embodiments, the amino acid sequence of the one or more hIg constant regions comprises or consists of the amino acid sequence of any one of SEQ ID NOS: 86-89, and further comprises 1 or more but less than 15% (less than 12%, less than 10%, less than 8%), amino acid modifications (e.g., amino acid substitutions, deletions, or additions). In some embodiments, the amino acid sequence of the one or more hIg constant regions comprises or consists of the amino acid sequence of any one of SEQ ID NOS: 86-89, comprising or consisting at least about 1, 2, 3, 4, 5, 6, 7, 8, 9, 10, or more amino acid modifications (e.g., amino acid substitutions, deletions, or additions). In some embodiments, the amino acid sequence of the one or more hIg constant regions comprises or consists of the amino acid sequence of any one of SEQ ID NOS: 86-89, comprising or consisting about 1, 2, 3, 4, 5, 6, 7, 8, 9, or 10 amino acid modifications (e.g., amino acid substitutions, deletions, or additions). In some embodiments, the amino acid sequence of the one or more hIg constant regions comprises or consists of the amino acid sequence of any one of SEQ ID NOS: 86-89, comprising or consisting of no more than about 1, 2, 3, 4, 5, 6, 7, 8, 9, or 10 amino acid modifications (e.g., amino acid substitutions, deletions, or additions).

In some embodiments, the amino acid sequence of the one or more hIg constant regions comprises or consists of the amino acid sequence of any one of SEQ ID NOS: 86-89, and further comprises 1 or more but less than 15% (less than 12%, less than 10%, less than 8%), amino acid substitutions. In some embodiments, the amino acid sequence of the one or more hIg constant regions comprises or consists of the amino acid sequence of any one of SEQ ID NOS: 86-89, comprising or consisting at least about 1, 2, 3, 4, 5, 6, 7, 8, 9, 10, or more amino acid substitutions. In some embodiments, the amino acid sequence of the one or more hIg constant regions comprises or consists of the amino acid sequence of any one of SEQ ID NOS: 86-89, comprising or consisting about 1, 2, 3, 4, 5, 6, 7, 8, 9, or 10 amino acid substitutions. In some embodiments, the amino acid sequence of the one or more hIg constant regions comprises or consists of the amino acid sequence of any one of SEQ ID NOS: 86-89, comprising or consisting of no more than about 1, 2, 3, 4, 5, 6, 7, 8, 9, or 10 amino acid substitutions.

In some embodiments, the amino acid sequence of the one or more hIg constant regions comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 103-104. In some embodiments, the amino acid sequence of the one or more hIg constant regions comprises or consists of the amino acid sequence of any one of SEQ ID NOS: 103-104.

In some embodiments, the amino acid sequence of the one or more hIg constant regions comprises or consists of the amino acid sequence of any one of SEQ ID NOS: 103-104, and further comprises 1 or more but less than 15% (less than 12%, less than 10%, less than 8%), amino acid modifications (e.g., amino acid substitutions, deletions, or additions). In some embodiments, the amino acid sequence of the one or more hIg constant regions comprises or consists of the amino acid sequence of any one of SEQ ID NOS: 103-104, comprising or consisting at least about 1, 2, 3, 4, 5, 6, 7, 8, 9, 10, or more amino acid modifications (e.g., amino acid substitutions, deletions, or additions). In some embodiments, the amino acid sequence of the one or more hIg constant regions comprises or consists of the amino acid sequence of any one of SEQ ID NOS: 103-104, comprising or consisting about 1, 2, 3, 4, 5, 6, 7, 8, 9, or 10 amino acid modifications (e.g., amino acid substitutions, deletions, or additions). In some embodiments, the amino acid sequence of the one or more hIg constant regions comprises or consists of the amino acid sequence of any one of SEQ ID NOS: 103-104, comprising or consisting of no more than about 1, 2, 3, 4, 5, 6, 7, 8, 9, or 10 amino acid modifications (e.g., amino acid substitutions, deletions, or additions).

In some embodiments, the amino acid sequence of the one or more hIg constant regions comprises or consists of the amino acid sequence of any one of SEQ ID NOS: 103-104, and further comprises 1 or more but less than 15% (less than 12%, less than 10%, less than 8%), amino acid substitutions. In some embodiments, the amino acid sequence of the one or more hIg constant regions comprises or consists of the amino acid sequence of any one of SEQ ID NOS: 103-104, comprising or consisting at least about 1, 2, 3, 4, 5, 6, 7, 8, 9, 10, or more amino acid substitutions. In some embodiments, the amino acid sequence of the one or more hIg constant regions comprises or consists of the amino acid sequence of any one of SEQ ID NOS: 103-104, comprising or consisting about 1, 2, 3, 4, 5, 6, 7, 8, 9, or 10 amino acid substitutions. In some embodiments, the amino acid sequence of the one or more hIg constant regions comprises or consists of the amino acid sequence of any one of SEQ ID NOS: 103-104, comprising or consisting of no more than about 1, 2, 3, 4, 5, 6, 7, 8, 9, or 10 amino acid substitutions.

### 5.5.2 Linkers

The moieties (e.g., EGFR (e.g., hEGFR) binding moiety and TGFβ (e.g., hTGFβ) binding moiety) of fusion proteins described herein can be directly operably connected, e.g., through a peptide bond, or indirectly operably connected, e.g., through a linker (e.g., a peptide linker). In some embodiments, one or more of the moieties of the fusion protein are directly operably connected through a peptide bond. In some embodiments, one or more of the moieties of the fusion protein are indirectly operably connected through a peptide linker.

In some embodiments, the peptide linker is one or any combination of a cleavable linker, a non-cleavable linker, a flexible linker, a rigid linker, a helical linker, and/or a non-helical linker.

In some embodiments, the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises or consists of glycine, serine, or both glycine and serine amino acid residues. In some embodiments, the amino acid sequence of the peptide linker comprises or consists of glycine, serine, and proline amino acid residues.

The amino acid sequence of exemplary peptide linkers, which can be incorporated in one or more of the embodiments described herein (e.g., fusion proteins and polypeptide), is set provided in Table 7.

TABLE 7
The Amino Acid Sequence of Exemplary
Peptide Linker.

| | | SEQ |
|---|---|---|
| Descrip- | | ID |
| tion | Amino Acid Sequence | NO |
| Linker A | GGGS | 105 |
| Linker B | GGGSGGGS | 106 |
| Linker C | GGGSGGGSGGGS | 107 |
| Linker D | GGGSGGGSGGGSGGGS | 108 |
| Linker E | GGGGS | 109 |
| Linker F | GGGGSGGGGS | 110 |
| Linker G | GGGGSGGGGSGGGGS | 111 |
| Linker H | GGGGSGGGGSGGGGSGGGGS | 112 |
| Linker I | GGGGGGGS | 113 |
| Linker J | GGGGGGGSGGGGGGGS | 114 |
| Linker K | GGGGGGGSGGGGGGGGGGGGGGS | 115 |
| Linker L | GGGGGGGSGGGGGGGSGGGGGGGSGGGGGGGS | 116 |
| Linker M | SGGGG | 117 |
| Linker N | SGGGGSGGGG | 118 |
| Linker O | SGGGGSGGGGSGGGG | 119 |
| Linker P | SGGGGSGGGGSGGGGSGGGG | 120 |
| Linker Q | GGGGGGS | 121 |
| Linker R | GGGGGGSGGGGGGS | 122 |
| Linker S | GGGGGGSGGGGGGSGGGGGGS | 123 |
| Linker T | GGGGGGSGGGGGGSGGGGGGSGGGGGGS | 124 |

In some embodiments, the amino acid sequence of the peptide linker comprises or consists of the amino acid sequence of any one of the linkers set forth in Table 7. In some embodiments, the amino acid sequence of the peptide linker is at least 85%, 86%, 87%, 88%, 89%, 90%, 91%, 92%, 93%, 94%, 95%, 96%, 97%, 98%, 99%, or 100% identical to the amino acid sequence of a peptide linker set forth in Table 7.

In some embodiments, the amino acid sequence of the peptide linker comprises or consists of the amino acid sequence of any one of the linkers set forth in Table 7, and further comprises 1 or more but less than 15% (less than 12%, less than 10%, less than 8%), amino acid modifications (e.g., amino acid substitutions, deletions, or additions). In some embodiments, the amino acid sequence of the peptide linker comprises or consists of the amino acid sequence of any one of the linkers set forth in Table 7, comprising 1, 2, or 3 amino acid modifications (e.g., substitutions, deletions, additions). In some embodiments, the amino acid sequence of the peptide linker comprises or consists of the amino acid sequence of any one of the linkers set forth in Table 7, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 7, comprising 1, 2, or 3 amino acid substitutions.

In some embodiments, the amino acid sequence of the peptide linker comprises or consists of the amino acid sequence of any one of SEQ ID NOS: 105-124. In some embodiments, the amino acid sequence of the peptide linker is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 105-124. In some embodiments, the amino acid sequence of the peptide linker comprises or consists of the amino acid sequence of any one of SEQ ID NO: 111. In some embodiments, the amino acid sequence of the peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111. In some embodiments, the amino acid sequence of the peptide linker comprises the amino acid sequence of any one of SEQ ID NO: 111. In some embodiments, the amino acid sequence of the peptide linker consists of the amino acid sequence of any one of SEQ ID NO: 111. In some embodiments, the amino acid sequence of the peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111.

In some embodiments, the amino acid sequence of the peptide linker comprises or consists of the amino acid sequence of any one of SEQ ID NOS: 105-124, and further comprises 1 or more but less than 15% (less than 12%, less than 10%, less than 8%), amino acid modifications (e.g., amino acid substitutions, deletions, or additions). In some embodiments, the amino acid sequence of the peptide linker comprises or consists of the amino acid sequence of any one of SEQ ID NOS: 105-124, comprising 1, 2, or 3 amino acid modifications (e.g., substitutions, deletions, additions). In some embodiments, the amino acid sequence of the peptide linker comprises or consists of the amino acid sequence of any one of SEQ ID NOS: 105-124, and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 105-124, comprising 1, 2, or 3 amino acid substitutions.

In some embodiments, the amino acid sequence of the peptide linker comprises or consists of the amino acid sequence of SEQ ID NO: 111, and further comprises 1 or more but less than 15% (less than 12%, less than 10%, less than 8%), amino acid substitutions. In some embodiments, the amino acid sequence of the peptide linker comprises or consists of the amino acid sequence of SEQ ID NO: 111. In some embodiments, the amino acid sequence of the peptide linker comprises or consists of the amino acid sequence of SEQ ID NO: 111, comprising 1, 2, or 3 amino acid modifications (e.g., substitutions, additions, deletions). In some embodiments, the amino acid sequence of the peptide linker comprises or consists of the amino acid sequence of SEQ ID NO: 111, comprising 1, 2, or 3 amino acid substitutions.

In some embodiments, the amino acid sequence of the peptide linker comprises the amino acid sequence of SEQ ID NO: 111, and further comprises 1 or more but less than 15% (less than 12%, less than 10%, less than 8%), amino acid substitutions. In some embodiments, the amino acid sequence of the peptide linker comprises the amino acid sequence of SEQ ID NO: 111. In some embodiments, the amino acid sequence of the peptide linker comprises the amino acid sequence of SEQ ID NO: 111, comprising 1, 2, or 3 amino acid modifications (e.g., substitutions, additions, deletions). In some embodiments, the amino acid sequence of the peptide linker comprises the amino acid sequence of SEQ ID NO: 111, comprising 1, 2, or 3 amino acid substitutions.

In some embodiments, the amino acid sequence of the peptide linker consists of the amino acid sequence of SEQ ID NO: 111, and further comprises 1 or more but less than 15% (less than 12%, less than 10%, less than 8%), amino acid substitutions. In some embodiments, the amino acid sequence of the peptide linker consists of the amino acid sequence of SEQ ID NO: 111. In some embodiments, the amino acid sequence of the peptide linker consists of the amino acid sequence of SEQ ID NO: 111, comprising 1, 2, or 3 amino acid modifications (e.g., substitutions, additions, deletions). In some embodiments, the amino acid sequence of the peptide linker consists the amino acid sequence of SEQ ID NO: 111, comprising 1, 2, or 3 amino acid substitutions.

### 5.5.3 Orientation

The moieties (e.g., EGFR (e.g., hEGFR) binding moiety (e.g., an EGFR (e.g., hEGFR) binding moiety described herein), TGFβ binding moiety (e.g., a TGFβ (e.g., hTGFβ) binding moiety described herein)) of a fusion protein (e.g., a fusion protein described herein) can be arranged in any configuration or order as long as each moiety maintains the ability to mediate its function (e.g., the EGFR (e.g., hEGFR) binding moiety maintains the ability to bind EGFR (e.g., hEGFR), and the TGFβ (e.g., hTGFβ) binding moiety maintains the ability to bind TGFβ (e.g., hTGFβ)).

Exemplary orientations of fusion proteins described herein are provided below. The exemplary orientations are not intended to be limiting but exemplary only.

### 5.5.3.1 Homodimeric Full-Length Antibody-C-Terminal Light Chain Fusion Proteins

In some embodiments, the fusion protein comprises an EGFR (e.g., hEGFR) binding moiety, a first TGFβ (e.g., hTGFβ) binding moiety, and a second TGFβ (e.g., hTGFβ) binding moiety, wherein (a) the EGFR (e.g., hEGFR) binding moiety is a full-length antibody that comprises (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer, (b) the N-terminus of the first TGFβ (e.g., hTGFβ) binding moiety (e.g., the ECD of a TGFβR (e.g., TGFβRII (e.g., hTGFβRII))) is operably connected to the C-terminus of the first Ig light chain of the full-length antibody; and (c) the N-terminus of the second TGFβ binding moiety (e.g., the ECD of a TGFβR (e.g., TGFβRII (e.g., hTGFβRII))) is operably connected to the C-terminus of the second Ig light chain of the full-length antibody.

In some embodiments, the first TGFβ (e.g., hTGFβ) binding moiety is operably connected to the C-terminus of the first Ig light chain of the full-length antibody through a first peptide linker (e.g., a peptide linker described herein, e.g., SEQ ID NO: 111); and the second TGFβ (e.g., hTGFβ) binding moiety is operably connected to the C-terminus of the second Ig light chain of the full-length antibody through a second peptide linker (e.g., a peptide linker described herein, e.g., SEQ ID NO: 111). In some embodiments, the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of the second peptide linker. In some embodiments, the amino acid sequence of the first peptide linker is 100% identical to the amino acid sequence of the second peptide linker.

In some embodiments, the amino acid sequence of the TGFβ (e.g., hTGFβ) binding moiety is at least 85%, 86%, 87%, 88%, 89%, 90%, 91%, 92%, 93%, 94%, 95%, 96%, 97%, 98%, 99%, or 100% identical to the amino acid sequence of the second TGFβ (e.g., hTGFβ) binding moiety. In some embodiments, the amino acid sequence of the TGFβ (e.g., hTGFβ) binding moiety is 100% identical to the amino acid sequence of the second TGFβ (e.g., hTGFβ) binding moiety. In some embodiments, the first TGFβ (e.g., hTGFβ) binding moiety comprises or consists of the ECD of a TGFβR (e.g., TGFβRII (e.g., hTGFβRII)); and the second TGFβ (e.g., hTGFβ) binding moiety comprises or consists of the ECD of a TGFβR (e.g., TGFβRII (e.g., hTGFβRII)).

In some embodiments, the fusion protein comprises (a) a first polypeptide that comprises from N- to C-terminus a first light chain variable region (VL), a first light chain constant region (CL), a first optional peptide linker, and a first TGFβ binding moiety (e.g., the ECD of a TGFβR (e.g., TGFβRII (e.g., hTGFβRII))); (b) a second polypeptide that comprises from N- to C-terminus a first heavy chain variable region (VH) and a first heavy chain constant region (CH); (c) a third polypeptide that comprises from N- to C-terminus a second VH and a second CH; and (d) a fourth polypeptide that comprises from N- to C-terminus a second VL, a second CL, a second optional peptide linker, and a second TGFβ binding moiety (e.g., the ECD of a TGFβR (e.g., TGFβRII (e.g., hTGFβRII))); wherein the first VL and the first VH form a first antigen binding domain that specifically binds EGFR (e.g., hEGFR); and wherein said second VL and said second VH form a second antigen binding domain that specifically binds EGFR (e.g., hEGFR). In some embodiments, the first polypeptide comprises an amino acid sequence at least 90%, 91%, 92%, 93%, 94%, 95%, 96%, 97%, 98, 99%, or 100% identical to the amino acid sequence of the fourth polypeptide. In some embodiments, the second polypeptide comprises an amino acid sequence at least 90%, 91%, 92%, 93%, 94%, 95%, 96%, 97%, 98, 99%, or 100% identical to the amino acid sequence of the third polypeptide. In some embodiments, the amino acid sequence of said first TGFβ binding moiety (e.g., the ECD of a TGFβR (e.g., TGFβRII (e.g., hTGFβRII))) is at least 90%, 91%, 92%, 93%, 94%, 95%, 96%, 97%, 98, 99%, or 100% identical to the amino acid sequence of the second TGFβ binding moiety (e.g., the ECD of a TGFβR (e.g., TGFβRII (e.g., hTGFβRII))). In some embodiments, the first CH region comprises from N- to C-terminus a first CH1 domain, a first hinge domain, a first CH2 domain, and a first CH3 domain. In some embodiments, the second CH region comprises from N- to C-terminus a second CH1 domain, a second hinge domain, a second CH2 domain, and a second CH3 domain.

In some embodiments, the fusion protein comprises (a) a first moiety that comprises or consists of a full-length antibody that specifically binds EGFR (e.g., hEGFR); (b) a second moiety that comprises a comprises or consists of the ECD of TGFβRII (e.g., hTGFβRII); (c) a third moiety that comprises a comprises or consists of the ECD of TGFβRII (e.g., hTGFβRII); wherein the second moiety is operably connected to the first moiety through a first peptide linker; wherein the third moiety is operably connected to the first moiety through a second peptide linker, and wherein the wherein the N-terminus of the second moiety is operably connected to the C-terminus of the first light chain of the full-length antibody and the N-terminus of the third moiety is operably connected to the C-terminus of the second light chain of the full-length antibody.

### 5.5.3.2 Homodimeric Full-Length Antibody-C-Terminal Heavy Chain Fusion Proteins

In some embodiments, the fusion protein (e.g., described herein) comprises an EGFR (e.g., hEGFR) binding moiety, a first TGFβ (e.g., hTGFβ) binding moiety, and a second TGFβ (e.g., hTGFβ) binding moiety, wherein (a) the EGFR (e.g., hEGFR) binding moiety is a full-length antibody that comprises (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer, (b) the N-terminus of the first TGFβ binding moiety (e.g., the ECD of a TGFβR (e.g., TGFβRII (e.g., hTGFβRII))) is operably connected to the C-terminus of the first Ig heavy chain of the full-length antibody; and (c) the N-terminus of the second TGFβ (e.g., hTGFβ) binding moiety (e.g., the ECD of a TGFβR (e.g., TGFβRII (e.g., hTGFβRII))) is operably connected to the C-terminus of the second Ig heavy chain of the full-length antibody.

In some embodiments, the first TGFβ (e.g., hTGFβ) binding moiety is operably connected to the C-terminus of the first Ig heavy chain of the full-length antibody through a first peptide linker (e.g., a peptide linker described herein, e.g., SEQ ID NO: 111); and the second TGFβ (e.g., hTGFβ) binding moiety is operably connected to the C-terminus of the second Ig heavy chain of the full-length antibody through a second peptide linker (e.g., a peptide linker described herein, e.g., SEQ ID NO: 111). In some embodiments, the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of the second peptide linker. In some embodiments, the amino acid sequence of the first peptide linker is 100% identical to the amino acid sequence of the second peptide linker.

In some embodiments, the amino acid sequence of the TGFβ (e.g., hTGFβ) binding moiety is at least 85%, 86%, 87%, 88%, 89%, 90%, 91%, 92%, 93%, 94%, 95%, 96%, 97%, 98%, 99%, or 100% identical to the amino acid sequence of the second TGFβ (e.g., hTGFβ) binding moiety. In some embodiments, the amino acid sequence of the TGFβ (e.g., hTGFβ) binding moiety is 100% identical to the amino acid sequence of the second TGFβ (e.g., hTGFβ) binding moiety. In some embodiments, the first TGFβ (e.g., hTGFβ) binding moiety comprises or consists of the ECD of a TGFβR (e.g., TGFβRII (e.g., hTGFβRII)); and the second TGFβ (e.g., hTGFβ) binding moiety comprises or consists of the ECD of a TGFβR (e.g., TGFβRII (e.g., hTGFβRII)).

In some embodiments, the fusion protein comprises (a) a first polypeptide that comprises from N- to C-terminus a first light chain variable region (VL) and a first light chain constant region (CL); (b) a second polypeptide that comprises from N- to C-terminus a first heavy chain variable region (VH), a first heavy chain constant region (CH), a first optional peptide linker, and a first TGFβ (e.g., hTGFβ) binding moiety (e.g., the ECD of a TGFβR (e.g., TGFβRII (e.g., hTGFβRII))); (c) a third polypeptide that comprises from N- to C-terminus a second VH, a second CH, a second optional peptide linker, and a second TGFβ (e.g., hTGFβ) binding moiety (e.g., the ECD of a TGFβR (e.g., TGFβRII (e.g., hTGFβRII))); and (d) a fourth polypeptide that comprises from N- to C-terminus a second VL and a second CL; wherein the first VL and the first VH form a first antigen binding domain that specifically binds EGFR (e.g., hEGFR); and wherein said second VL and said second VH form a second antigen binding domain that specifically binds

EGFR (e.g., hEGFR). In some embodiments, the first polypeptide comprises an amino acid sequence at least 90%, 91%, 92%, 93%, 94%, 95%, 96%, 97%, 98, 99%, or 100% identical to the amino acid sequence of the fourth polypeptide. In some embodiments, the second polypeptide comprises an amino acid sequence at least 90%, 91%, 92%, 93%, 94%, 95%, 96%, 97%, 98, 99%, or 100% identical to the amino acid sequence of the third polypeptide. In some embodiments, the amino acid sequence of said first TGFβ (e.g., hTGFβ) binding moiety (e.g., the ECD of a TGFβR (e.g., TGFβRII (e.g., hTGFβRII))) is at least 90%, 91%, 92%, 93%, 94%, 95%, 96%, 97%, 98, 99%, or 100% identical to the amino acid sequence of the second TGFβ (e.g., hTGFβ) binding moiety (e.g., the ECD of a TGFβR (e.g., TGFβRII (e.g., hTGFβRII))). In some embodiments, the first CH region comprises from N- to C-terminus a first CH1 domain, a first hinge domain, a first CH2 domain, and a first CH3 domain. In some embodiments, the second CH region comprises from N- to C-terminus a second CH1 domain, a second hinge domain, a second CH2 domain, and a second CH3 domain.

### 5.5.4 Exemplary Fusion Proteins

The amino acid sequence of exemplary fusion proteins described herein is provided in Table 8. Each of the fusion proteins comprises an EGFR (e.g., hEGFR) binding moiety and two TGFβ binding moieties, wherein the EGFR (e.g., hEGFR) binding moiety comprises a full-length antibody comprising two EGFR (e.g., hEGFR) binding domains; and each TGFβ (e.g., hTGFβ) binding moiety comprises the ECD of TGFβRII (e.g., hTGFβRII). The fusion proteins provided in Table 8 are exemplary only, and not intended to be limiting.

TABLE 8
The Amino Acid Sequence of Exemplary Fusion Proteins.

| | | | SEQ |
|---|---|---|---|
| | | | ID |
| Description | | Amino Acid Sequence | NO |
| αEGFR x | Heavy | QVQLKQSGPGLVQPSQSLSITCTVSGFSLTNYGVHWVRQSP | 125 |
| TGFβRII ECD | Chain | GKGLEWLGVIWSGGNTDYNTPFTSRLSINKDNSKSQVFFKM | |
| C-terminal LC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| BCA101 | | LTSGVHTFPAVLQSSGLYSLSSVVTVPSSSLGTQTYICNVN | |
| | | HKPSNTKVDKRVEPKSCDKTHTCPPCPAPELLGGPSVELFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPG | |
| | Light | DILLTQSPVILSVSPGERVSFSCRASQSIGTNIHWYQQRTN | 126 |
| | Chain | GSPRLLIKYASESISGIPSRFSGSGSGTDFTLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGECGGGGSGGGGSGGGGSTIPPHVQKSVNNDMIVT | |
| | | DNNGAVKFPQLCKFCDVRFSTCDNQKSCMSNCSITSICEKP | |
| | | QEVCVAVWRKNDENITLETVCHDPKLPYHDFILEDAASPKC | |
| | | IMKEKKKPGETFFMCSCSSDECNDNIIFSEEYNTSNPD | |
| αEGFR x | Heavy | QVQLKQSGPGLVQPSQSLSITCTVSGFSLTNYGVHWVRQSP | 127 |
| TGFβRII ECD | Chain | GKGLEWLGVIWSGGNTDYNTPFTSRLSINKDNSKSQVFFKM | |
| C-terminal LC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPG | |
| | Light | DILLTQSPVILSVSPGERVSFSCRASQSIGTNIHWYQQRTN | 128 |
| | Chain | GSPRLLIKYASESISGIPSRFSGSGSGTDFTLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGECGGGGSGGGGSGGGGSTIPPHVQKSVNNDMIVT | |
| | | DNNGAVKFPQLCKFCDVRFSTCDNQKSCMSNCSITSICEKP | |
| | | QEVCVAVWRKNDENITLETVCHDPKLPYHDFILEDAASPKC | |
| | | IMKEKKKPGETFFMCSCSSDECNDNIIFSEEYNTSNPD | |
| αEGFR x | Heavy | QVQLKQSGPGLVQPSQSLSITCTVSGFSLTNYGVHWVRQSP | 129 |
| TGFβRII ECD | Chain | GKGLEWLGVIWSGGNTDYNTPFTSRLSINKDNSKSQVFFKM | |
| C-terminal LC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKRVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGK | |
| | Light | DILLTQSPVILSVSPGERVSFSCRASQSIGTNIHWYQQRTN | 130 |
| | Chain | GSPRLLIKYASESISGIPSRESGSGSGTDFTLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGECGGGGSGGGGSGGGGSTIPPHVQKSVNNDMIVT | |
| | | DNNGAVKFPQLCKFCDVRFSTCDNQKSCMSNCSITSICEKP | |
| | | QEVCVAVWRKNDENITLETVCHDPKLPYHDFILEDAASPKC | |
| | | IMKEKKKPGETFFMCSCSSDECNDNIIFSEEYNTSNPD | |
| αEGFR x | Heavy | QVQLKQSGPGLVQPSQSLSITCTVSGFSLTNYGVHWVRQSP | 131 |
| TGFβRII ECD | Chain | GKGLEWLGVIWSGGNTDYNTPFTSRLSINKDNSKSQVFFKM | |
| C-terminal LC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGK | |
| | Light | DILLTQSPVILSVSPGERVSFSCRASQSIGTNIHWYQQRTN | 132 |
| | Chain | GSPRLLIKYASESISGIPSRFSGSGSGTDETLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGECGGGGSGGGGSGGGGSTIPPHVQKSVNNDMIVT | |
| | | DNNGAVKFPQLCKFCDVRFSTCDNQKSCMSNCSITSICEKP | |
| | | QEVCVAVWRKNDENITLETVCHDPKLPYHDFILEDAASPKC | |
| | | IMKEKKKPGETFFMCSCSSDECNDNIIFSEEYNTSNPD | |
| αEGFR x | Heavy | QVQLKQSGPGLVQPSQSLSITCTVSGFSLTNYGVHWVRQSP | 133 |
| TGFβRII ECD | Chain | GKGLEWLGVIWSGGNTDYNTPFTSRLSINKDNSKSQVFFKM | |
| C-terminal HC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKRVEPKSCDKTHTCPPCPAPELLGGPSVFLFP | |
| | | PKPKDTLMISRTPEVTCVVVDVSHEDPEVKENWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGGGG | |
| | | GSGGGGSGGGGSTIPPHVQKSVNNDMIVTDNNGAVKFPQLC | |
| | | KFCDVRESTCDNQKSCMSNCSITSICEKPQEVCVAVWRKND | |
| | | ENITLETVCHDPKLPYHDFILEDAASPKCIMKEKKKPGETF | |
| | | FMCSCSSDECNDNIIFSEEYNTSNPD | |
| | Light | DILLTQSPVILSVSPGERVSFSCRASQSIGTNIHWYQQRTN | 134 |
| | Chain | GSPRLLIKYASESISGIPSRESGSGSGTDFTLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGEC | |
| αEGFR x | Heavy | QVQLKQSGPGLVQPSQSLSITCTVSGFSLTNYGVHWVRQSP | 135 |
| TGFβRII ECD | Chain | GKGLEWLGVIWSGGNTDYNTPFTSRLSINKDNSKSQVFFKM | |
| C-terminal HC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGGGG | |
| | | GSGGGGSGGGGSTIPPHVQKSVNNDMIVTDNNGAVKFPQLC | |
| | | KFCDVRFSTCDNQKSCMSNCSITSICEKPQEVCVAVWRKND | |
| | | ENITLETVCHDPKLPYHDFILEDAASPKCIMKEKKKPGETF | |
| | | FMCSCSSDECNDNIIFSEEYNTSNPD | |
| | Light | DILLTQSPVILSVSPGERVSFSCRASQSIGTNIHWYQQRTN | 136 |
| | Chain | GSPRLLIKYASESISGIPSRFSGSGSGTDFTLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGEC | |
| αEGFR x | Heavy | QVQLKQSGPGLVQPSQSLSITCTVSGFSLTNYGVHWVRQSP | 137 |
| TGβFRII ECD | Chain | GKGLEWLGVIWSGGNTDYNTPFTSRLSINKDNSKSQVFFKM | |
| C-terminal HC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKRVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGKGG | |
| | | GGSGGGGSGGGGSTIPPHVQKSVNNDMIVTDNNGAVKFPQL | |
| | | CKFCDVRFSTCDNQKSCMSNCSITSICEKPQEVCVAVWRKN | |
| | | DENITLETVCHDPKLPYHDFILEDAASPKCIMKEKKKPGET | |
| | | FFMCSCSSDECNDNIIFSEEYNTSNPD | |
| | Light | DILLTQSPVILSVSPGERVSFSCRASQSIGTNIHWYQQRTN | 138 |
| | Chain | GSPRLLIKYASESISGIPSRFSGSGSGTDFTLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGEC | |
| αEGFR x | Heavy | QVQLKQSGPGLVQPSQSLSITCTVSGFSLTNYGVHWVRQSP | 139 |
| TGFβRII ECD | Chain | GKGLEWLGVIWSGGNTDYNTPFTSRLSINKDNSKSQVFFKM | |
| C-terminal HC | | NSLQSNDTAIYYCARALTYYDYEFAYWGQGTLVTVSAASTK | |
| Fusion | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGKGG | |
| | | GGSGGGGSGGGGSTIPPHVQKSVNNDMIVTDNNGAVKFPQL | |
| | | CKFCDVRFSTCDNQKSCMSNCSITSICEKPQEVCVAVWRKN | |
| | | DENITLETVCHDPKLPYHDFILEDAASPKCIMKEKKKPGET | |
| | | FFMCSCSSDECNDNIIFSEEYNTSNPD | |
| | Light | DILLTQSPVILSVSPGERVSFSCRASQSIGTNIHWYQQRTN | 140 |
| | Chain | GSPRLLIKYASESISGIPSRFSGSGSGTDFTLSINSVESED | |
| | | IADYYCQQNNNWPTTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGEC | |

In some embodiments, the fusion protein comprises a heavy chain (HC) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a HC in Table 8; and a light chain (LC) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a LC in Table 8.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125. In some embodiments, the fusion protein comprises a HC consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126. In some embodiments, the fusion protein comprises a LC consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.

In some embodiments, the fusion protein comprises a HC consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; and a LC consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 127.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 128.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 127; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 128.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 129.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence SEQ ID NO: 130.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 129; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 130.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 131.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 132.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 131; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 132.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 133.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 134.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 133; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 134.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 135.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 136.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 135; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 136.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 137.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 138.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 137; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 138.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 139.

In some embodiments, the fusion protein comprises a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 140.

In some embodiments, the fusion protein comprises a HC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 139; and a LC comprising an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 140.

### 5.5.5 Affinity of Fusion Proteins and Antigen Binding Moieties Thereof

Binding affinity of any of the fusion proteins or components thereof (e.g., EGFR (e.g., hEGFR) binding moieties, TGFβ (e.g., hTGFβ) binding moieties) described herein can be measured by standard assays known in the art and described herein. For example, binding affinity can be measured by ELISA. Binding affinity can also be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated herein by reference for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip.

Other suitable assays for measuring the binding of a multispecific protein described herein for one or more of its cognate antigens include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

As described above, functionality of the fusion protein can be tested by any method known in the art, e.g., ELISA. For fusion proteins possessing more than one functionality, each functionality can be measured in a separate assay, e.g., TGFβ (e.g., hTGFβ) binding and EGFR (e.g., hEGFR) binding can be measured in two separate ELISAs. For example, an ELISA plate can be coated with EGFR (e.g., hEGFR) Fc chimera can be used to evaluate EGFR (e.g., hEGFR) binding; and a separate ELISA plate can be coated with TGFβ (e.g., TGFβ1 (e.g., hTGFβ1)) can be used to evaluate TGFβ (e.g., TGFβ1 (e.g., hTGFβ) 1) binding.

Both functionalities can also be evaluated in a bifunctional ELISA. For example, an anti-idiotype monoclonal antibody against the EGFR (e.g., hEGFR) antibody (e.g., cetuximab or a variant thereof) can be used to capture the EGFR antibody portion of the fusion protein (e.g., BCA101) and the bound fusion protein (e.g., BCA101) can be detected by an enzyme-labeled polyclonal antibody against the TGFβ (e.g., hTGFβ) binding moiety (e.g., the ECD of TGFβRII (e.g., hTGFβRII)). The concentration of fusion protein (e.g., BCA101) in samples can be back calculated from a fusion protein calibration curve.

Target binding can also be evaluated via Biocore, wherein EGFR (e.g., hEGFR) and TGFβ (e.g., TGFβ1 (e.g., hTGFβ1)) targets are immobilized on activated CM5 chips and then incubated with serial concentrations of the fusion protein (e.g., BCA101). Additional in vitro functional assays can also be performed to evaluate the fusion proteins, including for example, cell surface binding by flow cytometry, inhibition of cell proliferation, ADCC assay, neutralization of TGFβ (e.g., TGFβ1 (e.g., hTGFβ1)) induced IL-11 release; neutralization of TGFβ (e.g., TGFβ1 (e.g., hTGFβ1)) induced SMAD signaling.

### 5.6 Methods of Making Fusion Proteins & Components Thereof

The fusion proteins (and polypeptides thereof) and components thereof (e.g., moieties thereof) described herein may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the fusion protein (or one or more polypeptide thereof). Such general methods are common knowledge in the art. The expression vector typically contains an expression cassette that includes nucleic acid sequences capable of bringing about expression of the nucleic acid molecule encoding the protein or polypeptide of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein or polypeptide of interest are cultured under conditions conducive to expression of the nucleic acid molecule encoding the antigenic peptide or protein. Culture media is available from various vendors, and a suitable medium can be routinely chosen for a host cell to express a protein or polypeptide of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein or polypeptide may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein or polypeptide may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes. The fusion proteins (and polypeptides thereof) described herein and the components thereof (e.g., moieties thereof) described herein may be produced synthetically.

In embodiments, the disclosure features methods of making the fusion proteins (and polypeptides thereof) and components thereof (e.g., moieties thereof) described herein. The method includes (a) recombinantly expressing a fusion protein (or polypeptide thereof) or a component thereof (e.g., a moiety thereof) described herein; (b) enriching, e.g., purifying, the fusion protein (or polypeptide thereof) or the component thereof (e.g., the moiety thereof) described herein; (c) evaluating the fusion protein (or polypeptide thereof) or the component thereof (e.g., the moiety thereof) described herein for the presence of a process impurity or contaminant, and (d) formulating the fusion protein (or polypeptide thereof) or the component thereof (e.g., the moiety thereof) described herein as a pharmaceutical composition if the fusion protein (or polypeptide thereof) or the component thereof (e.g., the moiety thereof) described herein meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.7 Combination Regimens

In one aspect, provided herein are combination regimens comprising (i) (a) a KRAS (e.g., hKRAS) inhibitor (e.g., a KRAS (e.g., hKRAS) inhibitor described herein) (e.g., a pharmaceutical composition comprising a KRAS (e.g., hKRAS) inhibitor (e.g., a KRAS (e.g., hKRAS) inhibitor described herein)); in combination with (ii) (a) a fusion protein (or polypeptides thereof) described herein, (b) a polynucleotide encoding a fusion protein (or polypeptides thereof) described herein, (c) a vector comprising a polynucleotide described herein (e.g., a polynucleotide encoding a fusion protein (or polypeptides thereof) described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), (e) a carrier described herein (e.g., a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), or (f) a pharmaceutical composition described herein (e.g., a pharmaceutical composition comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein)), or a carrier described herein (e.g., a carrier a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein).

The components (i) and (ii) set forth above of the combination regimen may or may not be combined in a single dosage form. In some embodiments, the components (i) and (ii) set forth above of the combination regimen are not combined in a single dosage form. In some embodiments, the components (i) and (ii) set forth above of the combination regimen are combined in a single dosage form.

The components (i) and (ii) set forth above of the combination regimen may be formulated in separate formulations or in single formulation. In some embodiments, the components (i) and (ii) set forth above of the combination regimen are formulated in separate formulations. In some embodiments, the components (i) and (ii) set forth above of the combination regimen are formulated in a single formulation.

The components (i) and (ii) set forth above of the combination regimen may be formulated in separate pharmaceutical formulations or in single pharmaceutical formulation. In some embodiments, the components (i) and (ii) set forth above of the combination regimen are formulated in separate pharmaceutical formulations. In some embodiments, the components (i) and (ii) set forth above of the combination regimen are formulated in a single pharmaceutical formulation.

The combination regimens described herein can be utilized, e.g., in any of the combination compositions described herein, see e.g., § 5.8; in any of the pharmaceutical compositions described herein see e.g., § 5.10; in any of the methods of use described herein, see e.g., § 5.11; in any of the kits described herein see e.g., § 5.12.

### 5.8 Combination Compositions

In one aspect, provided herein are combination compositions comprising (i) (a) a KRAS (e.g., hKRAS) inhibitor (e.g., a KRAS (e.g., hKRAS) inhibitor described herein) (e.g., a pharmaceutical composition comprising a KRAS (e.g., hKRAS) inhibitor (e.g., a KRAS (e.g., hKRAS) inhibitor described herein)); and (ii) (a) a fusion protein (or polypeptides thereof) described herein, (b) a polynucleotide encoding a fusion protein (or polypeptides thereof) described herein, (c) a vector comprising a polynucleotide described herein (e.g., a polynucleotide encoding a fusion protein (or polypeptides thereof) described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), (e) a carrier described herein (e.g., a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), or (f) a pharmaceutical composition described herein (e.g., a pharmaceutical composition comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein)), or a carrier described herein (e.g., a carrier a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein).

The combination compositions described herein can be utilized, e.g., in any of the combination compositions described herein, see e.g., § 5.8; in any of the pharmaceutical compositions described herein see e.g., § 5.10; in any of the methods of use described herein, see e.g., § 5.11; in any of the kits described herein see e.g., § 5.12.

### 5.9 Polynucleotides, Vectors, Carriers, & Host Cells

In one aspect, provided herein are polynucleotides (e.g., DNA, RNA) encoding a fusion protein (or any (e.g., one or more)) polypeptide thereof) described herein and components thereof (e.g., moieties thereof (e.g., an EGFR binding moiety, TGFβ binding moiety)) described herein. In some embodiments, the polynucleotide is a DNA polynucleotide or an RNA polynucleotide. In some embodiments, the polynucleotide is an mRNA polynucleotide. In some embodiments, the polynucleotide is codon optimized. Codon optimization, may be used to match codon frequencies in target and host organisms to ensure proper folding; bias guanosine (G) and/or cytosine I content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

In one aspect, provided herein are vectors comprising a polynucleotide (e.g., DNA, RNA) described herein (e.g., a polynucleotide encoding a fusion protein (or one or more polypeptide thereof) described herein or components thereof (e.g., moieties thereof (e.g., an EGFR binding moiety, TGFβ binding moiety)) described herein. In some embodiments, the vector is a viral vector. In some embodiments, the vector is a non-viral vector (e.g., a plasmid).

In one aspect, provided herein are carriers comprising (a) a fusion protein (or one or more polypeptide thereof) described herein or components thereof (e.g., moieties thereof (e.g., an EGFR binding moiety, TGFβ binding moiety)) described herein, a polynucleotide described herein (e.g., a polynucleotide encoding comprising a fusion protein (or any (e.g., one or more)) polypeptide thereof) described herein and components thereof (e.g., moieties thereof (e.g., an EGFR binding moiety, TGFβ binding moiety)) described herein, or a vector described herein (e.g., a vector comprising a polynucleotide described herein); and/or (b) a KRAS inhibitor (e.g., a KRAS inhibitor described herein). Carriers include, but are not limited to, lipid-based carriers such as lipid nanoparticles (LNPs), liposomes, lipoplexes, or nanoliposomes. In some embodiments, the carrier is an LNP, e.g., an LNP described herein.

In one aspect, provided herein are host cells comprising (a) a fusion protein (or one or more polypeptide thereof) described herein or components thereof (e.g., moieties thereof (e.g., an EGFR binding moiety, TGFβ binding moiety)) described herein, a polynucleotide described herein (e.g., a polynucleotide encoding a fusion protein (or any (e.g., one or more)) polypeptide thereof) described herein and components thereof (e.g., moieties thereof (e.g., an EGFR binding moiety, TGFβ binding moiety)) described herein, a vector described herein (e.g., a vector comprising a polynucleotide described herein), or a carrier described herein (e.g., a carrier comprising a fusion protein (or any (e.g., one or more)) polypeptide thereof) described herein and components thereof (e.g., moieties thereof (e.g., an EGFR binding moiety, TGFβ binding moiety)) described herein); and/or (b) a KRAS inhibitor (e.g., a KRAS inhibitor described herein).

### 5.10 Pharmaceutical Compositions

In one aspect, provided herein are pharmaceutical compositions comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), a host cell described herein (e.g., a host cell comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein))), or a carrier described herein (e.g., a carrier comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), or a host cell described herein), and (b) a pharmaceutically acceptable excipient (see, e.g., Remington's Pharmaceutical Sciences (1990) Mack Publishing Co., Easton, PA, the entire contents of which is incorporated by reference herein for all purposes).

In one aspect, provided herein are pharmaceutical compositions comprising (a) a KRAS inhibitor (e.g., a KRAS inhibitor described herein), and (b) a pharmaceutically acceptable excipient (see, e.g., Remington's Pharmaceutical Sciences (1990) Mack Publishing Co., Easton, PA, the entire contents of which is incorporated by reference herein for all purposes).

In one aspect, provided herein are pharmaceutical compositions comprising (a) a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), a host cell described herein (e.g., a host cell comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein))), or a carrier described herein (e.g., a carrier comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), or a host cell described herein); (b) a KRAS inhibitor (e.g., a KRAS inhibitor described herein), and (c) a pharmaceutically acceptable excipient (see, e.g., Remington's Pharmaceutical Sciences (1990) Mack Publishing Co., Easton, PA, the entire contents of which is incorporated by reference herein for all purposes).

Also provided herein are pharmaceutical compositions comprising a fusion protein (or polypeptide thereof) described herein or a component thereof (e.g., a moiety thereof) described herein, a polynucleotide described herein, a vector described herein, a host cell described herein, or a carrier described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

In one aspect, also provided herein are methods of making pharmaceutical compositions described herein comprising providing (a) a fusion protein (or one or more polypeptide thereof) described herein, a polynucleotide described herein, a vector described herein, a host cell described herein, or a carrier described herein, and (b) a KRAS inhibitor (e.g., a KRAS inhibitor described herein) and formulating (a) and (b) into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

In some embodiments, the fusion protein (e.g., BCA101) is formulated according to any of the aspects and embodiments described in WO2022132201 (PCT/US2021010066), the entire contents of which is incorporated by reference herein for all purposes.

For example, in some embodiments, the fusion protein (e.g., BCA101) is formulated in a liquid formulation comprising buffer present at a concentration from about 5 mM to about 30 mM; and a tonifying agent present at a concentration from about 4% w/v to about 10% w/v; wherein said liquid pharmaceutical composition has a pH from about 5.5 to about 7.0.

In some embodiments, the buffer is a citrate phosphate buffer, citrate buffer, succinate buffer, or histidine buffer. In some embodiments, the buffer is a citrate phosphate buffer. In some embodiments, the buffer is present at a concentration from about 5 mM to about 25 mM, 5 mM to about 20 mM. 5 mM to about 15 mM, 5 mM to about 10 mM, or 10 mM to about 30 mM. In some embodiments, the buffer is present at a concentration from about 5 mM to about 15 mM. In some embodiments, the buffer is present at a concentration of about 5 mM, 10 mM, 15 mM, 20 mM, 25 mM, or 30 mM. In some embodiments, wherein the buffer is present at a concentration of about 10 mM. In some embodiments, wherein the buffer comprises about 10 mM citrate phosphate.

In some embodiments, the tonifying agent is a saccharide. In some embodiments, the tonifying agent is a disaccharide. In some embodiments, the tonifying agent is sucrose or trehalose. In some embodiments, the tonifying agent is sucrose. In some embodiments, the tonifying agent present at a concentration from about 5% w/v to about 10% w/v, 6% w/v to about 10% w/v, 7% w/v to about 10% w/v, 8% w/v to about 10% w/v, 5% w/v to about 9% w/v, 5% w/v to about 8% w/v, 6% w/v to about 9% w/v, 6% w/v to about 8% w/v, 7% w/v to about 9% w/v, or 7% w/v to about 8% w/v. In some embodiments, the tonifying agent present at a concentration from about 5% w/v to about 8% w/v. In some embodiments, the tonifying agent present at a concentration of about 5% w/v, 6% w/v, 7% w/v, 8% w/v, 9% w/v, or 10% w/v. In some embodiments, the tonifying agent present at a concentration of about 8% w/v. In some embodiments, the tonifying agent is sucrose and is present at a concentration of about 8% w/v.

In some embodiments, the liquid pharmaceutical composition further comprising a surfactant. In some embodiments, the surfactant comprises polysorbate 20, polysorbate 40, polysorbate 60, or polysorbate 80. In some embodiments, the surfactant comprises polysorbate 20. In some embodiments, the surfactant comprises polysorbate 80. In some embodiments, the surfactant is present at a concentration from about 0.005-0.1% w/v. In some embodiments, the surfactant is present at a concentration from about 0.01-0.1% w/v, 0.02-0.1% w/v, 0.01-0.9% w/v, 0.01-0.8% w/v, 0.01-0.7% w/v, 0.01-0.6% w/v, 0.01-0.5% w/v, 0.01-0.4% w/v, 0.01-0.3% w/v, 0.01-0.2% w/v, 0.01-0.1% w/v, 0.02-0.9% w/v, 0.02-0.8% w/v, 0.02-0.7% w/v, 0.02-0.6% w/v, 0.02-0.5% w/v, 0.02-0.4% w/v, 0.02-0.3% w/v, 0.02-0.2% w/v, 0.02-0.1% w/v, 0.005-0.9% w/v, 0.005-0.8% w/v, 0.005-0.7% w/v, 0.005-0.6% w/v, 0.005-0.5% w/v, 0.005-0.4% w/v, 0.005-0.3% w/v, 0.005-0.2% w/v, or 0.005-0.1% w/v. In some embodiments, the surfactant is present at a concentration of about 0.01% w/v, 0.02% w/v, 0.03% w/v, 0.04% w/v, 0.05% w/v, 0.06% w/v, 0.07% w/v, 0.08% w/v, 0.09% w/v, or 0.1% w/v. In some embodiments, the surfactant is present at a concentration of about 0.02% w/v. In some embodiments, the surfactant is polysorbate 20 and is present at a concentration of about 0.02% w/v.

In some embodiments, the liquid pharmaceutical composition has a pH from about 5.5 to about 7.0, 6.0 to about 7.0, 5.5 to about 6.5, 5.5 to about 6.0, or 6.0 to about 6.5. In some embodiments, the liquid pharmaceutical composition has a pH from about 6.0 to about 6.5. In some embodiments, the liquid pharmaceutical composition has a pH of about 5.5, 6.0, 6.5. or 7.0. In some embodiments, the liquid pharmaceutical composition has a pH of about 6.0.

In some embodiments, the liquid pharmaceutical composition has an osmolality from about 150 mOsmol/kg to about 400 mOsmol/kg. In some embodiments, the liquid pharmaceutical composition has an osmolality from about 150 mOsmol/kg to about 350 mOsmol/kg, 150 mOsmol/kg to about 300 mOsmol/kg, 200 mOsmol/kg to about 400 mOsmol/kg, 250 mOsmol/kg to about 400 mOsmol/kg, 300 mOsmol/kg to about 400 mOsmol/kg, 300 mOsmol/kg to about 350 mOsmol/kg, 250 mOsmol/kg to about 350 mOsmol/kg, or 250 mOsmol/kg to about 300 mOsmol/kg. In some embodiments, the liquid pharmaceutical composition has an osmolality from about 250 mOsmol/kg to about 350 mOsmol/kg. In some embodiments, the liquid pharmaceutical composition has an osmolality of about 250 mOsmol/kg, 300 mOsmol/kg, or 300 mOsmol/kg. In some embodiments, the liquid pharmaceutical composition has an osmolality of about 300 mOsmol/kg.

In some embodiments, the liquid pharmaceutical composition is stable for at least 12, 18, or 24 months when stored at −20° C. In some embodiments, the liquid pharmaceutical composition is stable for at least 12, 18, or 24 months when stored at 2-8° C.

In some embodiments, the concentration of the fusion protein in the liquid pharmaceutical composition is substantially the same for at least 12, 18, or 24 months when stored at −80° C. In some embodiments, the concentration of the fusion protein in the liquid pharmaceutical composition is substantially the same for at least 12, 18, or 24 months when stored at −20° C. In some embodiments, the concentration of the fusion protein in the liquid pharmaceutical composition is substantially the same for at least 12, 18, or 24 months when stored at 2-8° C.

In some embodiments, the concentration of the fusion protein in the liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at −80° C. In some embodiments, the concentration of the fusion protein in the liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at −20° C. In some embodiments, the concentration of the fusion protein in the liquid pharmaceutical composition does not decrease more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% after storage for 12, 18, or 24 months at 2-8° C.

In some embodiments, the liquid pharmaceutical composition is stable upon 1, 2, 3, 4, or 5 cycles of freezing and thawing.

In some embodiments, the fusion protein retains bifunctional activity as measured by bifunctional enzyme-linked immunosorbent assay (ELISA) for at least 12, 18, or 24 months when stored at −20° C. In some embodiments, the fusion protein retains bifunctional activity as measured by bifunctional ELISA for at least 12, 18, or 24 months when stored at −20° C. In some embodiments, the fusion protein retains bifunctional activity as measured by bifunctional ELISA for at least 12, 18, or 24 months when stored at 2-8° C.

In some embodiments, the liquid pharmaceutical composition comprises less than about 10%, 9%, 8%, 7%, 6%, 5%, 4%, 3%, 2%, or 1% of the fusion protein in aggregate form.

In some embodiments, the liquid pharmaceutical composition has at least one feature selected from the group consisting of (a) increased shelf life, (b) increased temperature stability, (c) decreased formation of aggregates, (d) increased chemical stability, (c) decreased fragmentation, and/or (I) decreased viscosity; after 12, 18, or 24 months of storage at −20° C. or 2-8° C., as compared to a reference formulation.

In some embodiments, the liquid pharmaceutical composition has at least one feature selected from the group consisting of: (a) decreased percentage of aggregates as measured by size exclusion chromatography (SEC), (b) higher percentage of monomers as measured by SEC, and/or (c) lower turbidity value in nephelometry units (NTU); after 12, 18, or 24 months of storage at −20° C. or 2-8° C., as compared to the reference formulation.

In some embodiments, the fusion protein is present at a concentration from about 5-50 mg/ml, 5-40 mg/ml, 5-30 mg/ml, 5-25 mg/ml, 10-50 mg/ml, 20-50 mg/ml, 25-50 mg/ml, 20-50 mg/ml, 20-40 mg/ml, 20-30 mg/ml, 25-50 mg/ml, 25-40 mg/ml, or 25-30 mg/ml. In some embodiments, the fusion protein is present at a concentration from about 20-30 mg/ml. In some embodiments, the fusion protein is present at a concentration of about 5 mg/ml, 10 mg/ml, 15 mg/ml, 20 mg/ml, 25 mg/ml, 30 mg/ml, 35 mg/ml, 40 mg/ml, 45 mg/ml, or 50 mg/ml. In some embodiments, the fusion protein is present at a concentration of about 25 mg/ml. In some embodiments, the fusion protein is present at a concentration of about 50 mg/ml.

In some embodiments, the fusion protein (e.g., BCA101) is formulated as follows: liquid formulation comprising or consisting of 50 mg/ml the fusion protein (e.g., BCA101), 8.0% w/v sucrose, 0.02% w/v polysorbate 80, and 10 mM citrate phosphate buffer at pH 6.0.

In some embodiments, wherein the KRAS (e.g., hKRAS) inhibitor is sotorasib, the sotorasib is in a tablet form. In some embodiments, wherein the KRAS (e.g., hKRAS) inhibitor is sotorasib, the sotorasib is in a tablet form for oral administration. In some embodiments, wherein the KRAS (e.g., hKRAS) inhibitor is sotorasib, the sotorasib is in a film coated tablet form. In some embodiments, the tablet core comprises or consists of any 1, 2, 3, or 4 of the following: microcrystalline cellulose, lactose monohydrate, croscarmellose sodium, and magnesium stearate.

In some embodiments, the tablet core comprises or consists of any 1, 2, 3, or 4 of the following: microcrystalline cellulose, lactose monohydrate, croscarmellose sodium, and magnesium stearate, in addition to the active ingredient (sotorasib). In some embodiments, the film coating material comprises or consists of any 1, 2, 3, 4, or 5 of the following: polyvinyl alcohol, titanium dioxide, polyethylene glycol, talc, and iron oxide yellow. In some embodiments, wherein the KRAS (e.g., hKRAS) inhibitor is sotorasib, the sotorasib is administered at 960 mg orally once a day. In some embodiments, the sotorasib is administered orally as a whole tablet with or without food.

### 5.11 Methods of Use

Provided herein are various methods of utilizing KRAS (e.g., hKRAS) inhibitors (e.g., KRAS inhibitors described herein), fusion proteins (and polypeptides thereof) described herein, moieties described herein (e.g., an EGFR (e.g., hEGFR) binding moiety, TGFβ (e.g., hTGFβ) binding moiety)), polynucleotides described herein, vectors described herein, host cells described herein, carriers described herein, and pharmaceutical compositions described herein. In some embodiments, the methods comprise administration of (i) a fusion protein (or polypeptides thereof) described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), a host cell described herein (e.g., a host cell comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein))), or a carrier described herein (e.g., a carrier comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), or a host cell described herein); and (ii) a KRAS inhibitor (e.g., a KRAS inhibitor described herein) or a pharmaceutical composition the KRAS inhibitor; or a pharmaceutical composition comprising (i) and (ii) to a subject. Exemplary subjects include mammals, e.g., humans, non-human mammals, e.g., non-human primates. In some embodiments, the subject is a human.

The dosage of the KRAS inhibitor, fusion protein or polypeptide described herein, a polynucleotide described herein, a vector described herein, a host cell described herein, a carrier described herein, or a pharmaceutical composition described herein to be administered to a subject in accordance with any of the methods described herein can be determined in accordance with standard techniques known to those of ordinary skill in the art, including the route of administration, the age and weight of the subject, and the type (if any) adjuvant is used. In some embodiments, each agent is administered in an amount effective to mediate the method (e.g., an effective amount).

### 5.11.1 Methods of Delivery

In one aspect, provided herein are methods of delivering a KRAS (e.g., hKRAS) inhibitor and a fusion protein to a subject, the method comprising administering to the subject (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor (e.g., a KRAS (e.g., hKRAS) inhibitor described herein); in combination with (ii) (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein described herein), (c) a carrier described herein (e.g., a carrier comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein), to thereby deliver the KRAS (e.g., hKRAS) inhibitor and the fusion protein to the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of delivering a KRAS (e.g., hKRAS) inhibitor and a fusion protein to a subject, the method comprising administering to the subject a pharmaceutical composition comprising (i) (a) a KRAS (e.g., hKRAS) inhibitor (e.g., a KRAS (e.g., hKRAS) inhibitor described herein); and (ii) (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), or (e) a carrier described herein (e.g., a carrier comprising a fusion protein described herein, a polynucleotide encoding a fusion protein described herein, a vector comprising a polynucleotide described herein (e.g., a vector comprising a polynucleotide encoding a fusion protein described herein), to thereby deliver the KRAS (e.g., hKRAS) inhibitor and the fusion protein to the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of delivering a KRAS (e.g., hKRAS) inhibitor and a fusion protein to a subject, the method comprising administering to the subject a combination composition described herein (see, e.g., § 5.8) or a combination regimen described herein (see, e.g., § 5.7), to thereby deliver the KRAS (e.g., hKRAS) inhibitor and the fusion protein to the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

### 5.11.2 Methods of Inhibiting the KRAS and TGFβ Pathways

In one aspect, provided herein are methods of inhibiting the KRAS (e.g., hKRAS) and TGFβ pathways in a subject in need thereof, the method comprising administering to the subject (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein; in combination with (ii) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein), to thereby inhibit the KRAS (e.g., hKRAS) and TGFβ pathways in the subject.

In some embodiments, the KRAS (e.g., hKRAS) inhibitor and the fusion protein are administered in an amount and for a time sufficient to inhibit the KRAS (e.g., hKRAS) pathway and the TGFβ pathway in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of inhibiting the KRAS (e.g., hKRAS) and TGFβ pathways in a subject in need thereof, the method comprising administering to the subject a combination composition described herein (see, e.g., § 5.8) or a combination regimen described herein (see, e.g., § 5.7), to thereby inhibit the KRAS (e.g., hKRAS) and TGFβ pathways in the subject.

In some embodiments, the KRAS (e.g., hKRAS) inhibitor and the fusion protein are administered in an amount and for a time sufficient to inhibit the KRAS (e.g., hKRAS) pathway and the TGFβ pathway in the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of inhibiting the KRAS (e.g., hKRAS) and TGFβ pathways in a subject in need thereof, the method comprising administering to a subject a pharmaceutical composition comprising (i) a KRAS (e.g., hKRAS) inhibitor (e.g., a KRAS (e.g., hKRAS) inhibitor described herein); and (ii) (a) a fusion protein (or polypeptides thereof) described herein, (b) a polynucleotide encoding a fusion protein (or polypeptides thereof) described herein, (c) a vector comprising a polynucleotide described herein (e.g., a polynucleotide encoding a fusion protein (or polypeptides thereof) described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), or (c) a carrier described herein (e.g., a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein).

In some embodiments, the KRAS (e.g., hKRAS) inhibitor and the fusion protein are administered in an amount and for a time sufficient to inhibit the KRAS (e.g., hKRAS) pathway and the TGFβ pathway in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are combination therapies comprising (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein and (ii) (a) a fusion protein (or polypeptides thereof) described herein, (b) a polynucleotide encoding a fusion protein (or polypeptides thereof) described herein, (c) a vector comprising a polynucleotide described herein (e.g., a polynucleotide encoding a fusion protein (or polypeptides thereof) described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), (e) a carrier described herein (e.g., a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), or (f) a pharmaceutical composition described herein (e.g., a pharmaceutical composition comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein)), or a carrier described herein (e.g., a carrier a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein) for use in the manufacture of a medicament for inhibiting the KRAS (e.g., hKRAS) and TGFβ pathways in a subject in need thereof, e.g., comprising administering to the subject the combination therapy.

In some embodiments, the KRAS (e.g., hKRAS) inhibitor and the fusion protein are administered in an amount and for a time sufficient to inhibit the KRAS (e.g., hKRAS) pathway and the TGFβ pathway in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are combination therapies comprising (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein and (ii) (a) a fusion protein (or polypeptides thereof) described herein, (b) a polynucleotide encoding a fusion protein (or polypeptides thereof) described herein, (c) a vector comprising a polynucleotide described herein (e.g., a polynucleotide encoding a fusion protein (or polypeptides thereof) described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), (e) a carrier described herein (e.g., a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), or (f) a pharmaceutical composition described herein (e.g., a pharmaceutical composition comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein)), or a carrier described herein (e.g., a carrier a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein) for use in a method of inhibiting the KRAS (e.g., hKRAS) and TGFβ pathways in a subject in need thereof, the method comprising administering to the subject the combination therapy.

In some embodiments, the KRAS (e.g., hKRAS) inhibitor and the fusion protein are administered in an amount and for a time sufficient to inhibit the KRAS (e.g., hKRAS) pathway and the TGFβ pathway in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for use in combination with (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for inhibiting the KRAS (e.g., hKRAS) and TGFβ pathways in a subject in need thereof.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for inhibiting the KRAS (e.g., hKRAS) and TGFβ pathways in a subject in need thereof, wherein the medicament is administered in combination with or comprises (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of a combination composition described herein (see, e.g., § 5.8) or a combination regimen described herein (see, e.g., § 5.7) for the manufacture of a medicament for inhibiting the KRAS (e.g., hKRAS) and TGFβ pathways in a subject in need thereof.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein); and (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for use in a method of inhibiting the KRAS (e.g., hKRAS) and TGFβ pathways in a subject in need thereof, the method comprising administering to the subject (i) and (ii), to thereby inhibit the KRAS (e.g., hKRAS) and TGFβ pathways in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination composition described herein (see, e.g., § 5.8) or a combination regimen described herein (see, e.g., § 5.7) for use in a method of inhibiting the KRAS (e.g., hKRAS) and TGFβ pathways in a subject in need thereof, the method comprising administering to the subject the combination composition described herein (see, e.g., § 5.8) or the combination regimen described herein (see, e.g., § 5.7), to thereby inhibit the KRAS (e.g., hKRAS) and TGFβ pathways in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

### 5.11.3 Methods of Restoring Sensitivity to a KRAS Inhibitor

In one aspect, provided herein are methods of restoring sensitivity to a KRAS (e.g., hKRAS) inhibitor in a subject in need thereof, the method comprising administering to the subject (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein (described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein), to thereby restore sensitivity to a KRAS (e.g., hKRAS) inhibitor in the subject.

In some embodiments, the fusion protein is administered in an amount and for a time sufficient to restore sensitivity to the KRAS (e.g., hKRAS) inhibitor in the subject. In some embodiments, the cancer in the subject is resistant to the KRAS (e.g., hKRAS) inhibitor. In some embodiments, the cancer has been determined to be resistant to the KRAS (e.g., hKRAS) inhibitor. In some embodiments, the method further comprising administering the KRAS (e.g., hKRAS) inhibitor to the subject in combination with the fusion protein.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of restoring sensitivity to a KRAS (e.g., hKRAS) inhibitor in a subject in need thereof, the method comprising administering to the subject (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein; in combination with (ii) (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein), to thereby restore sensitivity to a KRAS (e.g., hKRAS) inhibitor in the subject.

In some embodiments, the fusion protein is administered in an amount and for a time sufficient to restore sensitivity to the KRAS (e.g., hKRAS) inhibitor in the subject. In some embodiments, the cancer in the subject is resistant to the KRAS (e.g., hKRAS) inhibitor. In some embodiments, the cancer has been determined to be resistant to the KRAS (e.g., hKRAS) inhibitor.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of restoring sensitivity to a KRAS (e.g., hKRAS) inhibitor in a subject in need thereof, the method comprising administering to the subject a combination composition described herein (see, e.g., § 5.8) or a combination regimen described herein (see, e.g., § 5.7), to thereby restore sensitivity to a KRAS (e.g., hKRAS) inhibitor in the subject.

In some embodiments, the cancer in the subject is resistant to the KRAS (e.g., hKRAS) inhibitor. In some embodiments, the cancer has been determined to be resistant to the KRAS (e.g., hKRAS) inhibitor.

In some embodiments, the subject has been previously administered a KRAS (e.g., hKRAS) inhibitor (e.g., the KRAS (e.g., hKRAS) inhibitor). In some embodiments, the KRAS inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of restoring sensitivity to a KRAS (e.g., hKRAS) inhibitor in a subject in need thereof, the method comprising administering to the subject a pharmaceutical composition comprising (i) a KRAS (e.g., hKRAS) inhibitor (e.g., a KRAS (e.g., hKRAS) inhibitor described herein); and (ii) (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), or (e) a carrier described herein (e.g., a carrier comprising a fusion protein described herein, a polynucleotide encoding a fusion protein described herein, a vector comprising a polynucleotide described herein (e.g., a vector comprising a polynucleotide encoding a fusion protein described herein), to thereby restore sensitivity to a KRAS (e.g., hKRAS) inhibitor in the subject.

In some embodiments, the fusion protein is administered in an amount and for a time sufficient to restore sensitivity to the KRAS (e.g., hKRAS) inhibitor in the subject. In some embodiments, the cancer in the subject is resistant to the KRAS (e.g., hKRAS) inhibitor. In some embodiments, the cancer has been determined to be resistant to the KRAS (e.g., hKRAS) inhibitor.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (c) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for restoring sensitivity to a KRAS (e.g., hKRAS) inhibitor in a subject in need thereof.

In some embodiments, the fusion protein is administered in an amount and for a time sufficient to restore sensitivity to the KRAS (e.g., hKRAS) inhibitor in the subject. In some embodiments, the cancer in the subject is resistant to the KRAS (e.g., hKRAS) inhibitor. In some embodiments, the cancer has been determined to be resistant to the KRAS (e.g., hKRAS) inhibitor. In some embodiments, the method further comprising administering the KRAS (e.g., hKRAS) inhibitor to the subject in combination with the fusion protein.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for use in combination with (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for restoring sensitivity to a KRAS (e.g., hKRAS) inhibitor in a subject in need thereof.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the fusion protein is administered in an amount and for a time sufficient to restore sensitivity to the KRAS (e.g., hKRAS) inhibitor in the subject. In some embodiments, the cancer in the subject is resistant to the KRAS (e.g., hKRAS) inhibitor. In some embodiments, the cancer has been determined to be resistant to the KRAS (e.g., hKRAS) inhibitor. In some embodiments, the method further comprising administering the KRAS (e.g., hKRAS) inhibitor to the subject in combination with the fusion protein.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (c) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for restoring sensitivity to a KRAS (e.g., hKRAS) inhibitor in a subject in need thereof, wherein the medicament is administered in combination with or comprises (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the fusion protein is administered in an amount and for a time sufficient to restore sensitivity to the KRAS (e.g., hKRAS) inhibitor in the subject. In some embodiments, the cancer in the subject is resistant to the KRAS (e.g., hKRAS) inhibitor. In some embodiments, the cancer has been determined to be resistant to the KRAS (e.g., hKRAS) inhibitor. In some embodiments, the method further comprising administering the KRAS (e.g., hKRAS) inhibitor to the subject in combination with the fusion protein.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of a combination composition described herein (see, e.g., § 5.8) or a combination regimen described herein (see, e.g., § 5.7) for the manufacture of a medicament for restoring sensitivity to a KRAS (e.g., hKRAS) inhibitor in a subject in need thereof.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the fusion protein is administered in an amount and for a time sufficient to restore sensitivity to the KRAS (e.g., hKRAS) inhibitor in the subject. In some embodiments, the cancer in the subject is resistant to the KRAS (e.g., hKRAS) inhibitor. In some embodiments, the cancer has been determined to be resistant to the KRAS (e.g., hKRAS) inhibitor. In some embodiments, the method further comprising administering the KRAS (e.g., hKRAS) inhibitor to the subject in combination with the fusion protein.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein); and (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for use in a method of restoring sensitivity to a KRAS (e.g., hKRAS) inhibitor in a subject in need thereof, the method comprising administering to the subject (i) and (ii), to thereby restore sensitivity to a KRAS (e.g., hKRAS) inhibitor in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination composition described herein (see, e.g., § 5.8) or a combination regimen described herein (see, e.g., § 5.7) for use in a method of restoring sensitivity to a KRAS (e.g., hKRAS) inhibitor in a subject in need thereof, the method comprising administering to the subject the combination composition described herein (see, e.g., § 5.8) or the combination regimen described herein (see, e.g., § 5.7), to thereby restore sensitivity to a KRAS (e.g., hKRAS) inhibitor in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

### 5.11.4 Methods of Suppressing or Preventing Resistance to a KRAS Inhibitor

In one aspect, provided herein are methods of suppressing or preventing resistance to a KRAS inhibitor in a subject in need thereof, the method comprising administering to the subject (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein; in combination with (ii) (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (c) a carrier described herein (e.g., a carrier comprising a fusion protein described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein), to thereby suppress or prevent resistance to a KRAS inhibitor in the subject.

In some embodiments, the fusion protein is administered in an amount and for a time sufficient to suppress the development of resistance to the KRAS (e.g., hKRAS) inhibitor in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the KRAS (e.g., hKRAS) inhibitor was previously administered to the subject. In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of suppressing or preventing resistance to a KRAS inhibitor in a subject in need thereof, the method comprising administering to the subject a combination composition described herein (see, e.g., § 5.8) or a combination regimen described herein (see, e.g., § 5.7), to thereby suppress or prevent resistance to a KRAS inhibitor in the subject.

In some embodiments, the KRAS (e.g., hKRAS) inhibitor was previously administered to the subject. In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of suppressing or preventing resistance to a KRAS inhibitor in a subject in need thereof, the method comprising administering to the subject a pharmaceutical composition comprising (i) a KRAS (e.g., hKRAS) inhibitor (e.g., a KRAS (e.g., hKRAS) inhibitor described herein); and (ii) (a) a fusion protein (or polypeptides thereof) described herein, (b) a polynucleotide encoding a fusion protein (or polypeptides thereof) described herein, (c) a vector comprising a polynucleotide described herein (e.g., a polynucleotide encoding a fusion protein (or polypeptides thereof) described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), or (e) a carrier described herein (e.g., a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), to thereby suppress or prevent resistance to a KRAS inhibitor in the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for use in combination with (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for suppressing or preventing resistance to a KRAS inhibitor in a subject in need thereof.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the KRAS (e.g., hKRAS) inhibitor was previously administered to the subject. In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for suppressing or preventing resistance to a KRAS inhibitor in a subject in need thereof, wherein the medicament is administered in combination with or comprises (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the KRAS (e.g., hKRAS) inhibitor was previously administered to the subject. In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of a combination composition described herein (see, e.g., § 5.8) or a combination regimen described herein (see, e.g., § 5.7) for the manufacture of a medicament for suppressing or preventing resistance to a KRAS inhibitor in a subject in need thereof.

In some embodiments, the KRAS (e.g., hKRAS) inhibitor was previously administered to the subject. In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein); and (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for use in a method of suppressing or preventing resistance to a KRAS inhibitor in a subject in need thereof, the method comprising administering to the subject (i) and (ii), to thereby suppress or prevent resistance to a KRAS inhibitor in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination composition described herein (see, e.g., § 5.8) or a combination regimen described herein (see, e.g., § 5.7) for use in a method of suppressing or preventing resistance to a KRAS inhibitor in a subject in need thereof, the method comprising administering to the subject the combination composition described herein (see, e.g., § 5.8) or the combination regimen described herein (see, e.g., § 5.7), to thereby suppress or prevent resistance to a KRAS inhibitor in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

### 5.11.5 Methods of Treating Cancer

In one aspect, provided herein are methods of treating cancer in a subject in need thereof, the method comprising administering to the subject (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein; in combination with (ii) (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein), to thereby treat cancer in the subject.

In some embodiments, the KRAS (e.g., hKRAS) inhibitor and the fusion protein are administered in an amount and for a time sufficient to treat the KRAS (e.g., hKRAS) variant cancer in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating cancer in a subject in need thereof, the method comprising administering to the subject (i) (a) a KRAS (e.g., hKRAS) inhibitor (e.g., a KRAS (e.g., hKRAS) inhibitor described herein); in combination with (ii) (a) a fusion protein described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), or (c) a carrier described herein (e.g., a carrier comprising a fusion protein described herein, a polynucleotide encoding a fusion protein described herein, a vector comprising a polynucleotide described herein (e.g., a vector comprising a polynucleotide encoding a fusion protein described herein), to thereby treat cancer in the subject.

In some embodiments of any of the foregoing aspects, the KRAS (e.g., hKRAS) inhibitor and the fusion protein are administered in an amount and for a time sufficient to treat or prevent the cancer in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating cancer in a subject in need thereof, the method comprising administering to the subject a combination composition described herein (see, e.g., § 5.8) or a combination regimen described herein (see, e.g., § 5.7), to thereby treat cancer in the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating a KRAS (e.g., hKRAS) variant cancer in a subject in need thereof, the method comprising: (a) receiving test results showing that the KRAS (e.g., hKRAS) variant cancer is resistant to a KRAS (e.g., hKRAS) inhibitor; and (b) administering to the subject (i) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein; in combination with (ii) (a) a fusion protein (or polypeptides thereof) described herein, (b) a polynucleotide encoding a fusion protein (or polypeptides thereof) described herein, (c) a vector comprising a polynucleotide described herein (e.g., a polynucleotide encoding a fusion protein (or polypeptides thereof) described herein), (d) a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), (e) a carrier described herein (e.g., a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), or (f) a pharmaceutical composition described herein (e.g., a pharmaceutical composition comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), a host cell described herein (e.g., a host cell comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein)), or a carrier described herein (e.g., a carrier a carrier comprising a fusion protein (or polypeptides thereof) described herein, a polynucleotide encoding a fusion protein (or polypeptides thereof) described herein, a vector comprising a polynucleotide described herein (e.g., a vector comprising a polynucleotide encoding a fusion protein (or polypeptides thereof) described herein), to thereby treat a KRAS (e.g., hKRAS) variant cancer in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the KRAS (e.g., hKRAS) inhibitor has been previously administered to the subject.

In some embodiments, the KRAS (e.g., hKRAS) inhibitor and the fusion protein are administered in an amount and for a time sufficient to treat the KRAS (e.g., hKRAS) variant cancer in the subject.

In some embodiments, the subject has been previously administered a KRAS (e.g., hKRAS) inhibitor (e.g., the KRAS (e.g., hKRAS) inhibitor). In some embodiments, the KRAS inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating a KRAS (e.g., hKRAS) variant cancer in a subject in need thereof, the method comprising: (a) receiving test results showing that the KRAS (e.g., hKRAS) variant cancer is resistant to a KRAS (e.g., hKRAS) inhibitor; and (b) administering to the subject a combination composition described herein (see, e.g., § 5.8) or a combination regimen described herein (see, e.g., § 5.7), to thereby treat a KRAS (e.g., hKRAS) variant cancer in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the KRAS (e.g., hKRAS) inhibitor has been previously administered to the subject.

In some embodiments, the KRAS (e.g., hKRAS) inhibitor and the fusion protein are administered in an amount and for a time sufficient to treat the KRAS (e.g., hKRAS) variant cancer in the subject.

In some embodiments, the subject has been previously administered a KRAS (e.g., hKRAS) inhibitor (e.g., the KRAS (e.g., hKRAS) inhibitor). In some embodiments, the KRAS inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating a KRAS (e.g., hKRAS) variant cancer in a subject in need thereof, the method comprising: (a) administering to the subject a KRAS (e.g., hKRAS) inhibitor (e.g., a KRAS (e.g., hKRAS) inhibitor described herein); (b) receiving testing results showing that the KRAS (e.g., hKRAS) variant cancer has developed resistance to the KRAS (e.g., hKRAS) inhibitor; and (c) administering to the subject the KRAS (e.g., hKRAS) inhibitor; and a fusion protein comprising: (i) a first moiety that specifically binds EGFR (e.g., hEGFR) operably connected to (ii) a second moiety that specifically binds TGFβ (e.g., hTGFβ), to thereby treat a KRAS (e.g., hKRAS) variant cancer in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the KRAS (e.g., hKRAS) inhibitor and the fusion protein are administered in an amount and for a time sufficient to treat the KRAS (e.g., hKRAS) variant cancer in the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating a KRAS (e.g., hKRAS) variant cancer in a subject in need thereof, the method comprising: (a) administering to the subject a KRAS (e.g., hKRAS) inhibitor (e.g., a KRAS (e.g., hKRAS) inhibitor described herein); (b) receiving testing results showing that the KRAS (e.g., hKRAS) variant cancer has developed resistance to the KRAS (e.g., hKRAS) inhibitor; and (c) administering to the subject a combination composition described herein (see, e.g., § 5.8) or a combination regimen described herein (see, e.g., § 5.7), to thereby treat a KRAS (e.g., hKRAS) variant cancer in the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating a KRAS (e.g., hKRAS) variant cancer in a subject in need thereof, the method comprising: (a) determining that the KRAS (e.g., hKRAS) variant cancer is resistant to a KRAS (e.g., hKRAS) inhibitor (e.g., a KRAS (e.g., hKRAS) inhibitor described herein); and (b) administering to the subject a KRAS (e.g., hKRAS) inhibitor (e.g., a KRAS (e.g., hKRAS) inhibitor described herein); in combination with a fusion protein comprising: (i) a first moiety that specifically binds EGFR (e.g., hEGFR) operably connected to (ii) a second moiety that specifically binds TGFβ (e.g., hTGFβ), to thereby treat a KRAS (e.g., hKRAS) variant cancer in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously treated with the KRAS (e.g., hKRAS) inhibitor.

In some embodiments, the KRAS (e.g., hKRAS) inhibitor and the fusion protein are administered in an amount and for a time sufficient to treat the KRAS (e.g., hKRAS) variant cancer in the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein are methods of treating a KRAS (e.g., hKRAS) variant cancer in a subject in need thereof, the method comprising: (a) determining that the KRAS (e.g., hKRAS) variant cancer is resistant to a KRAS (e.g., hKRAS) inhibitor (e.g., a KRAS (e.g., hKRAS) inhibitor described herein); and (b) administering to the subject a combination composition described herein (see, e.g., § 5.8) or a combination regimen described herein (see, e.g., § 5.7), to thereby treat a KRAS (e.g., hKRAS) variant cancer in the subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously treated with the KRAS (e.g., hKRAS) inhibitor.

In some embodiments, the KRAS (e.g., hKRAS) inhibitor and the fusion protein are administered in an amount and for a time sufficient to treat the KRAS (e.g., hKRAS) variant cancer in the subject.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for use in combination with (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for treating cancer in a subject in need thereof.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein) for the manufacture of a medicament for treating cancer in a subject in need thereof, wherein the medicament is administered in combination with or comprises (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a use of a combination composition described herein (see, e.g., § 5.8) or a combination regimen described herein (see, e.g., § 5.7) for the manufacture of a medicament for treating cancer in a subject in need thereof.

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination of (i) (a) a fusion protein (or polypeptides thereof) described herein, (b) a polynucleotide encoding a fusion protein described herein, (c) a vector comprising a polynucleotide described herein (e.g., a polynucleotide encoding a fusion protein described herein), (d) a host cell described herein (e.g., a host cell comprising a fusion protein described herein, a polynucleotide encoding a fusion protein described herein, a vector comprising a polynucleotide described herein (e.g., a vector comprising a polynucleotide encoding a fusion protein described herein), (e) a carrier described herein (e.g., a carrier comprising a fusion protein (or polypeptides thereof) described herein, a polynucleotide encoding a fusion protein described herein, a vector comprising a polynucleotide described herein (e.g., a vector comprising a polynucleotide encoding a fusion protein described herein), or (f) a pharmaceutical composition described herein (e.g., a pharmaceutical composition comprising a fusion protein described herein, a polynucleotide encoding a fusion protein described herein, a vector comprising a polynucleotide described herein (e.g., a vector comprising a polynucleotide encoding a fusion protein described herein), a host cell described herein (e.g., a host cell comprising a fusion protein described herein, a polynucleotide encoding a fusion protein (or polypeptides thereof) described herein, a vector comprising a polynucleotide described herein (e.g., a vector comprising a polynucleotide encoding a fusion protein described herein)), or a carrier described herein (e.g., a carrier a carrier comprising a fusion protein described herein, a polynucleotide encoding a fusion protein described herein, a vector comprising a polynucleotide described herein (e.g., a vector comprising a polynucleotide encoding a fusion protein described herein); and (ii) (a) a KRAS (e.g., hKRAS) inhibitor (e.g., a KRAS (e.g., hKRAS) inhibitor described herein) or (b) a pharmaceutical composition comprising a KRAS (e.g., hKRAS) inhibitor described herein for use in a method of treating cancer in a subject in need thereof, the method comprising administering to the subject (i) and (ii), to thereby treat cancer in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

In one aspect, provided herein is a combination composition described herein (see, e.g., § 5.8) or a combination regimen described herein (see, e.g., § 5.7) for use in a method of treating cancer in a subject in need thereof, the method comprising administering to the subject the combination composition described herein (see, e.g., § 5.8) or the combination regimen described herein (see, e.g., § 5.7), to thereby treat cancer in a subject.

As described herein (i) and (ii) may be administered to the subject concurrently or sequentially. In some embodiments, (i) and (ii) are administered concurrently. In some embodiments, (i) and (ii) are administered sequentially. In some embodiments, (i) is administered prior to (ii). In some embodiments, (ii) is administered prior to (i).

In some embodiments, the subject has been previously administered a KRAS (e.g., hKRAS) inhibitor (e.g., the KRAS (e.g., hKRAS) inhibitor). In some embodiments, the KRAS (e.g., hKRAS) inhibitor has not been previously administered to the subject. In some embodiments, the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor. In some embodiments, the cancer in the subject is resistant to a KRAS (e.g., hKRAS) inhibitor (e.g., the KRAS (e.g., hKRAS) inhibitor).

### 5.11.5.1 Cancers

Various aspect and embodiments described herein involve cancer, (e.g., the treatment of a cancer). For example, § 5.11.5 describes various methods of treating cancer. This § 5.11.5.1, details exemplary cancers that can be treated according to any of the methods in § 5.11.5. The cancers are intended to be exemplary only and not limiting.

In some embodiments, the cancer is a KRAS (e.g., hKRAS) variant cancer. In some embodiments, the cancer has been determined to contain a KRAS (e.g., hKRAS) variant.

In some embodiments, the KRAS (e.g., hKRAS) variant comprises a KRAS (e.g., hKRAS) activating amino acid modification (e.g., substitution). In some embodiments, the KRAS (e.g., hKRAS) variant comprises a KRAS (e.g., hKRAS) activating amino acid substitution.

In some embodiments, the KRAS (e.g., hKRAS) variant comprises an amino acid modification (e.g., substitution) at amino acid position G12, G13, or Q61, numbering relative to the amino acid sequence of SEQ ID NO: 3. In some embodiments, the KRAS (e.g., hKRAS) variant comprises an amino acid substitution at amino acid position G12, G13, or Q61, numbering relative to the amino acid sequence of SEQ ID NO: 3.

In some embodiments, the KRAS (e.g., hKRAS) variant comprises an amino acid modification (e.g., substitution) at amino acid position G12 or G13, numbering relative to the amino acid sequence of SEQ ID NO: 3. In some embodiments, the KRAS (e.g., hKRAS) variant comprises an amino acid substitution at amino acid position G12 or G13, numbering relative to the amino acid sequence of SEQ ID NO: 3.

In some embodiments, the KRAS variant comprises any one or more of the following amino acid substitutions G12C, G12V, G12R, G12D, G12A, G13D, or Q61H, numbering relative to the amino acid sequence of SEQ ID NO: 3. In some embodiments, the KRAS (e.g., hKRAS) variant comprises any one or more of the following amino acid substitutions G12C, G12V, G12R, G12D, G12A, or G13D, numbering relative to the amino acid sequence of SEQ ID NO: 3. In some embodiments, the KRAS (e.g., hKRAS) variant comprises any one of the following amino acid substitutions G12C, G12V, G12R, G12A, or G12D, numbering relative to the amino acid sequence of SEQ ID NO: 3. In some embodiments, the KRAS (e.g., hKRAS) variant comprises a G12C amino acid substitution, numbering relative to the amino acid sequence of SEQ ID NO: 3.

Determining whether a cancer comprises a KRAS (e.g., hKRAS) variant (e.g., hKRAS G12C) can be assessed by conventional methods known in the art. For example, the nucleotide sequence encoding the KRAS (e.g., hKRAS) protein can be assessed, the amino acid sequence of the KRAS protein can be assessed, and/or the functional characteristics of the KRAS (e.g., hKRAS) protein can be assessed. The sequence of reference wildtype hKRAS is known in the art and provided herein as SEQ ID NO: 3. Methods for detecting an amino acid modification, e.g., substitution, in a KRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS amino acid substitutions by real-time PCR. For example, in real-time PCR, fluorescent probes specific for the KRAS amino acid substitution are utilized. When a substitution is present, the probe binds and fluorescence is detected. In some embodiments, the KRAS (e.g., hKRAS) substitution is identified using a direct sequencing method of specific regions (e.g., specific exons) in the KRAS (e.g., hKRAS) gene. This technique will identify all possible amino acid substitutions in the region sequenced. Methods for detecting an amino acid substitution in a KRAS (e.g., hKRAS) protein are known by those of skill in the art. These methods include, but are not limited to, detection of a KRAS (e.g., hKRAS) variant using a binding agent (e.g. an antibody) specific for the variant protein, protein electrophoresis, Western blotting and direct peptide sequencing.

Methods for determining whether a cancer comprises a KRAS (e.g., hKRAS) variant can use a variety of samples. In some embodiments, the sample is taken from a subject having a cancer. In some embodiments, the sample is a fresh cancer sample. In some embodiments, the sample is a frozen cancer sample. In some embodiments, the sample is a formalin-fixed paraffin-embedded sample. In some embodiments, the sample is processed to a cell lysate. In some embodiments, the sample is processed to DNA or RNA. In some embodiments, the sample is a biopsy. In some embodiments, the sample is further processed.

In some embodiments, the cancer is resistant to a KRAS (e.g., hKRAS) inhibitor (e.g., a KRAS (e.g., hKRAS) inhibitor described herein). In some embodiments, the cancer has been determined to be resistant to a KRAS (e.g., hKRAS) inhibitor (e.g., a KRAS (e.g., hKRAS) inhibitor described herein). In some embodiments, the KRAS (e.g., hKRAS) inhibitor has previously been administered to the subject. In some embodiments, the KRAS (e.g., hKRAS) inhibitor has previously been administered to the subject in the absence of a fusion protein described herein.

In some embodiments, the cancer overexpressed EGFR (e.g., hEGFR) relative to the expression level of EGFR (e.g., hEGFR) on non-cancer cells. In some embodiments, the cancer has been determined to overexpress EGFR (e.g., hEGFR) relative to the expression level of EGFR (e.g., hEGFR) on non-cancer cells. Methods of determining the expression of EGFR (e.g., hEGFR) in a cancer relative to non-cancer cells are known in the art. For example, immunohistochemistry (or other staining methods) can be utilized to assess the level of EGFR expressed on a cancer sample from a subject (e.g., versus a non-cancer sample from the subject). For example, see, e.g., Hirsch, Fred R et al. “Epidermal growth factor receptor immunohistochemistry: comparison of antibodies and cutoff points to predict benefit from gefitinib in a phase 3 placebo-controlled study in advanced nonsmall-cell lung cancer.” Cancer vol. 112, 5 (2008): 1114-21. doi: 10.1002/cncr.23282, the entire contents of which are incorporated herein by reference for all purposes.

In some embodiments, the cancer expresses a variant EGFR (e.g., hEGFR). In some embodiments, the cancer has been determined to express a variant EGFR (e.g., hEGFR). Methods of determining whether a cancer expresses a variant EGFR (e.g., hEGFR) protein are known in the art. Exemplary methods are detailed above with reference variant KRAS (e.g., hKRAS) proteins. The same general methods can be employed to assess whether a cancer expresses a variant KRAS (e.g., hKRAS).

In some embodiments, the cancer cells overexpress TGFβ (e.g., hTGFβ) relative to non-cancer cells. In some embodiments, the cancer cells have been determined to overexpress TGFβ (e.g., hTGFβ) relative to non-cancer cells. Methods of determining the expression of TGFβ (e.g., hTGFβ) in a cancer relative to non-cancer cells are known in the art. For example, an ELISA or other immunoassay can be utilized to assess the level of TGFβ (e.g., hTGFβ) in a sample (e.g., a tissue sample, blood sample, serum sample) from a subject having cancer.

In some embodiments, the cancer is local, locally advanced, or metastatic.

In some embodiments, the cancer is a carcinoma (e.g., adenocarcinoma, squamous cell carcinoma).

In some embodiments, the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), pancreatic cancer (e.g., pancreatic ductal adenocarcinoma), breast cancer (e.g., invasive ductal carcinoma), stomach cancer (e.g., stomach adenocarcinoma), endometrial cancer (e.g., undifferentiated endometrial carcinoma), uterine cancer (e.g., uterine endometrial carcinoma), testicular cancer (e.g., testicular germ cell cancer), cervical cancer (e.g., cervical squamous carcinoma), bile duct cancer (e.g., cholangiocarcinoma), a myelodysplastic cancer, or esophageal cancer (e.g., esophageal adenocarcinoma, gastroesophageal junction cancer).

In some embodiments, the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), or a pancreatic cancer (e.g., pancreatic ductal adenocarcinoma).

In some embodiments, the cancer is a non-small cell lung cancer (NSCLC), lung adenocarcinoma, lung squamous carcinoma, colorectal adenocarcinoma, or pancreatic ductal adenocarcinoma.

### 5.12 Kits

In a one aspect, provided herein are kits comprising (i) a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), a host cell described herein (e.g., a host cell comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein))), or a carrier described herein (e.g., a carrier comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), or a host cell described herein); or a pharmaceutical composition described herein comprising any of the foregoing; and (ii) a KRAS (e.g., hKRAS) inhibitor (e.g., a KRAS (e.g., hKRAS) inhibitor described herein) or a pharmaceutical composition comprising the KRAS (e.g., hKRAS) inhibitor. In some embodiments, (i) and (ii) are part of the same pharmaceutical composition. In some embodiments, (i) and (ii) are provided in the kit as separate pharmaceutical compositions. Also provided herein are kits comprising a combination regimen described herein (or pharmaceutical composition(s)) thereof. Also provided herein are kits comprising a combination composition described herein (or a pharmaceutical composition thereof).

In addition, the kit may comprise a liquid vehicle for solubilizing or diluting, and/or technical instructions. The technical instructions of the kit may contain information about administration and dosage and subject groups.

In some embodiments, the fusion protein described herein, the polynucleotide described herein (e.g., a polynucleotide encoding a fusion protein described herein), the vector described herein (e.g., a vector comprising a polynucleotide described herein (e.g., a polynucleotide encoding a fusion protein described herein)), the host cell described herein (e.g., a host cell comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein))), the carrier described herein (e.g., a carrier comprising a fusion protein described herein, a polynucleotide described herein (e.g., a polynucleotide encoding a fusion protein described herein), a vector described herein (e.g., a vector comprising a polynucleotide described herein (e.g., a polynucleotide encoding a fusion protein described herein)), or a host cell described herein); or the pharmaceutical composition described herein comprising any of the foregoing is lyophilized, spray-dried, or spray-freeze dried. The kit may further contain as a part a vehicle (e.g., buffer solution) for solubilizing the dried or lyophilized fusion protein (or polypeptides thereof) described herein, polynucleotide described herein, vector described herein, host cell described herein, carrier described herein, or pharmaceutical composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container.

Any of the kits described herein may be used in any of the methods described herein (see, e.g., § 5.11).

### 5.13 Exemplary Embodiments

The following provides exemplary embodiments (Es) of the disclosure. The embodiments are exemplary only and are in no way limited.
E1. A method of treating cancer in a subject in need thereof, the method comprising: administering to the subject (a) a kirsten rat sarcoma virus homolog (KRAS) (e.g., human KRAS (hKRAS)) inhibitor; in combination with (b) a fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)), to thereby treat the cancer in the subject.
E2. The method of Embodiment 1 (E1), wherein the KRAS (e.g., hKRAS) inhibitor and the fusion protein are administered in an amount and for a time sufficient to treat or prevent the cancer in the subject.
E3. A method of treating a KRAS (e.g., hKRAS) variant cancer in a subject in need thereof, the method comprising: (a) receiving test results showing that the KRAS (e.g., hKRAS) variant cancer is resistant to a KRAS inhibitor; and (b) administering to the subject the KRAS (e.g., hKRAS) inhibitor in combination with a fusion protein comprising: (i) a first moiety that specifically binds EGFR (e.g., hEGFR) operably connected to (ii) a second moiety that specifically binds TGFβ (e.g., hTGFβ), to thereby treat the KRAS (e.g., hKRAS) variant cancer in the subject.
E4. The method of E3, wherein the KRAS (e.g., hKRAS) inhibitor has been previously administered to the subject.
E5. The method of E3 or E4, wherein the KRAS (e.g., hKRAS) inhibitor and the fusion protein are administered in an amount and for a time sufficient to treat the KRAS (e.g., hKRAS) variant cancer in the subject.
E6. A method of treating a KRAS (e.g., hKRAS) variant cancer in a subject in need thereof, the method comprising: (a) administering to the subject a KRAS (e.g., hKRAS) inhibitor; (b) receiving testing results showing that the KRAS (e.g., hKRAS) variant cancer has developed resistance to the KRAS (e.g., hKRAS) inhibitor; and (c) administering to the subject the KRAS (e.g., hKRAS) inhibitor in combination with a fusion protein comprising: (i) a first moiety that specifically binds EGFR (e.g., hEGFR) operably connected to (ii) a second moiety that specifically binds TGFβ (e.g., hTGFβ), to thereby treat the KRAS (e.g., hKRAS) variant cancer in the subject.
E7. The method of E6, wherein the KRAS (e.g., hKRAS) inhibitor and the fusion protein are administered in an amount and for a time sufficient to treat the KRAS (e.g., hKRAS) variant cancer in the subject.
E8. A method of treating a KRAS (e.g., hKRAS) variant cancer in a subject in need thereof, the method comprising: (a) determining that the KRAS (e.g., hKRAS) variant cancer is resistant to a KRAS (e.g., hKRAS) inhibitor; and (b) administering to the subject a KRAS (e.g., hKRAS) inhibitor in combination with a fusion protein comprising: (i) a first moiety that specifically binds EGFR (e.g., hEGFR) operably connected to (ii) a second moiety that specifically binds TGFβ (e.g., hTGFβ), to thereby treat the KRAS (e.g., hKRAS) variant cancer in the subject.
E9. The method of E8, wherein the subject has been previously treated with the KRAS (e.g., hKRAS) inhibitor.
E10. The method of E8 or E9, wherein the KRAS (e.g., hKRAS) inhibitor and the fusion protein are administered in an amount and for a time sufficient to treat the KRAS (e.g., hKRAS) variant cancer in the subject.
E11. The method of any one of the preceding embodiments, wherein the cancer is a KRAS (e.g., hKRAS) variant cancer.
E12. The method of any one of the preceding embodiments, wherein the cancer has been determined to contain a KRAS (e.g., hKRAS) variant.
E13. The method of E11 or E12, wherein the KRAS (e.g., hKRAS) variant comprises a KRAS (e.g., hKRAS) activating amino acid modification (e.g., substitution).
E14. The method of any one of E11-E13, wherein the KRAS (e.g., hKRAS) variant comprises an amino acid modification (e.g., substitution) at amino acid position G12, G13, or Q61, numbering relative to the amino acid sequence of SEQ ID NO: 3.
E15. The method of any one of E11-E14, wherein the KRAS (e.g., hKRAS) variant comprises an amino acid modification (e.g., substitution) at amino acid position G12 or G13, numbering relative to the amino acid sequence of SEQ ID NO: 3.
E16. The method of any one of E11-E15, wherein the KRAS (e.g., hKRAS) variant comprises any one or more of the following amino acid substitutions G12C, G12V, G12R, G12D, G12A, G12S, G13D, or Q61H, numbering relative to the amino acid sequence of SEQ ID NO: 3.
E17. The method of any one of E11-E16, wherein the KRAS (e.g., hKRAS) variant comprises any one or more of the following amino acid substitutions G12C, G12V, G12R, G12D, G12A, G12S, or G13D, numbering relative to the amino acid sequence of SEQ ID NO: 3.
E18. The method of any one of E11-E17, wherein the KRAS (e.g., hKRAS) variant comprises any one of the following amino acid substitutions G12C, G12V, G12R, G12A, G12S, or G12D, numbering relative to the amino acid sequence of SEQ ID NO: 3.
E19. The method of any one of E11-E18, wherein the KRAS (e.g., hKRAS) variant comprises a G12C amino acid substitution, numbering relative to the amino acid sequence of SEQ ID NO: 3.
E20. The method of any one of the preceding embodiments, wherein the cancer is resistant to the KRAS (e.g., hKRAS) inhibitor.
E21. The method of any one of the preceding embodiments, wherein the cancer has been determined to be resistant to the KRAS (e.g., hKRAS) inhibitor.
E22. The method of any one of the preceding embodiments, wherein the KRAS (e.g., hKRAS) inhibitor has previously been administered to the subject.
E23. The method of any one of the preceding embodiments, wherein the KRAS (e.g., hKRAS) inhibitor has previously been administered to the subject in the absence of the fusion protein.
E24. The method of any one of the preceding embodiments, wherein the cancer cells overexpress EGFR (e.g., hEGFR) relative to non-cancer cells.
E25. The method of any one of the preceding embodiments, wherein the cancer cells have been determined to overexpress EGFR (e.g., hEGFR) relative to non-cancer cells.
E26. The method of any one of the preceding embodiments, wherein the cancer cells express an EGFR (e.g., hEGFR) variant.
E27. The method of any one of the preceding embodiments, wherein the cancer cells have been determined to express an EGFR (e.g., hEGFR) variant.
E28. The method of any one of the preceding embodiments, wherein the cancer cells overexpress hTGFβ (e.g., hTGFβ) relative to non-cancer cells.
E29. The method of any one of the preceding embodiments, wherein the cancer cells have been determined to overexpress TGFβ (e.g., hTGFβ) relative to non-cancer cells.
E30. The method of any one of the preceding embodiments, wherein the cancer is local, locally advanced, or metastatic.
E31. The method of any one of the preceding embodiments, wherein the cancer is a carcinoma (e.g., adenocarcinoma, squamous cell carcinoma).
E32. The method of any one of the preceding embodiments, wherein the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), pancreatic cancer (e.g., pancreatic ductal adenocarcinoma), breast cancer (e.g., invasive ductal carcinoma), stomach cancer (e.g., stomach adenocarcinoma), endometrial cancer (e.g., undifferentiated endometrial carcinoma), uterine cancer (e.g., uterine endometrial carcinoma), testicular cancer (e.g., testicular germ cell cancer), cervical cancer (e.g., cervical squamous carcinoma), bile duct cancer (e.g., cholangiocarcinoma), a myelodysplastic cancer, or esophageal cancer (e.g., esophageal adenocarcinoma, gastroesophageal junction cancer).
E33. The method of any one of the preceding embodiments, wherein the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), or a pancreatic cancer (e.g., pancreatic ductal adenocarcinoma).
E34. The method of any one of the preceding embodiments, wherein the cancer is a non-small cell lung cancer (NSCLC), lung adenocarcinoma, lung squamous carcinoma, colorectal adenocarcinoma, or pancreatic ductal adenocarcinoma.
E35. A method of delivering a KRAS (e.g., hKRAS) inhibitor and a fusion protein to a subject in need thereof, the method comprising administering (a) a KRAS (e.g., hKRAS) inhibitor; in combination with (b) a fusion protein comprising: (i) a first moiety that specifically binds EGFR) (e.g., hEGFR) operably connected to (ii) a second moiety that specifically binds TGFβ (e.g., hTGFβ), to thereby deliver the KRAS (e.g., hKRAS) inhibitor and a fusion protein to a subject.
E36. A method of inhibiting the KRAS (e.g., hKRAS) pathway and the TGFβ (e.g., hTGFβ) pathway in a subject in need thereof, the method comprising administering (a) a KRAS (e.g., hKRAS) inhibitor; in combination with (b) a fusion protein comprising: (i) a first moiety that specifically binds EGFR (e.g., hEGFR) operably connected to (ii) a second moiety that specifically binds TGFβ (e.g., hTGFβ), to thereby inhibit the KRAS (e.g., hKRAS) pathway and the TGFβ (e.g., hTGFβ) pathway in the subject.
E37. The method of E36, wherein the KRAS (e.g., hKRAS) inhibitor and the fusion protein are administered in an amount and for a time sufficient to inhibit the KRAS (e.g., hKRAS) pathway and the TGFβ (e.g., hTGFβ) pathway in the subject.
E38. A method of restoring sensitivity to a KRAS (e.g., hKRAS) inhibitor in a subject in need thereof, the method comprising administering to the subject a fusion protein comprising: (i) a first moiety that specifically binds EGFR (e.g., hEGFR) operably connected to (ii) a second moiety that specifically binds TGFβ (e.g., hTGFβ), to thereby restore sensitivity to the KRAS (e.g., hKRAS) inhibitor in the subject.
E39. The method of E38, wherein the fusion protein is administered in an amount and for a time sufficient to restore sensitivity to the KRAS (e.g., hKRAS) inhibitor in the subject.
E40. The method of any one of E38-E39, wherein the cancer in the subject is resistant to the KRAS (e.g., hKRAS) inhibitor.
E41. The method of any one of E38-E40, wherein the cancer has been determined to be resistant to the KRAS (e.g., hKRAS) inhibitor.
E42. The method of any one of E38-E41, further comprising administering the KRAS (e.g., hKRAS) inhibitor to the subject in combination with the fusion protein.
E43. A method of suppressing or preventing resistance to a KRAS (e.g., hKRAS) inhibitor in a subject in need thereof, the method comprising administering to the subject (a) a KRAS (e.g., hKRAS) inhibitor; in combination with (b) a fusion protein comprising: (i) a first moiety that specifically binds EGFR (e.g., hEGFR) operably connected to (ii) a second moiety that specifically binds TGFβ (e.g., hTGFβ), to thereby suppress or prevent resistance to the KRAS (e.g., hKRAS) inhibitor in the subject.
E44. The method of E43, wherein the fusion protein is administered in an amount and for a time sufficient to suppress the development of resistance to the KRAS (e.g., hKRAS) inhibitor in the subject.
E45. The method of any one of E35-E44, wherein the KRAS (e.g., hKRAS) inhibitor was previously administered to the subject.
E46. The method of any one of the preceding embodiments, wherein the subject has been previously administered a KRAS (e.g., hKRAS) inhibitor (e.g., the KRAS (e.g., hKRAS) inhibitor).
E47. The method of any one of the preceding embodiments, wherein the KRAS (e.g., hKRAS) inhibitor has not been previously administered to the subject.
E48. The method of any one of the preceding embodiments, wherein the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor.
E49. The method of any one of the preceding embodiments, wherein the cancer in the subject is resistant to a KRAS (e.g., hKRAS) inhibitor (e.g., the KRAS (e.g., hKRAS) inhibitor).
E50. The method of any one of the preceding embodiments, wherein the KRAS (e.g., hKRAS) inhibitor is capable of selectively inhibiting a KRAS (e.g., hKRAS) variant.
E51. The method of any one of the preceding embodiments, wherein the KRAS (e.g., hKRAS) variant comprises a KRAS (e.g., hKRAS) activating amino acid modification.
E52. The method of any one of the preceding embodiments, wherein the KRAS (e.g., hKRAS) variant comprises an amino acid modification at amino acid position G12, G13, or Q61, numbering relative to the amino acid sequence of SEQ ID NO: 3.
E53. The method of any one of the preceding embodiments, wherein the KRAS (e.g., hKRAS) variant comprises an amino acid modification at amino acid position G12 or G13, numbering relative to the amino acid sequence of SEQ ID NO: 3.
E54. The method of any one of the preceding embodiments, wherein the KRAS (e.g., hKRAS) variant comprises any one or more of the following amino acid substitutions G12C, G12V, G12R, G12D, G12A, G13D, or Q61H, numbering relative to the amino acid sequence of SEQ ID NO: 3.
E55. The method of any one of the preceding embodiments, wherein the KRAS (e.g., hKRAS) variant comprises any one or more of the following amino acid substitutions G12C, G12V, G12R, G12D, G12A, or G13D, numbering relative to the amino acid sequence of SEQ ID NO: 3.
E56. The method of any one of the preceding embodiments, wherein the KRAS (e.g., hKRAS) variant comprises any one of the following amino acid substitutions G12C, G12V, G12R, G12A, or G12D, numbering relative to the amino acid sequence of SEQ ID NO: 3.
E57. The method of any one of the preceding embodiments, wherein the KRAS (e.g., hKRAS) variant comprises a G12C amino acid substitution, numbering relative to the amino acid sequence of SEQ ID NO: 3.
E58. The method of any one of the preceding embodiments, wherein the KRAS (e.g., hKRAS) inhibitor comprises or consists of a small molecule, protein (e.g., an antibody or functional fragment or variant thereof), nucleic acid, carbohydrate, a lipid, a metal, or a toxin.
E59. The method of any one of the preceding embodiments, wherein the KRAS (e.g., hKRAS) inhibitor comprises or consists of a small molecule.
E60. The method of any one of the preceding embodiments, wherein the KRAS (e.g., hKRAS) inhibitor comprises a KRAS (e.g., hKRAS) inhibitor in Table 2.
E61. The method of any one of the preceding embodiments, wherein the KRAS (e.g., hKRAS) inhibitor comprises sotorasib (AMG-510).
E62. The method of any one of the preceding embodiments, wherein the first moiety comprises an antibody, or functional fragment or functional variant thereof.
E63. The method of any one of the preceding embodiments, wherein the first moiety comprises a full-length antibody, a single chain variable fragment (scFv), a Fab, or a single domain antibody (sdAb).
E64. The method of any one of the preceding embodiments, wherein the first moiety comprises a full-length antibody.
E65. The method of any one of the preceding embodiments, wherein the first moiety comprises a variable heavy chain (VH) region that comprises three complementarity determining regions: VH CDR1, VH CDR2, and VH CDR3; and a variable light chain (VL) region that comprises three complementarity determining regions: VL CDR1, VL CDR2, and VL CDR3.
E66. The method of any one of the preceding embodiments, wherein (a) the amino acid sequence of VH CDR1 comprises the amino acid sequence SEQ ID NO: 39, or the amino acid sequence of SEQ ID NO: 39 comprising 1, 2, or 3 amino acid modifications; (b) the amino acid sequence of VH CDR2 comprises the amino acid sequence SEQ ID NO: 40, or the amino acid sequence of SEQ ID NO: 40 comprising 1, 2, or 3 amino acid modifications; (c) the amino acid sequence of VH CDR3 comprises the amino acid sequence SEQ ID NO: 41, or the amino acid sequence of SEQ ID NO: 41 comprising 1, 2, or 3 amino acid modifications; and (d) the amino acid sequence of VL CDR1 comprises the amino acid sequence SEQ ID NO: 42, or the amino acid sequence of SEQ ID NO: 42 comprising 1, 2, or 3 amino acid modifications; (e) the amino acid sequence of VL CDR2 comprises the amino acid sequence SEQ ID NO: 43, or the amino acid sequence of SEQ ID NO: 43 comprising 1, 2, or 3 amino acid modifications; and (f) the amino acid sequence of VL CDR3 comprises the amino acid sequence SEQ ID NO: 44, or the amino acid sequence of SEQ ID NO: 44 comprising 1, 2, or 3 amino acid modifications.
E67. The method of any one of the preceding embodiments, wherein the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46.
E68. The method of any one of the preceding embodiments, wherein the first moiety comprises a heavy chain (HC) and a light chain (LC), wherein the amino acid sequence of the HC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and the amino acid sequence of the LC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49.
E69. The method of any one of the preceding embodiments, wherein the second moiety comprises an antibody, or functional fragment or functional variant thereof.
E70. The method of any one of the preceding embodiments, wherein the second moiety comprises a full-length antibody, a single chain variable fragment (scFv), a scFv2, a scFv-Fc, a Fab, a Fab′, a F(ab′)2, or a F(v).
E71. The method of any one of the preceding embodiments, wherein the second moiety comprises or consists of at least a portion of the extracellular domain (ECD) of a transforming growth factor-beta receptor (TGFβR) (e.g., hTGFβR).
E72. The method of any one of the preceding embodiments, wherein the second moiety comprises or consists of at least a portion of the ECD of transforming growth factor-beta receptor II (TGFβRII) (e.g., hTGFβRII).
E73. The method of any one of the preceding embodiments, wherein the second moiety comprises or consists of the ECD of TGFβRII (e.g., hTGFβRII).
E74. The method of any one of the preceding embodiments, wherein the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E75. The method of any one of the preceding embodiments, wherein the fusion protein comprises (iii) a third moiety that specifically binds TGFβ (e.g., hTGFβ) and is operably connected to the first moiety.
E76. The method of any one of the preceding embodiments, wherein the third moiety comprises an antibody, or functional fragment or functional variant thereof.
E77. The method of any one of the preceding embodiments, wherein the third moiety comprises a full-length antibody, a single chain variable fragment (scFv), a scFv2, a scFv-Fc, a Fab, a Fab′, a F(ab′)2, or a F(v).
E78. The method of any one of the preceding embodiments, wherein the third moiety comprises or consists of at least a portion of the extracellular domain (ECD) of a TGFβR (e.g., hTGFβR).
E79. The method of any one of the preceding embodiments, wherein the third moiety comprises or consists of at least a portion of the ECD of TGFβRII (e.g., hTGFβRII).
E80. The method of any one of the preceding embodiments, wherein the third moiety comprises or consists of the ECD of TGFβRII (e.g., hTGFβRII).
E81. The method of any one of the preceding embodiments, wherein the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E82. The method of any one of the preceding embodiments, wherein the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73; and the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E83. The method of any one of the preceding embodiments, wherein the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of the third moiety.
E84. The method of any one of the preceding embodiments, wherein the first moiety comprises a full-length antibody and wherein the N-terminus of a second moiety is operably connected (e.g., optionally via a linker) to the C-terminus of the first light chain.
E85. The method of any one of the preceding embodiments, wherein the first moiety comprises a full-length antibody and wherein the N-terminus of a second moiety is operably connected (e.g., optionally via a linker) to the C-terminus of the first heavy chain.
E86. The method of any one of the preceding embodiments, wherein the first moiety comprises a full-length antibody and wherein the N-terminus of a second moiety is operably connected (e.g., optionally via a linker) to the C-terminus of the first light chain and the N-terminus of the third moiety is operably connected (e.g., optionally via a linker) to the C-terminus of the second light chain.
E87. The method of any one of the preceding embodiments, wherein the first moiety comprises a full-length antibody and wherein the N-terminus of a second moiety is operably connected (e.g., optionally via a linker) to the C-terminus of the first heavy chain and the N-terminus of the third moiety is operably connected (e.g., optionally via a linker) to the C-terminus of the second heavy chain.
E88. The method of any one of the preceding embodiments, wherein the first moiety is directly operably connected to the second moiety.
E89. The method of any one of the preceding embodiments, wherein the first moiety is indirectly operably connected to the second moiety.
E90. The method of any one of the preceding embodiments, wherein the first moiety is indirectly operably connected to the second moiety through a first peptide linker.
E91. The method of any one of the preceding embodiments, wherein the first peptide linker comprises or consists of glycine or glycine and serine amino acid residues.
E92. The method of any one of the preceding embodiments, wherein the amino acid sequence of the first peptide linker comprises or consists of (a) the amino acid sequence of any one of SEQ ID NOS: 105-124; or (b) the amino acid sequence of any one of SEQ ID NOS: 105-124 comprising or consisting of 1, 2, or 3 amino acid modifications.
E93. The method of any one of the preceding embodiments, wherein the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.
E94. The method of any one of the preceding embodiments, wherein the first moiety is directly operably connected to the third moiety.
E95. The method of any one of the preceding embodiments, wherein the first moiety is indirectly operably connected to the third moiety.
E96. The method of any one of the preceding embodiments, wherein the first moiety is indirectly operably connected to the third moiety through a second peptide linker.
E97. The method of E96, wherein the second peptide linker comprises or consists of glycine or glycine and serine amino acid residues.
E98. The method of E96 or E97, wherein the amino acid sequence of the second peptide linker comprises or consists of (a) the amino acid sequence of any one of SEQ ID NO: 111; or (b) the amino acid sequence of any one of SEQ ID NO: 111 comprising or consisting of 1, 2, or 3 amino acid modifications.
E99. The method of any one of E96-E98, wherein the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.
E100. The method of any one of E96-E99, wherein the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of the second peptide linker.
E101. The method of any one of E96-E100, wherein the amino acid sequence of the first peptide linker is 100% identical to the amino acid sequence of the second peptide linker.
E102. The method of any one of the preceding embodiments, wherein (a) the first moiety comprises or consists of a full-length antibody; (b) the second moiety comprises a comprises or consists of the ECD of TGFβRII (e.g., hTGFβRII); (c) the third moiety comprises a comprises or consists of the ECD of TGFβRII (e.g., hTGFβRII); (d) the second moiety is operably connected to the first moiety through a first peptide linker; and (c) the third moiety is operably connected to the first moiety through a second peptide linker.
E103. The method of E102, wherein the N-terminus of the second moiety is operably connected to the C-terminus of the first light chain of the full-length antibody and the N-terminus of the third moiety is operably connected to the C-terminus of the second light chain of the full-length antibody.
E104. The method of any one of E102-E103, wherein (a) (i) the amino acid sequence of VH CDR1 comprises the amino acid sequence SEQ ID NO: 39, or the amino acid sequence of SEQ ID NO: 39 comprising 1, 2, or 3 amino acid modifications, the amino acid sequence of VH CDR2 comprises the amino acid sequence SEQ ID NO: 40, or the amino acid sequence of SEQ ID NO: 40 comprising 1, 2, or 3 amino acid modifications; the amino acid sequence of VH CDR3 comprises the amino acid sequence SEQ ID NO: 41, or the amino acid sequence of SEQ ID NO: 41 comprising 1, 2, or 3 amino acid modifications; the amino acid sequence of VL CDR1 comprises the amino acid sequence SEQ ID NO: 42, or the amino acid sequence of SEQ ID NO: 42 comprising 1, 2, or 3 amino acid modifications; the amino acid sequence of VL CDR2 comprises the amino acid sequence SEQ ID NO: 43, or the amino acid sequence of SEQ ID NO: 43 comprising 1, 2, or 3 amino acid modifications; and the amino acid sequence of VL CDR3 comprises the amino acid sequence SEQ ID NO: 44, or the amino acid sequence of SEQ ID NO: 44 comprising 1, 2, or 3 amino acid modifications; (ii) the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46; and/or (iii) the amino acid sequence of the heavy chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and wherein the amino acid sequence of the light chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49; (b) the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (c) the amino acid sequence of the third moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (d) the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111; and (e) the amino acid sequence of the second peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111.
E105. The method of any one of the preceding embodiments, wherein the fusion protein comprises: (a) a first polypeptide, wherein the amino acid sequence of the first polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (b) a second polypeptide, wherein the amino acid sequence of the second polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (c) a third polypeptide, wherein the amino acid sequence of the third polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126; and (d) a fourth polypeptide, wherein the amino acid sequence of the fourth polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.
E106. A combination regimen comprising: (a) a KRAS (e.g., hKRAS) inhibitor (e.g., a KRAS (e.g., hKRAS) inhibitor described herein); and (b) a fusion protein comprising: (i) a first moiety that specifically binds EGFR (e.g., hEGFR) operably connected to (ii) a second moiety that specifically binds TGFβ (e.g., hTGFβ) (e.g., a fusion protein described herein).
E107. A pharmaceutical composition comprising (a) a KRAS (e.g., hKRAS) inhibitor (e.g., a KRAS (e.g., hKRAS) inhibitor described herein); and (b) a fusion protein comprising: (i) a first moiety that specifically binds EGFR (e.g., hEGFR) operably connected to (ii) a second moiety that specifically binds TGFβ (e.g., hTGFβ) (e.g., a fusion protein described herein); and (c) a pharmaceutically acceptable excipient.
E108. A kit comprising a (i) (a) a KRAS (e.g., hKRAS) inhibitor (e.g., a KRAS (e.g., hKRAS) inhibitor described herein), or a pharmaceutical composition thereof; and (b) a fusion protein comprising: (i) a first moiety that specifically binds EGFR (e.g., hEGFR) operably connected to (ii) a second moiety that specifically binds TGFβ (e.g., hTGFβ) (e.g., a fusion protein described herein), or a pharmaceutical composition thereof; (ii) the combination therapy of E106; or (iii) the pharmaceutical composition of E107.
E109. A use of a fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)); for the manufacture of a medicament for treating cancer in a subject in need thereof, wherein the medicament is administered in combination with or further comprises a kirsten rat sarcoma virus homolog (KRAS) (e.g., human KRAS (hKRAS)) inhibitor.
E110. The use of E109, wherein the fusion protein and the KRAS (e.g., hKRAS) inhibitor are administered in an amount and for a time sufficient to treat or prevent the cancer in the subject.
E111. The use of E109 or E110, comprising determining that the cancer is resistant to a KRAS (e.g., hKRAS) inhibitor.
E112. The use of any one of E109-E111, wherein the cancer is resistant to a KRAS (e.g., hKRAS) inhibitor.
E113. The use of any one of E109-E112, wherein the subject has been previously treated with the KRAS (e.g., hKRAS) inhibitor.
E114. The use of any one of E109-E113, wherein the subject has been previously treated with a KRAS (e.g., hKRAS) inhibitor.
E115. The use of any one of E109-E114, wherein the cancer is a KRAS (e.g., hKRAS) variant cancer.
E116. The use of any one of E109-E115, wherein the cancer has been determined to contain a KRAS (e.g., hKRAS) variant.
E117. The use of E116, wherein the KRAS (e.g., hKRAS) variant comprises a KRAS (e.g., hKRAS) activating amino acid modification (e.g., substitution).
E118. The use of any one of E116-E117, wherein the KRAS (e.g., hKRAS) variant comprises an amino acid modification (e.g., substitution) at amino acid position G12, G13, or Q61, numbering relative to the amino acid sequence of SEQ ID NO: 3.
E119. The use of any one of E116-E118, wherein the KRAS (e.g., hKRAS) variant comprises an amino acid modification (e.g., substitution) at amino acid position G12 or G13, numbering relative to the amino acid sequence of SEQ ID NO: 3.
E120. The use of any one of E116-E119, wherein the KRAS (e.g., hKRAS) variant comprises any one or more of the following amino acid substitutions G12C, G12V, G12R, G12D, G12A, G12S, G13D, or Q61H, numbering relative to the amino acid sequence of SEQ ID NO: 3.
E121. The use of any one of E116-E120, wherein the KRAS (e.g., hKRAS) variant comprises any one or more of the following amino acid substitutions G12C, G12V, G12R, G12D, G12A, G12S, or G13D, numbering relative to the amino acid sequence of SEQ ID NO: 3.
E122. The use of any one of E116-E121, wherein the KRAS (e.g., hKRAS) variant comprises any one of the following amino acid substitutions G12C, G12V, G12R, G12A, G12S, or G12D, numbering relative to the amino acid sequence of SEQ ID NO: 3.
E123. The use of any one of E116-E122, wherein the KRAS (e.g., hKRAS) variant comprises a G12C amino acid substitution, numbering relative to the amino acid sequence of SEQ ID NO: 3.
E124. The use of any one of E109-E123, wherein the cancer is resistant to the KRAS (e.g., hKRAS) inhibitor.
E125. The use of any one of E109-E124, wherein the cancer has been determined to be resistant to the KRAS (e.g., hKRAS) inhibitor.
E126. The use of any one of E109-E125, wherein the KRAS (e.g., hKRAS) inhibitor has previously been administered to the subject.
E127. The use of any one of E109-E126, wherein the KRAS (e.g., hKRAS) inhibitor has previously been administered to the subject in the absence of the fusion protein.
E128. The use of any one of E109-E127, wherein the cancer cells overexpress EGFR (e.g., hEGFR) relative to non-cancer cells.
E129. The use of any one of E109-E128, wherein the cancer cells have been determined to overexpress EGFR (e.g., hEGFR) relative to non-cancer cells.
E130. The use of any one of E109-E129, wherein the cancer cells express an EGFR (e.g., hEGFR) variant.
E131. The use of any one of E109-E130, wherein the cancer cells have been determined to express an EGFR (e.g., hEGFR) variant.
E132. The use of any one of E109-E131, wherein the cancer cells overexpress hTGFβ (e.g., hTGFβ) relative to non-cancer cells.
E133. The use of any one of E109-E132, wherein the cancer cells have been determined to overexpress TGFβ (e.g., hTGFβ) relative to non-cancer cells.
E134. The use of any one of E109-E133, wherein the cancer is local, locally advanced, or metastatic.
E135. The use of any one of E109-E134, wherein the cancer is a carcinoma (e.g., adenocarcinoma, squamous cell carcinoma).
E136. The use of any one of E109-E135, wherein the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), pancreatic cancer (e.g., pancreatic ductal adenocarcinoma), breast cancer (e.g., invasive ductal carcinoma), stomach cancer (e.g., stomach adenocarcinoma), endometrial cancer (e.g., undifferentiated endometrial carcinoma), uterine cancer (e.g., uterine endometrial carcinoma), testicular cancer (e.g., testicular germ cell cancer), cervical cancer (e.g., cervical squamous carcinoma), bile duct cancer (e.g., cholangiocarcinoma), a myelodysplastic cancer, or esophageal cancer (e.g., esophageal adenocarcinoma, gastroesophageal junction cancer).
E137. The use of any one of E109-E136, wherein the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), or a pancreatic cancer (e.g., pancreatic ductal adenocarcinoma).
E138. The use of any one of E109-E137, wherein the cancer is a non-small cell lung cancer (NSCLC), lung adenocarcinoma, lung squamous carcinoma, colorectal adenocarcinoma, or pancreatic ductal adenocarcinoma.
E139. A use of a fusion protein comprising: (i) a first moiety that specifically binds EGFR (e.g., hEGFR) operably connected to (ii) a second moiety that specifically binds TGFβ (e.g., hTGFβ) in the manufacture of a medicament for inhibiting the KRAS (e.g., hKRAS) pathway and the TGFβ (e.g., hTGFβ) pathway in a subject in need thereof, wherein the medicament is administered in combination with or further comprises a KRAS (e.g., hKRAS) inhibitor.
E140. The use of E139, wherein the KRAS (e.g., hKRAS) inhibitor and the fusion protein are administered in an amount and for a time sufficient to inhibit the KRAS (e.g., hKRAS) pathway and the TGFβ (e.g., hTGFβ) pathway in the subject.
E141. A use of a fusion protein comprising: (i) a first moiety that specifically binds EGFR (e.g., hEGFR) operably connected to (ii) a second moiety that specifically binds TGFβ (e.g., hTGFβ) in the manufacture of a medicament for restoring sensitivity to a KRAS (e.g., hKRAS) inhibitor in a subject in need thereof.
E142. The use of E140, wherein the fusion protein is administered in an amount and for a time sufficient to restore sensitivity to the KRAS (e.g., hKRAS) inhibitor in the subject.
E143. The use of any one of E140-E142, wherein the cancer in the subject is resistant to the KRAS (e.g., hKRAS) inhibitor.
E144. The use of any one of E140-E143, wherein the cancer has been determined to be resistant to the KRAS (e.g., hKRAS) inhibitor.
E145. The use of any one of E140-E144, wherein the medicament is administered in combination with or comprises the KRAS (e.g., hKRAS) inhibitor to the subject.
E146. A use of a fusion protein comprising: (i) a first moiety that specifically binds EGFR (e.g., hEGFR) operably connected to (ii) a second moiety that specifically binds TGFβ (e.g., hTGFβ) in the manufacture of a medicament for restoring sensitivity to a KRAS (e.g., hKRAS) inhibitor in a subject in need thereof, wherein the medicament is administered in combination with or further comprises a KRAS (e.g., hKRAS) inhibitor.
E147. The method of E146, wherein the KRAS (e.g., hKRAS) inhibitor and the fusion protein are administered in an amount and for a time sufficient to inhibit the KRAS (e.g., hKRAS) pathway and the TGFβ (e.g., hTGFβ) pathway in the subject.
E148. A use of a fusion protein comprising: (i) a first moiety that specifically binds EGFR (e.g., hEGFR) operably connected to (ii) a second moiety that specifically binds TGFβ (e.g., hTGFβ) in the manufacture of a medicament for suppressing or preventing resistance to a KRAS (e.g., hKRAS) inhibitor in a subject in need thereof, wherein the medicament is administered in combination with or further comprises a KRAS (e.g., hKRAS) inhibitor.
E149. The use of E148, wherein the fusion protein is administered in an amount and for a time sufficient to suppress the development of resistance to the KRAS (e.g., hKRAS) inhibitor in the subject.
E150. The use of any one of E148-E149, wherein the KRAS (e.g., hKRAS) inhibitor was previously administered to the subject.
E151. The use of any one of E109-E150, wherein the subject has been previously administered a KRAS (e.g., hKRAS) inhibitor (e.g., the KRAS (e.g., hKRAS) inhibitor).
E152. The use of any one of E109-E151, wherein the KRAS (e.g., hKRAS) inhibitor has not been previously administered to the subject.
E153. The use of any one of E109-E152, wherein the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor.
E154. The use of any one of E109-E153, wherein the subject has cancer.
E155. The use of any one of E109-E154, wherein the cancer in the subject is resistant to a KRAS (e.g., hKRAS) inhibitor (e.g., the KRAS (e.g., hKRAS) inhibitor).
E156. The use of any one of E109-E155, wherein the KRAS (e.g., hKRAS) inhibitor is capable of selectively inhibiting a KRAS (e.g., hKRAS) variant.
E157. The use of any one of E109-E156, wherein the KRAS (e.g., hKRAS) variant comprises a KRAS (e.g., hKRAS) activating amino acid modification.
E158. The use of any one of E109-E157, wherein the KRAS (e.g., hKRAS) variant comprises an amino acid modification at amino acid position G12, G13, or Q61, numbering relative to the amino acid sequence of SEQ ID NO: 3.
E159. The use of any one of E109-E158, wherein the KRAS (e.g., hKRAS) variant comprises an amino acid modification at amino acid position G12 or G13, numbering relative to the amino acid sequence of SEQ ID NO: 3.
E160. The use of any one of E109-E159, wherein the KRAS (e.g., hKRAS) variant comprises any one or more of the following amino acid substitutions G12C, G12V, G12R, G12D, G12A, G13D, or Q61H, numbering relative to the amino acid sequence of SEQ ID NO: 3.
E161. The use of any one of E109-E160, wherein the KRAS (e.g., hKRAS) variant comprises any one or more of the following amino acid substitutions G12C, G12V, G12R, G12D, G12A, or G13D, numbering relative to the amino acid sequence of SEQ ID NO: 3.
E162. The use of any one of E109-E161, wherein the KRAS (e.g., hKRAS) variant comprises any one of the following amino acid substitutions G12C, G12V, G12R, G12A, or G12D, numbering relative to the amino acid sequence of SEQ ID NO: 3.
E163. The use of any one of E109-E162, wherein the KRAS (e.g., hKRAS) variant comprises a G12C amino acid substitution, numbering relative to the amino acid sequence of SEQ ID NO: 3.
E164. The use of any one of E109-E163, wherein the KRAS (e.g., hKRAS) inhibitor comprises or consists of a small molecule, protein (e.g., an antibody or functional fragment or variant thereof), nucleic acid, carbohydrate, a lipid, a metal, or a toxin.
E165. The use of any one of E109-E164, wherein the KRAS (e.g., hKRAS) inhibitor comprises or consists of a small molecule.
E166. The use of any one of E109-E165, wherein the KRAS (e.g., hKRAS) inhibitor comprises a KRAS (e.g., hKRAS) inhibitor in Table 2.
E167. The use of any one of E109-E166, wherein the KRAS (e.g., hKRAS) inhibitor comprises sotorasib (AMG-510).
E168. The use of any one of E109-E167, wherein the first moiety comprises an antibody, or functional fragment or functional variant thereof.
E169. The use of any one of E109-E168, wherein the first moiety comprises a full-length antibody, a single chain variable fragment (scFv), a Fab, or a single domain antibody (sdAb).
E170. The use of any one of E109-E169, wherein the first moiety comprises a full-length antibody.
E171. The use of any one of E109-E170, wherein the first moiety comprises a variable heavy chain (VH) region that comprises three complementarity determining regions: VH CDR1, VH CDR2, and VH CDR3; and a variable light chain (VL) region that comprises three complementarity determining regions: VL CDR1, VL CDR2, and VL CDR3.
E172. The use of any one of E109-E171, wherein (a) the amino acid sequence of VH CDR1 comprises the amino acid sequence SEQ ID NO: 39, or the amino acid sequence of SEQ ID NO: 39 comprising 1, 2, or 3 amino acid modifications; (b) the amino acid sequence of VH CDR2 comprises the amino acid sequence SEQ ID NO: 40, or the amino acid sequence of SEQ ID NO: 40 comprising 1, 2, or 3 amino acid modifications; (c) the amino acid sequence of VH CDR3 comprises the amino acid sequence SEQ ID NO: 41, or the amino acid sequence of SEQ ID NO: 41 comprising 1, 2, or 3 amino acid modifications; and (d) the amino acid sequence of VL CDR1 comprises the amino acid sequence SEQ ID NO: 42, or the amino acid sequence of SEQ ID NO: 42 comprising 1, 2, or 3 amino acid modifications; (e) the amino acid sequence of VL CDR2 comprises the amino acid sequence SEQ ID NO: 43, or the amino acid sequence of SEQ ID NO: 43 comprising 1, 2, or 3 amino acid modifications; and (f) the amino acid sequence of VL CDR3 comprises the amino acid sequence SEQ ID NO: 44, or the amino acid sequence of SEQ ID NO: 44 comprising 1, 2, or 3 amino acid modifications.
E173. The use of any one of E109-E172, wherein the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46.
E174. The use of any one of E109-E173, wherein the first moiety comprises a heavy chain (HC) and a light chain (LC), wherein the amino acid sequence of the HC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and the amino acid sequence of the LC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49.
E175. The use of any one of E109-E174, wherein the second moiety comprises an antibody, or functional fragment or functional variant thereof.
E176. The use of any one of E109-E175, wherein the second moiety comprises a full-length antibody, a single chain variable fragment (scFv), a scFv2, a scFv-Fc, a Fab, a Fab′, a F(ab′)2, or a F(v).
E177. The use of any one of E109-E176, wherein the second moiety comprises or consists of at least a portion of the extracellular domain (ECD) of a transforming growth factor-beta receptor (TGFβR) (e.g., hTGFβR).
E178. The use of any one of E109-E177, wherein the second moiety comprises or consists of at least a portion of the ECD of transforming growth factor-beta receptor II (TGFβRII) (e.g., hTGFβRII).
E179. The use of any one of E109-E178, wherein the second moiety comprises or consists of the ECD of TGFβRII (e.g., hTGFβRII).
E180. The use of any one of E109-E179, wherein the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E181. The use of any one of E109-E180, wherein the fusion protein comprises (iii) a third moiety that specifically binds TGFβ (e.g., hTGFβ) and is operably connected to the first moiety.
E182. The use of any one of E109-E181, wherein the third moiety comprises an antibody, or functional fragment or functional variant thereof.
E183. The use of any one of E109-E182, wherein the third moiety comprises a full-length antibody, a single chain variable fragment (scFv), a scFv2, a scFv-Fc, a Fab, a Fab′, a F(ab′)2, or a F(v).
E184. The use of any one of E109-E183, wherein the third moiety comprises or consists of at least a portion of the extracellular domain (ECD) of a TGFβR (e.g., hTGFβR).
E185. The use of any one of E109-E184, wherein the third moiety comprises or consists of at least a portion of the ECD of TGFβRII (e.g., hTGFβRII).
E186. The use of any one of E109-E185, wherein the third moiety comprises or consists of the ECD of TGFβRII (e.g., hTGFβRII).
E187. The use of any one of E109-E186, wherein the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E188. The use of any one of E109-E187, wherein the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73; and the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E189. The use of any one of E109-E188, wherein the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of the third moiety.
E190. The use of any one of E109-E189, wherein the first moiety comprises a full-length antibody and wherein the N-terminus of a second moiety is operably connected (e.g., optionally via a linker) to the C-terminus of the first light chain.
E191. The use of any one of E109-E190, wherein the first moiety comprises a full-length antibody and wherein the N-terminus of a second moiety is operably connected (e.g., optionally via a linker) to the C-terminus of the first heavy chain.
E192. The use of any one of E109-E191, wherein the first moiety comprises a full-length antibody and wherein the N-terminus of a second moiety is operably connected (e.g., optionally via a linker) to the C-terminus of the first light chain and the N-terminus of the third moiety is operably connected (e.g., optionally via a linker) to the C-terminus of the second light chain.
E193. The use of any one of E109-E192, wherein the first moiety comprises a full-length antibody and wherein the N-terminus of a second moiety is operably connected (e.g., optionally via a linker) to the C-terminus of the first heavy chain and the N-terminus of the third moiety is operably connected (e.g., optionally via a linker) to the C-terminus of the second heavy chain.
E194. The use of any one of E109-E193, wherein the first moiety is directly operably connected to the second moiety.
E195. The use of any one of E109-E194, wherein the first moiety is indirectly operably connected to the second moiety.
E196. The use of any one of E109-E195, wherein the first moiety is indirectly operably connected to the second moiety through a first peptide linker.
E197. The use of any one of E109-E196, wherein the first peptide linker comprises or consists of glycine or glycine and serine amino acid residues.
E198. The use of any one of E109-E197, wherein the amino acid sequence of the first peptide linker comprises or consists of (a) the amino acid sequence of any one of SEQ ID NOS: 105-124; or (b) the amino acid sequence of any one of SEQ ID NOS: 105-124 comprising or consisting of 1, 2, or 3 amino acid modifications.
E199. The use of any one of E109-E198, wherein the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.
E200. The use of any one of E109-E199, wherein the first moiety is directly operably connected to the third moiety.
E201. The use of any one of E109-E200, wherein the first moiety is indirectly operably connected to the third moiety.
E202. The use of any one of E109-E201, wherein the first moiety is indirectly operably connected to the third moiety through a second peptide linker.
E203. The use of E202, wherein the second peptide linker comprises or consists of glycine or glycine and serine amino acid residues.
E204. The use of E202 or E203, wherein the amino acid sequence of the second peptide linker comprises or consists of (a) the amino acid sequence of any one of SEQ ID NO: 111; or (b) the amino acid sequence of any one of SEQ ID NO: 111 comprising or consisting of 1, 2, or 3 amino acid modifications.
E205. The use of any one of E202-E204, wherein the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.
E206. The use of any one of E202-E205, wherein the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of the second peptide linker.
E207. The use of any one of E202-E206, wherein the amino acid sequence of the first peptide linker is 100% identical to the amino acid sequence of the second peptide linker.
E208. The use of any one of E109-E, wherein (a) the first moiety comprises or consists of a full-length antibody; (b) the second moiety comprises a comprises or consists of the ECD of TGFβRII (e.g., hTGFβRII); (c) the third moiety comprises a comprises or consists of the ECD of TGFβRII (e.g., hTGFβRII); (d) the second moiety is operably connected to the first moiety through a first peptide linker; and (e) the third moiety is operably connected to the first moiety through a second peptide linker.
E209. The use of claim E208, wherein the N-terminus of the second moiety is operably connected to the C-terminus of the first light chain of the full-length antibody and the N-terminus of the third moiety is operably connected to the C-terminus of the second light chain of the full-length antibody.
E210. The use of any one of E208-E209, wherein (a) (i) the amino acid sequence of VH CDR1 comprises the amino acid sequence SEQ ID NO: 39, or the amino acid sequence of SEQ ID NO: 39 comprising 1, 2, or 3 amino acid modifications, the amino acid sequence of VH CDR2 comprises the amino acid sequence SEQ ID NO: 40, or the amino acid sequence of SEQ ID NO: 40 comprising 1, 2, or 3 amino acid modifications; the amino acid sequence of VH CDR3 comprises the amino acid sequence SEQ ID NO: 41, or the amino acid sequence of SEQ ID NO: 41 comprising 1, 2, or 3 amino acid modifications; the amino acid sequence of VL CDR1 comprises the amino acid sequence SEQ ID NO: 42, or the amino acid sequence of SEQ ID NO: 42 comprising 1, 2, or 3 amino acid modifications; the amino acid sequence of VL CDR2 comprises the amino acid sequence SEQ ID NO: 43, or the amino acid sequence of SEQ ID NO: 43 comprising 1, 2, or 3 amino acid modifications; and the amino acid sequence of VL CDR3 comprises the amino acid sequence SEQ ID NO: 44, or the amino acid sequence of SEQ ID NO: 44 comprising 1, 2, or 3 amino acid modifications; (ii) the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46; and/or (iii) the amino acid sequence of the heavy chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and wherein the amino acid sequence of the light chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49; (b) the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (c) the amino acid sequence of the third moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (d) the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111; and (e) the amino acid sequence of the second peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111.
E211. The use of any one of E109-E210, wherein the fusion protein comprises: (a) a first polypeptide, wherein the amino acid sequence of the first polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (b) a second polypeptide, wherein the amino acid sequence of the second polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (c) a third polypeptide, wherein the amino acid sequence of the third polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126; and (d) a fourth polypeptide, wherein the amino acid sequence of the fourth polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.
E212. A combination regimen comprising: (a) a KRAS (e.g., hKRAS) inhibitor (e.g., a KRAS (e.g., hKRAS) inhibitor described herein); and (b) a fusion protein comprising: (i) a first moiety that specifically binds EGFR (e.g., hEGFR) operably connected to (ii) a second moiety that specifically binds TGFβ (e.g., hTGFβ) (e.g., a fusion protein described herein).
E213. A fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)) for use in a method of treating cancer, wherein the method comprises administering to a subject in need thereof the fusion protein in combination with a kirsten rat sarcoma virus homolog (KRAS) (e.g., human KRAS (hKRAS)) inhibitor.
E214. The fusion protein for use of E213, wherein the KRAS (e.g., hKRAS) inhibitor and the fusion protein are administered in an amount and for a time sufficient to treat or prevent the cancer in the subject.
E215. A fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)) for use in a method of treating a KRAS (e.g., hKRAS) variant cancer in a subject in need thereof, the method comprising: (a) receiving test results showing that the KRAS (e.g., hKRAS) variant cancer is resistant to a KRAS inhibitor; and (b) administering to the subject the KRAS (e.g., hKRAS) inhibitor in combination with the fusion protein.
E216. The fusion protein for use of E215, wherein the KRAS (e.g., hKRAS) inhibitor has been previously administered to the subject.
E217. The fusion protein for use of E215 or E216, wherein the KRAS (e.g., hKRAS) inhibitor and the fusion protein are administered in an amount and for a time sufficient to treat the KRAS (e.g., hKRAS) variant cancer in the subject.
E218. A fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)) for use in a method of treating a KRAS (e.g., hKRAS) variant cancer in a subject in need thereof, the method comprising: (a) administering to the subject a KRAS (e.g., hKRAS) inhibitor; (b) receiving testing results showing that the KRAS (e.g., hKRAS) variant cancer has developed resistance to the KRAS (e.g., hKRAS) inhibitor; and (c) administering to the subject the KRAS (e.g., hKRAS) inhibitor in combination with the fusion protein.
E219. The fusion protein for use of E218, wherein the KRAS (e.g., hKRAS) inhibitor and the fusion protein are administered in an amount and for a time sufficient to treat the KRAS (e.g., hKRAS) variant cancer in the subject.
E220. A fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)) for use in a method of treating a KRAS (e.g., hKRAS) variant cancer in a subject in need thereof, the method comprising: (a) determining that the KRAS (e.g., hKRAS) variant cancer is resistant to a KRAS (e.g., hKRAS) inhibitor; and (b) administering to the subject a KRAS (e.g., hKRAS) inhibitor in combination with the fusion protein.
E221. The method of E220, wherein the subject has been previously treated with the KRAS (e.g., hKRAS) inhibitor.
E222. The fusion protein for use of E220 or E221, wherein the KRAS (e.g., hKRAS) inhibitor and the fusion protein are administered in an amount and for a time sufficient to treat the KRAS (e.g., hKRAS) variant cancer in the subject.
E223. The fusion protein for use of any one of the preceding embodiments, wherein the cancer is a KRAS (e.g., hKRAS) variant cancer.
E224. The fusion protein for use of any one of the preceding embodiments, wherein the cancer has been determined to contain a KRAS (e.g., hKRAS) variant.
E225. The fusion protein for use of E223 or E224, wherein the KRAS (e.g., hKRAS) variant comprises a KRAS (e.g., hKRAS) activating amino acid modification (e.g., substitution).
E226. The fusion protein for use of E223-E225, wherein the KRAS (e.g., hKRAS) variant comprises an amino acid modification (e.g., substitution) at amino acid position G12, G13, or Q61, numbering relative to the amino acid sequence of SEQ ID NO: 3.
E227. The fusion protein for use of E223-E226, wherein the KRAS (e.g., hKRAS) variant comprises an amino acid modification (e.g., substitution) at amino acid position G12 or G13, numbering relative to the amino acid sequence of SEQ ID NO: 3.
E228. The fusion protein for use of E223-E227, wherein the KRAS (e.g., hKRAS) variant comprises any one or more of the following amino acid substitutions G12C, G12V, G12R, G12D, G12A, G12S, G13D, or Q61H, numbering relative to the amino acid sequence of SEQ ID NO: 3.
E229. The fusion protein for use of E223-E228, wherein the KRAS (e.g., hKRAS) variant comprises any one or more of the following amino acid substitutions G12C, G12V, G12R, G12D, G12A, G12S, or G13D, numbering relative to the amino acid sequence of SEQ ID NO: 3.
E230. The fusion protein for use of E223-E229, wherein the KRAS (e.g., hKRAS) variant comprises any one of the following amino acid substitutions G12C, G12V, G12R, G12A, G12S, or G12D, numbering relative to the amino acid sequence of SEQ ID NO: 3.
E231. The fusion protein for use of E223-E230, wherein the KRAS (e.g., hKRAS) variant comprises a G12C amino acid substitution, numbering relative to the amino acid sequence of SEQ ID NO: 3.
E232. The fusion protein for use of any one of the preceding embodiments, wherein the cancer is resistant to the KRAS (e.g., hKRAS) inhibitor.
E233. The fusion protein for use of any one of the preceding embodiments, wherein the cancer has been determined to be resistant to the KRAS (e.g., hKRAS) inhibitor.
E234. The fusion protein for use of any one of the preceding embodiments, wherein the KRAS (e.g., hKRAS) inhibitor has previously been administered to the subject.
E235. The fusion protein for use of any one of the preceding embodiments, wherein the KRAS (e.g., hKRAS) inhibitor has previously been administered to the subject in the absence of the fusion protein.
E236. The fusion protein for use of any one of the preceding embodiments, wherein the cancer cells overexpress EGFR (e.g., hEGFR) relative to non-cancer cells.
E237. The fusion protein for use of any one of the preceding embodiments, wherein the cancer cells have been determined to overexpress EGFR (e.g., hEGFR) relative to non-cancer cells.
E238. The fusion protein for use of any one of the preceding embodiments, wherein the cancer cells express an EGFR (e.g., hEGFR) variant.
E239. The fusion protein for use of any one of the preceding embodiments, wherein the cancer cells have been determined to express an EGFR (e.g., hEGFR) variant.
E240. The fusion protein for use of any one of the preceding embodiments, wherein the cancer cells overexpress hTGFβ (e.g., hTGFβ) relative to non-cancer cells.
E241. The fusion protein for use of any one of the preceding embodiments, wherein the cancer cells have been determined to overexpress TGFβ (e.g., hTGFβ) relative to non-cancer cells.
E242. The fusion protein for use of any one of the preceding embodiments, wherein the cancer is local, locally advanced, or metastatic.
E243. The fusion protein for use of any one of the preceding embodiments, wherein the cancer is a carcinoma (e.g., adenocarcinoma, squamous cell carcinoma).
E244. The fusion protein for use of any one of the preceding embodiments, wherein the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), pancreatic cancer (e.g., pancreatic ductal adenocarcinoma), breast cancer (e.g., invasive ductal carcinoma), stomach cancer (e.g., stomach adenocarcinoma), endometrial cancer (e.g., undifferentiated endometrial carcinoma), uterine cancer (e.g., uterine endometrial carcinoma), testicular cancer (e.g., testicular germ cell cancer), cervical cancer (e.g., cervical squamous carcinoma), bile duct cancer (e.g., cholangiocarcinoma), a myelodysplastic cancer, or esophageal cancer (e.g., esophageal adenocarcinoma, gastroesophageal junction cancer).
E245. The fusion protein for use of any one of the preceding embodiments, wherein the cancer is a lung cancer (e.g., non-small cell lung cancer (NSCLC), lung adenocarcinoma, lung squamous carcinoma), colorectal cancer (e.g., colorectal adenocarcinoma), colon cancer (e.g., colon adenocarcinoma), rectal cancer (e.g., rectal adenocarcinoma), or a pancreatic cancer (e.g., pancreatic ductal adenocarcinoma).
E246. The fusion protein for use of any one of the preceding embodiments, wherein the cancer is a non-small cell lung cancer (NSCLC), lung adenocarcinoma, lung squamous carcinoma, colorectal adenocarcinoma, or pancreatic ductal adenocarcinoma.
E247. A fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)) for use in a method of restoring sensitivity to a KRAS (e.g., hKRAS) inhibitor in a subject in need thereof, the method comprising: administering to the subject the fusion.
E248. The fusion protein for use of E247, wherein the fusion protein is administered in an amount and for a time sufficient to restore sensitivity to the KRAS (e.g., hKRAS) inhibitor in the subject.
E249. The fusion protein for use of E247-E248, wherein the cancer in the subject is resistant to the KRAS (e.g., hKRAS) inhibitor.
E250. The fusion protein for use of E247-E249, wherein the cancer has been determined to be resistant to the KRAS (e.g., hKRAS) inhibitor.
E251. The fusion protein for use of E247-E250, further comprising administering the KRAS (e.g., hKRAS) inhibitor to the subject in combination with the fusion protein.
E252. A fusion protein comprising: (i) a first moiety that specifically binds epidermal growth factor receptor (EGFR) (e.g., human EGFR (hEGFR)) operably connected to (ii) a second moiety that specifically binds transforming growth factor β (TGFβ) (e.g., human TGFβ (hTGFβ)) for use in a method of suppressing or preventing resistance to a KRAS (e.g., hKRAS) inhibitor in a subject in need thereof, the method comprising administering to the subject (a) a KRAS (e.g., hKRAS) inhibitor; in combination with the fusion protein.
E253. The fusion protein for use of E252, wherein the fusion protein is administered in an amount and for a time sufficient to suppress the development of resistance to the KRAS (e.g., hKRAS) inhibitor in the subject.
E254. The fusion protein for use of E252-E253, wherein the KRAS (e.g., hKRAS) inhibitor was previously administered to the subject.
E255. The fusion protein for use of any one of the preceding embodiments, wherein the subject has been previously administered a KRAS (e.g., hKRAS) inhibitor (e.g., the KRAS (e.g., hKRAS) inhibitor).
E256. The fusion protein for use of any one of the preceding embodiments, wherein the KRAS (e.g., hKRAS) inhibitor has not been previously administered to the subject.
E257. The fusion protein for use of any one of the preceding embodiments, wherein the subject has been previously administered a KRAS (e.g., hKRAS) inhibitor (e.g., the KRAS (e.g., hKRAS) inhibitor) and the cancer in the subject developed resistance to the KRAS (e.g., hKRAS) inhibitor.
E258. The fusion protein for use of any one of the preceding embodiments, wherein the cancer in the subject is resistant to a KRAS (e.g., hKRAS) inhibitor (e.g., the KRAS (e.g., hKRAS) inhibitor).
E259. The fusion protein for use of any one of the preceding embodiments, wherein the KRAS (e.g., hKRAS) inhibitor is capable of selectively inhibiting a KRAS (e.g., hKRAS) variant.
E260. The fusion protein for use of any one of the preceding embodiments, wherein the KRAS (e.g., hKRAS) variant comprises a KRAS (e.g., hKRAS) activating amino acid modification.
E261. The fusion protein for use of any one of the preceding embodiments, wherein the KRAS (e.g., hKRAS) variant comprises an amino acid modification at amino acid position G12, G13, or Q61, numbering relative to the amino acid sequence of SEQ ID NO: 3.
E262. The fusion protein for use of any one of the preceding embodiments, wherein the KRAS (e.g., hKRAS) variant comprises an amino acid modification at amino acid position G12 or G13, numbering relative to the amino acid sequence of SEQ ID NO: 3.
E263. The fusion protein for use of any one of the preceding embodiments, wherein the KRAS (e.g., hKRAS) variant comprises any one or more of the following amino acid substitutions G12C, G12V, G12R, G12D, G12A, G13D, or Q61H, numbering relative to the amino acid sequence of SEQ ID NO: 3.
E264. The fusion protein for use of any one of the preceding embodiments, wherein the KRAS (e.g., hKRAS) variant comprises any one or more of the following amino acid substitutions G12C, G12V, G12R, G12D, G12A, or G13D, numbering relative to the amino acid sequence of SEQ ID NO: 3.
E265. The fusion protein for use of any one of the preceding embodiments, wherein the KRAS (e.g., hKRAS) variant comprises any one of the following amino acid substitutions G12C, G12V, G12R, G12A, or G12D, numbering relative to the amino acid sequence of SEQ ID NO: 3.
E266. The fusion protein for use of any one of the preceding embodiments, wherein the KRAS (e.g., hKRAS) variant comprises a G12C amino acid substitution, numbering relative to the amino acid sequence of SEQ ID NO: 3.
E267. The fusion protein for use of any one of the preceding embodiments, wherein the KRAS (e.g., hKRAS) inhibitor comprises or consists of a small molecule, protein (e.g., an antibody or functional fragment or variant thereof), nucleic acid, carbohydrate, a lipid, a metal, or a toxin.
E268. The fusion protein for use of any one of the preceding embodiments, wherein the KRAS (e.g., hKRAS) inhibitor comprises or consists of a small molecule.
E269. The fusion protein for use of any one of the preceding embodiments, wherein the KRAS (e.g., hKRAS) inhibitor comprises a KRAS (e.g., hKRAS) inhibitor in Table 2.
E270. The fusion protein for use of any one of the preceding embodiments, wherein the KRAS (e.g., hKRAS) inhibitor comprises sotorasib (AMG-510).
E271. The fusion protein for use of any one of the preceding embodiments, wherein the first moiety comprises an antibody, or functional fragment or functional variant thereof.
E272. The fusion protein for use of any one of the preceding embodiments, wherein the first moiety comprises a full-length antibody, a single chain variable fragment (scFv), a Fab, or a single domain antibody (sdAb).
E273. The fusion protein for use of any one of the preceding embodiments, wherein the first moiety comprises a full-length antibody.
E274. The fusion protein for use of any one of the preceding embodiments, wherein the first moiety comprises a variable heavy chain (VH) region that comprises three complementarity determining regions: VH CDR1, VH CDR2, and VH CDR3; and a variable light chain (VL) region that comprises three complementarity determining regions: VL CDR1, VL CDR2, and VL CDR3.
E275. The fusion protein for use of any one of the preceding embodiments, wherein (a) the amino acid sequence of VH CDR1 comprises the amino acid sequence SEQ ID NO: 39, or the amino acid sequence of SEQ ID NO: 39 comprising 1, 2, or 3 amino acid modifications; (b) the amino acid sequence of VH CDR2 comprises the amino acid sequence SEQ ID NO: 40, or the amino acid sequence of SEQ ID NO: 40 comprising 1, 2, or 3 amino acid modifications; (c) the amino acid sequence of VH CDR3 comprises the amino acid sequence SEQ ID NO: 41, or the amino acid sequence of SEQ ID NO: 41 comprising 1, 2, or 3 amino acid modifications; and (d) the amino acid sequence of VL CDR1 comprises the amino acid sequence SEQ ID NO: 42, or the amino acid sequence of SEQ ID NO: 42 comprising 1, 2, or 3 amino acid modifications; (e) the amino acid sequence of VL CDR2 comprises the amino acid sequence SEQ ID NO: 43, or the amino acid sequence of SEQ ID NO: 43 comprising 1, 2, or 3 amino acid modifications; and (f) the amino acid sequence of VL CDR3 comprises the amino acid sequence SEQ ID NO: 44, or the amino acid sequence of SEQ ID NO: 44 comprising 1, 2, or 3 amino acid modifications.
E276. The fusion protein for use of any one of the preceding embodiments, wherein the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46.
E277. The fusion protein for use of any one of the preceding embodiments, wherein the first moiety comprises a heavy chain (HC) and a light chain (LC), wherein the amino acid sequence of the HC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and the amino acid sequence of the LC is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49.
E278. The fusion protein for use of any one of the preceding embodiments, wherein the second moiety comprises an antibody, or functional fragment or functional variant thereof.
E279. The fusion protein for use of any one of the preceding embodiments, wherein the second moiety comprises a full-length antibody, a single chain variable fragment (scFv), a scFv2, a scFv-Fc, a Fab, a Fab′, a F(ab′)2, or a F(v).
E280. The fusion protein for use of any one of the preceding embodiments, wherein the second moiety comprises or consists of at least a portion of the extracellular domain (ECD) of a transforming growth factor-beta receptor (TGFβR) (e.g., hTGFβR).
E281. The fusion protein for use of any one of the preceding embodiments, wherein the second moiety comprises or consists of at least a portion of the ECD of transforming growth factor-beta receptor II (TGFβRII) (e.g., hTGFβRII).
E282. The fusion protein for use of any one of the preceding embodiments, wherein the second moiety comprises or consists of the ECD of TGFβRII (e.g., hTGFβRII).
E283. The fusion protein for use of any one of the preceding embodiments, wherein the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E284. The fusion protein for use of any one of the preceding embodiments, wherein the fusion protein comprises (iii) a third moiety that specifically binds TGFβ (e.g., hTGFβ) and is operably connected to the first moiety.
E285. The fusion protein for use of any one of the preceding embodiments, wherein the third moiety comprises an antibody, or functional fragment or functional variant thereof.
E286. The fusion protein for use of any one of the preceding embodiments, wherein the third moiety comprises a full-length antibody, a single chain variable fragment (scFv), a scFv2, a scFv-Fc, a Fab, a Fab′, a F(ab′)2, or a F(v).
E287. The fusion protein for use of any one of the preceding embodiments, wherein the third moiety comprises or consists of at least a portion of the extracellular domain (ECD) of a TGFβR (e.g., hTGFβR).
E288. The fusion protein for use of any one of the preceding embodiments, wherein the third moiety comprises or consists of at least a portion of the ECD of TGFβRII (e.g., hTGFβRII).
E289. The fusion protein for use of any one of the preceding embodiments, wherein the third moiety comprises or consists of the ECD of TGFβRII (e.g., hTGFβRII).
E290. The fusion protein for use of any one of the preceding embodiments, wherein the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E291. The fusion protein for use of any one of the preceding embodiments, wherein the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73; and the amino acid sequence of the third moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.
E292. The fusion protein for use of any one of the preceding embodiments, wherein the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of the third moiety.
E293. The fusion protein for use of any one of the preceding embodiments, wherein the first moiety comprises a full-length antibody and wherein the N-terminus of a second moiety is operably connected (e.g., optionally via a linker) to the C-terminus of the first light chain.
E294. The fusion protein for use of any one of the preceding embodiments, wherein the first moiety comprises a full-length antibody and wherein the N-terminus of a second moiety is operably connected (e.g., optionally via a linker) to the C-terminus of the first heavy chain.
E295. The fusion protein for use of any one of the preceding embodiments, wherein the first moiety comprises a full-length antibody and wherein the N-terminus of a second moiety is operably connected (e.g., optionally via a linker) to the C-terminus of the first light chain and the N-terminus of the third moiety is operably connected (e.g., optionally via a linker) to the C-terminus of the second light chain.
E296. The fusion protein for use of any one of the preceding embodiments, wherein the first moiety comprises a full-length antibody and wherein the N-terminus of a second moiety is operably connected (e.g., optionally via a linker) to the C-terminus of the first heavy chain and the N-terminus of the third moiety is operably connected (e.g., optionally via a linker) to the C-terminus of the second heavy chain.
E297. The fusion protein for use of any one of the preceding embodiments, wherein the first moiety is directly operably connected to the second moiety.
E298. The fusion protein for use of any one of the preceding embodiments, wherein the first moiety is indirectly operably connected to the second moiety.
E299. The fusion protein for use of any one of the preceding embodiments, wherein the first moiety is indirectly operably connected to the second moiety through a first peptide linker.
E300. The fusion protein for use of any one of the preceding embodiments, wherein the first peptide linker comprises or consists of glycine or glycine and serine amino acid residues.
E301. The fusion protein for use of any one of the preceding embodiments, wherein the amino acid sequence of the first peptide linker comprises or consists of (a) the amino acid sequence of any one of SEQ ID NOS: 105-124; or (b) the amino acid sequence of any one of SEQ ID NOS: 105-124 comprising or consisting of 1, 2, or 3 amino acid modifications.
E302. The fusion protein for use of any one of the preceding embodiments, wherein the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.
E303. The fusion protein for use of any one of the preceding embodiments, wherein the first moiety is directly operably connected to the third moiety.
E304. The fusion protein for use of any one of the preceding embodiments, wherein the first moiety is indirectly operably connected to the third moiety.
E305. The fusion protein for use of any one of the preceding embodiments, wherein the first moiety is indirectly operably connected to the third moiety through a second peptide linker.
E306. The fusion protein for use of E305, wherein the second peptide linker comprises or consists of glycine or glycine and serine amino acid residues.
E307. The fusion protein for use of E305 or E306, wherein the amino acid sequence of the second peptide linker comprises or consists of (a) the amino acid sequence of any one of SEQ ID NO: 111; or (b) the amino acid sequence of any one of SEQ ID NO: 111 comprising or consisting of 1, 2, or 3 amino acid modifications.
E308. The fusion protein for use of any one of E305-E307, wherein the amino acid sequence of the first peptide linker comprises or consists of (a) the amino acid sequence of SEQ ID NO: 111; or (b) the amino acid sequence of SEQ ID NO: 111 comprising or consisting of 1, 2, or 3 amino acid modifications.
E309. The fusion protein for use of any one of E305-E308, wherein the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of the second peptide linker.
E310. The method of any one of E305-E309, wherein the amino acid sequence of the first peptide linker is 100% identical to the amino acid sequence of the second peptide linker.
E311. The fusion protein for use of any one of the preceding embodiments, wherein (a) the first moiety comprises or consists of a full-length antibody; (b) the second moiety comprises a comprises or consists of the ECD of TGFβRII (e.g., hTGFβRII); (c) the third moiety comprises a comprises or consists of the ECD of TGFβRII (e.g., hTGFβRII); (d) the second moiety is operably connected to the first moiety through a first peptide linker; and (c) the third moiety is operably connected to the first moiety through a second peptide linker.
E312. The method of E311, wherein the N-terminus of the second moiety is operably connected to the C-terminus of the first light chain of the full-length antibody and the N-terminus of the third moiety is operably connected to the C-terminus of the second light chain of the full-length antibody.
E313. The fusion protein for use of E311-E312, wherein (a) (i) the amino acid sequence of VH CDR1 comprises the amino acid sequence SEQ ID NO: 39, or the amino acid sequence of SEQ ID NO: 39 comprising 1, 2, or 3 amino acid modifications, the amino acid sequence of VH CDR2 comprises the amino acid sequence SEQ ID NO: 40, or the amino acid sequence of SEQ ID NO: 40 comprising 1, 2, or 3 amino acid modifications; the amino acid sequence of VH CDR3 comprises the amino acid sequence SEQ ID NO: 41, or the amino acid sequence of SEQ ID NO: 41 comprising 1, 2, or 3 amino acid modifications; the amino acid sequence of VL CDR1 comprises the amino acid sequence SEQ ID NO: 42, or the amino acid sequence of SEQ ID NO: 42 comprising 1, 2, or 3 amino acid modifications; the amino acid sequence of VL CDR2 comprises the amino acid sequence SEQ ID NO: 43, or the amino acid sequence of SEQ ID NO: 43 comprising 1, 2, or 3 amino acid modifications; and the amino acid sequence of VL CDR3 comprises the amino acid sequence SEQ ID NO: 44, or the amino acid sequence of SEQ ID NO: 44 comprising 1, 2, or 3 amino acid modifications; (ii) the VH region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 45; and the VL region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 46; and/or (iii) the amino acid sequence of the heavy chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 48; and wherein the amino acid sequence of the light chain of the full-length antibody is at least 85%, 86%, 87%, 88%, 89%, 90%, 91%, 92%, 93%, 94%, 95%, 96%, 97%, 98%, 99%, or 100% identical to the amino acid sequence of SEQ ID NO: 49; (b) the amino acid sequence of the second moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (c) the amino acid sequence of the third moiety is at least 85%, 86%, 87%, 88%, 89%, 90%, 91%, 92%, 93%, 94%, 95%, 96%, 97%, 98%, 99%, or 100% identical to the amino acid sequence of SEQ ID NO: 72; (d) the amino acid sequence of the first peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111; and (e) the amino acid sequence of the second peptide linker is at least 85%, 86%, 87%, 88%, 89%, 90%, 91%, 92%, 93%, 94%, 95%, 96%, 97%, 98%, 99%, or 100% identical to the amino acid sequence of SEQ ID NO: 111.
E314. The fusion protein for use of any one of the preceding embodiments, wherein the fusion protein comprises: (a) a first polypeptide, wherein the amino acid sequence of the first polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (b) a second polypeptide, wherein the amino acid sequence of the second polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125; (c) a third polypeptide, wherein the amino acid sequence of the third polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126; and (d) a fourth polypeptide, wherein the amino acid sequence of the fourth polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.

### 6. EXAMPLES

Table of Contents

| | |
|---|---|
| 6.1 | Example 1. BCA101 Expression and Purification. |
| 6.2 | Example 2. hKRAS G12C Inhibitors. |
| 6.3 | Example 3. BCA101 synergizes hKRAS G12C Inhibitor |
| | cytotoxicity in in vitro lung and colon cancer models. |
| 6.4 | Example 4. BCA101 restores TGFβ-induced hKRAS G12C |
| | inhibitor sensitivity in in vitro lung cancer model. |
| 6.5 | Example 5. Induction of long-term hKRAS G12C inhibitor |
| | treatment-induced resistance in in vitro lung cancer cell model. |
| 6.6 | Example 6. Rescue of long-term KRAS G12C inhibitor treatment- |
| | induced resistance by BCA101. |
| 6.7 | Example 7. EGFR Expression by KRASG12C Mutated Lung and |
| | Colon Cancer Cells. |
| 6.8 | Example 8. TGFβ Expression by KRASG12C Mutated Lung and |
| | Colon Cancer Cells. |
| 6.9 | Example 9. TGFβ and EGFR Expression by KRAS-G12Ci |
| | Resistant KRASG12C Mutated Lung and Colon Cancer Cells. |
| 6.10 | Example 10. Susceptibility of KRAS-G12Ci Resistant |
| | KRASG12C Mutated Lung and Colon Cancer Cells to |
| | KRAS-G12Ci + BCA101 Combinations. |
| 6.11 | Example 11. In Vivo Treatment of KRAS-G12Ci Resistant |
| | KRASG12C Mutated Lung with KRAS-G12Ci + BCA101 |
| | Combination. |
| 6.1 | Example 1. BCA101 Expression and Purification. |

BCA101 was generated using standard methods known in the art. Briefly, DNA polynucleotides encoding each of the polypeptides of the BCA101 protein was synthesized and inserted into an expression plasmid. CHO cells were transfected using the using either lipid based or electroporation-based transfection kits (according to the manufacturer's protocol). Briefly, the CHO cells were grown in chemically defined CHO cell culture medium at 37° C. for 2 to 5 passages. Prior to transfection, the cells were counted using automated cell counter to ensure a cell count of about 1 million cells/ml and >95 percent viability. Transfections were performed in 30 ml of cell containing medium (20-30 million cells per reaction). 1.6 to 3 μg/ml of plasmid DNA was used for transfection. The amino acid sequence of BCA101 is set forth in SEQ ID NOS: 125-126) and in Table 9 below.

TABLE 9
The Amino Acid Sequence of Exemplary Fusion
Proteins

| | | | SEQ |
|---|---|---|---|
| | | | ID |
| Description | | Amino Acid Sequence | NO |
| BCA101 | Heavy | QVQLKQSGPGLVQPSQSLSITCTVS | 125 |
| αEGFR x | Chain | GFSLINYGVHWVRQSPGKGLEWLGV | |
| TGFβRII | | IWSGGNTDYNTPFTSRLSINKDNSK | |
| ECD | | SQVFFKMNSLQSNDTAIYYCARALT | |
| C- | | YYDYEFAYWGQGTLVTVSAASTKGP | |
| terminal | | SVFPLAPSSKSTSGGTAALGCLVKD | |
| LC | | YFPEPVTVSWNSGALTSGVHTFPAV | |
| Fusion | | LQSSGLYSLSSVVTVPSSSLGTQTY | |
| | | ICNVNHKPSNTKVDKRVEPKSCDKT | |
| | | HTCPPCPAPELLGGPSVFLFPPKPK | |
| | | DTLMISRTPEVTCVVVDVSHEDPEV | |
| | | KFNWYVDGVEVHNAKTKPREEQYNS | |
| | | TYRVVSVLTVLHQDWLNGKEYKCKV | |
| | | SNKALPAPIEKTISKAKGQPREPQV | |
| | | YTLPPSRDELTKNQVSLTCLVKGFY | |
| | | PSDIAVEWESNGQPENNYKTTPPVL | |
| | | DSDGSFFLYSKLTVDKSRWQQGNVF | |
| | | SCSVMHEALHNHYTQKSLSLSPG | |
| | Light | DILLTQSPVILSVSPGERVSFSCRA | 126 |
| | Chain | SQSIGTNIHWYQQRTNGSPRLLIKY | |
| | | ASESISGIPSRFSGSGSGTDFTLSI | |
| | | NSVESEDIADYYCQQNNNWPTTFGA | |
| | | GTKLELKRTVAAPSVFIFPPSDEQL | |
| | | KSGTASVVCLLNNFYPREAKVQWKV | |
| | | DNALQSGNSQESVTEQDSKDSTYSL | |
| | | SSTLTLSKADYEKHKVYACEVTHQG | |
| | | LSSPVTKSFNRGECGGGGSGGGGSG | |
| | | GGGSTIPPHVQKSVNNDMIVTDNNG | |
| | | AVKFPQLCKFCDVRFSTCDNQKSCM | |
| | | SNCSITSICEKPQEVCVAVWRKNDE | |
| | | NITLETVCHDPKLPYHDFILEDAAS | |
| | | PKCIMKEKKKPGETFFMCSCSSDEC | |
| | | NDNIIFSEEYNTSNPD | |

The following reference proteins were also generated according to the methods described above: Cetixumab and TGFβRII-FC. The amino acid sequence of each of the reference proteins is provided in Table 10 below.

TABLE 10
The Amino Acid Sequence of Exemplary
Fusion Proteins

| | | | SEQ |
|---|---|---|---|
| | | | ID |
| Description | | Amino Acid Sequence | NO |
| Anti-EGFR | Heavy | QVQLKQSGPGLVQPSQSLSITCT | 48 |
| Antibody | Chain | VSGFSLTNYGVHWVRQSPGKGLE | |
| Cetixumab | | WLGVIWSGGNTDYNTPFTSRLSI | |
| Variant | | NKDNSKSQVFFKMNSLQSNDTAI | |
| | | YYCARALTYYDYEFAYWGQGTLV | |
| | | TVSAASTKGPSVFPLAPSSKSTS | |
| | | GGTAALGCLVKDYFPEPVTVSWN | |
| | | SGALTSGVHTFPAVLQSSGLYSL | |
| | | SSVVTVPSSSLGTQTYICNVNHK | |
| | | PSNTKVDKRVEPKSCDKTHTCPP | |
| | | CPAPELLGGPSVFLFPPKPKDTL | |
| | | MISRTPEVTCVVVDVSHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSRDELTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSPG | |
| | Light | DILLTQSPVILSVSPGERVSFSC | 49 |
| | Chain | RASQSIGTNIHWYQQRTNGSPRL | |
| | | LIKYASESISGIPSRFSGSGSGT | |
| | | DFTLSINSVESEDIADYYCQQNN | |
| | | NWPTTFGAGTKLELKRTVAAPSV | |
| | | FIFPPSDEQLKSGTASVVCLLNN | |
| | | FYPREAKVQWKVDNALQSGNSQE | |
| | | SVTEQDSKDSTYSLSSTLTLSKA | |
| | | DYEKHKVYACEVTHQGLSSPVTK | |
| | | SFNRGEC | |

| | | |
|---|---|---|
| TGFβRII-FC | TIPPHVQKSVNNDMIVTDNNGAV | 141 |
| | KFPQLCKFCDVRFSTCDNQKSCM | |
| | SNCSITSICEKPQEVCVAVWRKN | |
| | DENITLETVCHDPKLPYHDFILE | |
| | DAASPKCIMKEKKKPGETFFMCS | |
| | CSSDECNDNIIFSEEYNTSNPDG | |
| | GGGSGGGGSGGGGSTHTCPPCPA | |
| | PELLGGPSVFLFPPKPKDTLMIS | |
| | RTPEVTCVVVDVSHEDPEVKFNW | |
| | YVDGVEVHNAKTKPREEQYNSTY | |
| | RVVSVLTVLHQDWLNGKEYKCKV | |
| | SNKALPAPIEKTISKAKGQPREP | |
| | QVYTLPPSRDELTKNQVSLTCLV | |
| | KGFYPSDIAVEWESNGQPENNYK | |
| | TTPPVLDSDGSFFLYSKLTVDKS | |
| | RWQQGNVFSCSVMHEALHNHYTQ | |
| | KSLSLSPG | |

### 6.2 Example 2. hKRAS G12C Inhibitors

The hKRAS G12C inhibitor termed AMG510 was utilized in one or more example described herein. The structure of the hKRAS inhibitor AMG510 is as follows:

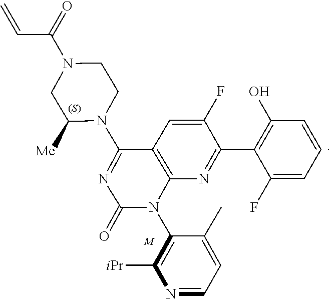

The chemical name of sotorasib is 6-fluoro-7-(2-fluoro-6-hydroxyphenyl)-(1M)-1-[4-methyl-2-(propan-2-yl) pyridin-3-yl]-4-[(2S)-2-methyl-4-(prop-2enoyl) piperazin-1-yl]pyrido[2,3-d]pyrimidin-2 (1H)-one.

### 6.3 Example 3. BCA101 Synergizes hKRAS G12C Inhibitor Cytotoxicity in In Vitro Lung and Colon Cancer Models

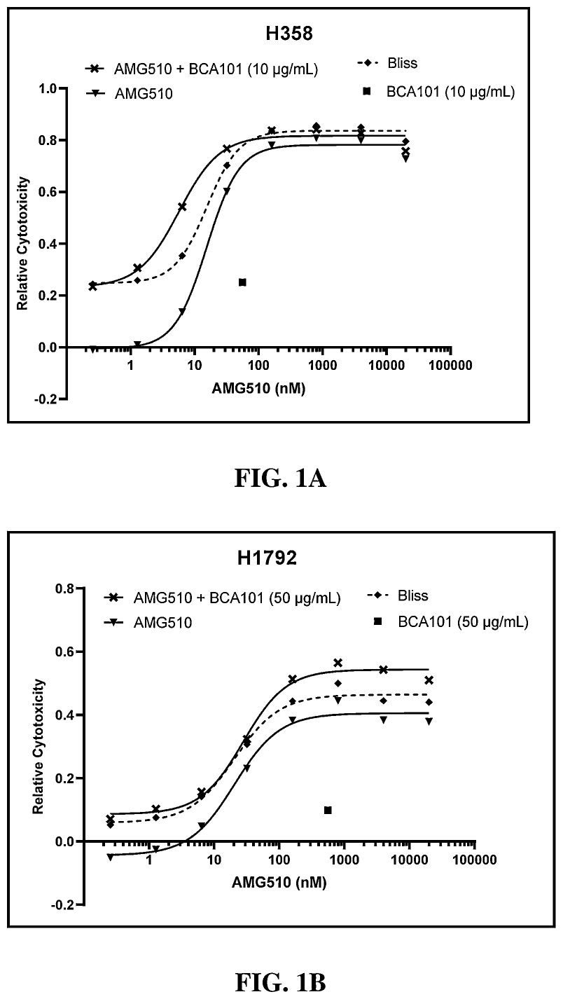

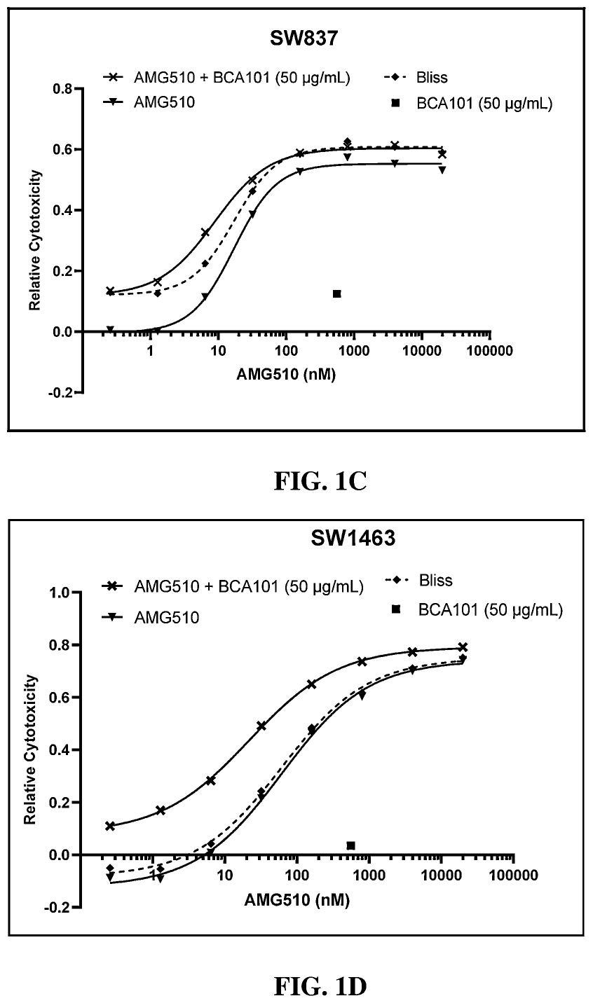

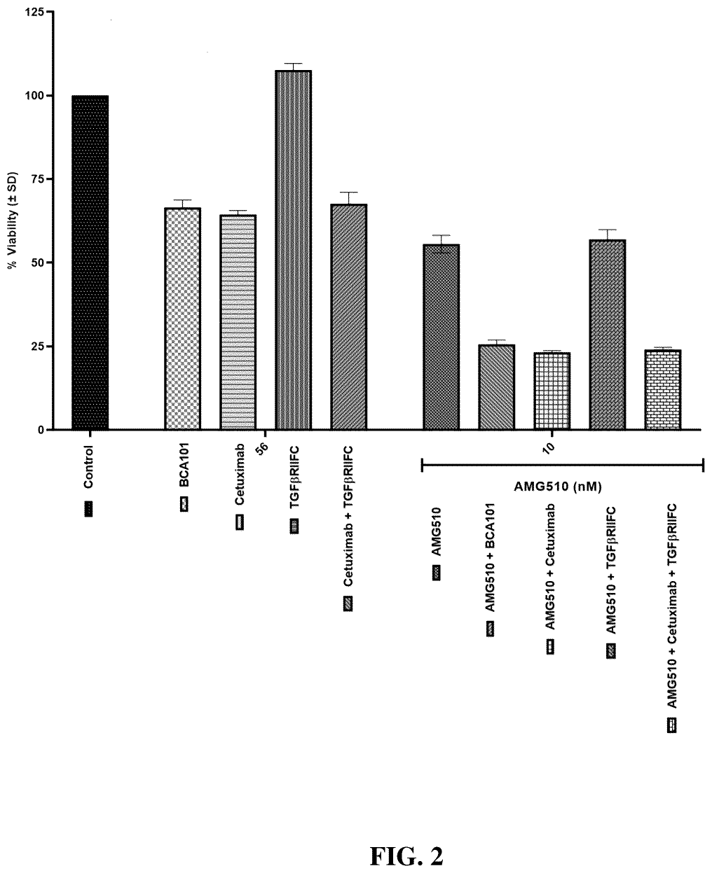

The effect of combining BCA101 and a hKRAS G12C inhibitor in the treatment of cancer was assessed using in vitro lung (H358 and H1792) and colorectal (SW837 and SW1463) cancer cell lines. Both of the lung and colon cancer cell lines were treated with combinations of titrating concentrations of the hKRAS G12C inhibitor AMG510 (as indicated in FIGS. 1A-1D and FIG. 2) and a fixed dose (10 or 50 μg/ml) of BCA101 for 72 hours. Cytotoxicity was evaluated using the CellTiter Glo® Luminescent Cell Viability Assay according to manufacturer's instructions (Promega). The CellTiter-Glo assay determines the number of viable cells in culture based on the quantitation of ATP, an indicator of metabolically active cells. The amount of ATP is directly proportional to the number of cells present in culture. Briefly, the CellTiter-Glo® Reagent was added to the cultured cells and incubated for approximately 10 minutes. The luminescent signal was subsequently detected using a luminometer. As shown in FIGS. 1A-1D and FIG. 2, treatment of both the lung and colon cancer cell lines with BCA101 and the hKRAS G12C inhibitor AMG510 resulted in enhanced tumor cell killing compared to the treatment with BCA101 or AMG510 alone. The combinatorial synergism of AMG510 and BCA101 was calculated using a Bliss statistical model. See, e.g., Berenbaum M. C. (1981). Criteria for analyzing interactions between biologically active agents. Advances in cancer research, 35, 269-335. https://doi.org/10.1016/s0065-230x (08) 60912-4; Borisy, A. A., Elliott, P. J., Hurst, N. W., Lee, M. S., Lehar, J., Price, E. R., Serbedzija, G., Zimmermann, G. R., Foley, M. A., Stockwell, B. R., & Keith, C. T. (2003). Systematic discovery of multicomponent therapeutics. Proceedings of the National Academy of Sciences of the United States of America, 100 (13), 7977-7982. https://doi.org/10.1073/pnas.1337088100; and Borisy, A. A., Elliott, P. J., Hurst, N. W., Lee, M. S., Lehar, J., Price, E. R., Serbedzija, G., Zimmermann, G. R., Foley, M. A., Stockwell, B. R., & Keith, C. T. (2003). Systematic discovery of multicomponent therapeutics. Proceedings of the National Academy of Sciences of the United States of America, 100 (13), 7977-7982. https://doi.org/10.1073/pnas.1337088100; the full contents of each of which is incorporated herein by reference for all purposes.

### 6.4 Example 4. BCA101 Restores TGFβ-Induced hKRAS G12C Inhibitor Sensitivity in in Vitro Lung Cancer Model

Reports have shown that hKRAS G12C inhibitor efficacy is higher in cancer cells with a low epithelial-mesenchymal transition (EMT) signature compared to a high EMT signature. hTGFβ is known to induce EMT through canonical and non-canonical pathways in various cancers. Since BCA101 neutralizes TGFβ through its TGFβRIIECD arm, the following study was conducted to evaluate whether BCA101 can restore hKRAS G12C sensitivity in a TGFβ-induced hKRAS G12C resistant lung cancer cell line (H1792).

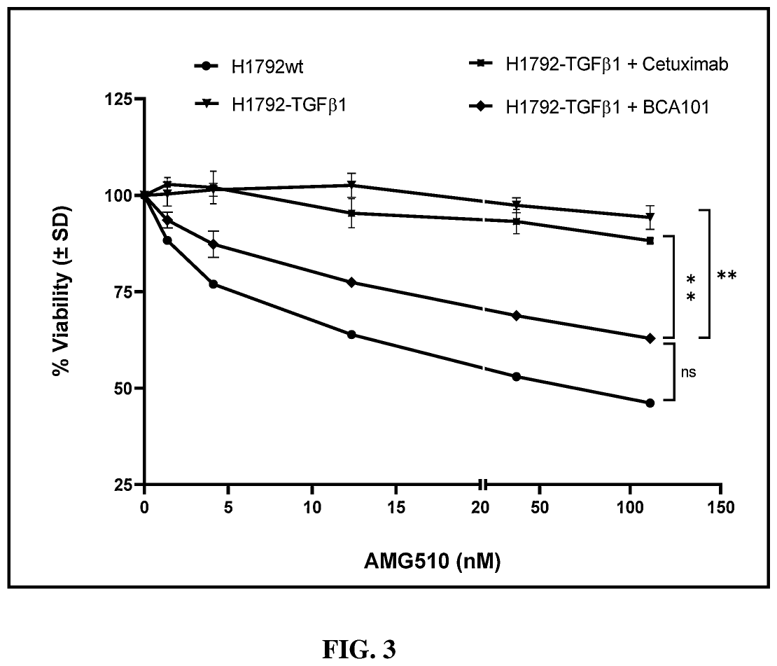

Briefly, hKRAS G12C inhibitor resistance was induced in H1792 cells by treating the cells with hTGFβ (4 ng/ml) for at least two weeks. The resultant hKRAS G12C inhibitor resistant H1792 cells were treated with titrating concentrations of a AMG510 and a fixed concentration (50 μg/ml) of BCA101 for 72 hours. TGFβ treatment induced a spindle-like morphology in the H1972 cell line, a hall mark of EMT, which was restored to normal morphology by treatment with BCA101. Cytotoxicity was evaluated using the CellTiter Glo® Luminescent Cell Viability Assay according to manufacturer's instructions (Promega) (as described in Example 6.3). Long-term TGFβ treated H1792 cells showed less cytotoxicity in response to AMG510 treatment compared to control H1792 cells (wild type), indicating the development of hKRAS G12C inhibitor resistance in H1792 cell line (FIG. 3). When hKRAS G12C inhibitor resistant H1972 cells were treated with a combination of BCA101 and AMG510, cytotoxicity of the hKRAS G12C inhibitor (AMG510) was restored to control (wild type) levels (FIG. 3), indicating that BCA101 rescues cancer cells from TGFβ-induced hKRAS G12C inhibitor resistance and restores hKRAS G12C inhibitor sensitivity.

### 6.5 Example 5. Induction of Long-Term hKRAS G12C Inhibitor Treatment-Induced Resistance in In Vitro Lung Cancer Cell Model

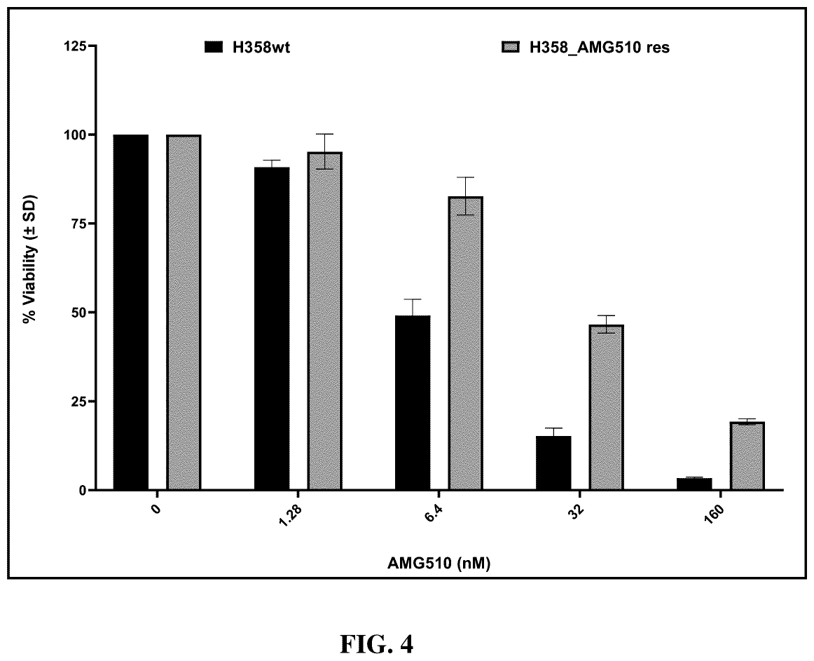

The effect of long duration treatment with a hKRAS G12C inhibitor on acquired hKRAS G12C inhibitor resistance was assessed utilizing an in vitro lung cancer model (H358). Briefly, H358 lung cancer cells were treated with increasing concentrations of the hKRAS G12C inhibitor AMG510 for about one month and cytotoxicity induced by AMG510 subsequently evaluated. As shown in FIG. 4, the H358 cells developed KRAS G12C inhibitor (AMG510) resistance compared to the control (wild type) H358 cells.

### 6.6 Example 6. Rescue of Long-Term KRAS G12C Inhibitor Treatment-Induced Resistance by BCA101

The evaluate whether BCA101 can rescue long-term KRAS G12C inhibitor treatment-induced resistance, an in vitro study utilizing a lung cancer cell line (H358) is employed. Briefly, the KRAS G12C inhibitor resistant H358 lung cancer cells are generated according to the methods described in Example 5. The cells are treated with titrating concentrations of a G12C inhibitor and a fixed concentration (50 μg/ml) of BCA101 for 72 hours. Cytotoxicity of the hKRAS G12C inhibitor (AMG510) is evaluated for restoration to wild type level, indicating that BCA101 is rescuing cancer cells from acquired hKRAS G12C inhibitor resistance and restoring sensitivity.

### 6.7 Example 7. EGFR Expression by KRASG12C Mutated Lung and Colon Cancer Cells

The cell surface expression of EGFR by KRASG12C mutated lung and colon cancer cell lines (NCI-H1792 (G12C homozygous) (lung cancer), NCI-H358 (G12C heterozygous) (lung cancer), and SW1463 (G12C homozygous) (colon cancer)) was assessed by flow cytometry.

Briefly, the cells were stained with anti-EGFR antibody (Cetuximab) or human IgG control antibody (Bioxcell) and detected using FITC labelled anti-human IgG Fc secondary antibody (Merck) using flow cytometry method.

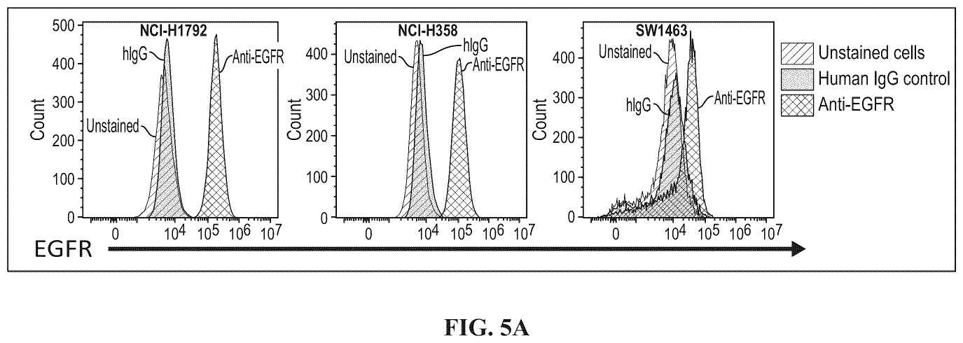

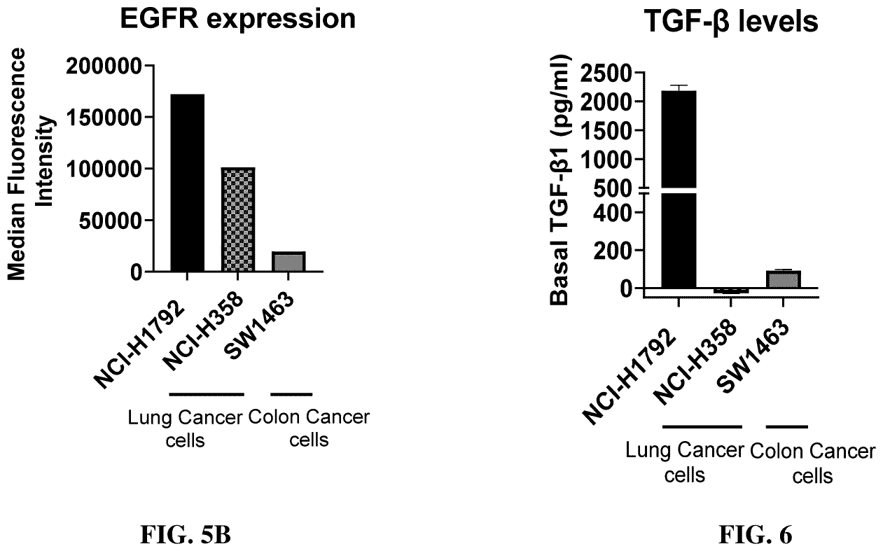

As shown in FIG. 5A and FIG. 5B, each of the KRASG12C mutated lung and colon cancer cell lines (NCI-H1792 (G12C homozygous) (lung cancer), NCI-H358 (G12C heterozygous) (lung cancer), and SW1463 (G12C homozygous) (colon cancer)) express EGFR.

### 6.8 Example 8. TGFβ Expression by KRASG12C Mutated Lung and Colon Cancer Cells

TGFβ expression by KRASG12C mutated lung and colon cancer cell lines (NCI-H1792 (G12C homozygous) (lung cancer), NCI-H358 (G12C heterozygous) (lung cancer), and SW1463 (G12C homozygous) (colon cancer)) was assessed by ELISA.

Briefly, 5×10⁶ cells (KRAS-G12Ci resistant NCI-H358, KRAS-G12Ci resistant NCI-H1792, and KRAS-G12Ci resistant SW1463) were seeded in 12-well multi-dish and cultured in cell culture media containing 1% fetal bovine serum (Gibco). The supernatant was collected after 72 hours and TGF-β1 secretion was evaluated using a standard ELISA (Quantikine ELISA, R&D Systems) according to Manufacturer's instructions.

Basal TGFβ1 expression from each of the KRASG12C mutated lung and colon cancer cell lines (NCI-H1792 (G12C homozygous) (lung cancer), NCI-H358 (G12C heterozygous) (lung cancer), and SW1463 (G12C homozygous) (colon cancer)) is shown in FIG. 6.

### 6.9 Example 9. TGFβ and EGFR Expression by KRAS-G12Ci Resistant KRASG12C Mutated Lung and Colon Cancer Cells

TGFβ expression by KRAS-G12Ci resistant KRASG12C mutated lung and colon cancer cell lines (NCI-H1792 (G12C homozygous) (lung cancer), NCI-H358 (G12C heterozygous) (lung cancer), and SW1463 (G12C homozygous) (colon cancer)) was assessed by ELISA.

Briefly, KRAS-G12Ci resistant KRASG12C mutated lung and colon cancer cell lines (NCI-H1792 (G12C homozygous) (lung cancer), NCI-H358 (G12C heterozygous) (lung cancer), and SW1463 (G12C homozygous) (colon cancer)) were generated through prolonged exposure (over 4-6 weeks) to KRAS-G12Ci. Subsequently, each KRAS-G12Ci resistant cell line was incubated with KRAS-G12C inhibitor for at least 4 weeks. Supernatants were collected after 72 hours from treated (‘KRAS (G12C) inhibitor’) and untreated (‘Control’) cells and TGFβ1 expression was measured using a standard ELISA. Cell surface EGFR expression was measured using flow cytometry (Quantikine ELISA, R&D Systems).

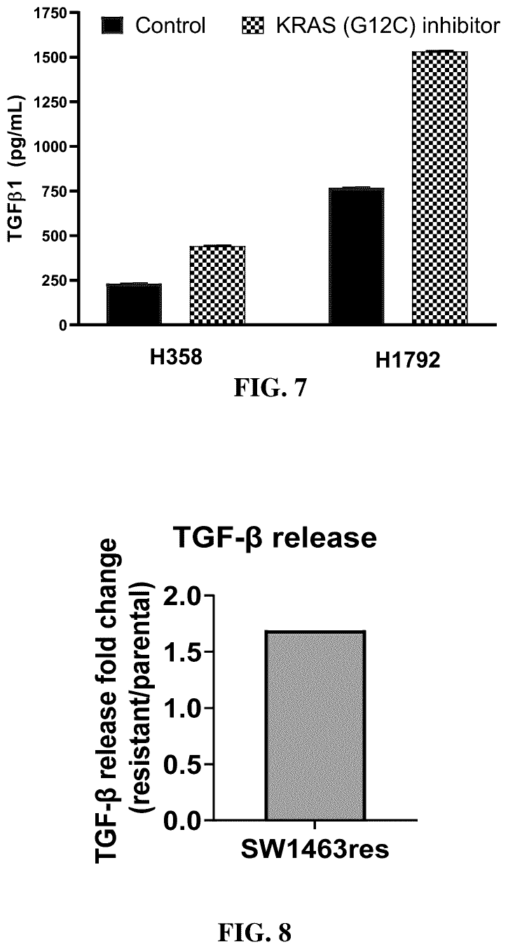

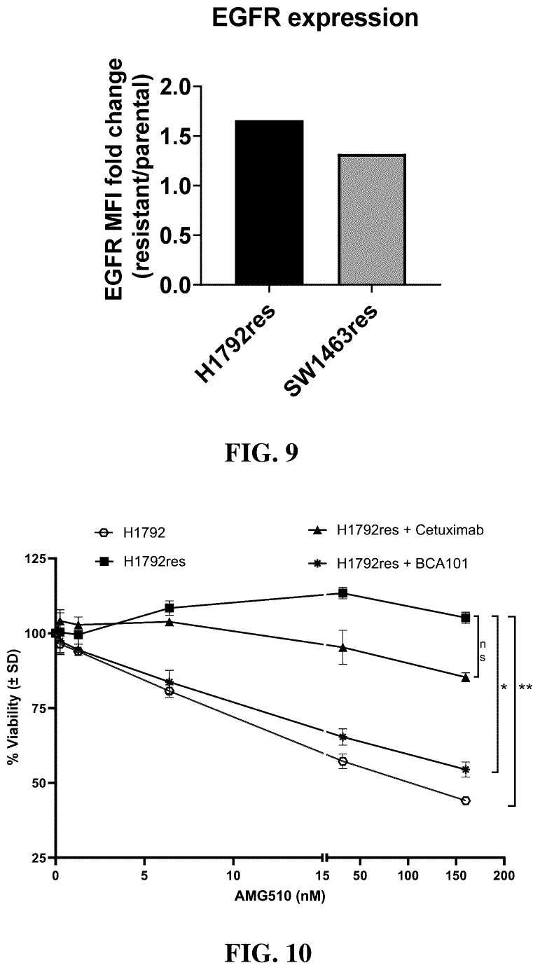

FIG. 7 shows an increase in TGFβ1 expression from the KRAS-G12Ci resistant NCI-H358 and NCI-H1792 cell cultures treated with the KRAS-G12Ci relative to the control. FIG. 8 shows an over 1.5-fold increase in TGFβ1 expression from the KRAS-G12Ci resistant SW1463 relative to the parental SW1463 cells treated with KRAS-G12Ci. FIG. 9 shows an over 1.5-fold increase in EGFR expression from the KRAS-G12Ci resistant NCI-H1792 relative to the parental NCI-H1792 cells treated with KRAS-G12Ci; and an almost 1.5-fold increase in EGFR expression from the KRAS-G12Ci resistant SW1463 relative to the parental SW1463 cells treated with KRAS-G12Ci.

### 6.10 Example 10. Susceptibility of KRAS-G12Ci Resistant KRASG12C Mutated Lung and Colon Cancer Cells to KRAS-G12Ci+BCA101 Combinations

The susceptibility of KRAS-G12Ci resistant KRASG12C mutated lung and colon cancer cells to KRAS-G12Ci+BCA101 combinations was assessed.

Briefly, H1792 lung cancer cells were treated with increasing concentrations of the hKRAS G12C inhibitor (GDC-6036) for about one month and cytotoxicity induced by AMG510 subsequently evaluated along with combination of AMG510 with BCA101 or Cetuximab. The cells were treated with titrating concentrations of AMG510 and a fixed concentration (50 μg/ml) of BCA101 or Cetuximab (nanomolar equivalent of 50 μg/mL BCA101) for 72 hours. Cytotoxicity of the hKRAS G12C inhibitor (AMG510) was evaluated using the CellTiter Glo® Luminescent Cell Viability Assay according to manufacturer's instructions (Promega) (as also described in Example 3). Statistical analysis (One way ANOVA) was performed using Graph pad v9. SW1463 colorectal cancer cells were treated with increasing concentrations of the hKRAS G12C inhibitor (AMG510) at increasing concentrations of up to 125 nM for 6 weeks and cytotoxicity induced by AMG510 subsequently evaluated. The cells were treated with titrating concentrations of AMG510 and a fixed concentration (50 μg/ml) of BCA101 for 120 hours. Cytotoxicity was evaluated using the CellTiter Glo® Luminescent Cell Viability Assay according to manufacturer's instructions (Promega) (as also described in Example 3). Statistical analysis (One way ANOVA) was performed using Graph pad v9.

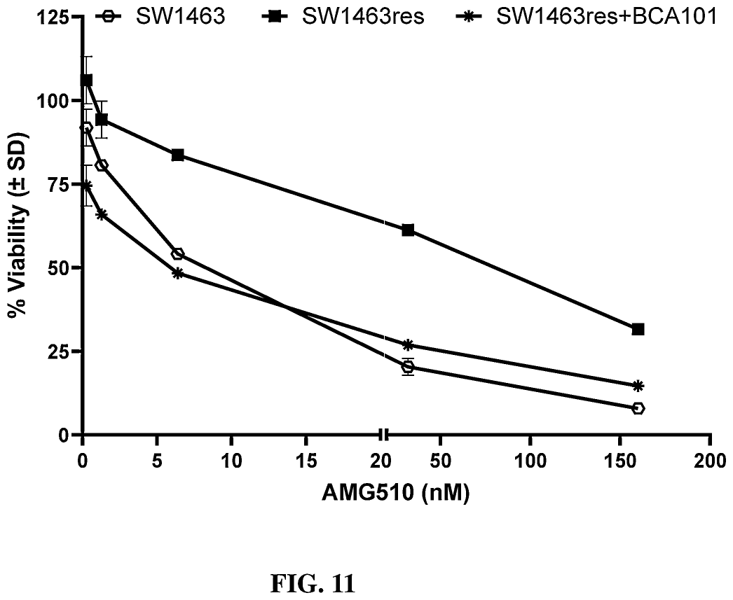

As shown in FIG. 10, KRAS-G12Ci resistant NCI-H1792 cells remain susceptible to AMG510+BCA101 combination treatments. FIG. 11, further shows KRAS-G12Ci resistant SW1463 cells remain susceptible to AMG510+BCA101 combination treatments.

### 6.11 Example 11. In Vivo Treatment of KRAS-G12Ci Resistant KRASG12C Mutated Lung with KRAS-G12Ci+BCA101 Combination

Tumor growth in KRAS-G12Ci resistant KRASG12C mutated lung treated with KRAS-G12Ci+BCA101 combination therapy was assessed.

Briefly, NCI-H358 cells (10×10⁶/mice) in 1:1 suspension with Matrigel were injected subcutaneously in the right flank region of athymic-Nude Foxn1^{Nu} mice (Hylasco, female, 8-week age, n=81) using 1 mL syringe. Post cell injections, at day 11, based on a mean tumor volume of 80 mm³, mice were randomized into 4 treatment groups (Placebo, AMG510 (5 mg/kg), BCA101 (5 mg/kg), and AMG510 (5 mg/kg)+BCA101 (5 mg/kg)) with 10 mice per group. Placebo (40% PEG300+10% DMSO+1% Tween 80+49% Sterile Saline) and BCA101 were administered intra-peritoneally weekly twice and AMG510 (GLXC-25372) was administered orally daily. Starting from the day of randomization (day 0), AMG510 was administered to respective groups for 7 days and then BCA101 treatment was started. In each group, individual mouse body weight, tumor length (L, mm), width (W, mm), and visible clinical signs were measured and recorded on day 0 and then weekly twice or at the day of tumor measurement till the end of the experiment. Tumor volumes (TV) were calculated using the following formula: TV (mm³)=(L×W²)/2. For each group, for different days, mean tumor volume+SEM (mm³) were plotted using GraphPad Prism (9.5.0). Statistical significance was calculated using GraphPad Prism by two-way ANOVA with Tuckey's multiple comparisons test comparing mean tumor volume for each treatment with every other treatment at day 31. *p≤0.05, **p≤0.01.

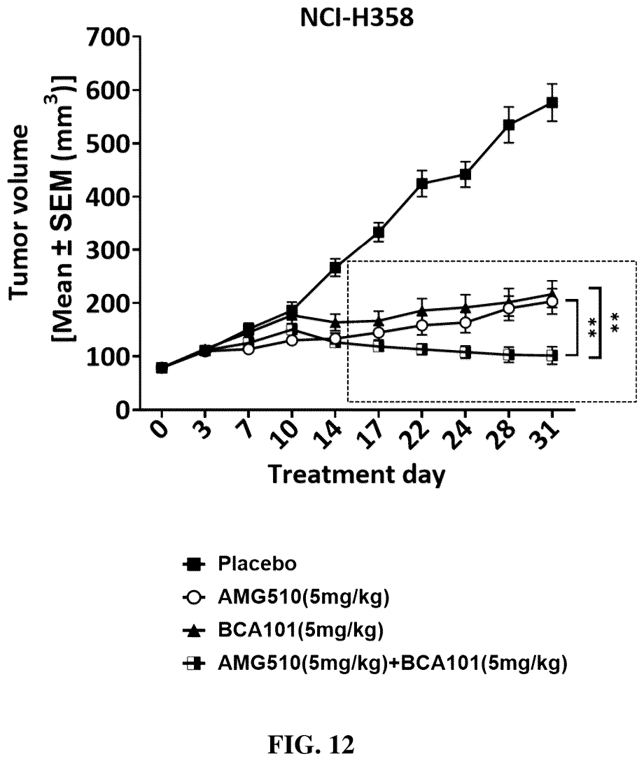

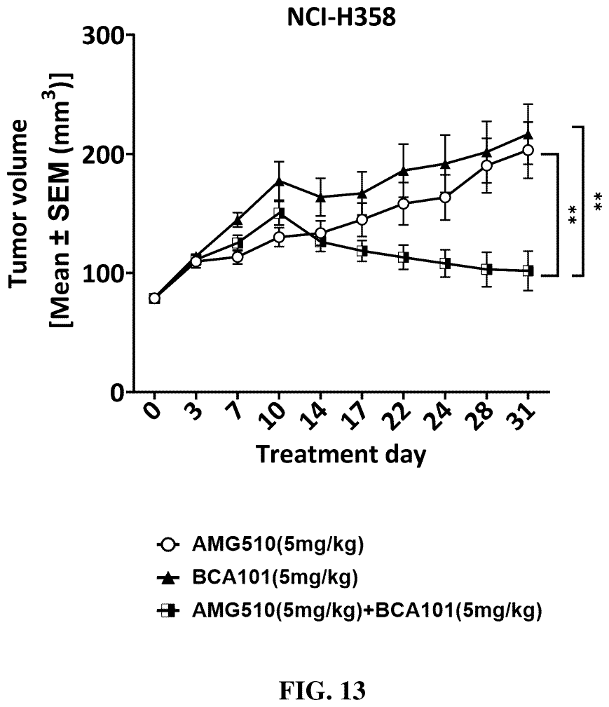

As shown in FIG. 12 and FIG. 13, treatment the BCA101+AMG510 showed a synergistic effect in inhibiting lung cancer tumor growth in vivo.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1.-108. (canceled)

109. A fusion protein comprising: (i) a first moiety that specifically binds epidermal growth factor receptor (EGFR) operably connected to (ii) a second moiety that specifically binds transforming growth factor β (TGFβ).

110. A method of treating cancer in a subject in need thereof, the method comprising: administering to the subject
(a) a kirsten rat sarcoma virus homolog (KRAS) inhibitor; in combination with
(b) the fusion protein of claim 109,
to thereby treat the cancer in the subject.

111. The method of claim 110, wherein the KRAS inhibitor comprises a KRAS inhibitor in Table 2.

112. The method of claim 110, wherein the KRAS inhibitor comprises sotorasib.

113. The method of claim 110, wherein the subject has been previously treated with the KRAS inhibitor.

114. The method of claim 110, wherein the cancer is a KRAS variant cancer.

115. The method of claim 110, wherein the KRAS variant comprises an amino acid modification (e.g., substitution) at amino acid position G12 or G13, numbering relative to the amino acid sequence of SEQ ID NO: 3.

116. The method of claim 110, wherein the first moiety comprises a variable heavy chain (VH) region that comprises three complementarity determining regions: VH CDR1, VH CDR2, and VH CDR3; and a variable light chain (VL) region that comprises three complementarity determining regions: VL CDR1, VL CDR2, and VL CDR3, wherein
(a) the amino acid sequence of VH CDR1 comprises the amino acid sequence SEQ ID NO: 39, or the amino acid sequence of SEQ ID NO: 39 comprising 1, 2, or 3 amino acid modifications;
(b) the amino acid sequence of VH CDR2 comprises the amino acid sequence SEQ ID NO: 40, or the amino acid sequence of SEQ ID NO: 40 comprising 1, 2, or 3 amino acid modifications;
(c) the amino acid sequence of VH CDR3 comprises the amino acid sequence SEQ ID NO: 41, or the amino acid sequence of SEQ ID NO: 41 comprising 1, 2, or 3 amino acid modifications; and
(d) the amino acid sequence of VL CDR1 comprises the amino acid sequence SEQ ID NO: 42, or the amino acid sequence of SEQ ID NO: 42 comprising 1, 2, or 3 amino acid modifications;
(e) the amino acid sequence of VL CDR2 comprises the amino acid sequence SEQ ID NO: 43, or the amino acid sequence of SEQ ID NO: 43 comprising 1, 2, or 3 amino acid modifications; and
(f) the amino acid sequence of VL CDR3 comprises the amino acid sequence SEQ ID NO: 44, or the amino acid sequence of SEQ ID NO: 44 comprising 1, 2, or 3 amino acid modifications.

117. The method of claim 110, wherein the amino acid sequence of the second moiety comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 72 or 73.

118. The method of claim 110, wherein the fusion protein comprises:
(a) a first polypeptide, wherein the amino acid sequence of the first polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125;
(b) a second polypeptide, wherein the amino acid sequence of the second polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 125;
(c) a third polypeptide, wherein the amino acid sequence of the third polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126; and
(d) a fourth polypeptide, wherein the amino acid sequence of the fourth polypeptide comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 126.

119. A method of treating a KRAS variant cancer in a subject in need thereof, the method comprising:
(a) receiving test results showing that the KRAS variant cancer is resistant to a KRAS inhibitor; and
(b) administering to the subject the KRAS inhibitor in combination with the fusion protein of claim 109,
to thereby treat the KRAS variant cancer in the subject.

120. A method of treating a KRAS variant cancer in a subject in need thereof, the method comprising:
(a) administering to the subject a KRAS inhibitor;
(b) receiving testing results showing that the KRAS variant cancer has developed resistance to the KRAS inhibitor; and
(c) administering to the subject the KRAS inhibitor in combination with the fusion protein of claim 109,
to thereby treat the KRAS variant cancer in the subject.

121. A method of treating a KRAS variant cancer in a subject in need thereof, the method comprising:
(a) determining that the KRAS variant cancer is resistant to a KRAS inhibitor; and
(b) administering to the subject a KRAS inhibitor in combination with the fusion protein of claim 109,
to thereby treat the KRAS variant cancer in the subject.

122. A method of delivering a KRAS inhibitor and a fusion protein to a subject in need thereof, the method comprising administering
(a) a KRAS inhibitor; in combination with
(b) the fusion protein of claim 109,
to thereby deliver the KRAS inhibitor and the fusion protein to a subject.

123. A method of inhibiting the KRAS pathway and the TGFβ pathway in a subject in need thereof, the method comprising administering
(a) a KRAS inhibitor; in combination with
(b) the fusion protein of claim 109,
to thereby inhibit the KRAS pathway and the TGFβ pathway in the subject.

124. A method of restoring sensitivity to a KRAS inhibitor in a subject in need thereof, the method comprising administering to the subject the fusion protein of claim 109, to thereby restore sensitivity to the KRAS inhibitor in the subject.

125. A method of suppressing or preventing resistance to a KRAS inhibitor in a subject in need thereof, the method comprising administering to the subject
(a) a KRAS inhibitor; in combination with
(b) the fusion protein of claim 109,
to thereby suppress or prevent resistance to the KRAS inhibitor in the subject.

126. A combination regimen comprising:
(a) a KRAS inhibitor; and
(b) the fusion protein of claim 109.

127. A pharmaceutical composition comprising
(a) a KRAS inhibitor; and
(b) the fusion protein of claim 109; and
(c) a pharmaceutically acceptable excipient.

128. A kit comprising a
(i) (a) a KRAS inhibitor or a pharmaceutical composition comprising the same; and (b) the fusion protein of claim 109 or a pharmaceutical composition comprising the same.
